(19) United States
(12) Patent Application Publication
(10) Pub. No.: US 2025/0353859 A1
(43) Pub. Date: Nov. 20, 2025

(54) COMPOUNDS AS CCR6 INHIBITORS

(71) Applicant: Hoffmann-La Roche Inc., Little Falls, NJ (US)

(72) Inventors: Lady Johanna BAUTISTA SARMIENTO, Oranjestad (AW); Joseph Maria Gerardus Barbara CALS, Oss (NL); Johanna Hendrika Angelique DE BROUWER, Tilburg (NL); Vera DE KIMPE, Oss (NL); Eugen DERETEY, Wijchen (NL); Alger Ayrton LAZO MORI, Rijswijk (NL); Daan Marten Jorrit MANDERS, Berghem (NL); Sander Bernardus NABUURS, Nijmegen (NL)

(73) Assignee: Hoffmann-La Roche Inc., Little Falls, NJ (US)

(21) Appl. No.: 19/291,334

(22) Filed: Aug. 5, 2025

Related U.S. Application Data
(63) Continuation of application No. PCT/EP2024/052678, filed on Feb. 5, 2024.

(30) Foreign Application Priority Data
Feb. 6, 2023 (EP) 23155010.4
Oct. 10, 2023 (EP) 23202551.0

(51) Int. Cl. C07D 487/04; A61K 31/275; A61K 31/404; A61K 31/437; A61K 31/4418; A61K 31/444; A61K 31/454; A61K 31/4545; A61K 31/4985; C07C 317/14; C07D 209/18; C07D 211/96; C07D 213/56; C07D 401/12; C07D 401/14; C07D 471/04

(52) U.S. Cl. CPC C07D 487/04; A61K 31/275; A61K 31/404; A61K 31/437; A61K 31/4418; A61K 31/444; A61K 31/454; A61K 31/4545; A61K 31/4985; C07C 317/14; C07D 209/18; C07D 211/96; C07D 213/56; C07D 401/12; C07D 401/14; C07D 471/04

(57) ABSTRACT

The present invention provides new derivatives having the general formula (I)

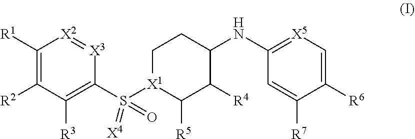

wherein R¹, R², R³, R⁴, R⁵, R⁶, R⁷, X¹, X², X³, X⁴, and X⁵ are as defined herein, compositions including the compounds, processes of manufacturing the compounds and methods of using the compounds.

### BACKGROUND

Immune surveillance, the migration of immune cells throughout the body is a tightly regulated process that is involved in many aspects of health and disease. Chemokines, and their corresponding receptors, play critical roles in these trafficking patterns, they are responsible for getting the right cells into the right tissues (Griffith, J. W., Sokol, C. L. & Luster, A. D. (2014). Chemokines and Chemokine Receptors: Positioning Cells for Host Defense and Immunity. Immunology, 32(1), 659-702 and Zlotnik, A. & Yoshie, O. (2012). The Chemokine Superfamily Revisited. Immunity, 36(5), 705-716).

Chemokines, or chemotactic cytokines, are a family of around 50 small signaling proteins secreted by a variety of cell populations (David, B. A. & Kubes, P. (2019). Exploring the complex role of chemokines and chemoattractants in vivo on leukocyte dynamics. Immunological Reviews, 289(1), 9-30 and Griffith, J. W., Sokol, C. L. & Luster, A. D. (2014). Chemokines and Chemokine Receptors: Positioning Cells for Host Defense and Immunity. Immunology, 32(1), 659-702). Chemokines are divided into four main subfamilies, called CC, CXC, CX3C and C, based on the location of the canonical cysteine residues in the N-terminal region. Chemokine secretion and diffusion create concentration gradients that direct the migration of cells expressing the corresponding receptors. Chemokine receptors are a family of around 20 seven transmembrane proteins differentially expressed on the surface of immune cells and can be divided into two main subfamilies, the first is called G protein-coupled chemokine receptors, which mediate immune cell trafficking, and the second is called atypical chemokine receptors, which seem to be chemokine scavengers that influence the chemokine gradients. They are also grouped into four subfamilies according to the subfamily of their major chemokine ligands. In some cases, one chemokine can signal through multiple receptors and, in many cases, one receptor can be stimulated by multiple chemokines. These promiscuous interactions make pharmacological intervention of signaling more complicated.

CCR6, also called CD196, is a chemokine receptor expressed on a variety of adaptive and innate immune cells including B cells, T cells, dendritic cells and neutrophils. For example, TH17 cells, which play a critical role in the pathogenesis of multiple autoimmune diseases, express CCR6 and this signal has been shown to recruit these cells into inflamed peripheral tissues (Esplugues, E., Huber, S., Gagliani, N., Hauser, A. E., Town, T., Wan, Y. Y., O'Connor, W., Rongvaux, A., Rooijen, N. V., Haberman, A. M., Iwakura, Y., Kuchroo, V. K., Kolls, J. K., Bluestone, J. A., Herold, K. C. & Flavell, R. A. (2011). Control of TH17 cells occurs in the Small Intestine. Nature, 475(7357), 514-518 and Singh, S. P., Zhang, H. H., Foley, J. F., Hedrick, M. N. & Farber, J. M. (2008). Human T Cells That Are Able to Produce IL-17 Express the Chemokine Receptor CCR6. The Journal of Immunology, 180(1), 214-221). The ligand for CCR6 is CCL20, also called macrophage inflammatory protein 3 alpha (MIP-3 alpha) and liver and activation-regulated chemokine (LARC). The CCR6/CCL20 pair is somewhat unique because they have only one binding partner and therefore form a pharmacologically selective receptor-ligand pair (Schutyser, E., Struyf, S. & Damme, J. V. (2003). The CC chemokine CCL20 and its receptor CCR6. Cytokine & Growth Factor Reviews, 14(5), 409-426).

CCL20 expression and secretion is increased in the presence of inflammatory stimuli. High levels of CCL20 can be found in the inflamed tissue associated with multiple inflammatory autoimmune diseases including psoriatic diseases, asthma, Crohn's disease, ulcerative colitis, rheumatoid arthritis, systemic lupus erythematosus and multiple sclerosis (Richmond, J. M., Strassner, J. P., Essien, K. I. & Harris, J. E. (2019). T-cell positioning by chemokines in autoimmune skin diseases. Immunological Reviews, 289(1), 186-204, Lee, A. Y. & Korner, H. (2014). CCR6 and CCL20: emerging players in the pathogenesis of rheumatoid arthritis. Immunology and Cell Biology, 92(4), 354-358, Raman, D., Sobolik-Delmaire, T. & Richmond, A. (2011). Chemokines in health and disease. Experimental Cell Research, 317(5), 575-589, Pene, J., Chevalier, S., Preisser, L., Venereau, E., Guilleux, M.-H., Ghannam, S., Moles, J.-P., Danger, Y., Ravon, E., Lesaux, S., Yssel, H. & Gascan, H. (2008). Chronically Inflamed Human Tissues Are Infiltrated by Highly Differentiated Th17 Lymphocytes. The Journal of Immunology, 180(11), 7423-7430 and Schutyser, E., Struyf, S. & Damme, J. V. (2003). The CC chemokine CCL20 and its receptor CCR6. Cytokine & Growth Factor Reviews, 14(5), 409-426).

Genetic linkage, clinical association and preclinical studies highlight a critical role for CCR6 in these inflammatory diseases (Hamburg, J. P. van & Tas, S. W. (2018). Molecular mechanisms underpinning T helper 17 cell heterogeneity and functions in rheumatoid arthritis. Journal of Autoimmunity, 87, 69-81 and Kurkó, J., Besenyei, T., Laki, J., Glant, T. T., Mikecz, K. & Szekanecz, Z. (2013). Genetics of Rheumatoid Arthritis—A Comprehensive Review. Clinical Reviews in Allergy & Immunology, 45(2), 170-179). For example, CCR6 gene variants have the highest risk association for Crohn's disease (CD) amongst the chemokine receptor family (Lee, A. Y. S., Eri, R., Lyons, A. B., Grimm, M. C. & Korner, H. (2013). CC Chemokine Ligand 20 and Its Cognate Receptor CCR6 in Mucosal T Cell Immunology and Inflammatory Bowel Disease: Odd Couple or Axis of Evil Frontiers in Immunology, 4, 194).

This high selectivity renders CCR6 an attractive drug target. Selective CCR6 inhibitors would only result in on-target pharmacology.

### BRIEF SUMMARY

A first object of the present invention is a compound of formula (I)

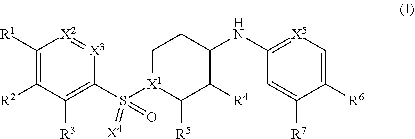

wherein
X¹ is CH or N;
X² is CH or N;
X³ is CH or N;
X⁴ is O or NH;
X⁵ is CH or N;
R¹ is aryl, heterocyclyl, or heteroaryl, wherein aryl, heterocyclyl, and heteroaryl are optionally substituted with one or more R^{1a};
R^{1a} is C₁₋₆alkyl, oxo, cyano, carbamoyl, C₁₋₆alkylcarbamoyl-, C₁₋₆alkoxyC₁₋₆alkyl-, C₃₋₆cycloalkyl, or heterocyclyl;
R² is hydrogen or halogen;
R³ is hydrogen or halogen;
R⁴ is hydrogen, halogen or C₁₋₆alkyl;
R⁵ is hydrogen, halogen or C₁₋₆alkyl;
R⁶ is —OR^{6a}, —SR^{6b}, or hydrogen;
R^{6a} is C₁₋₆haloalkyl;
R^{6b} is C₁₋₆haloalkyl;
R⁷ is —OR^{7a}, —SR^{7b}, or hydrogen;
R^{7a} is C₁₋₆haloalkyl;
R^{7b} is C₁₋₆haloalkyl,
provided that R⁶ and R⁷ must be different, and R⁶ or R⁷ is hydrogen, and pharmaceutically acceptable salts thereof.

A second object of the present invention is a process of preparation of a compound of formula (I) as defined above, or a pharmaceutically acceptable salt thereof, wherein X¹ is N, and X⁴ is O, comprising:
reacting compound of formula (II), wherein R⁵ and R⁴ are as defined above,

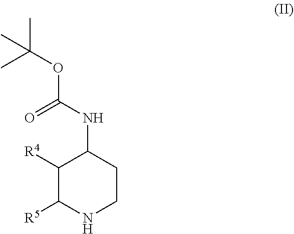

with compound of formula (III), wherein X², X³, R², and R³, are as defined above,

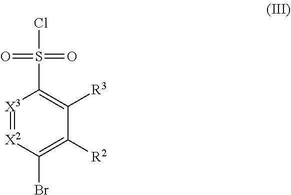

to form compound (IV), wherein R², R³, R⁴, R⁵, X², and X³ are as defined above,

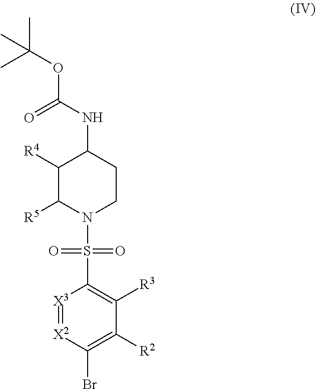

reacting said compound (IV) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

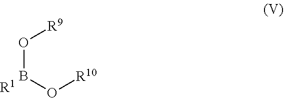

to form compound of formula (VI), wherein R¹, R², R³, R⁴, R⁵, X², and X³ are as defined above,

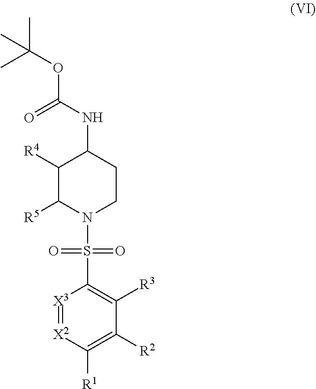

reacting said compound of formula (VI) with an acid, to form compound of formula (VII), wherein R¹, R², R³, R⁴, R⁵, X², and X³ are as defined above,

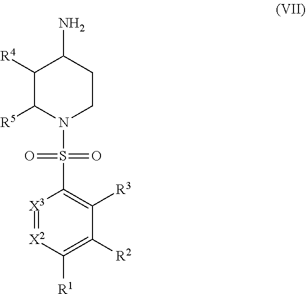

reacting said compound of formula (VII) with compound of formula (VIII), wherein R⁸ is a halogen, and R⁶, R⁷, and X⁵ are as defined above,

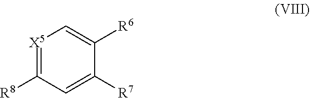

to form compound of formula (I).

A third object of the present invention a process of preparation of a compound of formula (I) as defined above, or a pharmaceutically acceptable salt thereof, wherein X¹ is N and X⁴ is O, comprising:
reacting compound of formula (IX), wherein R⁴ and R⁵ are as defined above,

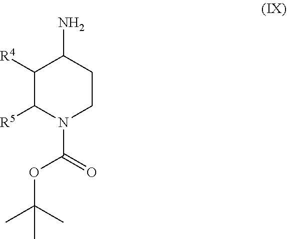

with compound of formula (VIII), wherein R⁸ is a halogen and R⁶, R⁷, and X⁵ are as defined above,

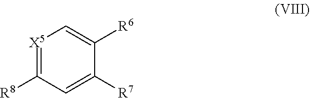

to form compound of formula (X), wherein wherein R⁴ R⁶, R⁷, X⁵, and R⁵ are as defined above,

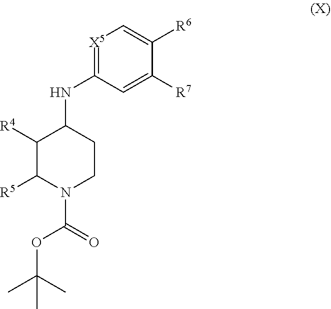

reacting said compound of formula (X) with acid to form compound of formula (XI), wherein R⁴ R⁶, R⁷, X⁵, and R⁵ are as defined above,

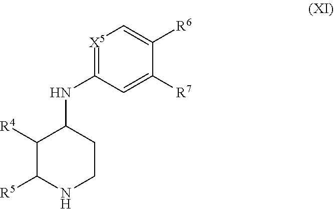

reacting said compound of formula (XI) with compound of formula (III), wherein X², X³, R², and R³, are as defined above,

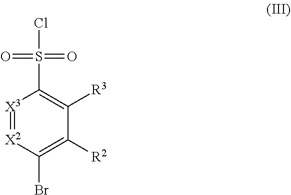

to form compound of formula (XII), wherein X², X³, R², R³, R⁴ R⁶, R⁷, X⁵, and R⁵ are as defined above,

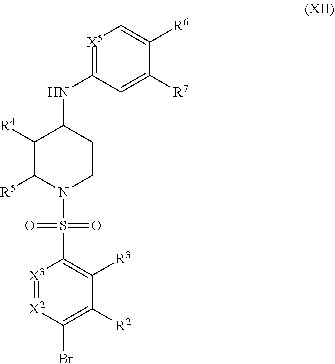

reacting said compound of formula (XII) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

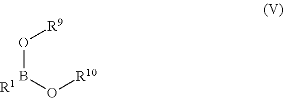

to form compound of formula (I).

A fourth object of the present invention a process of preparation of a compound of formula (I) as defined above, or a pharmaceutically acceptable salt thereof, wherein X¹ is N and X⁴ is O, comprising:
reacting compound of formula (XVIII), wherein R⁵ and R⁴ are as defined above,

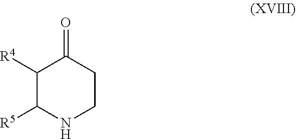

with compound of formula (XVII), wherein R¹, R², R³, X², and X³ are as defined above,

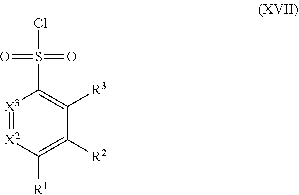

to form compound of formula (XX), wherein R¹, R², R³, R⁴, R⁵, X², and X³ are as defined above,

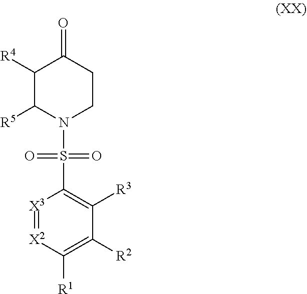

reacting said compound of formula (XX) with compound of formula (XVI), wherein X⁵, R⁶, and R⁷ are as defined above,

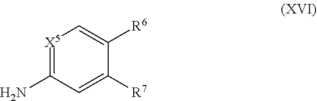

to form compound of formula (I);
or
reacting compound of formula (XVIII), wherein R⁵ and R⁴ are as defined above,

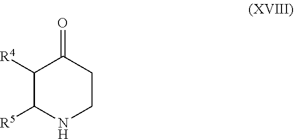

with compound of formula (III), wherein X², X³, R², and R³, are as defined above,

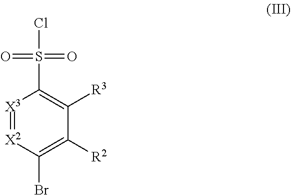

to form compound of formula (XIX), wherein R², R³, R⁴, R⁵, X², and X³ are as defined above,

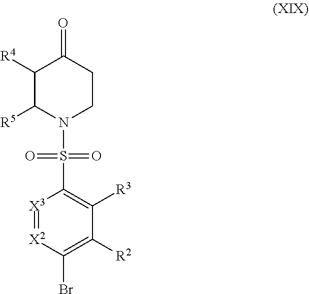

reacting said compound of formula (XIX) with said compound of formula (XVI), to form compound of formula (XII), wherein X², X³, R², R³, R⁴ R⁶, R⁷, X⁵, and R⁵ are as defined above,

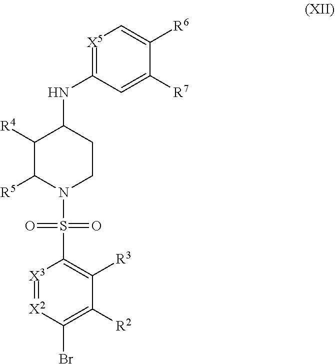

reacting said compound of formula (XII) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

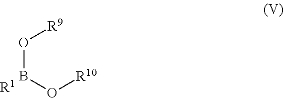

to form compound of formula (I).

A fifth object of the present invention a process of preparation of a compound of formula (I) as defined above, or a pharmaceutically acceptable salt thereof, wherein X¹ is C and X⁴ is O, comprising:
reacting compound of formula (XXI), wherein R⁴ and R⁵ are as defined above,

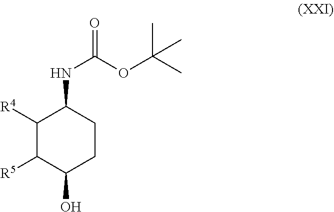

with masyl chloride, to form compound of formula (XXII), wherein R⁴ and R⁵ are as defined above,

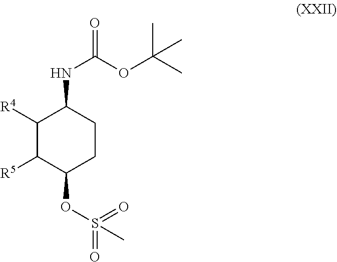

reacting said compound of formula (XXII) with compound of formula (XXIII), wherein R², R³, X², and X³ are as defined above,

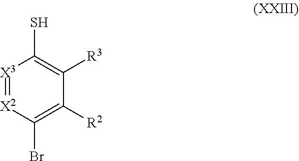

to form compound of formula (XXIV), wherein R², R³, R⁴, R⁵, X², and X³ are as defined above,

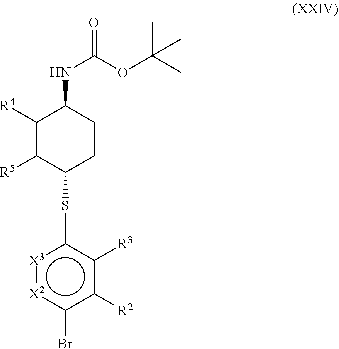

reacting said compound of formula (XXIV) with an oxidant, to form compound of formula (XXV), wherein R², R³, R⁴, R⁵, X², and X³ are as defined above,

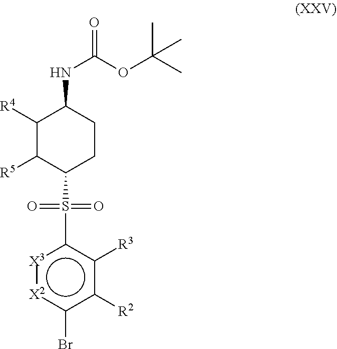

reacting said compound of formula (XXV) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

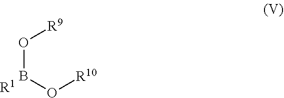

to form compound of formula (XXVI), wherein R¹, R², R³, R⁴, R⁵, X², and X³ are as defined above,

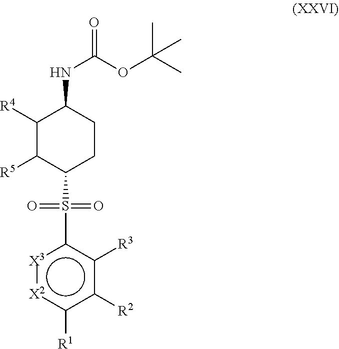

reacting said compound of formula (XXVI) with acid, to form compound of formula (XXVII), wherein R¹, R², R³, R⁴, R⁵, X², and X³ are as defined above,

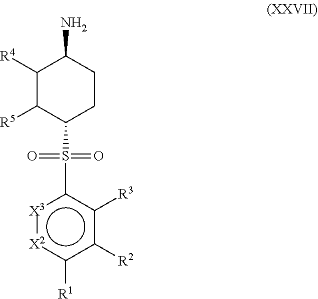

reacting said compound of formula (XXVII) with compound of formula (VIII), wherein R⁸ is a halogen and R⁶, R⁷, and X⁵ are as defined above,

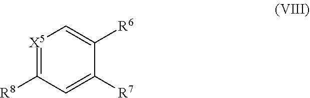

to form compound of formula (I).

A sixth object of the present invention a process of preparation of a compound of formula (I) as defined above, or a pharmaceutically acceptable salt thereof, wherein X¹ is C and X⁴ is O, comprising:
reacting compound of formula (XXV), wherein R², R³, R⁴, R⁵, X², and X³ are as defined above,

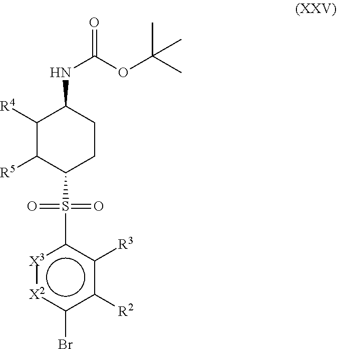

with acid to form compound of formula (XXVIII), wherein R², R³, R⁴, R⁵, X², and X³ are as defined above,

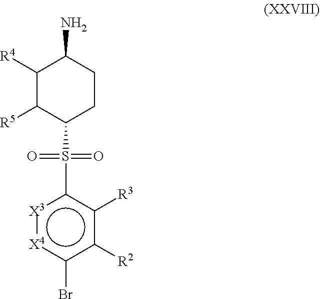

reacting said compound of formula (XXVIII) with compound of formula (VIII), wherein R⁸ is a halogen and R⁶, R⁷, and X⁵ are as defined above,

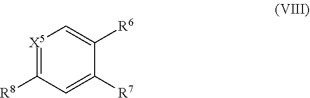

to form compound of formula (XXIX), wherein R², R³, R⁴, R⁵, R⁶, R⁷, X⁵, X², and X³ are as defined above,

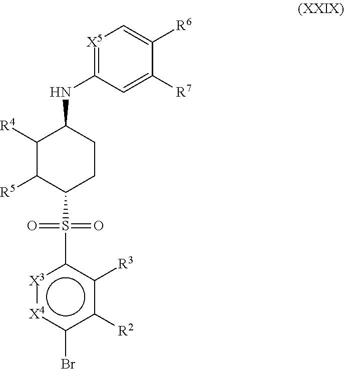

reacting said compound of formula (XXIX) with with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

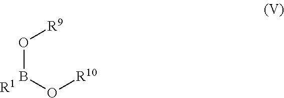

to form compound of formula (I).

A seventh object of the present invention a process of preparation of a compound of formula (I) as defined above, or a pharmaceutically acceptable salt thereof, wherein X¹ is C and X⁴ is O, comprising:
reacting compound of formula (XXXI), wherein R⁴ and R⁵ are as defined above,

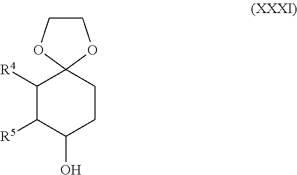

with mesyl chloride, to form compound of formula (XXXII), wherein R⁴ and R⁵ are as defined above,

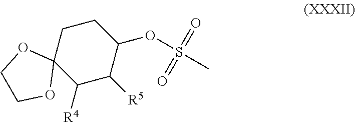

reacting said compound of formula (XXXII) with compound of formula (XXIII), wherein R², R³, X², and X³ are as defined above,

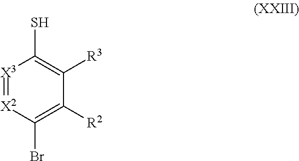

to form compound of formula (XXXIII), wherein R², R³, R⁴, R⁵, X², and X³ are as defined above,

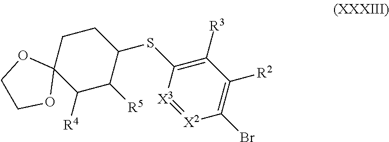

reacting said compound (XXXIII) with an oxidant, to form compound of formula (XXXIV), wherein R², R³, R⁴, R⁵, X², and X³ are as defined above,

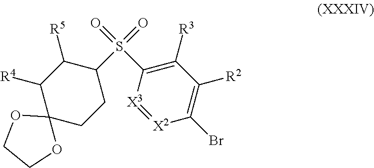

reacting said compound (XXXIV) with an acid, to form compound of formula (XXXV), wherein R², R³, R⁴, R⁵, X², and X³ are as defined above,

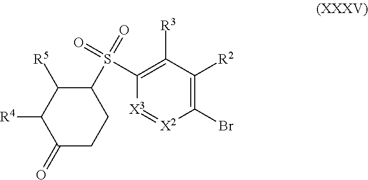

reacting said compound of formula (XXXV) with compound of formula (XVI), wherein X⁵, R⁶, and R⁷ are as defined above,

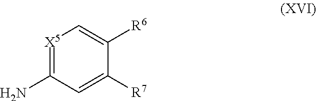

to form compound of formula (XXIX), wherein R², R³, R⁴, R⁵, R⁶, R⁷, X⁵, X², and X³ are as defined above,

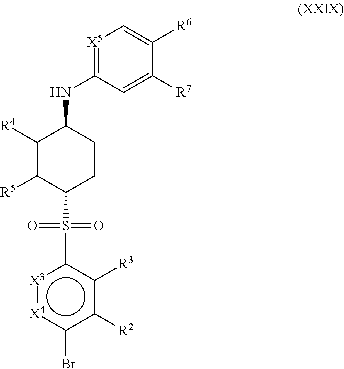

reacting said compound of formula (XXIX) with with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

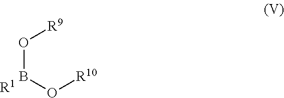

to form compound of formula (I).

A eighth object of the present invention a process of preparation of a compound of formula (I) as defined above, or a pharmaceutically acceptable salt thereof, wherein X¹ is C and X⁴ is N, comprising:
reacting compound of formula (XXXIII), wherein R², R³, R⁴, R⁵, X², and X³ are as defined above,

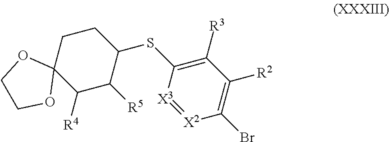

with an acid to form compound of formula (XXXVI), wherein R², R³, R⁴, R⁵, X², and X³ are as defined above,

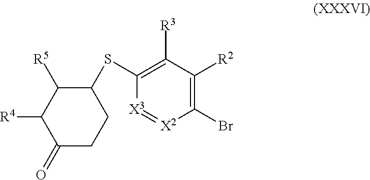

reacting said compound of formula (XXXVI) with compound of formula (XVI), wherein X⁵, R⁶, and R⁷ are as defined above,

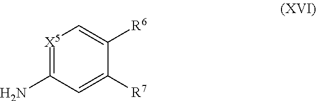

to form compound of formula (XXXVII), wherein R², R³, R⁴, R⁵, R⁶, R⁷, X⁵, X², and X³ are as defined above,

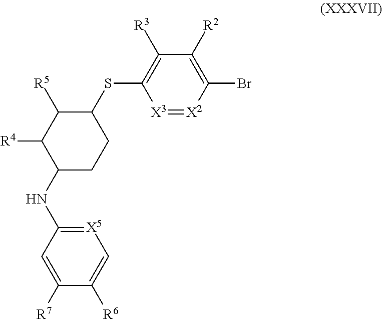

reacting said compound of formula (XXXVII) with ammonium carbamate and (diacetoxyiodo)benzene, to form compound of formula (XXXVIII), wherein R², R³, R⁴, R⁵, R⁶, R⁷, X⁵, X², and X³ are as defined above,

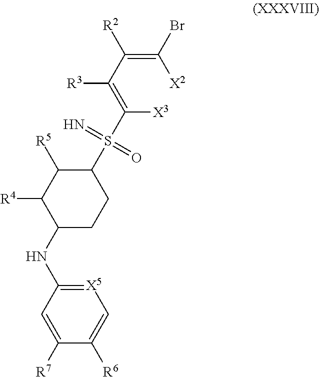

reacting said compound of formula (XXXVIII) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

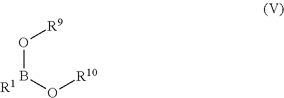

to form compound of formula (I).

A ninth object of the present invention a process of preparation of a compound of formula (I) as defined above, or a pharmaceutically acceptable salt thereof, wherein X¹ and X⁴ are N, comprising:
reacting compound of formula (XI), wherein R⁴ R⁶, R⁷, X⁵, and R⁵ are as defined above,

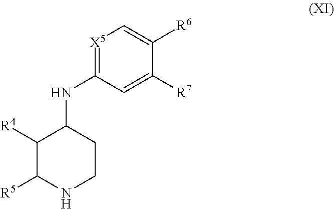

with compound of formula (XXIII), wherein R², R³, X², and X³ are as defined above,

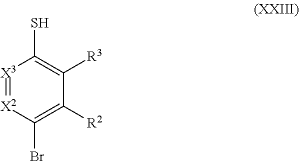

to form compound of formula (XL), wherein R², R³, X², X³, R⁴ R⁶, R⁷, X⁵, and R⁵ are as defined above,

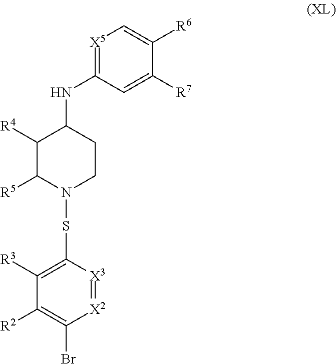

reacting said compound (XL) with ammonium carbamate and (diacetoxyiodo)benzene, to form compound of formula (XLI), wherein R², R³, X², X³, R⁴ R⁶, R⁷, X⁵, and R⁵ are as defined above,

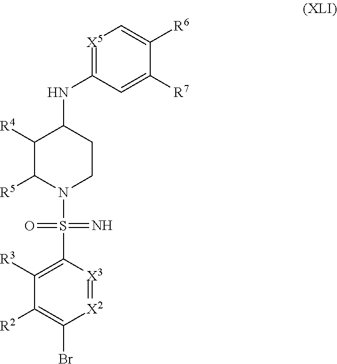

reacting said compound (XLI) with di-tert-butyl dicarbonate and a base, to form compound of formula (XLII), wherein R², R³, X², X³, R⁴ R⁶, R⁷, X⁵, and R⁵ are as defined above,

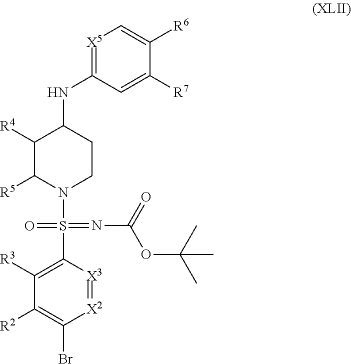

reacting said compound (XLII) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

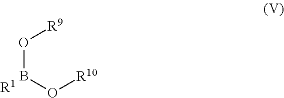

to form compound of formula (XLIII), wherein R¹, R², R³, X², X³, R⁴ R⁶, R⁷, X⁵, and R⁵ are as defined above,

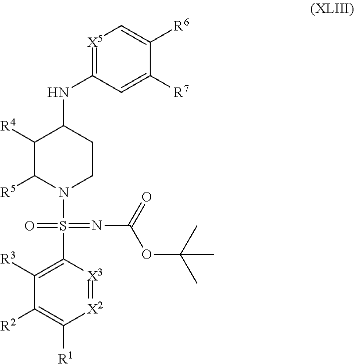

reacting said compound of formula (XLIII) with an acid to form compound of formula (I).

A tenth object of the present invention a process of preparation of a compound of formula (I) as defined above, or a pharmaceutically acceptable salt thereof, wherein X¹ is C and X⁴ is N, comprising:
reacting compound of formula (XXIV), wherein R², R³, R⁴, R⁵, X², and X³ are as defined above,

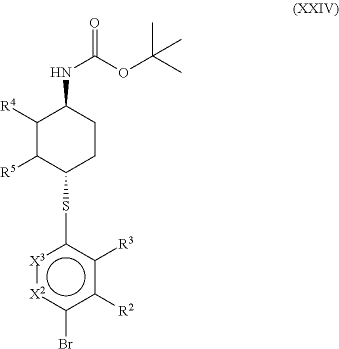

with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

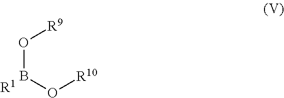

to form compound of formula (XLIV), wherein R¹, R², R³, R⁴, R⁵, X², and X³ are as defined above,

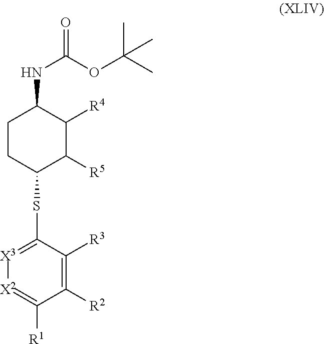

reacting said compound of formula (XLIV) with acid to form compound of formula (XLV), wherein R¹, R², R³, R⁴, R⁵, X², and X³ are as defined above,

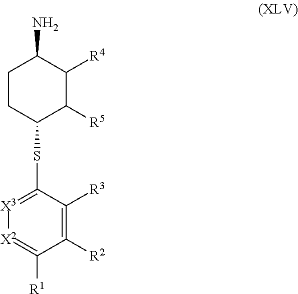

reacting said compound of formula (XLV) with compound of formula (VIII), wherein R⁸ is a halogen and R⁶, R⁷, and X⁵ are as defined above,

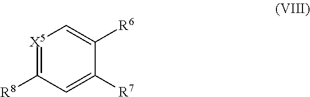

to form compound of formula (XLVI), wherein R¹, R², R³, R⁴, R⁵, R⁶, R⁷, X⁵, X², and X³ are as defined above,

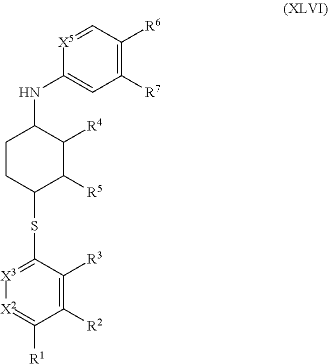

reacting said compound of formula (XLVI) with ammonium carbamate and (diacetoxyiodo)benzene to form compound of formula (I).

A eleventh object of the present invention is a pharmaceutical composition comprising a compound of formula (I) as described above, or a pharmaceutically acceptable salt thereof, and a pharmaceutically acceptable excipient.

A twelfth object of the current invention is a compound of formula (I), as described above, or a pharmaceutically acceptable salt thereof, for use in the treatment, prevention and/or delay of inflammatory autoimmune disease.

An thirteenth object of the current invention is a method for the treatment, prevention and/or delay of progression of inflammatory autoimmune disease, which method comprises administering a therapeutically effective amount of a compound of formula (I), or a pharmaceutically acceptable salt thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below.

All publications, patent applications, patents, and other references mentioned herein are incorporated by reference in their entirety.

The nomenclature used in this application is based on IUPAC systematic nomenclature, unless indicated otherwise.

### Definitions

“Acid” refers to a compound capable of giving proton of Broensted's definition, dissociating into proton and counter ion in water at 25° C. and giving a solution having neutral pH or below. Concrete examples of the acid are phosphoric acid (orthophosphoric acid), sulfuric acid, nitric acid, phosphinic acid, phosphonic acid, diphosphonic acid, hydrochloric acid, pyrophosphoric acid, metaphosphoric acid and nitrous 10 acid. These acids may be used in the form of metal salts, ammonium salts or the like; particularly acid means hydrochloric acid.

“C₁₋₆Alkoxy” refers to a C₁₋₆alkyl group, as previously defined, attached to the parent molecular moiety via an oxygen atom. Unless otherwise specified, the alkoxy group contains 1 to 6 carbon atoms (“C₁₋₆-alkoxy”). In some particular embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy.

“Amino”, alone or in combination with other groups, refers to NH₂.

“Aromatic” refers to the conventional idea of aromaticity as defined in the literature, in particular in IUPAC—Compendium of Chemical Terminology, 2^{nd} Edition, A. D. McNaught & A. Wilkinson (Eds). Blackwell Scientific Publications, Oxford (1997).

“Aryl” refers to a cyclic aromatic hydrocarbon moiety having a mono-, bi- or tricyclic aromatic ring of 5 to 14 carbon ring atoms (“C₅₋₁₄-aryl”). Bicyclic aryl ring systems include fused bicyclics having two fused five-membered aryl rings (denoted as 5-5), having a five-membered aryl ring and a fused six-membered aryl ring (denoted as 5-6 and as 6-5), and having two fused six-membered aryl rings (denoted as 6-6). The aryl group can be optionally substituted as defined herein. Examples of aryl moieties include, but are not limited to, phenyl, naphthyl, phenanthryl, fluorenyl, indenyl, pentalenyl, azulenyl, and the like. In particular aryl means phenyl.

“Base” refers to a chemical compound having at least one electronegative group capable of accepting an hydrogen ion.

“C₁₋₆alkyl” refers to a saturated linear (i.e. unbranched) or branched univalent hydrocarbon chain or combination thereof, having the number of carbon atoms designated (i.e., C₁₋₆ means one to ten carbon atoms). Particular C₁₋₆alkyl groups are those having 1 to 6 carbon atoms, having 2 to 6 carbon atoms (a “C₂₋₆alkyl”), or having 1 to 4 carbon atoms (a “C₁₋₄alkyl”). Examples of C₁₋₆alkyl group include, but are not limited to, groups such as methyl, ethyl, n-propyl, isopropyl, n-butyl, t-butyl, isobutyl, sec-butyl, homologs and isomers of, for example, n-pentyl, n-hexyl, and the like.

“Cyano”, alone or in combination with other groups, refers to CN (i.e. nitrile).

“C3-6Cycloalkyl” refers to a saturated or partially unsaturated carbocyclic moiety having mono-, bi-(including bridged bicyclic and cycloalkyl spiro moieties) or tricyclic rings and 3 to 10 carbon atoms i.e., (C₃-C₁₀)cycloalkyl) in the ring. The cycloalkyl moiety can optionally be substituted with one or more substituents. In particular aspects cycloalkyl contains from 3 to 8 carbon atoms (i.e., (C₃-C₈)cycloalkyl). In other particular aspects cycloalkyl contains from 3 to 6 carbon atoms (i.e., (C₃-C₆)cycloalkyl). Examples of cycloalkyl moieties include, but are not limited to, cyclopropyl, cyclobutyl, cyclopentyl, cyclohexyl, cycloheptyl, and partially unsaturated (cycloalkenyl) derivatives thereof (e.g. cyclopentenyl, cyclohexenyl, and cycloheptenyl), bicyclo[3.1.0]hexanyl, bicyclo[3.1.0]hexenyl, bicyclo[3.1.1]heptanyl, bicyclo[3.1.1]heptenyl and bicyclo[1.1.1]pentane. The cycloalkyl moiety can be attached in a “spiro-cycloalkyl” or “cycloalkyl spiro” fashion such as “spirocyclopropyl”.

“Halo” or “Halogen” means fluoro, chloro, bromo or iodo, particularly chloro or fluoro.

“Halo-C₁₋₆alkyl” refers to an C₁₋₆C₁₋₆alkyl, as defined above, substituted with one or more halogen atoms, particularly with one to three halogen atoms. More particularly halo-C₁₋₆alkyl is the chloro- and fluoro-C₁₋₆alkyl. In some particular embodiment halo-C₁₋₆alkyl refers to perhaloC₁₋₃C₁₋₆alkyl as defined herein. More particularly halo-C₁₋₆alkyl is trifluoromethyl, difluoromethyl or fluoromethyl. Most particularly halo-C₁₋₆alkyl is trifluoroalkyl (—CF₃).

“Haloalkoxy” refers to an alkoxy group in which at least one Halogen takes the place of each H in the hydrocarbon making up the C₁₋₆alkyl moiety of the alkoxy group. An example of a haloalkoxy group is difluoromethoxy (—OCHF₂), trifluoromethoxy (—OCF₃).

“Heteroaryl” refers to an aromatic heterocyclic mono-, bi- or tricyclic ring system of 5 to 14 ring atoms, preferably from 5 to 10 ring atoms, more preferably from 5 to 6 ring atoms, comprising 1, 2, 3 or 4 heteroatoms selected from N, O and S, the remaining ring atoms being carbon. In some aspects, monocyclic heteroaryl rings may be 5-6 membered. Bicyclic heteroaryl ring systems include fused bicyclics having two fused five-membered heteroaryl rings (denoted as 5-5), having a five-membered heteroaryl ring and a fused six-membered heteroaryl ring (denoted as 5-6 and 6-5), and having two fused six-membered heteroaryl rings (denoted as 6-6). The heteroaryl group can be optionally substituted as defined herein. Examples of heteroaryl moieties include indazolyl, indolyl, isoindolinyl, triazolopyridinyl, imidazopyridinyl, imidazopyrazinyl, indolinyl, pyridyl, triazolopyridazinyl, isoquinolinyl, pyridazinyl, triazolopyrazinyl, pyrrolotriazinyl, spirocyclopropaneindolinyl, pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, benzothiophenyl, indolyl, aza-indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, pyrrolopyridinyl, furopyridinyl, thienopyridinyl, pyrrolopyridazinyl, pyrrolopyrimidinyl, pyrrolopyrazinyl, thienopyridazinyl, thienopyrimidinyl, thienopyrazinyl, furopyridazinyl, furopyrimidinyl, and furopyrazinyl. Particular examples of heteroaryl are imidazopyridinyl, triazolopyridinyl, phenyl, indolyl, pyridyl, isoindolinyl, indazolyl; more particular indolyl, indazolyl, triazolopyridinyl, imidazopyridinyl.

“Heterocycle” or “heterocyclyl” refer to a 3, 4, 5, 6, 7, 8, 9, 10-membered monocyclic, 7, 8, 9 and 10-membered bicyclic (including bridged bicyclic and cycloalkyl spiro moieties) or 10, 11, 12, 13, 14 and 15-membered bicyclic heterocyclic moiety that is saturated or partially unsaturated, and has one or more (e.g., 1, 2, 3 or 4) heteroatoms selected from oxygen, nitrogen and sulfur in the ring with the remaining ring atoms being carbon. In some aspects, the heterocycle is a heterocycloalkyl. In particular aspects heterocycle or heterocyclyl refers to a 4, 5, 6 or 7-membered heterocycle. When used in reference to a ring atom of a heterocycle, a nitrogen or sulfur may also be in an oxidized form, and a nitrogen may be substituted with one or more (C₁-C₆)C₁₋₆alkyl or groups. The heterocycle can be attached to its pendant group at any heteroatom or carbon atom that results in a stable structure. Any of the heterocycle ring atoms can be optionally substituted with one or more substituents described herein.

The terms “inflammatory bowel disease” or “IBD” means several diseases associated with inflammation of the small intestine, large intestine (colon), rectum or anus (anal sphincter), and may particularly include ulcerative colitis and Crohn's disease, including proctitis in both cases. As used in the present context, the term “IBD” also includes gastrointestinal (GI) tract cancer, which is a likely result of Gl tract inflammation. As used in this specification, the terms “gastrointestinal tract” or “Gl tract” mean the small intestine, large intestine (colon), rectum or anus (anal sphincter).

“Moiety” and “Substituent” refer to an atom or group of chemically bonded atoms that is attached to another atom or molecule by one or more chemical bonds thereby forming part of a molecule.

When indicating the number of substituents, the term “one or more” refers to the range from one substituent to the highest possible number of substitution, i.e. replacement of one hydrogen up to replacement of all hydrogens by substituents, in particular wherein “one or more” refers to one, two or three, most particularly “one or more” refers to one or two.

“Obstructive pulmonary disease” refers to any disease that causes the airways of the lungs to become narrow or blocked so that a patient cannot exhale completely. Because of damage to the lungs or narrowing of the airways inside the lungs, exhaled air comes out more slowly than normal. At the end of a full exhalation, an abnormally high amount of air may still remain in the lungs. Examples of obstructive pulmonary diseases are asthma, bronchiectasis, bronchitis and chronic obstructive pulmonary disease (COPD).

“Optional” or “optionally” means that the subsequently described event or circumstance may but need not occur, and that the description includes instances where the event or circumstance occurs and instances in which it does not. For example, “aryl group optionally substituted with a C₁₋₆alkyl group” means that the C₁₋₆alkyl may but need not be present, and the description includes situations where the aryl group is substituted with a C₁₋₆alkyl group and situations where the aryl group is not substituted with the C₁₋₆alkyl group.

“Optionally substituted” means unsubstituted or substituted. Generally these substituents can be the same or different.

“Oxidant” refers to one or more suitable electron acceptors or electron sharers and may be an element, combination of elements, a compound, or combination of compounds including reducible compounds, and is a vapor, solid or liquid at the process conditions. An example of oxidant is mCPBA (meta-Chloroperoxybenzoic acid).

“Oxo”, alone or in combination with other groups, refers to ═O.

“Pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein.

More particularly pharmaceutically acceptable salts of compounds of formula (I) are the salts of hydrochloric acid, hydrobromic acid, sulfuric acid, phosphoric acid and methanesulfonic acid.

“Protecting group” refers to the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are Amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N.Y.

“Substituted” refers to the replacement of at least one of hydrogen atoms of a compound or moiety with another substituent or moiety. For example, the term “haloalkyl” refers to the fact that one or more hydrogen atoms of a C₁₋₆alkyl (as defined below) is replaced by one or more Halogen atoms (e.g., trifluoromethyl, difluoromethyl, fluoromethyl, chloromethyl, etc.). In one aspect, substituted as used herein can refer to replacement of at least one hydrogen atom of a compound or moiety described herein with Halogen or C₁₋₆alkyl.

“Therapeutically effective amount” refers to an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

### DETAILED DESCRIPTION

In one embodiment, the present invention relates a compound of formula (I),

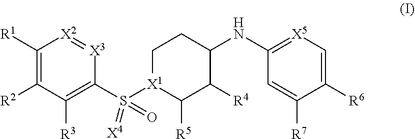

wherein
X¹ is CH or N;
X² is CH or N;
X³ is CH or N;
X⁴ is O or NH;
X⁵ is CH or N;
R¹ is aryl, heterocyclyl, or heteroaryl, wherein aryl, heterocyclyl, and heteroaryl are optionally substituted with one or more R^{1a};
R^{1a} is C₁₋₆alkyl, oxo, cyano, carbamoyl, C₁₋₆alkylcarbamoyl-, C₁₋₆alkoxyC₁₋₆alkyl-, C₃₋₆cycloalkyl, or heterocyclyl;
R² is hydrogen or halogen;
R³ is hydrogen or halogen;
R⁴ is hydrogen, halogen or C₁₋₆alkyl;
R⁵ is hydrogen, halogen or C₁₋₆alkyl;
R⁶ is —OR^{6a}, —SR^{6b}, or hydrogen;
R^{6a} is C₁₋₆haloalkyl;
R^{6b} is C₁₋₆haloalkyl;
R⁷ is —OR^{7a}, —SR^{7b}, or hydrogen;
R^{7a} is C₁₋₆haloalkyl;
R^{7b} is C₁₋₆haloalkyl,
provided that R⁶ and R⁷ must be different, and R⁶ or R⁷ is hydrogen,
and pharmaceutically acceptable salts thereof.

A particular embodiment of the present invention relates to a compound of formula (I) or (I′) as described herein, wherein
X² is CH;
X³ is CH;
R² is hydrogen;
R³ is hydrogen;
R⁴ is hydrogen;
R⁵ is hydrogen or C₁₋₆alkyl.

A particular embodiment of the present invention relates to a compound of formula (I) or (I′) as described herein, or a pharmaceutically acceptable salt thereof, wherein X¹ is CH.

A particular embodiment of the present invention relates to a compound of formula (I) or (I′) as described herein, or a pharmaceutically acceptable salt thereof, wherein X¹ is N.

A particular embodiment of the present invention relates to a compound of formula (I) or (I′) as described herein, or a pharmaceutically acceptable salt thereof, wherein X⁴ is O.

A particular embodiment of the present invention relates to a compound of formula (I) or (I′) as described herein, or a pharmaceutically acceptable salt thereof, wherein X⁴ is NH.

A particular embodiment of the present invention relates to a compound of formula (I) or (I′) as described herein, or a pharmaceutically acceptable salt thereof, wherein X⁵ is CH.

A particular embodiment of the present invention relates to a compound of formula (I) or (I′) as described herein, or a pharmaceutically acceptable salt thereof, wherein X⁵ is N.

A particular embodiment of the present invention relates to a compound of formula (I) or (I′) as described herein, or a pharmaceutically acceptable salt thereof, wherein R¹ is imidazopyridinyl, triazolopyridinyl, pyridyl, phenyl, indolyl, isoindolinyl, or indazolyl, wherein imidazopyridinyl, triazolopyridinyl, phenyl, indolyl, isoindolinyl, and indazolyl, are optionally substituted with one or more R¹.

A particular embodiment of the present invention relates to a compound of formula (I) or (I′) as described herein, or a pharmaceutically acceptable salt thereof, wherein R¹ is isopropylimidazopyridinyl, oxo-triazolopyridinyl, imidazopyridinyl, cyanophenyl, methylimidazopyridinyl, cyano-indolyl, oxoisoindolinyl, carbamoylphenyl, (methylcarbamoyl)phenyl, methyl-indazolyl, phenyl, cyano-indazolyl, cyanoimidazopyridinyl, methyl-triazolopyridinyl, (methoxymethyl)-triazolopyridinyl, or cyclopentyl-triazolopyridinyl.

A particular embodiment of the present invention relates to a compound of formula (I) or (I′) as described herein, or a pharmaceutically acceptable salt thereof, wherein R¹ is (3-isopropylimidazo[1,2-a]pyridin-6-yl), (3-oxo-2H-[1,2,4]triazolo[4,3-a]pyridin-6-yl), imidazo[1,2-a]pyridin-6-yl, (4-cyanophenyl), (3-methylimidazo[1,2-a]pyridin-6-yl), (3-cyano-1H-indol-5-yl), (3-isopropyl-8-methyl-imidazo[1,2-a]pyridin-6-yl), (2-carbamoyl-4-pyridyl), (3-oxoisoindolin-5-yl), (3-carbamoylphenyl), [3-(methylcarbamoyl)phenyl], (3-methyl-1H-indazol-5-yl), phenyl, (3-cyano-1H-indazol-5-yl), (3-cyanoimidazo[1,2-a]pyridin-6-yl), (2-methyl-[1,2,4]triazolo[1,5-a]pyridin-6-yl), (2-methyl-[1,2,4]triazolo[1,5-a]pyridin-7-yl), [3-(methoxymethyl)-[1,2,4]triazolo[4,3-a]pyridin-6-yl], or (3-cyclopentyl-[1,2,4]triazolo[4,3-a]pyridin-6-yl).

A particular embodiment of the present invention relates to a compound of formula (I) or (I′) as described herein, or a pharmaceutically acceptable salt thereof, wherein R¹ is cyano-indazolyl, cyano-indolyl, methyl-indazolyl, oxo-triazolopyridinyl, isopropylimidazopyridinyl, or cyclopentyl-triazolopyridinyl.

A particular embodiment of the present invention relates to a compound of formula (I) or (I′) as described herein, or a pharmaceutically acceptable salt thereof, wherein R¹ is cyano-indazolyl, cyano-indolyl, methyl-indazolyl, oxo-triazolopyridinyl, isopropylimidazopyridinyl, or cyclopentyl-triazolopyridinyl.

A particular embodiment of the present invention relates to a compound of formula (I) or (I′) as described herein, or a pharmaceutically acceptable salt thereof, wherein R¹ is indazolyl, indolyl, indazolyl, imidazopyridinyl, or triazolopyridinyl, wherein indazolyl, indolyl, indazolyl, imidazopyridinyl, and triazolopyridinyl, are optionally substituted with one or more R^{1a}.

A particular embodiment of the present invention relates to a compound of formula (I) or (I′) as described herein, or a pharmaceutically acceptable salt thereof, wherein R¹ is indazolyl, indolyl, indazolyl, imidazopyridinyl, or triazolopyridinyl, wherein indazolyl, indolyl, indazolyl, imidazopyridinyl, and triazolopyridinyl, are optionally substituted with one or more cyano, methyl, oxo, isopropyl, or cyclopentyl.

A particular embodiment of the present invention relates to a compound of formula (I) or (I′) as described herein, or a pharmaceutically acceptable salt thereof, wherein R¹ is (3-cyano-1H-indol-5-yl), (3-methyl-1H-indazol-5-yl), (3-cyano-1H-indazol-5-yl), (3-oxo-2H-[1,2,4]triazolo[4,3-a]pyridin-6-yl), (3-isopropylimidazo[1,2-a]pyridin-6-yl), or cyclopentyl-triazolopyridinyl.

A particular embodiment of the present invention relates to a compound of formula (I) or (I′) as described herein, or a pharmaceutically acceptable salt thereof, wherein R^{1a} is isopropyl, methyl, oxo, cyano, carbamoyl, methylcarbamoyl, or methoxymethyl.

A particular embodiment of the present invention relates to a compound of formula (I) or (I′) as described herein, or a pharmaceutically acceptable salt thereof, wherein R^{1a} is cyano, methyl, oxo, isopropyl, or cyclopentyl.

A particular embodiment of the present invention relates to a compound of formula (I) or (I′) as described herein, or a pharmaceutically acceptable salt thereof, wherein R⁶ is —SCF₃, —OCF₃, —OCHF₂, or hydrogen.

A particular embodiment of the present invention relates to a compound of formula (I) or (I′) as described herein, or a pharmaceutically acceptable salt thereof, wherein R⁶ is —SCF₃, or —OCF₃.

A particular embodiment of the present invention relates to a compound of formula (I) or (I′) as described herein, or a pharmaceutically acceptable salt thereof, wherein R^{6a} is —CF₃, or —CHF₂.

A particular embodiment of the present invention relates to a compound of formula (I) or (I′) as described herein, or a pharmaceutically acceptable salt thereof, wherein R^{6b} is —CF₃.

A particular embodiment of the present invention relates to a compound of formula (I) or (I′) as described herein, or a pharmaceutically acceptable salt thereof, wherein R⁷ is —OCF₃, —SCF₃, —OCHF₂, or hydrogen.

A particular embodiment of the present invention relates to a compound of formula (I) or (I′) as described herein, or a pharmaceutically acceptable salt thereof, wherein R⁷ is hydrogen.

A particular embodiment of the present invention relates to a compound of formula (I) or (I′) as described herein, or a pharmaceutically acceptable salt thereof, wherein R^{7a} is —CF₃, or —CHF₂.

A particular embodiment of the present invention relates to a compound of formula (I) or (I′) as described herein, or a pharmaceutically acceptable salt thereof, wherein R^{7b} is —CF₃.

A particular embodiment of the present invention relates to a compound of formula (I) or (I′) as described herein, or a pharmaceutically acceptable salt thereof, wherein R⁵ is hydrogen or methyl.

A particular embodiment of the present invention relates to a compound of formula (I) or (I′) as described herein, or a pharmaceutically acceptable salt thereof, wherein R¹ is indazolyl, indolyl, indazolyl, imidazopyridinyl, or triazolopyridinyl, wherein indazolyl, indolyl, indazolyl, imidazopyridinyl, and triazolopyridinyl, are optionally substituted with one or more cyano, methyl, oxo, isopropyl, or cyclopentyl, R⁶ is —SCF₃, or —OCF₃; and R², R³, R⁴, R⁵, R⁷, X¹, X², X³, X⁴, and X⁵ are as defined above.

A particular embodiment of the present invention relates to a compound of formula (I) as described herein, or a pharmaceutically acceptable salt thereof, wherein the compound is of formula (I′)

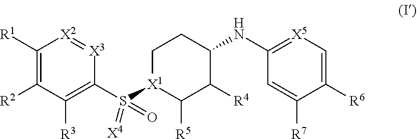

or a pharmaceutically acceptable salt thereof and R¹, R², R³, R⁴, R⁵, R⁶, R⁷, X², X³, X⁴, and X⁵ are as defined above and X¹ is CH.

A particular embodiment of the present invention relates to a compound of formula (I) as described herein, or a pharmaceutically acceptable salt thereof, wherein the compound is of formula (I″)

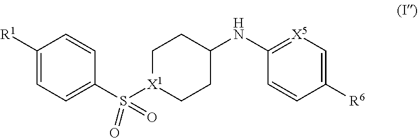

or a pharmaceutically acceptable salt thereof and R¹, X¹, and X⁵ are as defined above, and R⁶ is —SCF₃, or —OCF₃.

A particular embodiment of the present invention relaters to a compound of Formula (I), (I′), (I″), wherein:
R¹ is indazolyl, indolyl, indazolyl, imidazopyridinyl, or triazolopyridinyl, wherein indazolyl, indolyl, indazolyl, imidazopyridinyl, and triazolopyridinyl, are optionally substituted with one or more cyano, methyl, oxo, isopropyl, or cyclopentyl;
X¹ is CH or N;
X⁵ is CH or N; and
R⁶ is —SCF₃, or —OCF₃.

A particular embodiment of the present invention relaters to a compound of Formula (I), (I′), (I″), wherein:
R¹ is imidazopyridinyl, triazolopyridinyl, phenyl, indolyl, isoindolinyl, or indazolyl, wherein imidazopyridinyl, triazolopyridinyl, phenyl, indolyl, isoindolinyl, and indazolyl, are optionally substituted with one or more R^{1a};
X¹ is CH or N;
X⁵ is CH or N;
R⁷ is —OCF₃, —SCF₃, —OCHF₂, or hydrogen; and
R⁶ is —SCF₃, —OCF₃, —OCHF₂, or hydrogen;
provided that R⁶ and R⁷ must be different, and R⁶ or R⁷ is hydrogen.

A particular embodiment of the present invention relates to a compound of formula (I) as described herein, or a pharmaceutically acceptable salt thereof, selected from:
{4-[3-(propan-2-yl)imidazo[1,2-a]pyridin-6-yl]phenyl}[trans-4-({4-[(trifluoromethyl)sulfanyl]phenyl}Amino)cyclohexyl](imino)-λ⁶-sulfanone;
6-(4-{[trans-4-({4-[(trifluoromethyl)sulfanyl]phenyl}Amino)cyclohexyl]sulfonimidoyl}phenyl)-2H,3H-[1,2,4]triazolo[4,3-a]pyridin-3-one;
(4-{imidazo[1,2-a]pyridin-6-yl}phenyl)[trans-4-({4-[(trifluoromethyl)sulfanyl]phenyl}Amino)cyclohexyl](imino)-λ⁶-sulfanone;
(4-{imidazo[1,2-a]pyridin-6-yl}phenyl)[trans-4-{[5-(trifluoromethoxy)pyridin-2-yl]Amino}cyclohexyl](imino)-λ⁶-sulfanone;
4′-{[trans-4-{[4-(trifluoromethoxy)phenyl]Amino}cyclohexyl]sulfonyl}-[1,1′-biphenyl]-4-carbonitrile;
N-[trans-4-(4-{3-methylimidazo[1,2-a]pyridin-6-yl}benzenesulfonyl)cyclohexyl]-4-(trifluoromethoxy)aniline;
N-[trans-4-(4-{3-methylimidazo[1,2-a]pyridin-6-yl}benzenesulfonyl)cyclohexyl]-5-(trifluoromethoxy)pyridin-2-amine;
5-(4-{[trans-4-({4-[(trifluoromethyl)sulfanyl]phenyl}Amino)cyclohexyl]sulfonyl}phenyl)-1H-indole-3-carbonitrile;
N-[trans-4-(4-{imidazo[1,2-a]pyridin-6-yl}benzenesulfonyl)cyclohexyl]-4-(trifluoromethoxy)aniline;
6-(4-{[trans-4-({4-[(trifluoromethyl)sulfanyl]phenyl}Amino)cyclohexyl]sulfonyl}phenyl)-2H,3H-[1,2,4]triazolo[4,3-a]pyridin-3-one;
N-[trans-4-(4-{imidazo[1,2-a]pyridin-6-yl}benzenesulfonyl)cyclohexyl]-4-[(trifluoromethyl)sulfanyl]aniline;
N-[trans-4-{4-[8-methyl-3-(propan-2-yl)imidazo[1,2-a]pyridin-6-yl]benzenesulfonyl}cyclohexyl]-4-[(trifluoromethyl)sulfanyl]aniline;
4-(4-{[trans-4-({4-[(trifluoromethyl)sulfanyl]phenyl}Amino)cyclohexyl]sulfonyl}phenyl)pyridine-2-carboxamide;
4′-[(4-{[5-(trifluoromethoxy)pyridin-2-yl]Amino}piperidin-1-yl)sulfonyl]-[1,1′-biphenyl]-4-carbonitrile;
6-{4-[(4-{[5-(trifluoromethoxy)pyridin-2-yl]Amino}piperidin-1-yl)sulfonyl]phenyl}-2,3-dihydro-1H-isoindol-1-one;
4′-[(4-{[5-(trifluoromethoxy)pyridin-2-yl]Amino}piperidin-1-yl)sulfonyl]-[1,1′-biphenyl]-3-carboxamide;
5-{4-[(4-{[5-(trifluoromethoxy)pyridin-2-yl]Amino}piperidin-1-yl)sulfonyl]phenyl}-1H-indole-3-carbonitrile;
4′-[(4-{[4-(trifluoromethoxy)phenyl]Amino}piperidin-1-yl)sulfonyl]-[1,1′-biphenyl]-4-carbonitrile;
N-methyl-4′-[(4-{[4-(trifluoromethoxy)phenyl]Amino}piperidin-1-yl)sulfonyl]-[1,1′-biphenyl]-3-carboxamide;
6-{4-[(4-{[4-(trifluoromethoxy)phenyl]Amino}piperidin-1-yl)sulfonyl]phenyl}-2,3-dihydro-1H-isoindol-1-one;
4′-[(4-{[4-(trifluoromethoxy)phenyl]Amino}piperidin-1-yl)sulfonyl]-[1,1′-biphenyl]-3-carboxamide;
1-[4-(3-methyl-1H-indazol-5-yl)benzenesulfonyl]-N-[4-(trifluoromethoxy)phenyl]piperidin-4-amine;
5-{4-[(4-{[4-(trifluoromethoxy)phenyl]Amino}piperidin-1-yl)sulfonyl]phenyl}-1H-indole-3-carbonitrile;
5-{4-[(4-{[4-(trifluoromethoxy)phenyl]Amino}piperidin-1-yl)sulfonyl]phenyl}-1H-indazole-3-carbonitrile;
1-{[1,1′-biphenyl]-4-sulfonyl}-N-[4-(trifluoromethoxy)phenyl]piperidin-4-amine;
1-{[1,1′-biphenyl]-4-sulfonyl}-2-methyl-N-[4-(trifluoromethoxy)phenyl]piperidin-4-amine;
4′-[(2-methyl-4-{[4-(trifluoromethoxy)phenyl]Amino}piperidin-1-yl)sulfonyl]-[1,1′-biphenyl]-4-carbonitrile;
6-{4-[(2-methyl-4-{[4-(trifluoromethoxy)phenyl]Amino}piperidin-1-yl)sulfonyl]phenyl}-2,3-dihydro-1H-isoindol-1-one;
N-methyl-4′-[(2-methyl-4-{[4-(trifluoromethoxy)phenyl]Amino}piperidin-1-yl)sulfonyl]-[1,1′-biphenyl]-3-carboxamide;
N-[4-(difluoromethoxy)phenyl]-1-[4-(3-methyl-1H-indazol-5-yl)benzenesulfonyl]piperidin-4-amine;
6-{4-[(4-{[4-(difluoromethoxy)phenyl]Amino}piperidin-1-yl)sulfonyl]phenyl}-2,3-dihydro-1H-isoindol-1-one;
5-{4-[(4-{[4-(difluoromethoxy)phenyl]Amino}piperidin-1-yl)sulfonyl]phenyl}-1H-indole-3-carbonitrile;
1-{[1,1′-biphenyl]-4-sulfonyl}-N-[4-(difluoromethoxy)phenyl]piperidin-4-amine;
1-(4-{imidazo[1,2-a]pyridin-6-yl}benzenesulfonyl)-N-[4-(trifluoromethoxy)phenyl]piperidin-4-amine;
1-(4-{3-methylimidazo[1,2-a]pyridin-6-yl}benzenesulfonyl)-N-[4-(trifluoromethoxy)phenyl]piperidin-4-amine;
6-{4-[(4-{[4-(trifluoromethoxy)phenyl]Amino}piperidin-1-yl)sulfonyl]phenyl}imidazo[1,2-a]pyridine-3-carbonitrile;
6-{4-[(4-{[4-(trifluoromethoxy)phenyl]Amino}piperidin-1-yl)sulfonyl]phenyl}-2H,3H-[1,2,4]triazolo[4,3-a]pyridin-3-one;
1-(4-{2-methyl-[1,2,4]triazolo[1,5-a]pyridin-6-yl}benzenesulfonyl)-N-[4-(trifluoromethoxy)phenyl]piperidin-4-amine;
1-(4-{2-methyl-[1,2,4]triazolo[1,5-a]pyridin-7-yl}benzenesulfonyl)-N-[4-(trifluoromethoxy)phenyl]piperidin-4-amine;
1-{4-[3-(propan-2-yl)imidazo[1,2-a]pyridin-6-yl]benzenesulfonyl}-N-[4-(trifluoromethoxy)phenyl]piperidin-4-amine;
N-(1-{4-[3-(propan-2-yl)imidazo[1,2-a]pyridin-6-yl]benzenesulfonyl}piperidin-4-yl)-5-(trifluoromethoxy)pyridin-2-amine;
N-[1-(4-{imidazo[1,2-a]pyridin-6-yl}benzenesulfonyl)piperidin-4-yl]-5-(trifluoromethoxy)pyridin-2-amine;
1-{4-[3-(propan-2-yl)imidazo[1,2-a]pyridin-6-yl]benzenesulfonyl}-N-{4-[(trifluoromethyl)sulfanyl]phenyl}piperidin-4-amine;
1-{4-[3-(methoxymethyl)-[1,2,4]triazolo[4,3-a]pyridin-6-yl]benzenesulfonyl}-N-[4-(trifluoromethoxy)phenyl]piperidin-4-amine;
6-{4-[(4-{[5-(trifluoromethoxy)pyridin-2-yl]Amino}piperidin-1-yl)sulfonyl]phenyl}-2H,3H-[1,2,4]triazolo[4,3-a]pyridin-3-one;
1-(4-{imidazo[1,2-a]pyridin-6-yl}benzenesulfonyl)-N-{4-[(trifluoromethyl)sulfanyl]phenyl}piperidin-4-amine;
1-(4-{3-cyclopentyl-[1,2,4]triazolo[4,3-a]pyridin-6-yl}benzenesulfonyl)-N-[4-(trifluoromethoxy)phenyl]piperidin-4-amine;
1-{4-[8-methyl-3-(propan-2-yl)imidazo[1,2-a]pyridin-6-yl]benzenesulfonyl}-N-{4-[(trifluoromethyl)sulfanyl]phenyl}piperidin-4-amine;
4-(4-{[4-({4-[(trifluoromethyl)sulfanyl]phenyl}Amino)piperidin-1-yl]sulfonyl}phenyl)pyridine-2-carboxamide;
4′-{[trans-4-{[3-(trifluoromethoxy)phenyl]Amino}cyclohexyl]sulfonyl}-[1,1′-biphenyl]-4-carbonitrile;
N-[trans-4-(4-{imidazo[1,2-a]pyridin-6-yl}benzenesulfonyl)cyclohexyl]-3-[(trifluoromethyl)sulfanyl]aniline;
N-[trans-4-{4-[3-(propan-2-yl)imidazo[1,2-a]pyridin-6-yl]benzenesulfonyl}cyclohexyl]-3-[(trifluoromethyl)sulfanyl]aniline;
6-{4-[(4-{[3-(trifluoromethoxy)phenyl]Amino}piperidin-1-yl)sulfonyl]phenyl}-2,3-dihydro-1H-isoindol-1-one
4′-[(4-{[3-(difluoromethoxy)phenyl]Amino}piperidin-1-yl)sulfonyl]-[1,1′-biphenyl]-4-carbonitrile
N-(1-{4-[8-methyl-3-(propan-2-yl)imidazo[1,2-a]pyridin-6-yl]benzenesulfonyl}piperidin-4-yl)-5-(trifluoromethoxy)pyridin-2-amine
N-(1-{4-[3-(propan-2-yl)-[1,2,4]triazolo[4,3-a]pyridin-6-yl]benzenesulfonyl}piperidin-4-yl)-5-(trifluoromethoxy)pyridin-2-amine
N-(1-{4-[8-methyl-3-(propan-2-yl)-[1,2,4]triazolo[4,3-a]pyridin-6-yl]benzenesulfonyl}piperidin-4-yl)-5-(trifluoromethoxy)pyridin-2-amine
4-{4-[(4-{[5-(trifluoromethoxy)pyridin-2-yl]amino}piperidin-1-yl)sulfonyl]phenyl}pyridine-2-carboxamide
N-(1-{4-[3-(propan-2-yl)-[1,2,4]triazolo[4,3-a]pyrazin-6-yl]benzenesulfonyl}piperidin-4-yl)-5-(trifluoromethoxy)pyridin-2-amine
N-[1-(4-{3-methyl-[1,2,4]triazolo[4,3-a]pyridin-6-yl}benzenesulfonyl)piperidin-4-yl]-5-(trifluoromethoxy)pyridin-2-amine
N-(1-{4-[8-methyl-3-(propan-2-yl)imidazo[1,2-a]pyridin-6-yl]benzenesulfonyl}piperidin-4-yl)-5-[(trifluoromethyl)sulfanyl]pyridin-2-amine
N-[1-(4-{imidazo[1,2-a]pyridin-6-yl}benzenesulfonyl)piperidin-4-yl]-5-[(trifluoromethyl)sulfanyl]pyridin-2-amine
4-(4-{[4-({5-[(trifluoromethyl)sulfanyl]pyridin-2-yl}amino)piperidin-1-yl]sulfonyl}phenyl)pyridine-2-carboxamide
N-(1-{4-[3-(propan-2-yl)imidazo[1,2-a]pyridin-6-yl]benzenesulfonyl}piperidin-4-yl)-5-[(trifluoromethyl)sulfanyl]pyridin-2-amine
N-(1-{4-[3-(propan-2-yl)-[1,2,4]triazolo[4,3-a]pyridin-6-yl]benzenesulfonyl}piperidin-4-yl)-5-[(trifluoromethyl)sulfanyl]pyridin-2-amine
N-(1-{4-[3-(propan-2-yl)-[1,2,4]triazolo[4,3-a]pyrazin-6-yl]benzenesulfonyl}piperidin-4-yl)-5-[(trifluoromethyl)sulfanyl]pyridin-2-amine
N-[1-(4-{3-methyl-[1,2,4]triazolo[4,3-a]pyridin-6-yl}benzenesulfonyl)piperidin-4-yl]-5-[(trifluoromethyl)sulfanyl]pyridin-2-amine
N-(1-{4-[8-methyl-3-(propan-2-yl)-[1,2,4]triazolo[4,3-a]pyridin-6-yl]benzenesulfonyl}piperidin-4-yl)-5-[(trifluoromethyl)sulfanyl]pyridin-2-amine
N-(1-{4-[8-methyl-3-(propan-2-yl)imidazo[1,2-a]pyridin-6-yl]benzenesulfonyl}piperidin-4-yl)-4-(trifluoromethoxy)pyridin-2-amine
N-(1-{4-[3-(propan-2-yl)-[1,2,4]triazolo[4,3-a]pyridin-6-yl]benzenesulfonyl}piperidin-4-yl)-4-(trifluoromethoxy)pyridin-2-amine
N-(1-{4-[8-methyl-3-(propan-2-yl)-[1,2,4]triazolo[4,3-a]pyridin-6-yl]benzenesulfonyl}piperidin-4-yl)-4-(trifluoromethoxy)pyridin-2-amine
N-[trans-4-(4-{3-methyl-1H-pyrazolo[3,4-b]pyridin-5-yl}benzenesulfonyl)cyclohexyl]-4-[(trifluoromethyl)sulfanyl]aniline
N-[trans-4-(4-{imidazo[1,2-a]pyridin-6-yl}benzenesulfonyl)cyclohexyl]-5-(trifluoromethoxy)pyridin-2-amine or
6-(4-{[4-({5-[(trifluoromethyl)sulfanyl]pyridin-2-yl}amino)piperidin-1-yl]sulfonyl}phenyl)-2H,3H-[1,2,4]triazolo[4,3-a]pyridin-3-one.

A particular embodiment of the present invention relates to a compound of formula (I) as described herein, or a pharmaceutically acceptable salt thereof, selected from:
{4-[3-(propan-2-yl)imidazo[1,2-a]pyridin-6-yl]phenyl}[trans-4-({4-[(trifluoromethyl)sulfanyl]phenyl}Amino)cyclohexyl](imino)-λ⁶-sulfanone;
6-(4-{[trans-4-({4-[(trifluoromethyl)sulfanyl]phenyl}Amino)cyclohexyl]sulfonimidoyl}phenyl)-2H,3H-[1,2,4]triazolo[4,3-a]pyridin-3-one;
(4-{imidazo[1,2-a]pyridin-6-yl}phenyl)[trans-4-({4-[(trifluoromethyl)sulfanyl]phenyl}Amino)cyclohexyl](imino)-λ⁶-sulfanone;
(4-{imidazo[1,2-a]pyridin-6-yl}phenyl)[trans-4-{[5-(trifluoromethoxy)pyridin-2-yl]Amino}cyclohexyl](imino)-λ⁶-sulfanone;
4′-{[trans-4-{[4-(trifluoromethoxy)phenyl]Amino}cyclohexyl]sulfonyl}-[1,1′-biphenyl]-4-carbonitrile;
N-[trans-4-(4-{3-methylimidazo[1,2-a]pyridin-6-yl}benzenesulfonyl)cyclohexyl]-4-(trifluoromethoxy)aniline;
N-[trans-4-(4-{3-methylimidazo[1,2-a]pyridin-6-yl}benzenesulfonyl)cyclohexyl]-5-(trifluoromethoxy)pyridin-2-amine;
5-(4-{[trans-4-({4-[(trifluoromethyl)sulfanyl]phenyl}Amino)cyclohexyl]sulfonyl}phenyl)-1H-indole-3-carbonitrile;
N-[trans-4-(4-{imidazo[1,2-a]pyridin-6-yl}benzenesulfonyl)cyclohexyl]-4-(trifluoromethoxy)aniline;
6-(4-{[trans-4-({4-[(trifluoromethyl)sulfanyl]phenyl}Amino)cyclohexyl]sulfonyl}phenyl)-2H,3H-[1,2,4]triazolo[4,3-a]pyridin-3-one;
N-[trans-4-(4-{imidazo[1,2-a]pyridin-6-yl}benzenesulfonyl)cyclohexyl]-4-[(trifluoromethyl)sulfanyl]aniline;
N-[trans-4-{4-[8-methyl-3-(propan-2-yl)imidazo[1,2-a]pyridin-6-yl]benzenesulfonyl}cyclohexyl]-4-[(trifluoromethyl)sulfanyl]aniline;
4-(4-{[trans-4-({4-[(trifluoromethyl)sulfanyl]phenyl}Amino)cyclohexyl]sulfonyl}phenyl)pyridine-2-carboxamide
4′-[(4-{[5-(trifluoromethoxy)pyridin-2-yl]Amino}piperidin-1-yl)sulfonyl]-[1,1′-biphenyl]-4-carbonitrile;
6-{4-[(4-{[5-(trifluoromethoxy)pyridin-2-yl]Amino}piperidin-1-yl)sulfonyl]phenyl}-2,3-dihydro-1H-isoindol-1-one;
4′-[(4-{[5-(trifluoromethoxy)pyridin-2-yl]Amino}piperidin-1-yl)sulfonyl]-[1,1′-biphenyl]-3-carboxamide;
5-{4-[(4-{[5-(trifluoromethoxy)pyridin-2-yl]Amino}piperidin-1-yl)sulfonyl]phenyl}-1H-indole-3-carbonitrile;
4′-[(4-{[4-(trifluoromethoxy)phenyl]Amino}piperidin-1-yl)sulfonyl]-[1,1′-biphenyl]-4-carbonitrile;
N-methyl-4′-[(4-{[4-(trifluoromethoxy)phenyl]Amino}piperidin-1-yl)sulfonyl]-[1,1′-biphenyl]-3-carboxamide;
6-{4-[(4-{[4-(trifluoromethoxy)phenyl]Amino}piperidin-1-yl)sulfonyl]phenyl}-2,3-dihydro-1H-isoindol-1-one;
4′-[(4-{[4-(trifluoromethoxy)phenyl]Amino}piperidin-1-yl)sulfonyl]-[1,1′-biphenyl]-3-carboxamide;
1-[4-(3-methyl-1H-indazol-5-yl)benzenesulfonyl]-N-[4-(trifluoromethoxy)phenyl]piperidin-4-amine;
5-{4-[(4-{[4-(trifluoromethoxy)phenyl]Amino}piperidin-1-yl)sulfonyl]phenyl}-1H-indole-3-carbonitrile;
5-{4-[(4-{[4-(trifluoromethoxy)phenyl]Amino}piperidin-1-yl)sulfonyl]phenyl}-1H-indazole-3-carbonitrile;
1-{[1,1′-biphenyl]-4-sulfonyl}-N-[4-(trifluoromethoxy)phenyl]piperidin-4-amine;
1-{[1,1′-biphenyl]-4-sulfonyl}-2-methyl-N-[4-(trifluoromethoxy)phenyl]piperidin-4-amine;
4′-[(2-methyl-4-{[4-(trifluoromethoxy)phenyl]Amino}piperidin-1-yl)sulfonyl]-[1,1′-biphenyl]-4-carbonitrile;
6-{4-[(2-methyl-4-{[4-(trifluoromethoxy)phenyl]Amino}piperidin-1-yl)sulfonyl]phenyl}-2,3-dihydro-1H-isoindol-1-one;
N-methyl-4′-[(2-methyl-4-{[4-(trifluoromethoxy)phenyl]Amino}piperidin-1-yl)sulfonyl]-[1,1′-biphenyl]-3-carboxamide;
N-[4-(difluoromethoxy)phenyl]-1-[4-(3-methyl-1H-indazol-5-yl)benzenesulfonyl]piperidin-4-amine;
6-{4-[(4-{[4-(difluoromethoxy)phenyl]Amino}piperidin-1-yl)sulfonyl]phenyl}-2,3-dihydro-1H-isoindol-1-one;
5-{4-[(4-{[4-(difluoromethoxy)phenyl]Amino}piperidin-1-yl)sulfonyl]phenyl}-1H-indole-3-carbonitrile;
1-{[1,1′-biphenyl]-4-sulfonyl}-N-[4-(difluoromethoxy)phenyl]piperidin-4-amine;
1-(4-{imidazo[1,2-a]pyridin-6-yl}benzenesulfonyl)-N-[4-(trifluoromethoxy)phenyl]piperidin-4-amine;
1-(4-{3-methylimidazo[1,2-a]pyridin-6-yl}benzenesulfonyl)-N-[4-(trifluoromethoxy)phenyl]piperidin-4-amine;
6-{4-[(4-{[4-(trifluoromethoxy)phenyl]Amino}piperidin-1-yl)sulfonyl]phenyl}imidazo[1,2-a]pyridine-3-carbonitrile;
6-{4-[(4-{[4-(trifluoromethoxy)phenyl]Amino}piperidin-1-yl)sulfonyl]phenyl}-2H,3H-[1,2,4]triazolo[4,3-a]pyridin-3-one;
1-(4-{2-methyl-[1,2,4]triazolo[1,5-a]pyridin-6-yl}benzenesulfonyl)-N-[4-(trifluoromethoxy)phenyl]piperidin-4-amine;
1-(4-{2-methyl-[1,2,4]triazolo[1,5-a]pyridin-7-yl}benzenesulfonyl)-N-[4-(trifluoromethoxy)phenyl]piperidin-4-amine;
1-{4-[3-(propan-2-yl)imidazo[1,2-a]pyridin-6-yl]benzenesulfonyl}-N-[4-(trifluoromethoxy)phenyl]piperidin-4-amine;
N-(1-{4-[3-(propan-2-yl)imidazo[1,2-a]pyridin-6-yl]benzenesulfonyl}piperidin-4-yl)-5-(trifluoromethoxy)pyridin-2-amine;
N-[1-(4-{imidazo[1,2-a]pyridin-6-yl}benzenesulfonyl)piperidin-4-yl]-5-(trifluoromethoxy)pyridin-2-amine;
1-{4-[3-(propan-2-yl)imidazo[1,2-a]pyridin-6-yl]benzenesulfonyl}-N-{4-[(trifluoromethyl)sulfanyl]phenyl}piperidin-4-amine;
1-{4-[3-(methoxymethyl)-[1,2,4]triazolo[4,3-a]pyridin-6-yl]benzenesulfonyl}-N-[4-(trifluoromethoxy)phenyl]piperidin-4-amine;
6-{4-[(4-{[5-(trifluoromethoxy)pyridin-2-yl]Amino}piperidin-1-yl)sulfonyl]phenyl}-2H,3H-[1,2,4]triazolo[4,3-a]pyridin-3-one;
1-(4-{imidazo[1,2-a]pyridin-6-yl}benzenesulfonyl)-N-{4-[(trifluoromethyl)sulfanyl]phenyl}piperidin-4-amine;
1-(4-{3-cyclopentyl-[1,2,4]triazolo[4,3-a]pyridin-6-yl}benzenesulfonyl)-N-[4-(trifluoromethoxy)phenyl]piperidin-4-amine;
1-{4-[8-methyl-3-(propan-2-yl)imidazo[1,2-a]pyridin-6-yl]benzenesulfonyl}-N-{4-[(trifluoromethyl)sulfanyl]phenyl}piperidin-4-amine;
4-(4-{[4-({4-[(trifluoromethyl)sulfanyl]phenyl}Amino)piperidin-1-yl]sulfonyl}phenyl)pyridine-2-carboxamide;
4′-{[trans-4-{[3-(trifluoromethoxy)phenyl]Amino}cyclohexyl]sulfonyl}-[1,1′-biphenyl]-4-carbonitrile;
N-[trans-4-(4-{imidazo[1,2-a]pyridin-6-yl}benzenesulfonyl)cyclohexyl]-3-[(trifluoromethyl)sulfanyl]aniline;
N-[trans-4-{4-[3-(propan-2-yl)imidazo[1,2-a]pyridin-6-yl]benzenesulfonyl}cyclohexyl]-3-[(trifluoromethyl)sulfanyl]aniline;
6-{4-[(4-{[3-(trifluoromethoxy)phenyl]Amino}piperidin-1-yl)sulfonyl]phenyl}-2,3-dihydro-1H-isoindol-1-one or 4′-[(4-{[3-(difluoromethoxy)phenyl]Amino}piperidin-1-yl)sulfonyl]-[1,1′-biphenyl]-4-carbonitrile.
A particular embodiment of the present invention relates to a compound of formula (I) as described herein, or a pharmaceutically acceptable salt thereof, selected from:
5-[4-[4-[4-(trifluoromethylsulfanyl)anilino]cyclohexyl]sulfonylphenyl]-1H-indole-3-carbonitrile;
5-[4-[[4-[[5-(trifluoromethoxy)-2-pyridyl]Amino]-1-piperidyl]sulfonyl]phenyl]-1H-indole-3-carbonitrile;
1-[4-(3-methyl-1H-indazol-5-yl)phenyl]sulfonyl-N-[4-(trifluoromethoxy)phenyl]piperidin-4-amine;
5-[4-[[4-[4-(trifluoromethoxy)anilino]-1-piperidyl]sulfonyl]phenyl]-1H-indole-3-carbonitrile;
5-[4-[[4-[4-(trifluoromethoxy)anilino]-1-piperidyl]sulfonyl]phenyl]-1H-indazole-3-carbonitrile;
6-[4-[[4-[4-(trifluoromethoxy)anilino]-1-piperidyl]sulfonyl]phenyl]-2H-[1,2,4]triazolo[4,3-a]pyridin-3-one;
1-[4-(3-isopropylimidazo[1,2-a]pyridin-6-yl)phenyl]sulfonyl-N-[4-(trifluoromethoxy)phenyl]piperidin-4-amine;
1-[4-(3-cyclopentyl-[1,2,4]triazolo[4,3-a]pyridin-6-yl)phenyl]sulfonyl-N-[4-(trifluoromethoxy)phenyl]piperidin-4-amine
N-[1-(4-{3-methyl-[1,2,4]triazolo[4,3-a]pyridin-6-yl}benzenesulfonyl)piperidin-4-yl]-5-[(trifluoromethyl)sulfanyl]pyridin-2-amine or
6-(4-{[4-({5-[(trifluoromethyl)sulfanyl]pyridin-2-yl}amino)piperidin-1-yl]sulfonyl}phenyl)-2H,3H-[1,2,4]triazolo[4,3-a]pyridin-3-one.

A particular embodiment of the present invention relates to a compound of formula (I) as described herein, or a pharmaceutically acceptable salt thereof, selected from:
5-[4-[4-[4-(trifluoromethylsulfanyl)anilino]cyclohexyl]sulfonylphenyl]-1H-indole-3-carbonitrile;
5-[4-[[4-[[5-(trifluoromethoxy)-2-pyridyl]Amino]-1-piperidyl]sulfonyl]phenyl]-1H-indole-3-carbonitrile;
1-[4-(3-methyl-1H-indazol-5-yl)phenyl]sulfonyl-N-[4-(trifluoromethoxy)phenyl]piperidin-4-amine;
5-[4-[[4-[4-(trifluoromethoxy)anilino]-1-piperidyl]sulfonyl]phenyl]-1H-indole-3-carbonitrile;
5-[4-[[4-[4-(trifluoromethoxy)anilino]-1-piperidyl]sulfonyl]phenyl]-1H-indazole-3-carbonitrile;
6-[4-[[4-[4-(trifluoromethoxy)anilino]-1-piperidyl]sulfonyl]phenyl]-2H-[1,2,4]triazolo[4,3-a]pyridin-3-one;
1-[4-(3-isopropylimidazo[1,2-a]pyridin-6-yl)phenyl]sulfonyl-N-[4-(trifluoromethoxy)phenyl]piperidin-4-amine;
   or
1-[4-(3-cyclopentyl-[1,2,4]triazolo[4,3-a]pyridin-6-yl)phenyl]sulfonyl-N-[4-(trifluoromethoxy)phenyl]piperidin-4-amine.

Further objects of the present invention are all forms of optically pure enantiomers, racemates or diastereomeric mixtures for compounds of formula (I) or (I′).

### Process of Manufacturing

Processes for the manufacture of compounds of formula (I), or pharmaceutically acceptable salt thereof, as described herein are also an object of the invention.

The present invention provides a process of preparation of a compound as described herein, or a pharmaceutically acceptable salt thereof, wherein X¹ is N, and X⁴ is O, comprising:
reacting compound of formula (II), wherein R⁵ and R⁴ are as defined above,

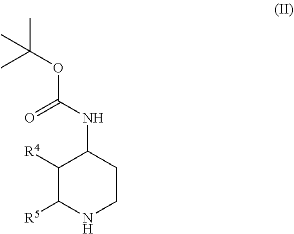

with compound of formula (III), wherein X², X³, R², and R³, are as defined above,

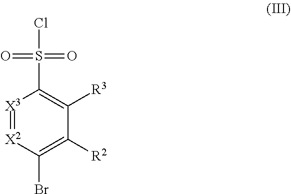

to form compound (IV), wherein R², R³, R⁴, R⁵, X², and X³ are as defined above,

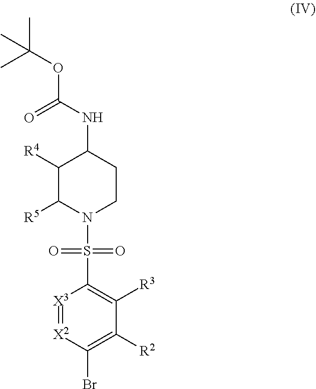

reacting said compound (IV) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

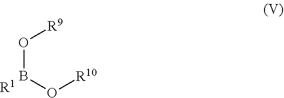

to form compound of formula (VI), wherein R¹, R², R³, R⁴, R⁵, X², and X³ are as defined above,

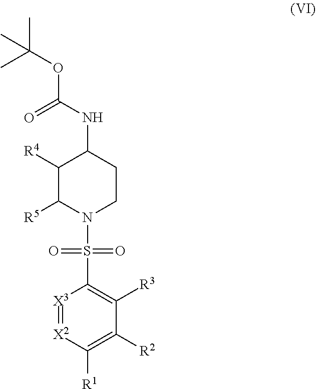

reacting said compound of formula (VI) with an acid, to form compound of formula (VII), wherein R¹, R², R³, R⁴, R⁵, X², and X³ are as defined above,

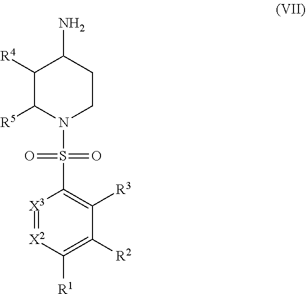

reacting said compound of formula (VII) with compound of formula (VIII), wherein R⁸ is a halogen and R⁶, R⁷, and X⁵ are as defined above,

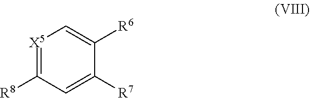

to form compound of formula (I).

The present invention provides a process of preparation of a compound as described herein, or a pharmaceutically acceptable salt thereof, wherein X¹ is N, and X⁴ is O, comprising:
reacting compound of formula (II), wherein R⁵ and R⁴ are as defined above,

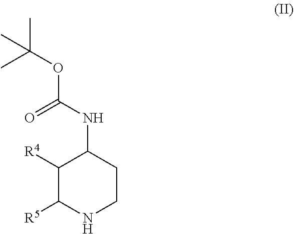

with compound of formula (III), wherein X², X³, R², and R³, are as defined above,

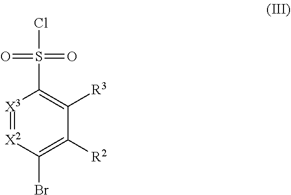

to form compound (IV), wherein R², R³, R⁴, R⁵, X², and X³ are as defined above,

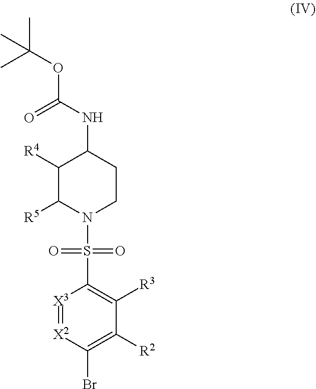

reacting said compound (IV) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

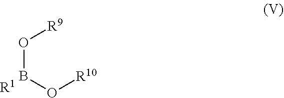

to form compound of formula (VI), wherein R¹, R², R³, R⁴, R⁵, X², and X³ are as defined above,

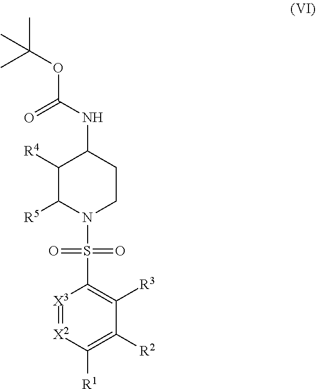

reacting said compound of formula (VI) with an acid, to form compound of formula (VII), wherein R¹, R², R³, R⁴, R⁵, X², and X³ are as defined above,

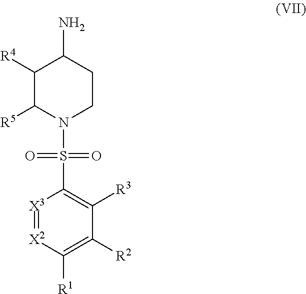

reacting said compound of formula (VII) with compound of formula (VIII), wherein R⁸ is a halogen and R⁶, R⁷, and X⁵ are as defined above,

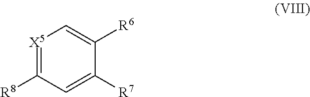

to form compound of formula (I).

The present invention provides a process of preparation of a compound as described herein, or a pharmaceutically acceptable salt thereof, wherein X¹ is N and X⁴ is O, comprising:
reacting compound of formula (IX), wherein R⁴ and R⁵ are as defined above,

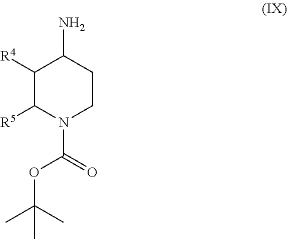

with compound of formula (VIII), wherein R⁸ is a halogen and R⁶, R⁷, and X⁵ are as defined above,

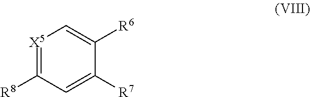

to form compound of formula (X), wherein wherein R⁴ R⁶, R⁷, X⁵, and R⁵ are as defined above,

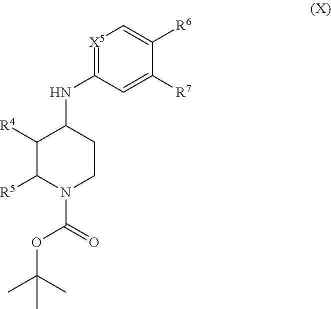

reacting said compound of formula (X) with acid to form compound of formula (XI), wherein R⁴ R⁶, R⁷, X⁵, and R⁵ are as defined above,

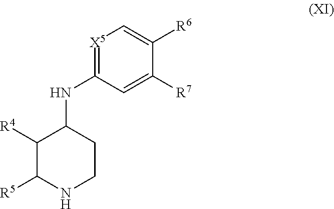

reacting said compound of formula (XI) with compound of formula (III), wherein X², X³, R², and R³, are as defined above,

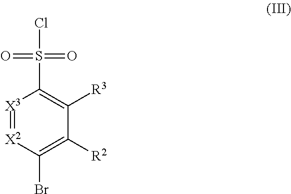

to form compound of formula (XII), wherein X², X³, R², R³, R⁴ R⁶, R⁷, X⁵, and R⁵ are as defined above,

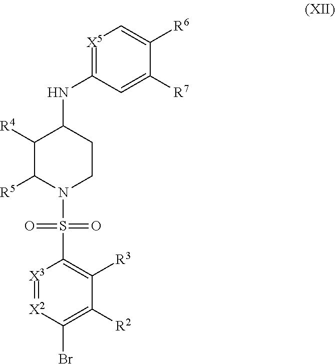

reacting said compound of formula (XII) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

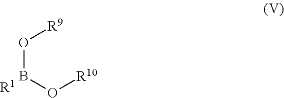

to form compound of formula (I).

The present invention provides a process of preparation of a compound as described herein, or a pharmaceutically acceptable salt thereof, wherein X¹ is N and X⁴ is O, comprising:
reacting compound of formula (XVIII), wherein R⁵ and R⁴ are as defined above,

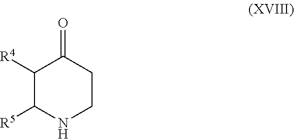

with compound of formula (XVII), wherein R¹, R², R³, X², and X³ are as defined above,

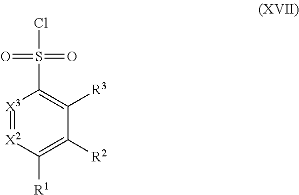

to form compound of formula (XX), wherein R¹, R², R³, R⁴, R⁵, X², and X³ are as defined above,

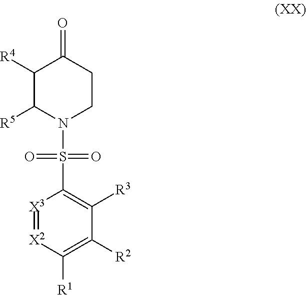

reacting said compound of formula (XX) with compound of formula (XVI), wherein X⁵, R⁶, and R⁷ are as defined above,

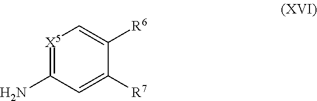

to form compound of formula (I);
or
reacting compound of formula (XVIII), wherein R⁵ and R⁴ are as defined above,

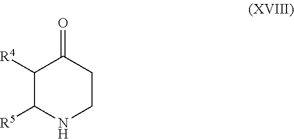

with compound of formula (III), wherein X², X³, R², and R³, are as defined above,

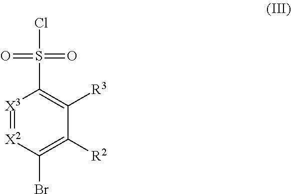

to form compound of formula (XIX), wherein R², R³, R⁴, R⁵, X², and X³ are as defined above,

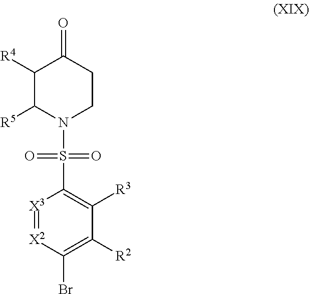

reacting said compound of formula (XIX) with said compound of formula (XVI), to form compound of formula (XII), wherein X², X³, R², R³, R⁴ R⁶, R⁷, X⁵, and R⁵ are as

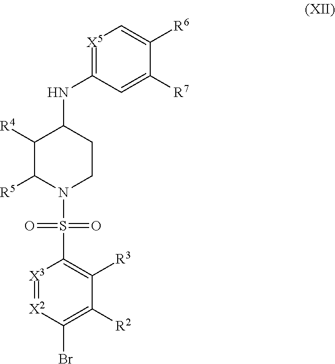

reacting said compound of formula (XII) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

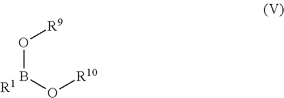

to form compound of formula (I).

The present invention provides a process of preparation of a compound as described herein, or a pharmaceutically acceptable salt thereof, wherein X¹ is C and X⁴ is O, comprising:
reacting compound of formula (XXI), wherein R⁴ and R⁵ are as defined above,

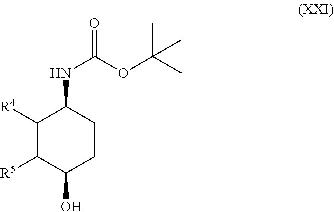

with masyl chloride, to form compound of formula (XXII), wherein R⁴ and R⁵ are as defined above,

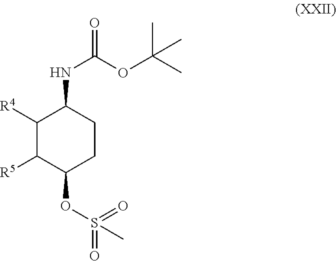

reacting said compound of formula (XXII) with compound of formula (XXIII), wherein R², R³, X², and X³ are as defined above,

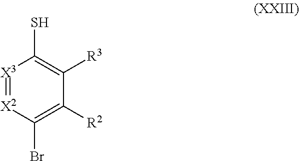

to form compound of formula (XXIV), wherein R², R³, R⁴, R⁵, X², and X³ are as defined above,

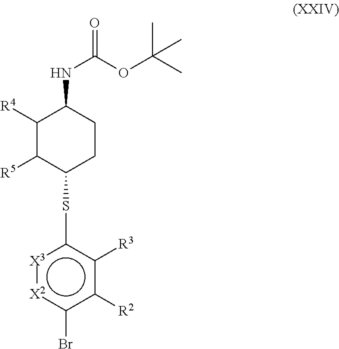

reacting said compound of formula (XXIV) with an oxidant, to form compound of formula (XXV), wherein R², R³, R⁴, R⁵, X², and X³ are as defined above,

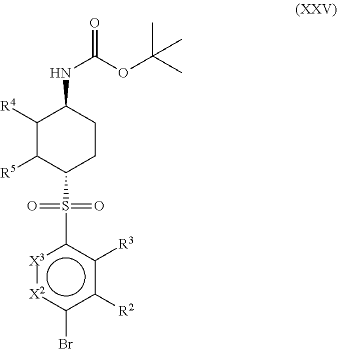

reacting said compound of formula (XXV) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

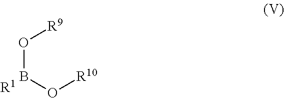

to form compound of formula (XXVI), wherein R¹, R², R³, R⁴, R⁵, X², and X³ are as defined above,

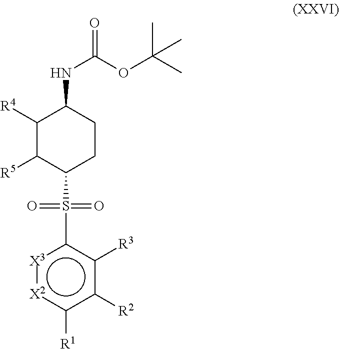

reacting said compound of formula (XXVI) with acid, to form compound of formula (XXVII), wherein R¹, R², R³, R⁴, R⁵, X², and X³ are as defined above,

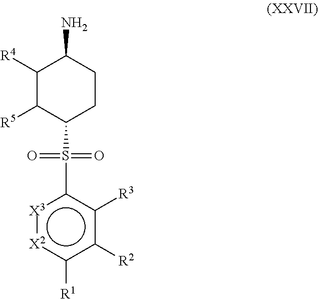

reacting said compound of formula (XXVII) with compound of formula (VIII), wherein R⁸ is a halogen and R⁶, R⁷, and X⁵ are as defined above,

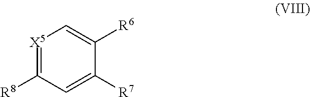

to form compound of formula (I).

The present invention provides a process of preparation of a compound as described herein, or a pharmaceutically acceptable salt thereof, wherein X¹ is C and X⁴ is O, comprising:
reacting compound of formula (XXV), wherein R², R³, R⁴, R⁵, X², and X³ are as defined above,

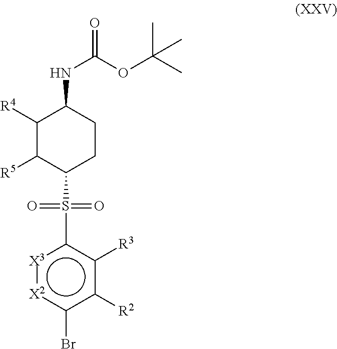

with acid to form compound of formula (XXVIII), wherein R², R³, R⁴, R⁵, X², and X³ are as defined above,

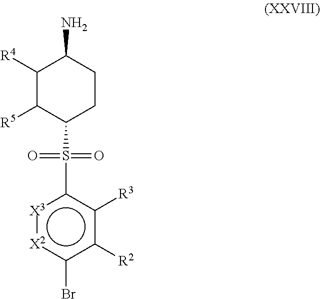

reacting said compound of formula (XXVIII) with compound of formula (VIII), wherein R⁸ is a halogen and R⁶, R⁷, and X⁵ are as defined above,

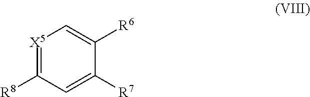

to form compound of formula (XXIX), wherein R², R³, R⁴, R⁵, R⁶, R⁷, X⁵, X², and X³ are as defined above,

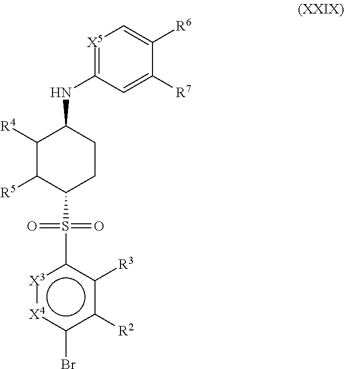

reacting said compound of formula (XXIX) with with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

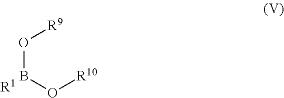

to form compound of formula (I).

The present invention provides a process of preparation of a compound as described herein, or a pharmaceutically acceptable salt thereof, wherein X¹ is C and X⁴ is O, comprising:
reacting compound of formula (XXXI), wherein R⁴ and R⁵ are as defined above,

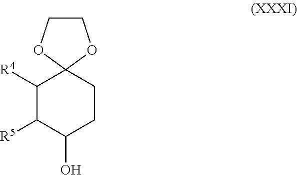

with mesyl chloride, to form compound of formula (XXXII), wherein R⁴ and R⁵ are as defined above,

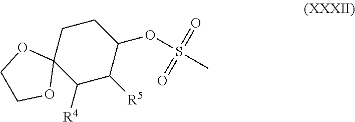

reacting said compound of formula (XXXII) with compound of formula (XXIII), wherein R², R³, X², and X³ are as defined above,

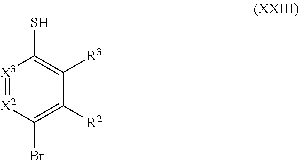

to form compound of formula (XXXIII), wherein R², R³, R⁴, R⁵, X², and X³ are as defined above,

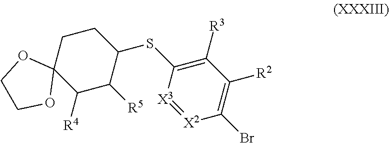

reacting said compound (XXXIII) with an oxidant, to form compound of formula (XXXIV), wherein R², R³, R⁴, R⁵, X², and X³ are as defined above,

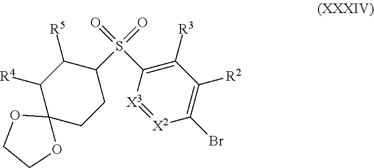

reacting said compound (XXXIV) with an acid, to form compound of formula (XXXV), wherein R², R³, R⁴, R⁵, X², and X³ are as defined above,

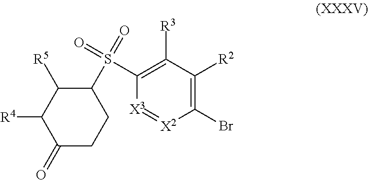

reacting said compound of formula (XXXV) with compound of formula (XVI), wherein X⁵, R⁶, and R⁷ are as defined above,

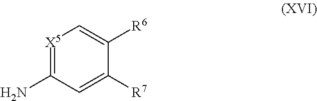

to form compound of formula (XXIX), wherein R², R³, R⁴, R⁵, R⁶, R⁷, X⁵, X², and X³ are as defined above,

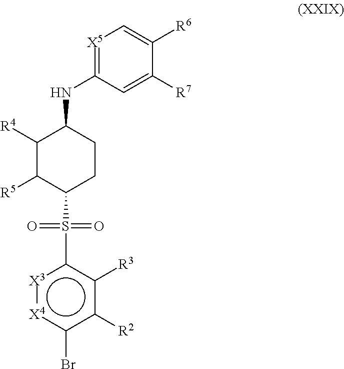

reacting said compound of formula (XXIX) with with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

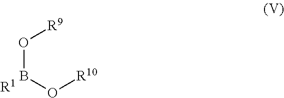

to form compound of formula (I).

The present invention provides a process of preparation of a compound as described herein, or a pharmaceutically acceptable salt thereof, wherein X¹ is C and X⁴ is N, comprising:
reacting compound of formula (XXXIII), wherein R², R³, R⁴, R⁵, X², and X³ are as defined above,

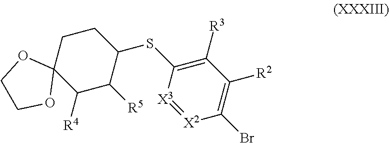

with an acid to form compound of formula (XXXVI), wherein R², R³, R⁴, R⁵, X², and X³ are as defined above,

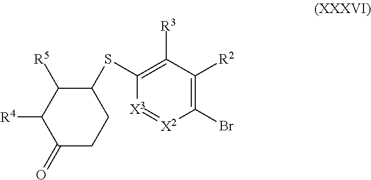

reacting said compound of formula (XXXVI) with compound of formula (XVI), wherein X⁵, R⁶, and R⁷ are as defined above,

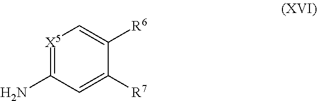

to form compound of formula (XXXVII), wherein R², R³, R⁴, R⁵, R⁶, R⁷, X⁵, X², and X³ are as defined above,

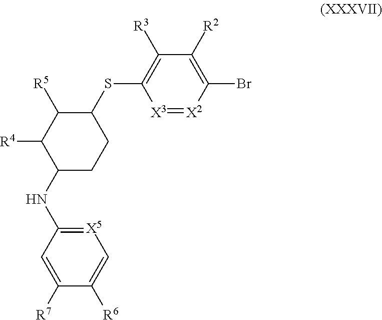

reacting said compound of formula (XXXVII) with ammonium carbamate and (diacetoxyiodo)benzene, to form compound of formula (XXXVIII), wherein R², R³, R⁴, R⁵, R⁶, R⁷, X⁵, X², and X³ are as defined above,

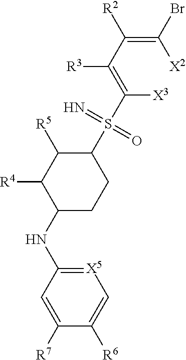

reacting said compound of formula (XXXVIII) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

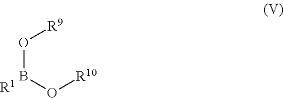

to form compound of formula (I).

The present invention provides a process of preparation of a compound as described herein, or a pharmaceutically acceptable salt thereof, wherein X¹ and X⁴ are N, comprising:
reacting compound of formula (XI), wherein R⁴ R⁶, R⁷, X⁵, and R⁵ are as defined above,

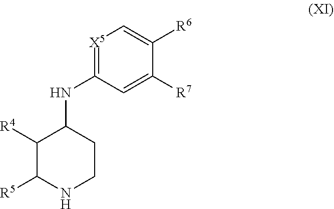

with compound of formula (XXIII), wherein R², R³, X², and X³ are as defined above,

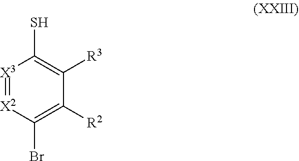

to form compound of formula (XL), wherein R², R³, X², X³, R⁴ R⁶, R⁷, X⁵, and R⁵ are as defined above,

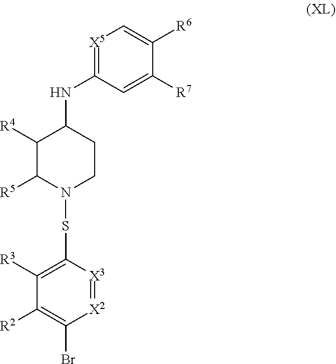

reacting said compound (XL) with ammonium carbamate and (diacetoxyiodo)benzene, to form compound of formula (XLI), wherein R², R³, X², X³, R⁴ R⁶, R⁷, X⁵, and R⁵ are as defined above,

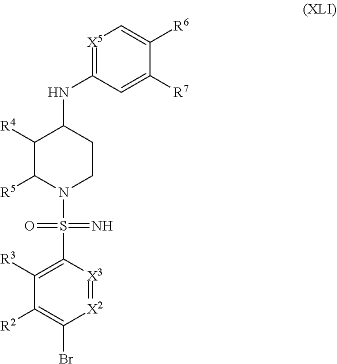

reacting said compound (XLI) with di-tert-butyl dicarbonate and a base, to form compound of formula (XLII), wherein R², R³, X², X³, R⁴ R⁶, R⁷, X⁵, and R⁵ are as defined above,

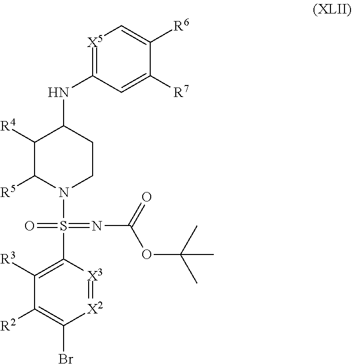

reacting said compound (XLII) with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

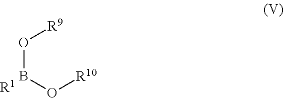

to form compound of formula (XLIII), wherein R¹, R², R³, X², X³, R⁴ R⁶, R⁷, X⁵, and R⁵ are as defined above,

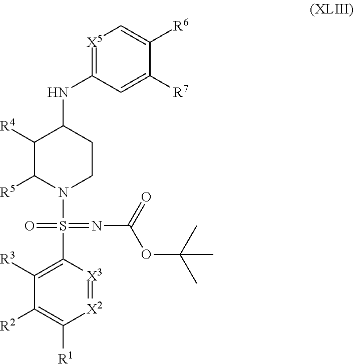

reacting said compound of formula (XLIII) with an acid to form compound of formula (I).

The present invention provides a process of preparation of a compound as described herein, or a pharmaceutically acceptable salt thereof, wherein X¹ is C and X⁴ is N, comprising:
reacting compound of formula (XXIV), wherein R², R³, R⁴, R⁵, X², and X³ are as defined above,

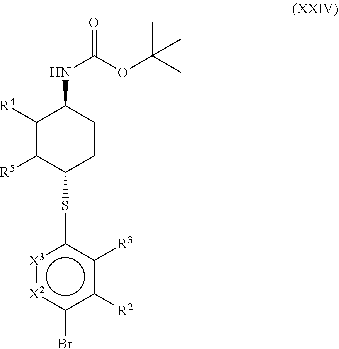

with compound of formula (V), wherein R¹ is as defined above, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

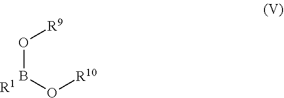

to form compound of formula (XLIV), wherein R¹, R², R³, R⁴, R⁵, X², and X³ are as defined above,

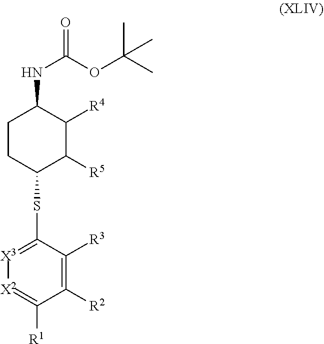

reacting said compound of formula (XLIV) with acid to form compound of formula (XLV), wherein R¹, R², R³, R⁴, R⁵, X², and X³ are as defined above,

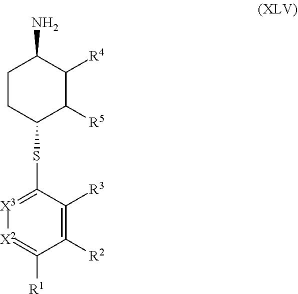

reacting said compound of formula (XLV) with compound of formula (VIII), wherein R⁸ is a halogen and R⁶, R⁷, and X⁵ are as defined above,

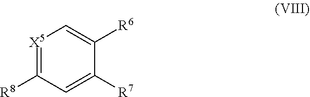

to form compound of formula (XLVI), wherein R¹, R², R³, R⁴, R⁵, R⁶, R⁷, X⁵, X², and X³ are as defined above,

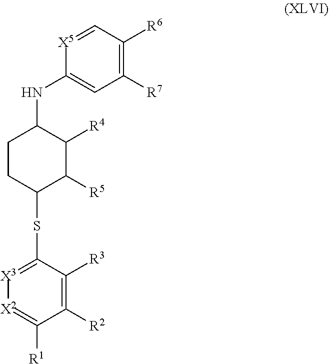

reacting said compound of formula (XLVI) with ammonium carbamate and (diacetoxyiodo)benzene to form compound of formula (I).

### EXEMPLIFICATION

As depicted in the Examples below, in certain exemplary embodiments, compounds are prepared according to the following general procedures. It will be appreciated that, although the general methods depict the synthesis of certain compounds of the invention, the following general methods, and other methods known to one skilled in the art, can be applied to all compounds and subclasses and species of each of these compounds, as described herein.

### General Methods of Preparation

The compounds described herein, including compounds of general Formula (I), can be readily prepared according to the following reaction schemes and Examples, or modifications thereof, using readily available starting materials, reagent and conventional synthesis procedures. Many of the reactions can also be carried out under microwave conditions or using conventional heating or utilizing other technologies such as solid phase reagents/scavengers or flow chemistry. In these reactions, it is also possible to make use of variants which are themselves known to those skilled in the art, but are not mentioned in greater detail. For Example, where specific acids, bases, reagents, coupling agents, solvents, etc. are mentioned, it is understood that other suitable acids, bases, reagents, coupling agents, solvents etc. may be used and are included within the scope of the present invention. Furthermore, other methods for preparing compounds of the invention will be readily apparent to a person of ordinary skill in the art in light of the following reaction schemes and Examples. In cases where synthetic intermediates and final products contain potentially reactive functional groups, for Example Amino, hydroxyl, thiol and carboxylic acid groups that may interfere with the desired reaction, it may be advantageous to employ protected forms of the intermediate. Methods for the selection, introduction and subsequent removal of protecting groups are well known to those skilled in the art. The compounds obtained by using the general reaction sequences may be of insufficient purity. The compounds can be purified by using any of the methods of purification of organic compounds, for Example, crystallization or silica gel, alumina or C18 column chromatography, using different solvents in suitable ratios. All possible stereoisomers are envisioned within the scope of the invention. In the discussion below variables have the meaning indicated above unless otherwise indicated. All final compounds have been characterized using for example LC-MS, NMR and/or Specific Optical Rotation.

The abbreviations used in these experimental details are listed below and additional ones should be considered known to a person skilled in the art of synthetic chemistry.

Abbreviations used herein are as follow: r.t.: room temperature; TFA: Trifluoroacetic acid; THF: Tetrahydrofuran; EtOH: Ethanol; EtOAc: ethyl acetate; TEA: triethyl amine; BINAP: 2,2′-bis(difenylfosfino)-1,1′-binaftyl; BOC: tert-Butyloxycarbonyl; t-BuONa: Sodium tertiar butoxide; N: Normal; DMF: Dimethylformamide; HOAc; Acetic acid.

Chemical names are preferred IUPAC names.

If a chemical compound is referred to using both a chemical structure and a chemical name, and an ambiguity exists between the structure and the name, the structure predominates.

### General Procedures

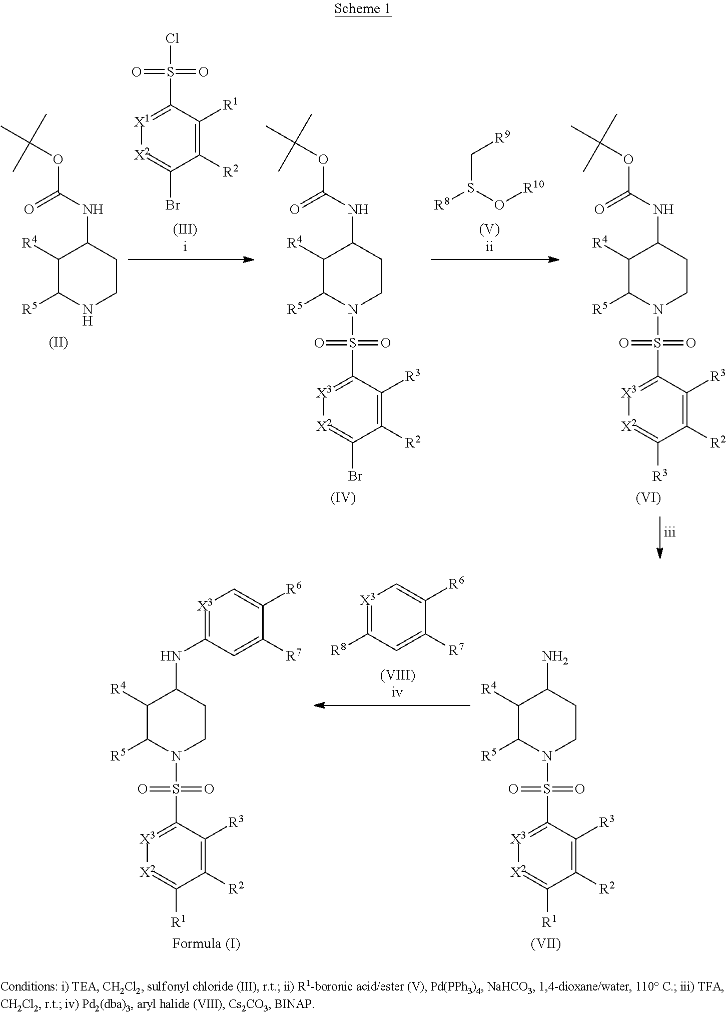

Scheme 1 describes a route to synthesize derivatives of the invention having Formula (I), when X₁=N and X₄=O.

These compounds can for example be obtained by starting from readily available protected 4-aminopiperidines of formula (II), wherein R⁴ and R⁵ have the meaning as previously described, which can be coupled with sulfonyl chlorides of formula (III), wherein X², X³, R² and R³ have the meaning as previously described, to form the corresponding sulfonamides of formula (IV). Derivatives of formula (VI) can be prepared by coupling of derivatives of formula (IV) with commercially available boronic acids or boronic esters of formula (V), wherein R₁ has the meaning as previously described, under Suzuki conditions using for example Pd(PPh₃)₄ and NaHCO₃ in dioxane/water mixture. After deprotection of the amine under acidic conditions, using for example TFA or an aqueous HCl solution, the obtained aminopiperidines derivatives of formula (VII) can be converted, under Buchwald conditions, using for example Pd₂(dba)₃, BINAP, and Cs₂CO₃ as the base, into derivatives of Formula (I), wherein X¹, X², X³, X⁴, X⁵, R¹, R², R³, R⁴, R⁵, R⁶ and R⁷ have the meaning as previously described

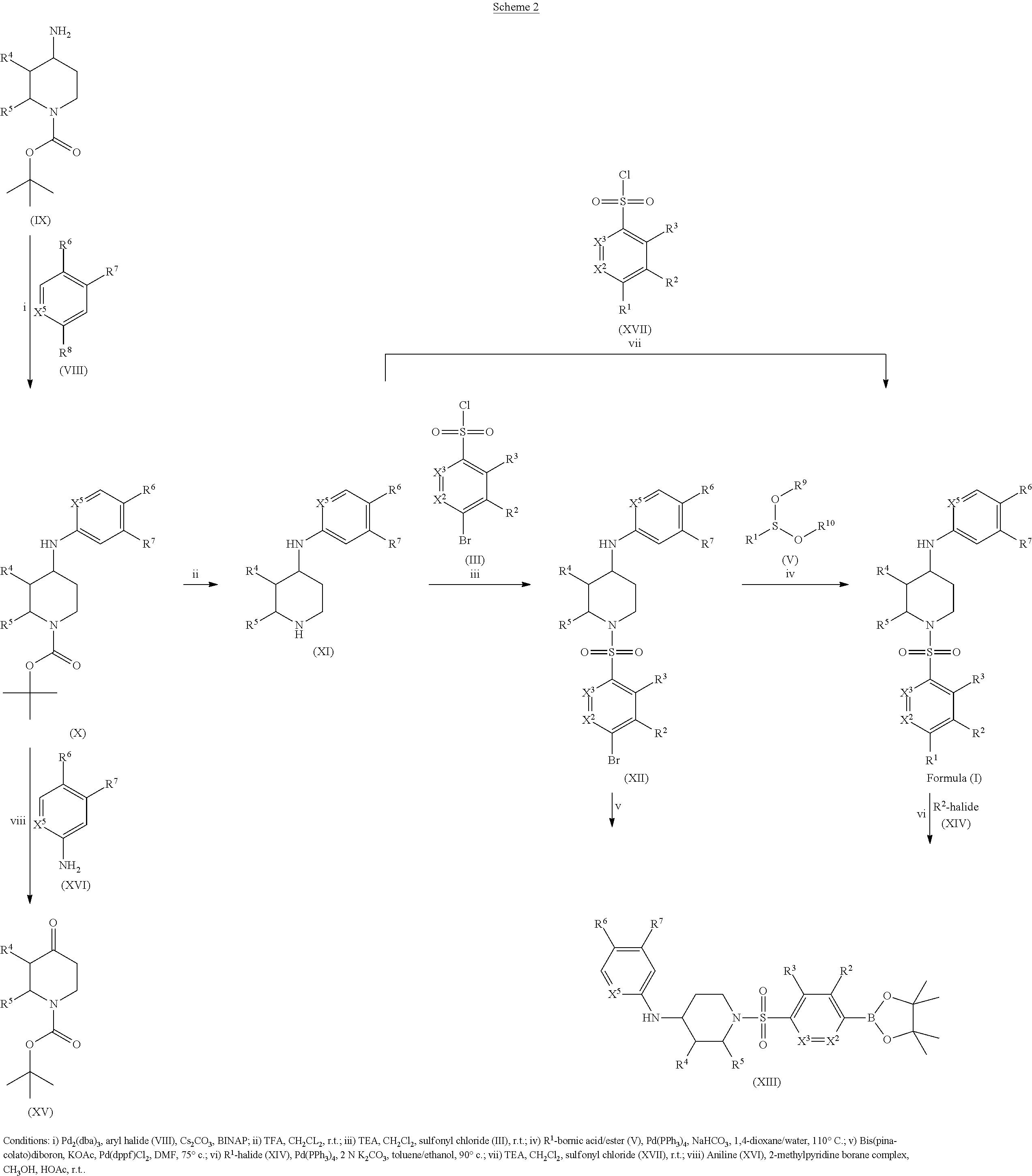

Scheme 2 describes an alternative route to synthesize derivatives of the invention having Formula (I), when X₁=N and X₄=O.

Compounds of the invention can for example be obtained by coupling under Buchwald conditions, of commercially available protected 4-aminopiperidine derivatives of formula (IX), wherein X², X³, R², R³, R⁴ and R⁵ have the meaning as previously described, and aryl halide derivatives of formula (VIII), wherein R⁸ is a halogen and X⁵, R⁶ and R⁷ have the meaning as previously described, to give the corresponding protected N-phenylpiperidin-4-amine derivatives of formula (X), which after deprotection under acidic conditions, using for example TFA, give 4-aminopiperidine derivatives of formula (XI). In an alternative way, protected N-phenylpiperidin-4-amine derivatives of formula (X) can be prepared under reductive amination conditions, starting from the N-protected piperidin-4-one derivatives of formula (XV), wherein R⁴ and R⁵ have the meaning as previously described, and an aniline derivative of formula (XVI), wherein X⁵, R⁶ and R⁷ have the meaning as previously described. Derivatives of Formula (I) can be prepared, by direct coupling of derivatives of 4-aminopiperidine derivatives (XI) with commercially available sulfonyl chlorides (XVII), wherein R¹, R², R³, X² and X³ have the meaning as previously described. Alternatively, derivatives of 4-aminopiperidine derivatives (XI) can be coupled with 4-bromosulfonyl derivatives of formula (III) to give 1-(4-bromobenzenesulfonyl)-N-phenylpiperidin-4-amine derivatives of formula (XII) from which derivatives of Formula (I) can be prepared, either by direct coupling of derivatives of formula (XII) with commercially available boronic acids or boronic esters of formula (V), wherein R¹ has the meaning as previously described, under Suzuki conditions using for example Pd(PPh₃)₄ and NaHCO₃ in dioxane/water mixture or, by first converting the piperidin derivatives of formula (XII) into the corresponding boronic ester derivatives of formula (XIII), which then can be reacted under Suzuki conditions, with R₁-halides of formula (XIV), wherein R₁ has the meaning as previously described

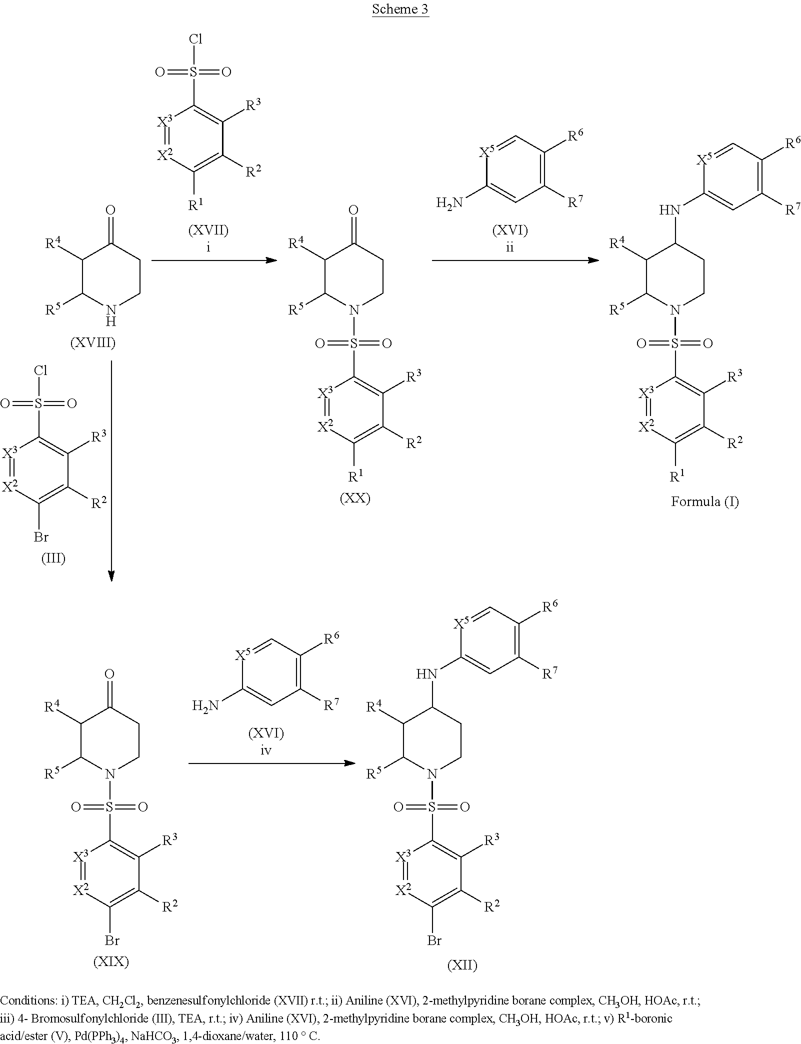

Scheme 3 describes an other route to synthesize derivatives of the invention having Formula (I), when X₁=N and X₄=O.

Starting from commercially available piperidine-4-one derivatives of formula (XVIII), wherein R⁴ and R⁵ have the meaning as previously described, 1-(benzenesulfonyl)piperidin-4-one derivatives of formula (XX) can be prepared by coupling derivatives of formula (XVIII) with benzenesulfonylchloride derivatives of formula (XVII), wherein R¹, R², R³, X² and X³ have the meaning as previously described. 1-(benzenesulfonyl)piperidin-4-one derivatives of formula (XX) can then be coupled under reductive amination conditions, with suitable aniline derivatives of formula (XVI), wherein X⁵, R⁶ and R⁷ have the meaning as previously described, to give the corresponding derivatives of Formula (I).

Alternatively, derivatives of piperidine-4-one derivatives of formula (XVIII) can be coupled with 4-bromosulfonyl derivatives of formula (III), wherein X², X³, R² and R³ have the meaning as previously described, to give 1-(4-bromobenzenesulfonyl)piperidin-4-one derivatives of formula (XIX), which, under reductive amination conditions, can be converted to the corresponding derivatives of formula (XII), using appropriate aniline derivatives of formula (XVI), wherein X⁵, R⁶ and R⁷ have the meaning as previously described. Under Suzuki conditions, using for example Pd(PPh₃)₄ and NaHCO₃ in dioxane/water mixture, the bromophenyl derivatives of formula (XII), can be coupled with commercially available boronic acids or boronic esters of formula (V), wherein R¹ has the meaning as previously described, to give the corresponding derivatives of Formula (I)

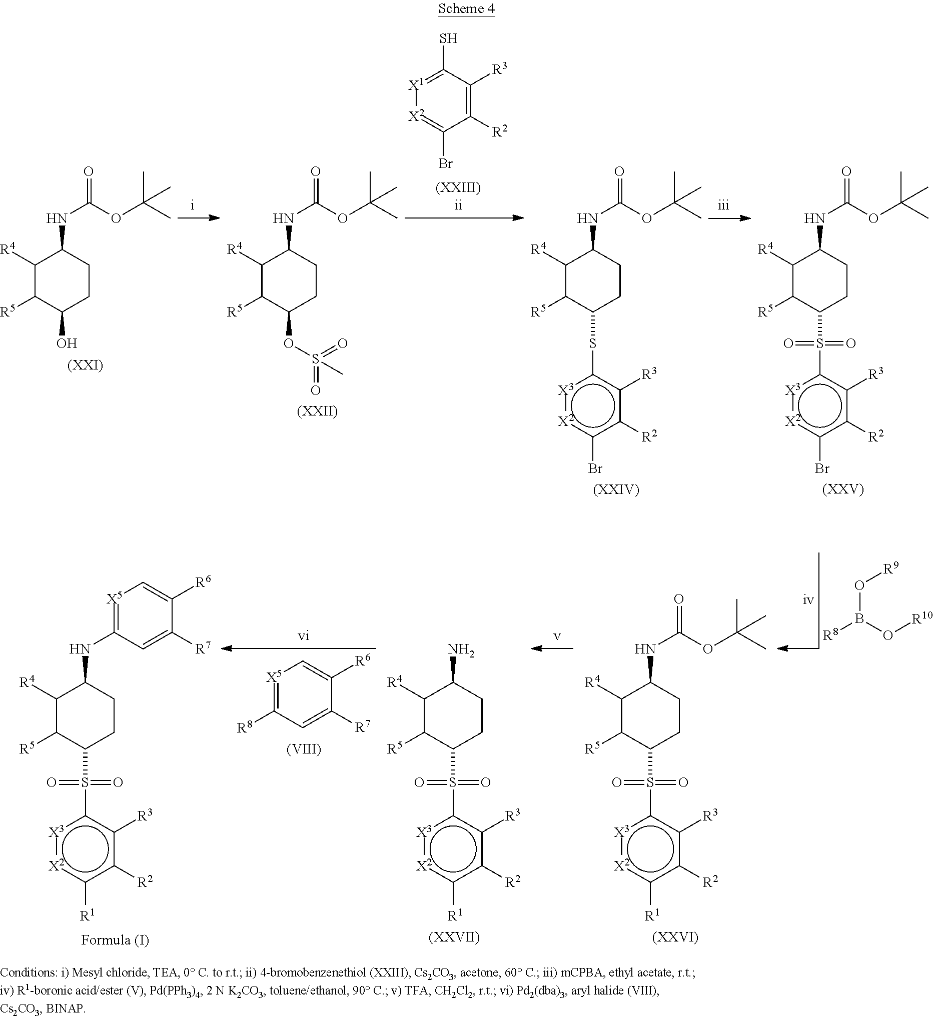

Scheme 4 describes a route to synthesize derivatives of the invention having Formula (I), when X₁=C and X₄=O.

As depicted in scheme 4, the derivatives of the invention having Formula (I) can be obtained by coupling of N-Boc protected cis-4-aminocyclohexan-1-ol derivatives of formula (XXI), wherein R⁴ and R⁵ have the meaning as previously described, which can easily be prepared by someone skilled in the art of organic chemistry, and mesylchloride under basic conditions, to give the corresponding N-Boc protected cis-4-aminocyclohexyl methanesulfonate derivatives of formula (XXII). These sulfonates derivatives of formula (XXII) can be converted into N-Boc protected 4-[(4-bromophenyl)sulfanyl]cyclohexan-1-amine derivatives of formula (XXIV) via a nucleophilic substitution reaction with 4-bromobenzene-1-thiol derivatives of formula (XXIII), using a suitable base e.g. Cs₂CO₃, which can be oxidized to the corresponding N-Boc protected sulfonyl derivatives of formula (XXV) using for example mCPBA as the oxidizing agent. These sulfonyl derivatives of formula (XXV) can then be coupled with the appropriate boronic acids or boronic esters of formula (V) under Suzuki conditions, using for example Pd(PPh₃)₄ as the catalyst, to form the corresponding derivatives of formula (XXVI). After removing the Boc-group of derivatives of formula (XXVI) under acidic conditions, using for example TFA, the obtained derivatives of formula (XXVII) can be converted, for example, under Buchwald conditions, using the appropriate aryl halide (VIII), wherein R⁸ is a halogen and X⁵, R⁶ and R⁷ have the meaning as previously described, using for example Pd₂(dba)₃, BINAP and a suitable base e.g. Cs₂CO₃, into derivatives of Formula (I), wherein X², X³, X⁵, R¹, R², R³, R⁴, R⁵, R⁶ and R⁷ have the meaning as previously described

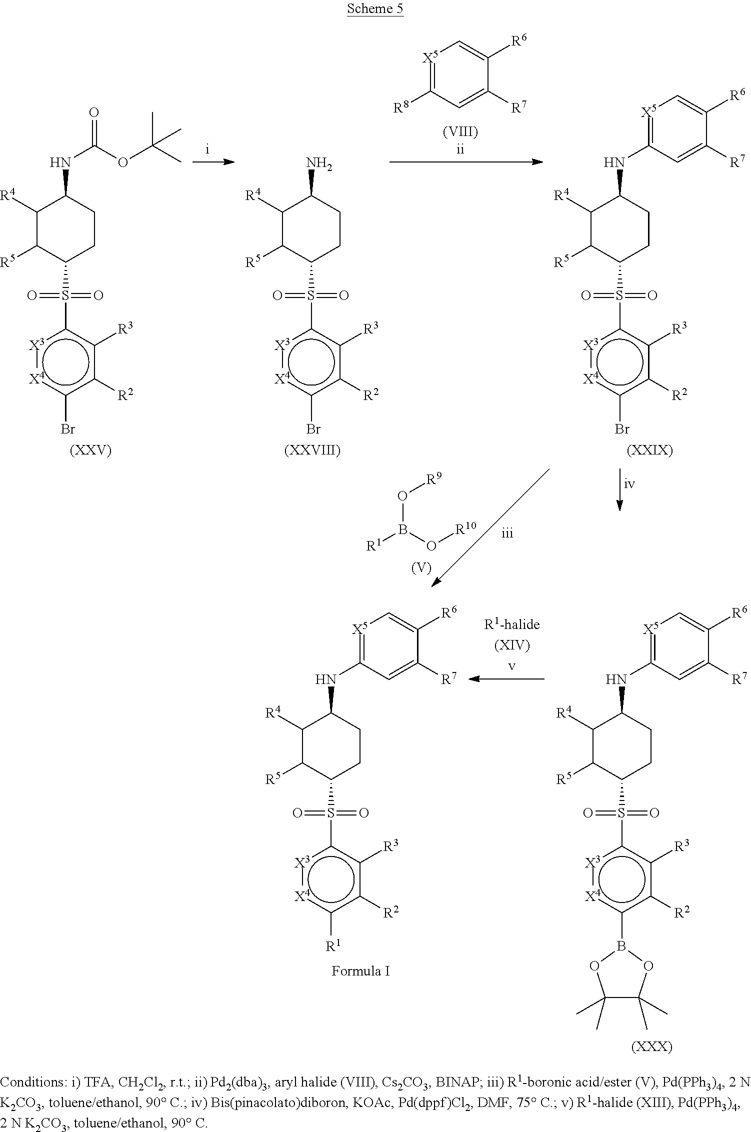

Scheme 5 describes an alternative route to synthesize derivatives of the invention having Formula (I), when X₁=C and X₄=O.

In an alternative way, first the Boc group of derivatives of formula (XXV) can be removed under acidic conditions, using for example TFA to obtain the Amino derivatives of formula (XXVIII) which can be converted, under Buchwald conditions, as previously described, into derivatives of formula (XXIX). Derivatives of formula (XXIX) can be converted into derivatives of Formula (I) either by reaction with suitable boronic acids or boronic esters of formula (V), wherein R¹ has the meaning as previously described, under Suzuki conditions using for example Pd(PPh₃)₄ as the catalyst or by first converting derivatives of formula (XXIX) into their corresponding boronic esters of formula (XXX) which can further be reacted with suitable R¹-halides of formula (XIV), under Suzuki conditions, as previously described

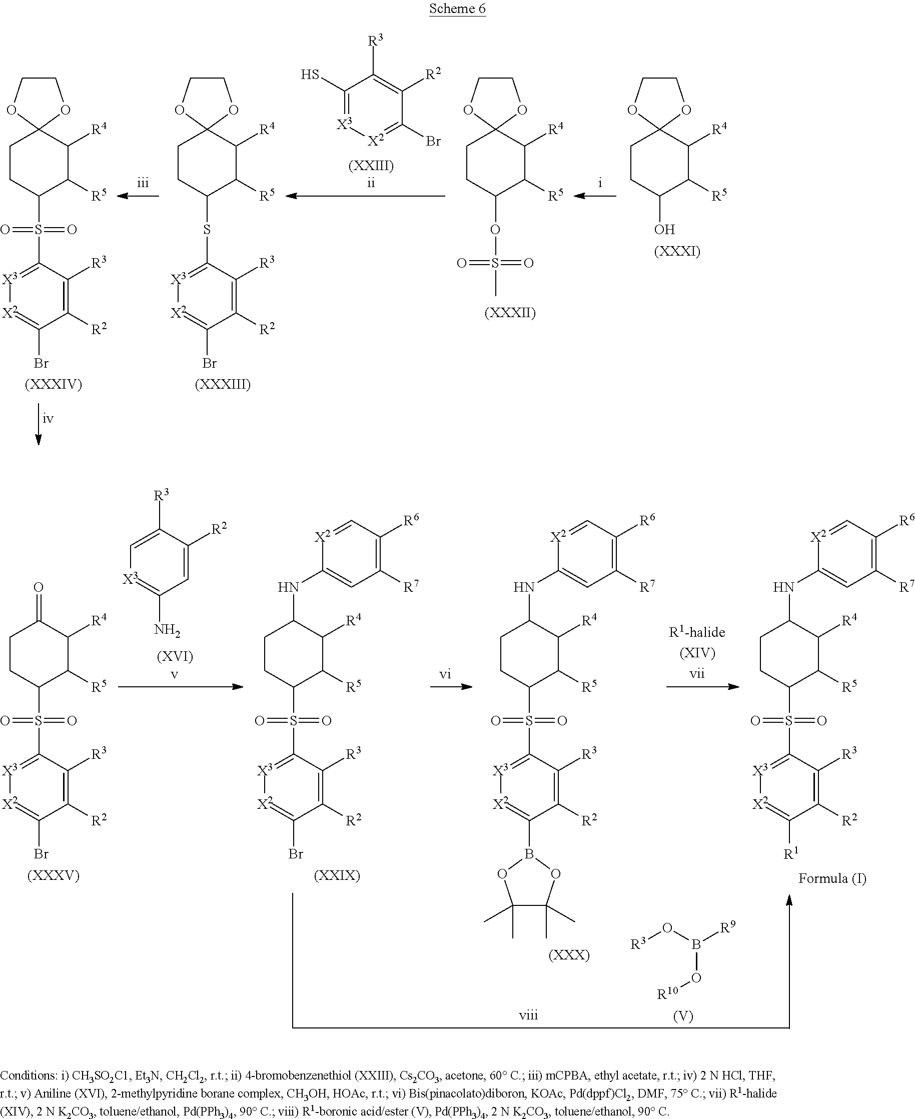

Scheme 6 describes an other route to synthesize derivatives of the invention having Formula (I), when X₁=C and X₄=O.

These compounds can for example be obtained by starting from readily available keto-protected 4-hydroxycyclohexan-1-ones derivatives of formula (XXXI), wherein R⁴ and R⁵ have the meaning as previously described, in which the hydroxyl group is converted into a suitable leaving group, e.g. a mesyl or tosyl group, to give derivates of formula (XXXII), which can react with 4-bromobenzenethiol derivatives of formula (XXIII), to form the phenylsulfanyl derivatives of formula (XXXIII). After oxidation of the sulfur, using for example mCPBA, the formed sulfonyl derivatives of formula (XXXIV) can then be deprotected under acid conditions to get the sulfonylcyclohexanone derivatives of formula (XXXV). These sulfonylcyclohexanone derivatives of formula (XXXV) can be coupled to an aniline derivative of formula (XVI), wherein X⁵, R⁶ and R⁷ have the meaning as previously described, under reductive amination conditions to give the corresponding N-phenylpiperidin-4-amine derivatives of formula (XXIX). Derivatives of Formula (I) can then be prepared, either by direct coupling of derivatives of formula (XXIX) with commercially available boronic acids or boronic esters of formula (V), wherein R¹ has the meaning as previously described, under Suzuki conditions using for example Pd(Ph₃)₄ and NaHCO₃ in dioxane/water mixture or, by first converting derivatives of formula (XXIX) into the corresponding boronic acids or boronic esters of formula (XXX), which then can be reacted under Suzuki conditions, with R₁-halides of formula (XIV), wherein R¹ has the meaning as previously described.

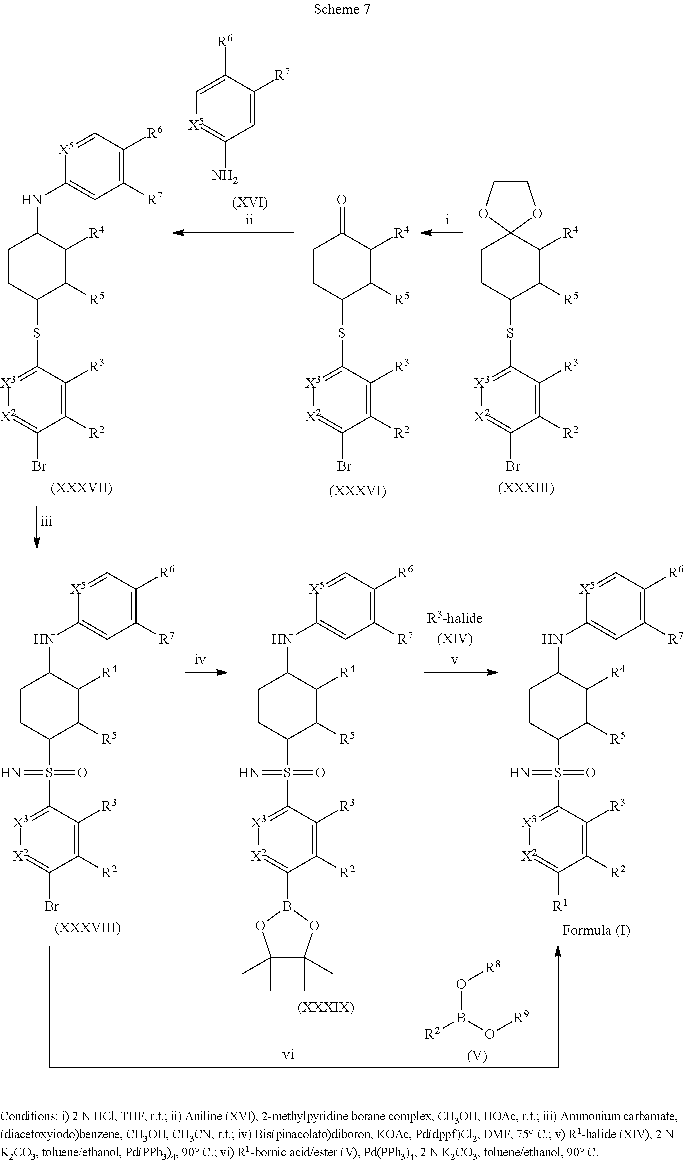

Scheme 7 describes a route to synthesize derivatives of the invention having Formula (I), when X₁=C and X₄=N.

Keto-protected sulfanyl derivatives of formula (XXXIII) can be deprotected under acidic conditions, using for example HCl or TFA, to give 4-(phenylsulfanyl)cyclohexan-1-one derivatives of formula (XXXVI). Under reductive amination conditions, derivatives of formula (XXXVI) can be coupled with suitable aniline derivatives of formula (XVI), wherein X⁵, R⁶ and R⁷ have the meaning as previously described, to give the corresponding N-[4-(phenylsulfanyl)cyclohexyl]aniline derivatives of formula (XXXVII). Derivatives of formula (XXXVIII) can be prepared by converting derivatives of formula (XXXVII) into the corresponding sulfoximines derivatives, by using ammonium carbamate, (diacetoxyiodo)benzene and CH₃CN in CH₃OH. Finally, derivatives of formula (XXXVIII) can be converted into derivatives of Formula (I) either by reaction with suitable boronic acids or boronic esters of formula (V), wherein R₁ has the meaning as previously described, under Suzuki conditions using for example Pd(PPh₃)₄ as the catalyst or by first converting derivatives of formula (XXXVIII) into their corresponding boronic ester of formula (XXXIX), which can further be reacted with suitable R¹-halides of formula (XIV) under Suzuki conditions, as previously described

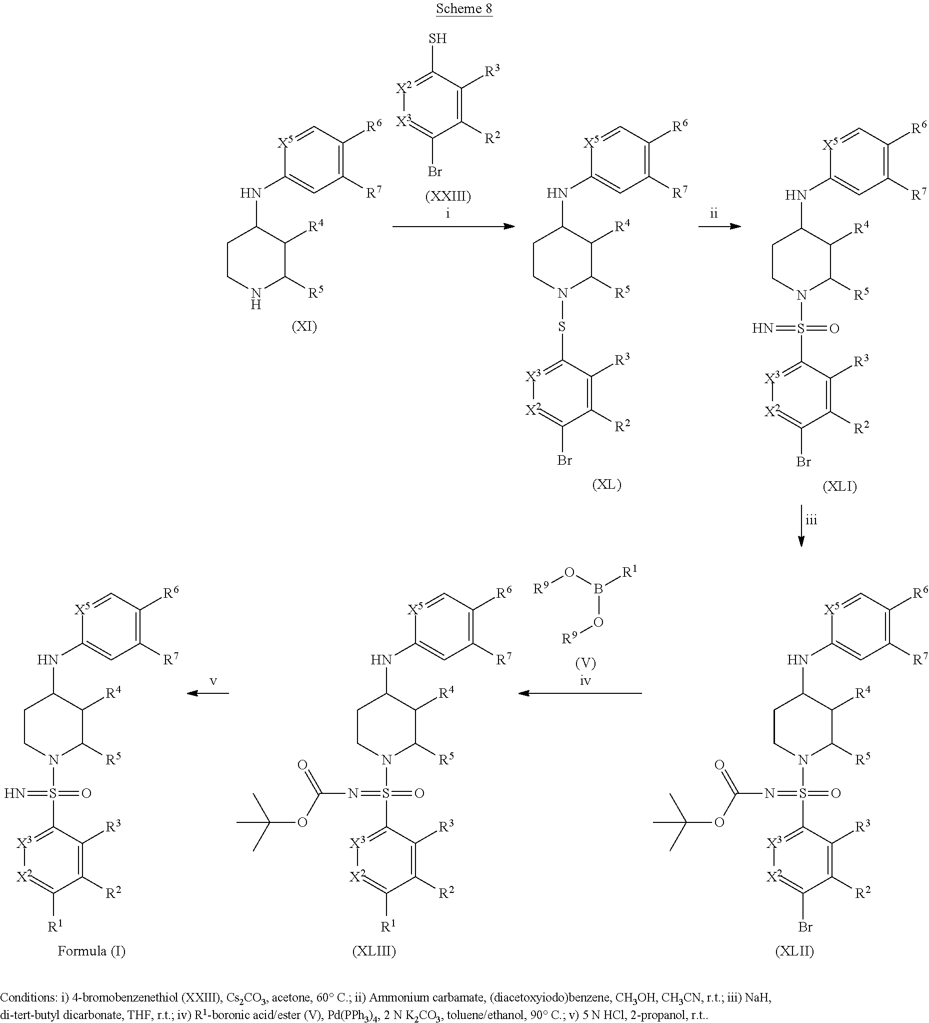

Scheme 8 describes a route to synthesize derivatives of the invention having Formula (I), when X₁ and X₄ are N.

N-phenylpiperidin-4-amine derivatives of formula (XI), wherein X⁵, R⁴, R⁵, R⁶ and R⁷ have the meaning as previously described, can be converted into the N-phenyl-1-(phenylsulfanyl)piperidin-4-amine derivatives of formula (XL) by reaction with 4-bromobenzene-1-thiol derivatives of formula (XXIII), wherein X², X³, R² and R³ have the meaning as previously described, using for example Cs₂CO₃ as the base. Derivatives of formula (XL) can be converted into the corresponding sulfoniminamide derivatives of formula (XLI), by using ammonium carbamate, (diacetoxyiodo)benzene and CH₃CN in CH₃OH. After protecting the sulfonimidamide nitrogen with for example a Boc-group, derivatives of formula (XLII) can react with suitable boronic acids or boronic acids of formula (V) under Suzuki conditions, as previously described, to give derivatives of formula (XLIII). In the final step, the Boc-group can be removed, under acidic conditions, to give the corresponding derivatives of Formula (I), wherein X², X³, X⁵, R¹, R², R³, R⁴, R⁵, R⁶ and R⁷ have the meaning as previously described

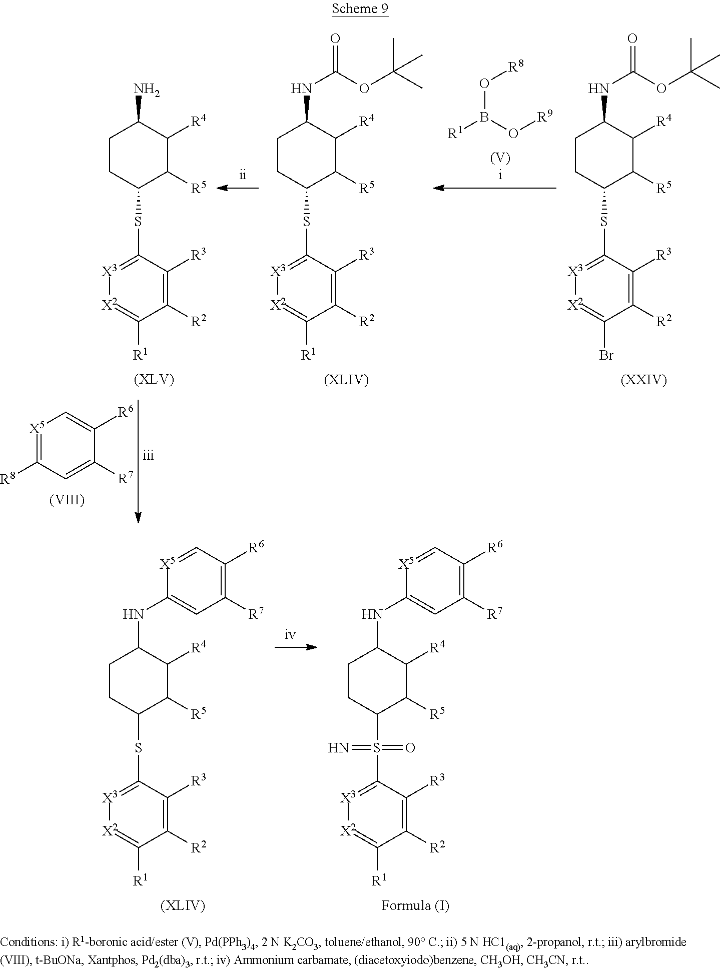

Scheme 9 describes an alternative route to synthesize derivatives of the invention having Formula (I), when X₁=C and X₄=N.

N-protected N-(4-bromophenyl)sulfanylcyclohexyl derivatives of formula (XXIV) can be first coupled under Suzuki conditions as previously described, to suitable boronic acids or boronic esters (V), wherein R¹ has the meaning as previously described, to obtain the corresponding N-protected phenylsulfanylcyclohexyl derivatives of formula (XLIV) which after removal of the protecting group under acidic conditions, using for example HCl or TFA, give phenylsulfanylcyclohexyl derivatives of formula (XLV). The derivatives of formula (XLV) can then be coupled to suitable aryl halides (VIII), wherein R⁸ is a halogen and X₅, R⁶ and R⁷ have the meaning as previously described, under Buchwald conditions, using Pd₂(dba)₃, Xantphos, and t-BuONa as the base, into derivatives of formula (XLVI), which finally can be converted into the corresponding sulfoximines derivatives of Formula (I), by using ammonium carbamate, (diacetoxyiodo)benzene and CH₃CN in CH₃OH.

### Pharmaceutical Compositions and Administration

Another object of the present invention is a pharmaceutical composition comprising a compound of formula (I) as described herein, or a pharmaceutically acceptable salt thereof, and a pharmaceutically acceptable excipient.

Another embodiment of the present invention is a pharmaceutical composition comprising a compound as described herein, or a pharmaceutically acceptable salt thereof, and a pharmaceutically acceptable excipient.

Another embodiment of the present invention is the pharmaceutical composition as described herein, further comprising an additional therapeutic agent.

### Indications

The compounds of formula (I), (I′) as described herein, can be used in an effective amount to treat a subject, in particular a human, affected by an inflammatory autoimmune disease.

In one embodiment of the present invention is the compound as described herein, or a pharmaceutically acceptable salt thereof, for use as therapeutically active substance.

In one embodiment of the present invention is the compound as described herein, or a pharmaceutically acceptable salt thereof, for use in the treatment, prevention and/or delay of an inflammatory autoimmune disease.

In one embodiment of the present invention is the compound as described herein, or a pharmaceutically acceptable salt thereof, for use in the treatment, prevention and/or delay of progression of psoriatic diseases, asthma, Inflammatory Bowel Diseases (IBD), Crohn's disease, Obstructive Lung Diseases ulcerative colitis, rheumatoid arthritis, systemic lupus erythematosus and multiple sclerosis.

In one embodiment of the present invention is the compound as described herein, or a pharmaceutically acceptable salt thereof, for use in the treatment, prevention and/or delay of progression of psoriatic diseases, asthma, Crohn's disease, ulcerative colitis, rheumatoid arthritis, systemic lupus erythematosus and multiple sclerosis.

In one embodiment of the present invention is the compound as described herein, or a pharmaceutically acceptable salt thereof, for the preparation of a medicament for the treatment, prevention and/or delay of progression of psoriatic diseases, asthma, Crohn's disease, ulcerative colitis, rheumatoid arthritis, systemic lupus erythematosus and multiple sclerosis.

In one embodiment of the present invention is the compound as described herein, or a pharmaceutically acceptable salt thereof, for the preparation of a medicament for the treatment, prevention and/or delay of an inflammatory autoimmune disease.

In a further embodiment, the present invention provides a method for the treatment, prevention and/or delay of progression of an inflammatory autoimmune disease, which method comprises administering a therapeutically effective amount of a compound as described herein, or a pharmaceutically acceptable salt thereof.

In a further embodiment, the present invention provides a method for the treatment, prevention and/or delay of progression of psoriatic diseases, asthma, Inflammatory Bowel Diseases (IBD), Crohn's disease, Obstructive Lung Diseases ulcerative colitis, rheumatoid arthritis, systemic lupus erythematosus or multiple, which method comprises administering a therapeutically effective amount of a compound as described herein, or a pharmaceutically acceptable salt thereof.

By the term “treatment” or “treating” and grammatical variations thereof as used herein, is meant therapeutic therapy. In reference to a particular condition, treating means: (1) to ameliorate the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy using the methods and/or compositions of the invention is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing psoriatic diseases, asthma, Inflammatory Bowel Diseases (IBD), Crohn's disease, Obstructive Lung Diseases ulcerative colitis, rheumatoid arthritis, systemic lupus erythematosus or multiple, such as when a subject has a strong family history of psoriatic diseases, asthma, Inflammatory Bowel Diseases (IBD), Crohn's disease, Obstructive Lung Diseases ulcerative colitis, rheumatoid arthritis, systemic lupus erythematosus or multiple.

### EXAMPLES

### Example 1: 4′-[(4-{[4-(trifluoromethoxy)phenyl]Amino}piperidin-1-yl)sulfonyl]-[1,1′-biphenyl]-4-carbonitrile

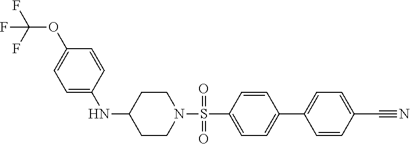 [SMILES: N#Cc1ccc(-c2ccc(S(=O)(=O)N3CCC(Nc4ccc(OC(F)(F)F)cc4)CC3)cc2)cc1]

i) To a solution of tert-butyl N-(piperidin-4-yl)carbamate (1.0 g). and triethylamine (2.1 mL) in THF (40 mL) was added at room temperature 4-bromobenzene-1-sulfonyl chloride (1.4 g). The reaction mixture was stirred overnight at room temperature and concentrated under reduced pressure to give tert-butyl N-[1-(4-bromobenzenesulfonyl)piperidin-4-yl]carbamate (2.0 g), which was used in the next step without further purification.
ii) Under a nitrogen atmosphere, Pd(PPh₃)₄ (14 mg) was added to a suspension of the product obtained in the previous step (500 mg), (4-cyanophenyl)boronic acid (180 mg), and an aqueous 2M K₂CO₃ solution (6.0 mL) in a mixture of toluene (13 mL) and ethanol (1.5 mL). The reaction mixture was stirred for 1 hour at 90° C. in a microwave. After cooling to room temperature, the reaction mixture was diluted with water and ethyl acetate and the product was extracted into ethyl acetate. The combined organic layers were washed with water, brine, dried over MgSO₄, filtered, and concentrated under reduced pressure to give tert-butyl N-[1-({4′-cyano-[1,1′-biphenyl]-4-yl}sulfonyl)piperidin-4-yl]carbamate (500 mg) as a brown oil, which was used in the next step without further purification.
iii) To a solution of the product obtained in the previous step (500 mg) was added at room temperature a 2N HCl solution in diethyl ether (5.7 mL). The reaction mixture was stirred overnight at room temperature and concentrated under reduced pressure to give 4′-[(4-(aminopiperidin)-1-yl)sulfonyl]-[1,1′-biphenyl]-4-carbonitrile hydrochloride (500 mg), which was used in the next step without further purification.
iv) Under a nitrogen atmosphere, Pd₂(dba)₃ (6.1 mg) was added to a suspension of the product obtained in the previous step (50 mg), 1-bromo-4-(trifluoromethoxy)benzene (32 mg), Cs₂CO₃ (215 mg) and BINAP (9.1 mg) in toluene (3 mL). The reaction mixture was stirred overnight at 90° C. in a microwave. After cooling to room temperature, the reaction mixture was dried over MgSO₄, filtered, and concentrated under reduced pressure. The residue was purified on C18, using 10% to 90% CH₃CN in water as the eluent, to give the title compound 4′-[(4-{[4-(trifluoromethoxy)phenyl]Amino}piperidin-1-yl)sulfonyl]-[1,1′-biphenyl]-4-carbonitrile (29 mg) as a white solid. MS(ES⁺) m/z 502.1 (M+H)⁺.

¹H NMR (400 MHz, DMSO) δ 8.08-8.03 (m, 2H), 8.03-7.97 (m, 4H), 7.90-7.85 (m, 2H), 7.03-6.97 (m, 2H), 6.61-6.55 (m, 2H), 5.82-5.77 (d, J=8.1 Hz, 1H), 3.66-3.55 (m, 2H), 3.31-3.20 (m, 1H), 2.63-2.54 (m, 2H), 2.05-1.88 (m, 2H), 1.49-1.34 (m, 2H).

Following a procedure analogous to that described for Example 1, using in step iv the appropriate aryl halide, Example 2 has been prepared.

### Example 2: 4′-[(4-{[3-(difluoromethoxy)phenyl]Amino}piperidin-1-yl)sulfonyl]-[1,1′-biphenyl]-4-carbonitrile

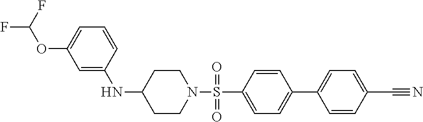 [SMILES: N#Cc1ccc(-c2ccc(S(=O)(=O)N3CCC(Nc4cccc(OC(F)F)c4)CC3)cc2)cc1]

MS(ES⁺) m/z 484.1 (M+H)⁺.

¹H NMR (400 MHz, DMSO) δ 8.08-8.03 (m, 2H), 8.03-7.96 (m, 4H), 7.91-7.85 (m, 2H), 7.28-6.87 (t, J=74.8 Hz, 1H), 7.06-6.99 (t, J=8.1 Hz, 1H), 6.44-6.38 (ddd, J=8.3, 2.1, 0.9 Hz, 1H), 6.33-6.27 (t, J=2.2 Hz, 1H), 6.28-6.22 (dd, J=7.9, 2.2 Hz, 1H), 5.86-5.80 (d, J=8.2 Hz, 1H), 3.67-3.55 (m, 2H), 3.30-3.23 (m, 1H), 2.62-2.54 (m, 2H), 2.02-1.89 (m, 2H), 1.49-1.32 (m, 2H).

Building block: step iv: 1-bromo-3-(difluoromethoxy)benzene.

### Example 3: 6-{4-[(4-{[4-(trifluoromethoxy)phenyl]Amino}piperidin-1-yl)sulfonyl]phenyl}-2,3-dihydro-1H-isoindol-1-one

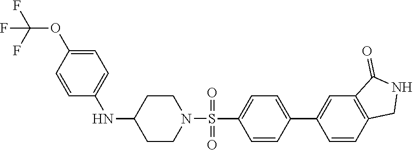 [SMILES: O=C1NCc2ccc(-c3ccc(S(=O)(=O)N4CCC(Nc5ccc(OC(F)(F)F)cc5)CC4)cc3)cc21]

i) Under a nitrogen atmosphere, Pd₂(dba)₃ (229 mg) was added to a suspension of tert-butyl 4-aminopiperidine-1-carboxylate (1.0 g), t-BuONa (1.2 g), 1-bromo-4-(trifluoromethoxy)benzene (890 uL) and Xantphos (347 mg) toluene (10 mL). The reaction mixture was stirred for 2.5 hours at 90° C. in a microwave. After cooling to room temperature, the reaction mixture was diluted with water and ethyl acetate and the product was extracted into ethyl acetate. The combined organic layers were washed with water, brine, dried over MgSO₄, filtered, and concentrated under reduced pressure. The residue was purified on SiO₂, using 0% to 100% ethyl acetate in heptane as the eluent, to give tert-butyl 4-{[4-(trifluoromethoxy)phenyl]Amino}piperidine-1-carboxylate (0.9 g) as a yellow solid.
ii) TFA (1.9 mL) was added to a solution of the product obtained in the previous step (0.9 g) in CH₂Cl₂ (10 mL) and the reaction mixture was stirred overnight at room temperature. After completion the reaction mixture was concentrated under reduced pressure to give N-[4-(trifluoromethoxy)phenyl]piperidin-4-amine; trifluoroacetic acid (1.7 g) as a white solid, which was used in the next step without further purification.
iii) To a suspension of the product obtained in the previous step (0.98 g) and triethyl amine (1.4 mL) in CH₂Cl₂ (25 mL) was added portion wise at room temperature 4-bromobenzene-1-sulfonyl chloride (613 mg). The reaction mixture was stirred overnight at room temperature and quenched by addition of water. The product was extracted into CH₂Cl₂ and the combined organic layers were dried over MgSO₄, filtered, and concentrated under reduced pressure. The obtained brown oil was dissolved in a little ethyl acetate and heptane was added. Overnight the product precipitated and the solid was filtered off and dried under reduced pressure to give 1-(4-bromobenzenesulfonyl)-N-[4-(trifluoromethoxy)phenyl]piperidin-4-amine (0.95 g) as a white solid.
iv) Under a nitrogen atmosphere, Pd(PPh₃)₄ (11 mg) was added to a suspension of the product obtained in the previous step (100 mg), 6-(4,4,5,5-tetramethyl-1,3,2-dioxaborolan-2-yl)-2,3-dihydro-1H-isoindol-1-one (62 mg), and an aqueous 2M K₂CO₃ solution (1.0 mL) in a mixture of toluene (9 mL) and ethanol (1 mL). The reaction mixture was stirred for 1 hour at 100° C. in a microwave. After cooling to room temperature, the reaction mixture was diluted with water and ethyl acetate and the product was extracted into ethyl acetate. The combined organic layers were washed with water, brine, dried over MgSO₄, filtered, and concentrated under reduced pressure. The residue was purified on C18, using 10% to 90% CH3CN in water as the eluent, to give the title compound 6-{4-[(4-{[4-(trifluoromethoxy)phenyl]Amino}piperidin-1-yl)sulfonyl]phenyl}2,3-dihydro-1H-isoindol-1-one (16 mg) as a white solid. MS(ES⁺) m z 532.2 (M+H)⁺.

¹H NMR (400 MHz, DMSO) δ 8.77-8.64 (s, 1H), 8.09-8.03 (m, 2H), 8.03-7.99 (m, 2H), 7.89-7.82 (m, 2H), 7.77-7.72 (m, 1H), 7.07-6.93 (dd, J=8.7, 1.3 Hz, 2H), 6.66-6.52 (m, 2H), 5.86-5.71 (d, J=8.0 Hz, 1H), 4.49-4.43 (s, 2H), 3.67-3.55 (m, 2H), 3.32-3.21 (m, 1H), 2.64-2.53 (m, 2H), 2.04-1.89 (m, 2H), 1.51-1.35 (m, 2H).

Following a procedure analogous to that described for Example 3, using in step i the appropriate (hetero)aryl halide and in step iv the appropriate boronic ester or boronic acid, Examples 4-12 have been prepared.

### Examples 4-12

### Example 4: N-methyl-4′-[(4-{[4-(trifluoromethoxy)phenyl]Amino}piperidin-1-yl)sulfonyl]-[1,1′-biphenyl]-3-carboxamide

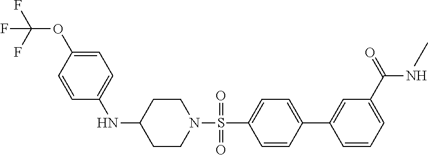

MS(ES⁺) m/z 534.2 (M+H)⁺.

¹H NMR (400 MHz, DMSO) δ 8.66-8.59 (m, 1H), 8.25-8.17 (t, J=1.8 Hz, 1H), 8.06-8.00 (m, 2H), 7.95-7.90 (m, 2H), 7.89-7.85 (m, 2H), 7.66-7.60 (t, J=7.8 Hz, 1H), 7.05-6.95 (m, 2H), 6.62-6.55 (m, 2H), 5.83-5.76 (d, J=8.0 Hz, 1H), 3.67-3.55 (m, 2H), 3.31-3.21 (m, 1H), 2.89-2.79 (d, J=4.5 Hz, 3H), 2.62-2.52 (m, 2H), 2.03-1.92 (m, 2H), 1.52-1.34 (m, 2H).

Building block: step i: 1-bromo-4-(trifluoromethoxy)benzene; step iv: [3-(methylcarbamoyl)phenyl]boronic acid.

### Example 5: 4′-[(4-{[4-(trifluoromethoxy)phenyl]Amino}piperidin-1-yl)sulfonyl]-[1,1′-biphenyl]-3-carboxamide

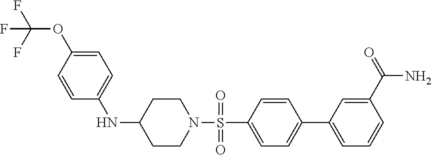 [SMILES: NC(=O)c1cccc(-c2ccc(S(=O)(=O)N3CCC(Nc4ccc(OC(F)(F)F)cc4)CC3)cc2)c1]

MS(ES⁺) m/z 520.2 (M+H)⁺.

¹H NMR (400 MHz, DMSO) δ 8.26 (t, J=1.8, 1.8 Hz, 1H), 8.16 (s, 1H), 8.06-8.01 (m, 2H), 7.94 (tdd, J=7.8, 7.8, 2.3, 1.2 Hz, 2H), 7.89-7.84 (m, 2H), 7.62 (t, J=7.8, 7.8 Hz, 1H), 7.49 (s, 1H), 7.00 (d, J=8.1 Hz, 2H), 6.62-6.54 (m, 2H), 5.79 (d, J=8.1 Hz, 1H), 3.64-3.56 (m, 2H), 3.31-3.22 (m, 1H), 2.60-2.54 (m, 2H), 2.00-1.93 (m, 2H), 1.49-1.34 (m, 2H).

Building block: step i: 1-bromo-4-(trifluoromethoxy)benzene; step iv: (3-carbamoylphenyl)boronic acid.

### Example 6: 1-[4-(3-methyl-1H-indazol-5-yl)benzenesulfonyl]-N-[4-(trifluoromethoxy)phenyl]piperidin-4-amine

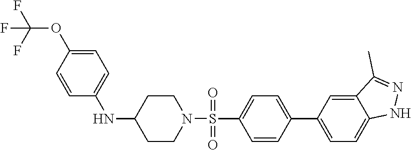 [SMILES: Cc1n[nH]c2ccc(-c3ccc(S(=O)(=O)N4CCC(Nc5ccc(OC(F)(F)F)cc5)CC4)cc3)cc12]

MS(ES⁺) m/z 531.2 (M+H)⁺.

¹H NMR (400 MHz, DMSO) δ 12.79 (s, 1H), 8.13 (dd, J=1.8, 0.9 Hz, 1H), 8.06-7.98 (m, 2H), 7.86-7.78 (m, 2H), 7.74 (dd, J=8.7, 1.7 Hz, 1H), 7.59 (d, J=8.8 Hz, 1H), 7.00 (d, J=8.2 Hz, 2H), 6.62-6.54 (m, 2H), 5.78 (d, J=8.0 Hz, 1H), 3.64-3.55 (m, 2H), 3.30-3.24 (m, 1H), 2.57 (s, 2H), 2.60-2.52 (m, 3H), 2.01-1.93 (m, 2H), 1.50-1.36 (m, 2H).

Building blocks: step i: 1-bromo-4-(trifluoromethoxy)benzene; step iv: (3-methyl-1H-indazol-5-yl)boronic acid.

### Example 7: 4′-[(4-{[5-(trifluoromethoxy)pyridin-2-yl]Amino}piperidin-1-yl)sulfonyl]-[1,1′-biphenyl]-4-carbonitrile

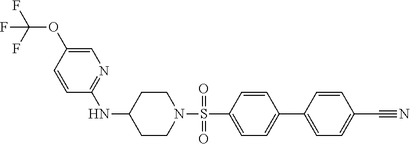 [SMILES: N#Cc1ccc(-c2ccc(S(=O)(=O)N3CCC(Nc4ccc(OC(F)(F)F)cn4)CC3)cc2)cc1]

MS(ES⁺) m/z 503.2 (M+H)⁺.

¹H NMR (400 MHz, DMSO) δ 8.06-8.01 (m, 2H), 8.00 (d, J=1.6 Hz, 3H), 7.94 (d, J=2.9 Hz, 1H), 7.90-7.85 (m, 2H), 7.45-7.36 (m, 1H), 6.85 (d, J=7.3 Hz, 1H), 6.50 (dd, J=9.1, 0.6 Hz, 1H), 3.75-3.63 (m, 1H), 3.61-3.53 (m, 2H), 2.65-2.57 (m, 2H), 2.04-1.89 (m, 2H), 1.56-1.42 (m, 2H).

Building blocks: step i: 2-chloro-5-(trifluoromethoxy)pyridine; step iv: 4-(4,4,5,5-tetramethyl-1,3,2-dioxaborolan-2-yl)benzonitrile.

### Example 8: 6-{4-[(4-{[5-(trifluoromethoxy)pyridin-2-yl]Amino}piperidin-1-yl)sulfonyl]phenyl}-2,3-dihydro-1H-isoindol-1-one

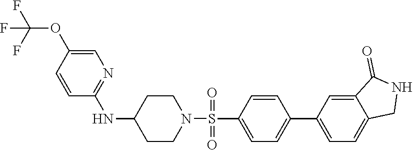 [SMILES: O=C1NCc2ccc(-c3ccc(S(=O)(=O)N4CCC(Nc5ccc(OC(F)(F)F)cn5)CC4)cc3)cc21]

MS(ES⁺) m/z 533.2 (M+H)⁺.

¹H NMR (400 MHz, DMSO) δ 8.73 (s, 1H), 8.12-8.04 (m, 4H), 7.99 (d, J=2.9 Hz, 1H), 7.94-7.86 (m, 2H), 7.83-7.76 (m, 1H), 7.46 (ddd, J=9.2, 3.0, 1.0 Hz, 1H), 6.91 (d, J=7.3 Hz, 1H), 6.55 (d, J=9.2 Hz, 1H), 4.51 (s, 2H), 3.81-3.69 (m, 1H), 3.66-3.58 (m, 2H), 2.66 (t, J=10.5, 10.5 Hz, 2H), 2.07-1.95 (m, 2H), 1.62-1.48 (m, 2H).

Building blocks: step i: 2-chloro-5-(trifluoromethoxy)pyridine; step iv: 6-(4,4,5,5-tetramethyl-1,3,2-dioxaborolan-2-yl)-2,3-dihydro-1H-isoindol-1-one.

### Example 9: 4′-[(4-{[5-(trifluoromethoxy)pyridin-2-yl]Amino}piperidin-1-yl)sulfonyl]-[1,1′-biphenyl]-3-carboxamide

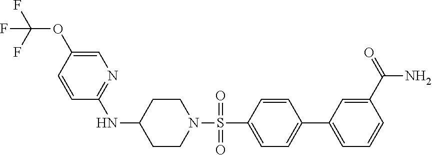 [SMILES: NC(=O)c1cccc(-c2ccc(S(=O)(=O)N3CCC(Nc4ccc(OC(F)(F)F)cn4)CC3)cc2)c1]

MS(ES⁺) m/z 521.2 (M+H)⁺.

¹H NMR (400 MHz, DMSO) δ 8.26 (t, J=1.8, 1.8 Hz, 1H), 8.15 (s, 1H), 8.05-8.00 (m, 2H), 7.98-7.91 (m, 3H), 7.90-7.83 (m, 2H), 7.62 (t, J=7.7, 7.7 Hz, 1H), 7.49 (s, 1H), 7.44-7.36 (m, 1H), 6.85 (d, J=7.2 Hz, 1H), 6.50 (d, J=9.2 Hz, 1H), 3.68 (s, 1H), 3.61-3.53 (m, 2H), 2.65-2.55 (m, 2H), 2.01-1.93 (m, 2H), 1.56-1.43 (m, 2H).

Building blocks: step i: 2-chloro-5-(trifluoromethoxy)pyridine; step iv: (3-carbamoylphenyl)boronic acid.

### Example 10: 6-{4-[(4-{[3-(trifluoromethoxy)phenyl]Amino}piperidin-1-yl)sulfonyl]phenyl}-2,3-dihydro-1H-isoindol-1-one

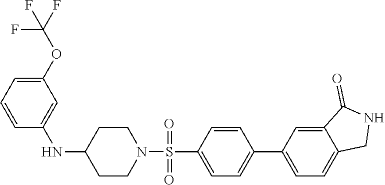 [SMILES: O=C1NCc2ccc(-c3ccc(S(=O)(=O)N4CCC(Nc5cccc(OC(F)(F)F)c5)CC4)cc3)cc21]

m/z 532.2 (M+H)⁺.

¹H NMR (400 MHz, DMSO) δ 8.69 (s, 1H), 8.03-7.93 (m, 4H), 7.84-7.77 (m, 2H), 7.77-7.72 (m, 1H), 7.57 (t, J=8.1, 8.1 Hz, 1H), 7.52-7.37 (m, 3H), 4.47 (s, 2H), 4.44-4.37 (m, 1H), 3.77 (d, J=12.0 Hz, 2H), 2.64-2.55 (m, 2H), 1.95 (d, J=12.2 Hz, 2H), 1.36-1.11 (m, 2H).

Building blocks: step i: 1-bromo-3-(trifluoromethoxy)benzene; step iv: 6-(4,4,5,5-tetramethyl-1,3,2-dioxaborolan-2-yl)-2,3-dihydro-1H-isoindol-1-one.

### Example 11: 1-(4-{imidazo[1,2-a]pyridin-6-yl}benzenesulfonyl)-N-[4-(trifluoromethoxy)phenyl]piperidin-4-amine

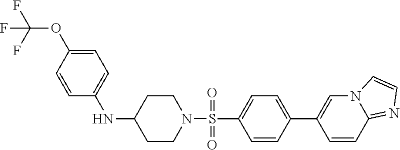 [SMILES: O=S(=O)(c1ccc(-c2ccc3nccn3c2)cc1)N1CCC(Nc2ccc(OC(F)(F)F)cc2)CC1]

MS(ES⁺) m/z 517.3 (M+H)⁺.

¹H NMR (400 MHz, DMSO) δ 9.13-9.07 (dd, J=1.9, 1.0 Hz, 1H), 8.04-7.99 (m, 3H), 7.92-7.83 (m, 2H), 7.74-7.69 (m, 1H), 7.69-7.64 (m, 2H), 7.04-6.96 (m, 2H), 6.65-6.54 (m, 2H), 5.83-5.77 (d, J=8.0 Hz, 1H), 3.66-3.55 (m, 2H), 3.31-3.22 (m, 1H), 2.63-2.52 (m, 2H), 2.01-1.92 (m, 2H), 1.50-1.34 (m, 2H).

Building blocks: step i: 1-bromo-4-(trifluoromethoxy)benzene; step iv: 6-(4,4,5,5-tetramethyl-1,3,2-dioxaborolan-2-yl)imidazo[1,2-a]pyridine.

### Example 12: N-[1-(4-{imidazo[1,2-a]pyridin-6-yl}benzenesulfonyl)piperidin-4-yl]-5-(trifluoromethoxy)pyridin-2-amine

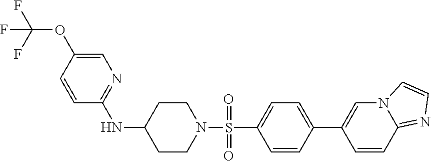 [SMILES: O=S(=O)(c1ccc(-c2ccc3nccn3c2)cc1)N1CCC(Nc2ccc(OC(F)(F)F)cn2)CC1]

MS(ES⁺) m/z 518.3 (M+H)⁺.

¹H NMR (400 MHz, DMSO) δ 9.10 (dd, J=1.9, 1.1 Hz, 1H), 8.05-7.97 (m, 3H), 7.94 (d, J=2.9 Hz, 1H), 7.91-7.83 (m, 2H), 7.72 (d, J=9.4 Hz, 1H), 7.67 (dd, J=10.7, 1.5 Hz, 2H), 7.45-7.37 (m, 1H), 6.86 (d, J=7.3 Hz, 1H), 6.50 (d, J=9.2 Hz, 1H), 3.74-3.63 (m, 1H), 3.61-3.53 (m, 2H), 2.66-2.56 (m, 2H), 2.01-1.93 (m, 2H), 1.57-1.43 (m, 2H).

Building blocks: step i: 2-chloro-5-(trifluoromethoxy)pyridine; step iv: 6-(4,4,5,5-tetramethyl-1,3,2-dioxaborolan-2-yl)imidazo[1,2-a]pyridine.

### Example 13: 5-{4-[(4-{[4-(trifluoromethoxy)phenyl]Amino}piperidin-1-yl)sulfonyl]phenyl}-1H-indole-3-carbonitrile

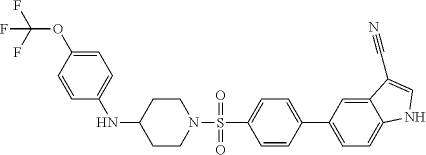 [SMILES: N#Cc1c[nH]c2ccc(-c3ccc(S(=O)(=O)N4CCC(Nc5ccc(OC(F)(F)F)cc5)CC4)cc3)cc12]

i) Following a procedure analogues to that described for Example 3, step i to step iii, 1-(4-bromobenzenesulfonyl)-N-[4-(trifluoromethoxy)phenyl]piperidin-4-amine (1.0 g) was prepared
ii) To a suspension of the product obtained in the previous step (1.0 g), bis(pinacolato)diboron (604 mg) and potassium acetate (538 mg) in DMF (15 mL), purged with N₂ gas, was added PdCl₂(dppf)·CH₂Cl₂ (73 mg). The reaction mixture was heated for 1 h at 100° C. in a microwave. After cooling to room temperature, the reaction mixture was poured into water and the product was extracted into ethyl acetate. The organic layer was washed with brine, dried over MgSO₄ and concentrated under reduced pressure giving 1-[4-(4,4,5,5-tetramethyl-1,3,2-dioxaborolan-2-yl)benzenesulfonyl]-N-[4-(trifluoromethoxy)phenyl]piperidin-4-amine as a brown oil (1.2 g) which was used in the next step without further purification.
iii) Under a nitrogen atmosphere, an aqueous 2M K₂CO₃ solution (1.1 mL) was added to a suspension of the product obtained in the previous step (238 mg), 5-bromo-1H-indole-3-carbonitrile (50 mg), and Pd(PPh₃)₄ (13 mg) in a mixture of toluene (4.5 mL) and ethanol (0.5 mL). The reaction mixture was stirred for 1 hour at 100° C. in a microwave. After cooling to room temperature, the reaction mixture was diluted with water and ethyl acetate and the product was extracted into ethyl acetate. The combined organic layers were washed with water, brine, dried over MgSO₄, filtered, concentrated under reduced pressure. The residue was purified on C18, using 10% to 90% acetonitrile in water as the eluent, to give the title compound 5-{4-[(4-{[4-(trifluoromethoxy)phenyl]Amino}piperidin-1-yl)sulfonyl]phenyl}-1H-indole-3-carbonitrile (20 mg) as a white solid. MS(ES⁺) m/z 531.2 (M+H)⁺.

¹H NMR (400 MHz, DMSO) δ 12.36 (s, 1H), 8.34 (s, 1H), 8.07-8.02 (m, 2H), 8.00 (t, J=1.3, 1.3 Hz, 1H), 7.87-7.79 (m, 2H), 7.74-7.63 (m, 2H), 6.99 (d, J=8.1 Hz, 2H), 6.63-6.54 (m, 2H), 5.78 (d, J=8.0 Hz, 1H), 3.64-3.57 (m, 2H), 3.31-3.21 (m, 1H), 2.62-2.52 (m, 2H), 2.01-1.93 (m, 2H), 1.50-1.36 (m, 2H).

Following a procedure analogous to that described for Example 13, using in step i and step iii the appropriate (hetero)aryl halide, Examples 14-22 have been prepared.

### Examples 14-22

### Example 14: 5-{4-[(4-{[4-(trifluoromethoxy)phenyl]Amino}piperidin-1-yl)sulfonyl]phenyl}-1H-indazole-3-carbonitrile

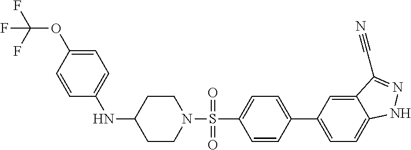 [SMILES: N#Cc1n[nH]c2ccc(-c3ccc(S(=O)(=O)N4CCC(Nc5ccc(OC(F)(F)F)cc5)CC4)cc3)cc12]

MS(ES⁺) m/z 542.2 (M+H)⁺.

¹H NMR (400 MHz, DMSO) δ 14.50 (s, 1H), 8.27 (dd, J=1.6, 0.9 Hz, 1H), 8.14-8.06 (m, 2H), 7.95 (dd, J=8.8, 1.6 Hz, 1H), 7.91 (dd, J=8.8, 0.9 Hz, 1H), 7.88-7.83 (m, 2H), 7.00 (dq, J=7.9, 1.0, 1.0, 1.0 Hz, 2H), 6.62-6.54 (m, 2H), 5.79 (d, J=8.0 Hz, 1H), 3.66-3.57 (m, 2H), 3.29-3.22 (m, OH), 2.64-2.54 (m, 2H), 1.97 (dd, J=13.4, 3.7 Hz, 2H), 1.49-1.37 (m, 2H).

Building block: step i: 1-bromo-4-(trifluoromethoxy)benzene; step iii: 5-bromo-1H-indazole-3-carbonitrile.

### Example 15: 5-{4-[(4-{[5-(trifluoromethoxy)pyridin-2-yl]Amino}piperidin-1-yl)sulfonyl]phenyl}-1H-indole-3-carbonitrile

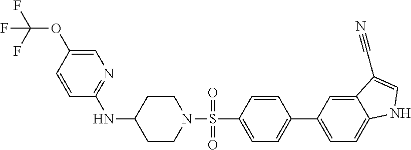 [SMILES: N#Cc1c[nH]c2ccc(-c3ccc(S(=O)(=O)N4CCC(Nc5ccc(OC(F)(F)F)cn5)CC4)cc3)cc12]

MS(ES⁺) m/z 542.2 (M+H)⁺.

¹H NMR (400 MHz, DMSO) δ 12.39-12.34 (s, 1H), 8.37-8.31 (s, 1H), 8.07-7.98 (m, 3H), 7.98-7.93 (d, J=2.9 Hz, 1H), 7.87-7.79 (m, 2H), 7.75-7.67 (d, J=1.3 Hz, 2H), 7.44-7.37 (ddd, J=9.0, 3.1, 1.1 Hz, 1H), 6.89-6.82 (d, J=7.2 Hz, 1H), 6.54-6.47 (d, J=9.2 Hz, 1H), 3.76-3.62 (m, 1H), 3.62-3.50 (m, 2H), 2.66-2.56 (m, 2H), 2.02-1.92 (m, 2H), 1.57-1.43 (m, 2H).

Building block: step i: 2-chloro-5-(trifluoromethoxy)pyridine; step iii: 5-bromo-1H-indole-3-carbonitrile.

### Example 16: 1-(4-{3-methylimidazo[1,2-a]pyridin-6-yl}benzenesulfonyl)-N-[4-(trifluoromethoxy)phenyl]piperidin-4-amine

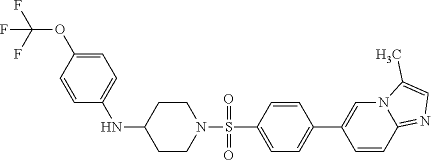 [SMILES: Cc1cnc2ccc(-c3ccc(S(=O)(=O)N4CCC(Nc5ccc(OC(F)(F)F)cc5)CC4)cc3)cn12]

MS(ES⁺) m/z 531.3 (M+H)⁺.

¹H NMR (400 MHz, DMSO) δ 9.02 (t, J=1.4, 1.4 Hz, 1H), 8.23 (dd, J=9.4, 1.7 Hz, 1H), 8.20-8.12 (m, 2H), 8.04 (dd, J=9.3, 0.9 Hz, 1H), 7.99-7.90 (m, 3H), 7.04-6.97 (m, 2H), 6.62-6.54 (m, 2H), 5.80 (s, 1H), 3.67-3.59 (m, 2H), 3.31-3.21 (m, 1H), 2.66 (s, 3H), 2.62-2.54 (m, 2H), 2.02-1.94 (m, 2H), 1.50-1.38 (m, 2H).

Building block: step i: 1-bromo-4-(trifluoromethoxy)benzene; step iii: 6-bromo-3-methylimidazo[1,2-a]pyridine.

### Example 17: 6-{4-[(4-{[4-(trifluoromethoxy)phenyl]Amino}piperidin-1-yl)sulfonyl]phenyl}imidazo[1,2-a]pyridine-3-carbonitrile

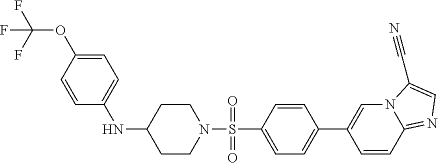 [SMILES: N#Cc1cnc2ccc(-c3ccc(S(=O)(=O)N4CCC(Nc5ccc(OC(F)(F)F)cc5)CC4)cc3)cn12]

MS(ES⁺) m/z 542.3 (M+H)⁺.

¹H NMR (400 MHz, DMSO) δ 8.98 (t, J=1.4, 1.4 Hz, 1H), 8.54 (s, 1H), 8.18-8.10 (m, 2H), 8.01 (t, J=1.4, 1.4 Hz, 2H), 7.93-7.81 (m, 2H), 7.00 (d, J=8.2 Hz, 2H), 6.62-6.54 (m, 2H), 5.79 (d, J=8.0 Hz, 1H), 3.65-3.58 (m, 2H), 3.30-3.22 (m, 1H), 2.62-2.54 (m, 2H), 2.01-1.93 (m, 2H), 1.49-1.37 (m, 2H).

Building block: step i: 1-bromo-4-(trifluoromethoxy)benzene; step iii: 6-bromoimidazo[1,2-a]pyridine-3-carbonitrile.

### Example 18: 1-(4-{2-methyl-[1,2,4]triazolo[1,5-a]pyridin-6-yl}benzenesulfonyl)-N-[4-(trifluoromethoxy)phenyl]piperidin-4-amine

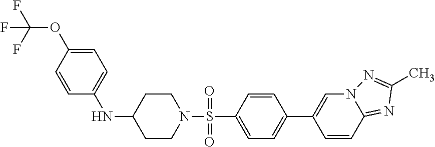 [SMILES: Cc1nc2ccc(-c3ccc(S(=O)(=O)N4CCC(Nc5ccc(OC(F)(F)F)cc5)CC4)cc3)cn2n1]

MS(ES⁺) m/z 532.3 (M+H)⁺.

¹H NMR (400 MHz, DMSO) δ 9.37 (dd, J=1.9, 0.9 Hz, 1H), 8.13-8.09 (m, 2H), 8.06 (dd, J=9.3, 1.9 Hz, 1H), 7.90-7.82 (m, 3H), 7.03-6.96 (m, 2H), 6.62-6.54 (m, 2H), 5.79 (d, J=8.1 Hz, 1H), 3.65-3.57 (m, 2H), 3.31-3.24 (m, 1H), 2.61-2.54 (m, 2H), 2.52 (s, 3H), 2.00-1.93 (m, 2H), 1.48-1.36 (m, 2H).

Building block: step i: 1-bromo-4-(trifluoromethoxy)benzene; step iii: 6-bromo-2-methyl-[1,2,4]triazolo[1,5-a]pyridine.

### Example 19: 1-(4-{2-methyl-[1,2,4]triazolo[1,5-a]pyridin-7-yl}benzenesulfonyl)-N-[4-(trifluoromethoxy)phenyl]piperidin-4-amine

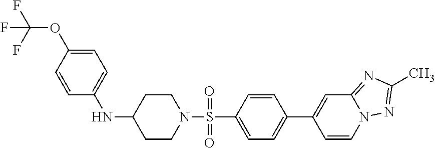 [SMILES: Cc1nc2cc(-c3ccc(S(=O)(=O)N4CCC(Nc5ccc(OC(F)(F)F)cc5)CC4)cc3)ccn2n1]

MS(ES⁺) m/z 532.3 (M+H)⁺.

¹H NMR (400 MHz, CDCl₃) δ 8.59 (dd, J=7.1, 0.9 Hz, 1H), 7.95-7.89 (m, 2H), 7.89-7.78 (m, 3H), 7.22 (dd, J=7.1, 1.9 Hz, 1H), 7.03-6.95 (m, 2H), 6.53-6.44 (m, 2H), 3.83-3.75 (m, 2H), 3.57-3.51 (m, 1H), 2.64 (s, 3H), 2.62-2.57 (m, 2H), 2.18-2.10 (m, 2H), 1.60-1.56 (m, 3H).

Building block: step i: 1-bromo-4-(trifluoromethoxy)benzene; step iii: 7-bromo-2-methyl-[1,2,4]triazolo[1,5-a]pyridine.

### Example 20: 1-{4-[3-(propan-2-yl)imidazo[1,2-a]pyridin-6-yl]benzenesulfonyl}-N-[4-(trifluoromethoxy)phenyl]piperidin-4-amine

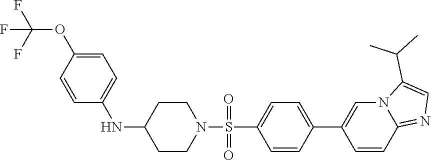 [SMILES: CC(C)c1cnc2ccc(-c3ccc(S(=O)(=O)N4CCC(Nc5ccc(OC(F)(F)F)cc5)CC4)cc3)cn12]

MS(ES⁺) m/z 559.4 (M+H)⁺.

¹H NMR (400 MHz, DMSO) δ 9.15 (t, J=1.3, 1.3 Hz, 1H), 8.30 (dd, J=9.4, 1.6 Hz, 1H), 8.20-8.12 (m, 2H), 8.13-8.05 (m, 2H), 7.99-7.90 (m, 2H), 7.07-6.96 (m, 2H), 6.64-6.53 (m, 2H), 5.82 (s, 1H), 3.75-3.58 (m, 3H), 3.32-3.20 (m, 1H), 2.58 (dd, J=12.4, 9.8 Hz, 2H), 2.04-1.92 (m, 2H), 1.45 (t, J=10.4, 10.4 Hz, 2H), 1.40 (d, J=6.8 Hz, 6H).

Building block: step i: 1-bromo-4-(trifluoromethoxy)benzene; step iii: 6-bromo-3-(propan-2-yl)imidazo[1,2-a]pyridine.

### Example 21: N-(1-{4-[3-(propan-2-yl)imidazo[1,2-a]pyridin-6-yl]benzenesulfonyl}piperidin-4-yl)-5-(trifluoromethoxy)pyridin-2-amine

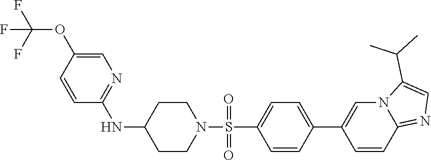 [SMILES: CC(C)c1cnc2ccc(-c3ccc(S(=O)(=O)N4CCC(Nc5ccc(OC(F)(F)F)cn5)CC4)cc3)cn12]

MS(ES⁺) m/z 560.3 (M+H)⁺.

¹H NMR (400 MHz, DMSO) δ 9.17 (s, 1H), 8.33 (dd, J=9.4, 1.6 Hz, 1H), 8.18-8.08 (m, 4H), 7.99-7.90 (m, 3H), 7.48-7.38 (m, 1H), 6.91 (s, 1H), 6.51 (d, J=9.2 Hz, 1H), 3.76-3.55 (m, 3H), 2.63-2.54 (m, 2H), 2.05-1.93 (m, 2H), 1.59-1.45 (m, 2H), 1.40 (d, J=6.8 Hz, 6H).

Building block: step i: 2-chloro-5-(trifluoromethoxy)pyridine; step iii: 6-bromo-3-(propan-2-yl)imidazo[1,2-a]pyridine.

### Example 22: 1-{4-[3-(methoxymethyl)-[1,2,4]triazolo[4,3-a]pyridin-6-yl]benzenesulfonyl}-N-[4-(trifluoromethoxy)phenyl]piperidin-4-amine

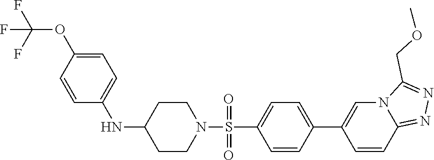 [SMILES: COCc1nnc2ccc(-c3ccc(S(=O)(=O)N4CCC(Nc5ccc(OC(F)(F)F)cc5)CC4)cc3)cn12]

MS(ES⁺) m/z 562.4 (M+H)⁺.

¹H NMR (400 MHz, DMSO) δ 8.85 (t, J=1.4, 1.4 Hz, 1H), 8.12-8.04 (m, 2H), 7.97 (dd, J=9.6, 1.1 Hz, 1H), 7.93-7.88 (m, 2H), 7.87 (dd, J=9.6, 1.7 Hz, 1H), 7.04-6.97 (m, 2H), 6.62-6.54 (m, 2H), 5.79 (d, J=8.0 Hz, 1H), 5.07 (s, 2H), 3.65-3.56 (m, 2H), 3.35 (s, 3H), 3.29-3.21 (m, OH), 2.62-2.52 (m, 3H), 2.02-1.93 (m, 2H), 1.50-1.36 (m, 2H).

Building block: step i: 1-bromo-4-(trifluoromethoxy)benzene; step iii: 6-bromo-3-(methoxymethyl)-[1,2,4]triazolo[4,3-a]pyridine.

### Example 23: 6-{4-[(4-{[4-(trifluoromethoxy)phenyl]Amino}piperidin-1-yl)sulfonyl]phenyl}-2H,3H-[1,2,4]triazolo[4,3-a]pyridin-3-one

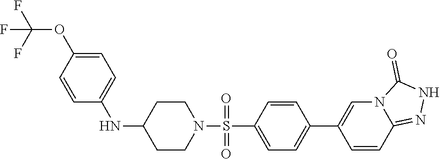 [SMILES: O=c1[nH]nc2ccc(-c3ccc(S(=O)(=O)N4CCC(Nc5ccc(OC(F)(F)F)cc5)CC4)cc3)cn12]

i) Following a procedure analogous to that described for Example 13, using in step iv 6-bromo-2-[(4-methoxyphenyl)methyl]-2H,3H-[1,2,4]triazolo[4,3-a]pyridin-3-one (78 mg), 2-[(4-methoxyphenyl)methyl]-6-{4-[(4-{[4-(trifluoromethoxy)phenyl]Amino}piperidin-1-yl)sulfonyl]phenyl}-2H,3H-[1,2,4]triazolo[4,3-a]pyridin-3-one (60 mg) has been prepared as a white solid, which was used in the next step without further purification.
ii) A solution of the product obtained in the previous step (60 mg) and L-cysteine (17 mg) in trifluoroacetic acid (5 mL) was stirred for 6 hours at 70° C. After cooling to room temperature the reaction mixture was concentrated under reduced pressure and the residue was purified on C18, using 10% to 90% acetonitrile in water as the eluent, giving the title compound 6-{4-[(4-{[4-(trifluoromethoxy)phenyl]Amino}piperidin-1-yl)sulfonyl]phenyl}2H,3H-[1,2,4]triazolo[4,3-a]pyridin-3-one (42 mg) as a white solid. MS(ES⁺) m/z 534.3 (M+H)⁺.

¹H NMR (400 MHz, DMSO) δ 12.62 (s, 1H), 8.23 (dd, J=1.8, 1.1 Hz, 1H), 8.07-7.99 (m, 2H), 7.85-7.78 (m, 2H), 7.66 (dd, J=9.8, 1.8 Hz, 1H), 7.40 (dd, J=9.8, 1.1 Hz, 1H), 7.03-6.96 (m, 2H), 6.62-6.53 (m, 2H), 5.78 (d, J=8.1 Hz, 1H), 3.63-3.55 (m, 2H), 3.33-3.18 (m, 1H), 2.60-2.52 (m, 2H), 1.99-1.92 (m, 2H), 1.48-1.35 (m, 2H).

Following a procedure analogous to that described for Example 23, using in Example 3, step i, the appropriate aryl halide, Example 24 has been prepared.

### Example 24: 6-{4-[(4-{[5-(trifluoromethoxy)pyridin-2-yl]Amino}piperidin-1-yl)sulfonyl]phenyl}-2H,3H-[1,2,4]triazolo[4,3-a]pyridin-3-one

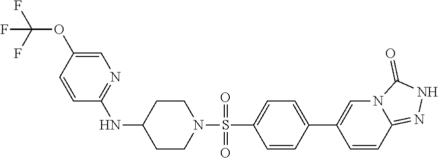 [SMILES: O=c1[nH]nc2ccc(-c3ccc(S(=O)(=O)N4CCC(Nc5ccc(OC(F)(F)F)cn5)CC4)cc3)cn12]

MS(ES⁺) m/z 535.3 (M+H)⁺.

¹H NMR (400 MHz, DMSO) δ 12.61 (s, 1H), 8.23 (t, J=1.5, 1.5 Hz, 1H), 8.05-7.98 (m, 2H), 7.94 (d, J=2.9 Hz, 1H), 7.85-7.78 (m, 2H), 7.66 (dd, J=9.8, 1.8 Hz, 1H), 7.45-7.36 (m, 2H), 6.87 (d, J=7.3 Hz, 1H), 6.50 (d, J=9.2 Hz, 1H), 3.71-3.64 (m, 1H), 3.60-3.52 (m, 2H), 2.64-2.55 (m, 2H), 2.00-1.92 (m, 2H), 1.56-1.42 (m, 2H).

Building block: step i: 2-chloro-5-(trifluoromethoxy)pyridine.

### Example 25: 1-{[1,1′-biphenyl]-4-sulfonyl}-N-[4-(trifluoromethoxy)phenyl]piperidin-4-amine

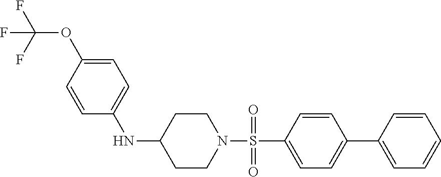 [SMILES: O=S(=O)(c1ccc(-c2ccccc2)cc1)N1CCC(Nc2ccc(OC(F)(F)F)cc2)CC1]

i) To a suspension of piperidin-4-one (1.0 g) and triethyl amine (4.2 mL) in CH₂Cl₂ (20 mL) was added portion wise at room temperature [1,1′-biphenyl]-4-sulfonyl chloride (2.8 g). The reaction mixture was stirred overnight at room temperature and quenched by addition of water. The product was extracted into ethyl acetate and the combined organic layers were washed with brine, dried over MgSO₄, filtered, and concentrated under reduced pressure. The obtained brown oil was dissolved in a little ethyl acetate and heptane was added. Overnight the product precipitated and the solid was filtered off and dried under reduced pressure to give 1-{[1,1′-biphenyl]-4-sulfonyl}piperidin-4-one (1.41 g) as a white solid.
ii) Under a nitrogen atmosphere, 2-Methylpyridine borane complex (17 mg) was added at 0° C., to a solution of the product obtained in the previous step (50 mg), 4-(trifluoromethoxy)aniline (21 uL) and trifluoroacetic acid (0.2 mL) in methanol (2 mL). The reaction mixture was stirred over night at room temperature. After the reaction mixture was concentrated under reduced pressure an aqueous 2N HCl solution (3 mL) was added at 0° C., and the resulting mixture was stirred for 1 hour at room temperature. The mixture was basified by the addition of an aqueous 6N NaOH solution (5 mL) and the product was extracted into CH₂C₂. The combined organic layers were washed with brine, dried over MgSO₄, filtered, and concentrated under reduced pressure. The residue was purified on C18, using 10% to 90% CH3CN in water as the eluent, to give the title compound 1-{[1,1′-biphenyl]-4-sulfonyl}-N-[4-(trifluoromethoxy)phenyl]piperidin-4-amine (28 mg) as a white solid. MS(ES⁺) m/z 477.1 (M+H)⁺.

¹H NMR (400 MHz, DMSO) δ 8.00-7.93 (m, 2H), 7.87-7.80 (m, 2H), 7.80-7.75 (m, 2H), 7.58-7.51 (m, 2H), 7.50-7.44 (m, 1H), 7.03-6.96 (m, 2H), 6.62-6.54 (m, 2H), 5.78 (d, J=8.1 Hz, 1H), 3.63-3.56 (m, 2H), 3.31-3.19 (m, 1H), 2.61-2.54 (m, 2H), 2.00-1.93 (m, 2H), 1.49-1.35 (m, 2H).

Following a procedure analogous to that described for Example 25, using in step ii the appropriate anilin, Examples 26-30 have been prepared.

### Example 26: 1-{[1,1′-biphenyl]-4-sulfonyl}-N-[4-(difluoromethoxy)phenyl]piperidin-4-amine

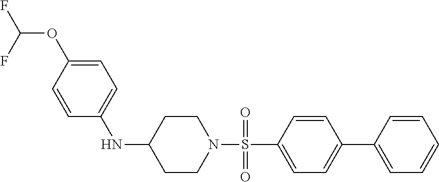 [SMILES: O=S(=O)(c1ccc(-c2ccccc2)cc1)N1CCC(Nc2ccc(OC(F)F)cc2)CC1]

m/z 514.3 (M+H)⁺.

¹H NMR (400 MHz, DMSO) δ 8.03-7.93 (m, 2H), 7.86-7.80 (m, 2H), 7.80-7.75 (m, 2H), 7.58-7.51 (m, 2H), 7.50-7.44 (m, 1H), 7.11-6.71 (t, J=75.3 Hz, 1H), 6.90-6.84 (m, 2H), 6.61-6.51 (m, 2H), 5.58-5.51 (d, J=8.2 Hz, 1H), 3.65-3.55 (m, 2H), 3.33-3.17 (m, 1H), 2.63-2.52 (m, 2H), 2.01-1.91 (m, 2H), 1.48-1.34 (m, 2H).

Building block: step ii: 4-(difluoromethoxy)aniline.

### Example 27: N-[4-(difluoromethoxy)phenyl]-1-[4-(3-methyl-1H-indazol-5-yl)benzenesulfonyl]piperidin-4-amine

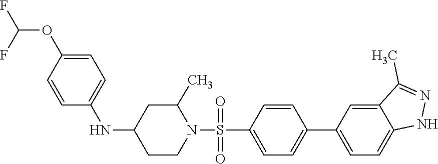 [SMILES: Cc1n[nH]c2ccc(-c3ccc(S(=O)(=O)N4CCC(Nc5ccc(OC(F)F)cc5)CC4C)cc3)cc12]

i) To a suspension of piperidin-4-one (10.0 g) and triethyl amine (27 mL) in CH₂Cl₂ (200 mL) was added portion wise at room temperature 4-bromobenzene-1-sulfonyl chloride (16.6 g). The reaction mixture was stirred over weekend at room temperature and quenched by addition of water. The product was extracted into CH₂Cl₂ and the combined organic layers were washed with brine, dried over MgSO₄, filtered and concentrated under reduced pressure. The obtained brown oil was dissolved in a little ethyl acetate and heptane was added. Overnight the product precipitated and the solid was filtered off and dried under reduced pressure to give 1-(4-bromobenzenesulfonyl)piperidin-4-one (15.1 g) as an off-white solid, which was used in the next step without further purification.
ii) Under a nitrogen atmosphere, 2-Methylpyridine borane complex (168 mg) was added at 0° C., to a solution of the product obtained in the previous step (500 mg), 4-(difluoromethoxy)aniline (300 mg) and trifluoroacetic acid (1.5 mL) in methanol (15 mL). The reaction mixture was stirred over night at room temperature. After the reaction mixture was concentrated under reduced pressure an aqueous 2N HCl solution (5 mL) was added at 0° C., and the resulting mixture was stirred for 0.5 hour at room temperature. The mixture was basified by the addition of an aqueous 6N NaOH solution (10 mL) and the product was extracted into CH₂Cl₂. The combined organic layers were washed with brine, dried over MgSO₄, filtered, and concentrated under reduced pressure to give 1-(4-bromobenzenesulfonyl)-N-[4-(difluoromethoxy)phenyl]piperidin-4-amine (766 mg) as a brown solid, which was used in the next step without further purification.
iii) Under a nitrogen atmosphere, Pd(PPh₃)₄ (12 mg) was added to a suspension of the product obtained in the previous step (100 mg), (3-methyl-1H-indazol-5-yl)boronic acid (55 mg), and NaHCO₃ (105 mg) in a mixture of 1,4-dioxane (4 mL) and water (1 mL). The reaction mixture was stirred for 1 hour at 110° C. in a microwave. After cooling to room temperature, the reaction mixture was diluted with water and ethyl acetate and the product was extracted into ethyl acetate. The combined organic layers were washed with water, brine, dried over MgSO₄, filtered, and concentrated under reduced pressure. The obtained brown oil was purified on C18, using 10% to 90% acetonitrile in water as the eluent, to give the title compound N-[4-(difluoromethoxy)phenyl]-2-methyl-1-[4-(3-methyl-1H-indazol-5-yl)benzenesulfonyl]piperidin-4-amine (25 mg) as a white solid.

MS(ES⁺) m/z 513.2 (M+H)⁺.

¹H NMR (400 MHz, DMSO) δ 12.81-12.76 (s, 1H), 8.16-8.10 (dd, J=1.8, 0.8 Hz, 1H), 8.06-7.98 (m, 2H), 7.86-7.78 (m, 2H), 7.78-7.71 (dd, J=8.8, 1.7 Hz, 1H), 7.63-7.56 (dd, J=8.6, 0.8 Hz, 1H), 7.11-6.70 (t, J=75.3 Hz, 1H), 6.89-6.83 (m, 2H), 6.60-6.51 (m, 2H), 5.58-5.51 (d, J=8.2 Hz, 1H), 3.65-3.55 (m, 2H), 3.31-3.17 (m, 1H), 2.58-2.56 (s, 3H), 2.60-2.52 (m, 2H), 2.01-1.92 (m, 2H), 1.49-1.34 (m, 2H).

Following a procedure analogous to that described for Example 27, using in step ii the appropriate aniline and in step iii the appropriate boronic ester or boronic acid, Examples 28-30 have been prepared.

### Examples 28-30

### Example 28: 6-{4-[(4-{[4-(difluoromethoxy)phenyl]Amino}piperidin-1-yl)sulfonyl]phenyl}-2,3-dihydro-1H-isoindol-1-one

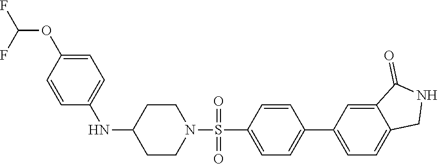 [SMILES: O=C1NCc2ccc(-c3ccc(S(=O)(=O)N4CCC(Nc5ccc(OC(F)F)cc5)CC4)cc3)cc21]

MS(ES⁺) m/z 514.3 (M+H)⁺.

¹H NMR (400 MHz, DMSO) δ 8.71-8.66 (s, 1H), 8.10-7.95 (m, 5H), 7.89-7.81 (m, 2H), 7.78-7.71 (d, J=8.5 Hz, 1H), 7.11-6.70 (t, J=75.3 Hz, 1H), 6.89-6.84 (m, 2H), 6.60-6.50 (m, 2H), 5.58-5.51 (d, J=8.2 Hz, 1H), 4.49-4.44 (s, 2H), 3.64-3.57 (m, 2H), 3.29-3.21 (m, 1H), 2.62-2.51 (m, 2H), 2.00-1.92 (m, 2H), 1.48-1.34 (m, 2H).

Building block: step ii: 4-(difluoromethoxy)aniline; step iii: 6-(4,4,5,5-tetramethyl-1,3,2-dioxaborolan-2-yl)-2,3-dihydro-11H-isoindol-1-one.

### Example 29: 5-{4-[(4-{[4-(difluoromethoxy)phenyl]Amino}piperidin-1-yl)sulfonyl]phenyl}-1H-indole-3-carbonitrile

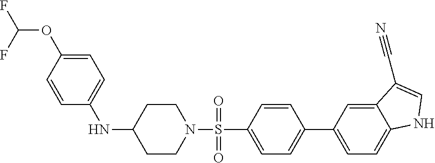 [SMILES: N#Cc1c[nH]c2ccc(-c3ccc(S(=O)(=O)N4CCC(Nc5ccc(OC(F)F)cc5)CC4)cc3)cc12]

MS(ES⁺) m/z 523.3 (M+H)⁺.

¹H NMR (400 MHz, DMSO) δ 13.22-11.37 (s, 1H), 8.38-8.31 (s, 1H), 8.07-8.01 (m, 2H), 8.01-7.98 (t, J=1.3 Hz, 1H), 7.87-7.78 (m, 2H), 7.78-7.65 (m, 2H), 7.11-6.71 (t, J=75.3 Hz, 1H), 6.89-6.84 (m, 2H), 6.60-6.51 (m, 2H), 5.58-5.51 (d, J=8.1 Hz, 11H), 3.65-3.55 (m, 2H), 3.30-3.17 (m, 11H), 2.59-2.53 (m, 2H), 2.01-1.92 (m, 2H), 1.49-1.34 (m, 2H).

Building block: step ii: 4-(difluoromethoxy)aniline; step iii: 5-(4,4,5,5-tetramethyl-1,3,2-dioxaborolan-2-yl)-1H-indole-3-carbonitrile.

### Example 30: 1-(4-{imidazo[1,2-a]pyridin-6-yl}benzenesulfonyl)-N-{4-[(trifluoromethyl)sulfanyl]phenyl}piperidin-4-amine

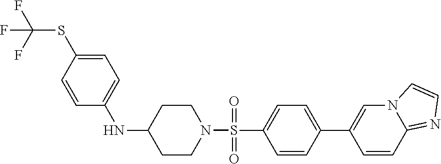 [SMILES: O=S(=O)(c1ccc(-c2ccc3nccn3c2)cc1)N1CCC(Nc2ccc(SC(F)(F)F)cc2)CC1]

MS(ES⁺) m/z 533.3 (M+H)⁺.

¹H NMR (400 MHz, DMSO) δ 9.11 (t, J=1.5, 1.5 Hz, 1H), 8.04-8.00 (m, 3H), 7.95-7.82 (m, 2H), 7.73 (d, J=9.4 Hz, 1H), 7.71-7.66 (m, 2H), 7.35-7.27 (m, 2H), 6.68-6.57 (m, 2H), 6.32 (d, J=7.9 Hz, 1H), 3.66-3.58 (m, 2H), 3.38-3.35 (m, 1H), 2.62-2.54 (m, 2H), 2.02-1.93 (m, 2H), 1.52-1.38 (m, 2H).

Building block: step ii: 4-[(trifluoromethyl)sulfanyl]aniline; step iii: 6-(4,4,5,5-tetramethyl-1,3,2-dioxaborolan-2-yl)imidazo[1,2-a]pyridine.

### Example 31: 1-{4-[3-(propan-2-yl)imidazo[1,2-a]pyridin-6-yl]benzenesulfonyl}-N-{4-[(trifluoromethyl)sulfanyl]phenyl}piperidin-4-amine

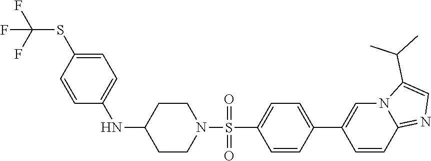 [SMILES: CC(C)c1cnc2ccc(-c3ccc(S(=O)(=O)N4CCC(Nc5ccc(SC(F)(F)F)cc5)CC4)cc3)cn12]

i) Following a procedure analogous to that described for Example 27, step ii, using 4-[(trifluoromethyl)sulfanyl]aniline (97 mg), 1-(4-bromobenzenesulfonyl)-N-{4-[(trifluoromethyl)sulfanyl]phenyl}piperidin-4-amine (2.58 g) has been prepared as a white solid.
ii) To a suspension of the product obtained in the previous step (200 mg), bis(pinacolato)diboron (110 mg) and potassium acetate (97 mg) in cyclopentyl methyl ether (2 mL), purged with N₂ gas, was added PdCl₂(dppf)·CH₂Cl₂ (19 mg). The reaction mixture was heated for 2 hours at 100° C. in a microwave. After cooling to room temperature, the reaction mixture was poured into water and the product was extracted into ethyl acetate. The organic layer was washed with brine, dried over MgSO₄ and concentrated under reduced pressure giving 1-[4-(4,4,5,5-tetramethyl-1,3,2-dioxaborolan-2-yl)benzenesulfonyl]-N-{4-[(trifluoromethyl)sulfanyl]phenyl}piperidin-4-amine as a brown oil (290 mg) which was used in the next step without further purification.
iii) Under a nitrogen atmosphere, Pd(PPh₃)₄ (17 mg) was added to a suspension of the product obtained in the previous step (290 mg), 6-bromo-3-(propan-2-yl)imidazo[1,2-a]pyridine (41 mg), and NaHCO₃ (73 mg) in a mixture of 1,4-dioxane (4 mL) and water (1 mL). The reaction mixture was stirred for 3 hours at 100° C. in a microwave. After cooling to room temperature, the reaction mixture was diluted with water and ethyl acetate and the product was extracted into ethyl acetate. The combined organic layers were washed with water, brine, dried over MgSO₄, filtered, concentrated under reduced pressure. The residue was purified on C18, using 20% to 50% acetonitrile in water as the eluent, to give the title compound 1-{4-[3-(propan-2-yl)imidazo[1,2-a]pyridin-6-yl)benzenesulfonyl]-N-{4-[trifluoromethyl)sulfanyl]phenyl}piperidin-4-amine (5 mg) as a white solid. MS(ES⁺) m/z 575.3 (M+H)⁺.

¹H NMR (400 MHz, DMSO) δ 8.74-8.69 (m, 1H), 8.13-8.05 (m, 2H), 7.90-7.82 (m, 2H), 7.70 (dd, J=9.4, 0.9 Hz, 1H), 7.63 (dd, J=9.5, 1.9 Hz, 1H), 7.47 (d, J=0.8 Hz, 1H), 7.35-7.27 (m, 2H), 6.68-6.59 (m, 2H), 6.32 (d, J=7.8 Hz, 1H), 3.66-3.59 (m, 2H), 3.52 (hept, J=6.8, 6.8, 6.8, 6.8, 6.8, 6.8 Hz, 1H), 3.39-3.33 (m, 1H), 2.62-2.52 (m, 2H), 2.01-1.94 (m, 2H), 1.53-1.44 (m, 2H), 1.37 (d, J=6.8 Hz, 6H).

Following a procedure analogous to that described for Example 31, using in step i the appropriate aniline and in step iii the appropriate (hetero)aryl halide, Examples 32-34 have been prepared.

### Example 32: 1-(4-{3-cyclopentyl-[1,2,4]triazolo[4,3-a]pyridin-6-yl}benzenesulfonyl)-N-[4-(trifluoromethoxy)phenyl]piperidin-4-amine

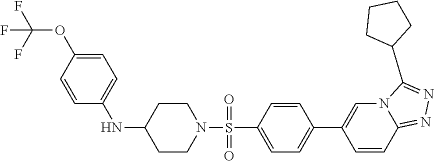 [SMILES: O=S(=O)(c1ccc(-c2ccc3nnc(C4CCCC4)n3c2)cc1)N1CCC(Nc2ccc(OC(F)(F)F)cc2)CC1]

MS(ES⁺) m/z 586.3 (M+H)⁺.

¹H NMR (400 MHz, DMSO) δ 8.84 (t, J=1.5, 1.5 Hz, 1H), 8.14-8.06 (m, 2H), 7.92-7.82 (m, 3H), 7.77 (dd, J=9.6, 1.7 Hz, 1H), 7.04-6.97 (m, 2H), 6.62-6.54 (m, 2H), 5.79 (d, J=8.0 Hz, 1H), 3.81 (p, J=7.9, 7.9, 7.9, 7.9 Hz, 1H), 3.65-3.57 (m, 2H), 3.28-3.23 (m, 1H), 2.62-2.53 (m, 2H), 2.29-2.16 (m, 2H), 2.03-1.90 (m, 4H), 1.88-1.67 (m, 4H), 1.50-1.36 (m, 2H).

Building blocks: step i: 4-(trifluoromethoxy)aniline; step iii: 6-bromo-3-cyclopentyl-[1,2,4]triazolo[4,3-a]pyridine.

### Example 33: 1-{4-[8-methyl-3-(propan-2-yl)imidazo[1,2-a]pyridin-6-yl]benzenesulfonyl}-N-{4-[(trifluoromethyl)sulfanyl]phenyl}piperidin-4-amine

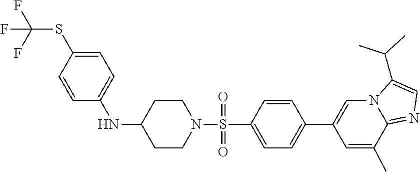 [SMILES: Cc1cc(-c2ccc(S(=O)(=O)N3CCC(Nc4ccc(SC(F)(F)F)cc4)CC3)cc2)cn2c(C(C)C)cnc12]

MS(ES⁺) m/z 589.3 (M+H)⁺

Building blocks: step i: 4-[(trifluoromethyl)sulfanyl]aniline; step iii: 6-bromo-8-methyl-3-(propan-2-yl)imidazo[1,2-a]pyridine.

### Example 34: 4-(4-{[4-({4-[(trifluoromethyl)sulfanyl]phenyl}Amino)piperidin-1-yl]sulfonyl}phenyl)pyridine-2-carboxamide

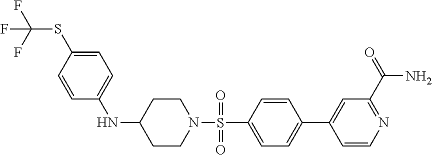 [SMILES: NC(=O)c1cc(-c2ccc(S(=O)(=O)N3CCC(Nc4ccc(SC(F)(F)F)cc4)CC3)cc2)ccn1]

MS(ES⁺) m/z 537.4 (M+H)⁺

Building blocks: step i: 4-[(trifluoromethyl)sulfanyl]aniline; step iii: 6 4-bromopyridine-2-carboxamide.

### Example 35: 1-{[1,1′-biphenyl]-4-sulfonyl}-2-methyl-N-[4-(trifluoromethoxy)phenyl]piperidin-4-amine

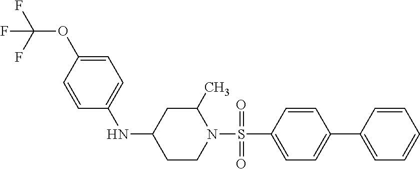 [SMILES: CC1CC(Nc2ccc(OC(F)(F)F)cc2)CCN1S(=O)(=O)c1ccc(-c2ccccc2)cc1]

i) Under a nitrogen atmosphere, 2-Methylpyridine borane complex (17 mg) was added at 0° C., to a suspension of tert-butyl 2-methyl-4-oxopiperidine-1-carboxylate (500 mg), 4-(trifluoromethoxy)aniline (21 uL) and trifluoroacetic acid (1 mL) in methanol (10 mL). The reaction mixture was stirred overnight at room temperature. After the reaction mixture was concentrated under reduced pressure, an aqueous 2N HCl solution (3 mL) was added at 0° C., and the resulting mixture was stirred for 1 hour at room temperature. The mixture was basified by the addition of an aqueous 6N NaOH solution (5 mL) and the product was extracted into CH₂Cl₂. The combined organic layers were washed with brine, dried over MgSO₄, filtered, and concentrated under reduced pressure. The residue was purified on SiO₂, using 0% to 100% ethyl acetate in heptane as the eluent, to give tert-butyl 2-methyl-4-{[4-trifluoromethoxy)phenyl]Amino}piperidine-1-carboxylate (694 mg) as a white solid.
ii) To a solution of the product obtained in the previous step (694 mg) was added at room temperature a 4N HCl solution in 2-propanol. The reaction mixture was stirred overnight at room temperature and concentrated under reduced pressure to give 2-methyl-N-[4-(trifluoromethoxy)phenyl]piperidin-4-amine hydrochloride (836 mg), which was used in the next step without further purification.
iii) To a suspension of the product obtained in the previous step (100 mg) and triethyl amine (105 uL) in CH₂Cl₂ (5 mL) was added portion wise at room temperature, a solution of [1,1′-biphenyl]-4-sulfonyl chloride (70 mg) in CH₂Cl₂ (5 mL). The reaction mixture was stirred overnight at room temperature and quenched by addition of water. The product was extracted into CH₂Cl₂ and the combined organic layers were washed with brine, dried over MgSO₄, filtered and concentrated under reduced pressure. The residue was purified on C18, using 10% to 90% CH3CN in water as the eluent, to give the title compound 1-{[1,1′-biphenyl]-4-sulfonyl}-2-methyl-N-[4-(trifluoromethoxy)phenyl]piperidin-4-amine (1.41 g) as a white solid. MS(ES⁺) m/z 491.2 (M+H)⁺.

¹H NMR (400 MHz, DMSO) δ 7.99-7.86 (m, 4H), 7.80-7.72 (m, 2H), 7.56-7.49 (m, 2H), 7.48-7.42 (m, 1H), 7.00-6.95 (d, J=8.8 Hz, 2H), 6.56-6.46 (m, 2H), 5.81-5.74 (d, J=7.4 Hz, 1H), 3.83-3.68 (m, 1H), 3.54-3.41 (m, 1H), 3.29-3.12 (m, 2H), 1.96-1.86 (ddt, J=12.8, 9.0, 4.6 Hz, 1H), 1.83-1.75 (dt, J=14.1, 4.1 Hz, OH), 1.51-1.39 (m, 2H), 1.29-1.22 (d, J=6.6 Hz, 3H).

### Example 36: 4′-[(2-methyl-4-{[4-(trifluoromethoxy)phenyl]Amino}piperidin-1-yl)sulfonyl]-[1,1′-biphenyl]-4-carbonitrile

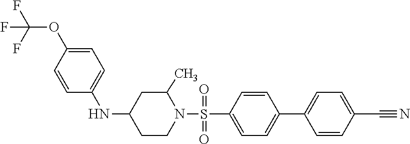 [SMILES: CC1CC(Nc2ccc(OC(F)(F)F)cc2)CCN1S(=O)(=O)c1ccc(-c2ccc(C#N)cc2)cc1]

i) To a suspension of 2-methyl-N-[4-(trifluoromethoxy)phenyl]piperidin-4-amine hydrochloride (example 35, step ii, 700 mg) and triethyl amine (736 uL) in CH₂Cl₂ (40 mL) was added portion wise at room temperature, 4-bromobenzene-1-sulfonyl chloride (495 mg. The reaction mixture was stirred overnight at room temperature and quenched by addition of water. The product was extracted into CH₂Cl₂ and the combined organic layers were washed with brine, dried over MgSO₄, filtered and concentrated under reduced pressure. The residue was purified on C18, using 10% to 90% CH3CN in water as the eluent, to give 1-(4-bromobenzenesulfonyl)-2-methyl-N-[4-(trifluoromethoxy)phenyl]piperidin-4-amine (385 mg) as a white solid.
ii) Under a nitrogen atmosphere, an aqueous 2N NaHCO₃ solution (1.2 mL) was added to a suspension of the product obtained in the previous step (128 mg), (4-cyanophenyl)boronic acid (37 mg), and Pd(PPh₃)₄ (13 mg) in a mixture of toluene (9 mL) and ethanol (1 mL). The reaction mixture was stirred for 1 hour at 100° C. in a microwave. After cooling to room temperature, the reaction mixture was diluted with water and ethyl acetate and the product was extracted into ethyl acetate. The combined organic layers were washed with water, brine, dried over MgSO₄, filtered, and concentrated under reduced pressure. The obtained brown oil was purified on C18₂, using 10% to 90% acetonitrile in water as the eluent, to give the title compound 4′-[(2-methyl-4-{[4-(trifluoromethoxy)phenyl]Amino}piperidin-1-yl)sulfonyl]-[1,1′-biphenyl]-4-carbonitrile (53 mg) as a white solid. MS(ES⁺) m/z 516.3 (M+H)⁺.

¹H NMR (400 MHz, DMSO) δ 8.09-7.90 (m, 8H), 7.00-6.95 (d, J=8.4 Hz, 3H), 6.55-6.47 (m, 2H), 5.82-5.73 (d, J=7.4 Hz, 1H), 3.80-3.69 (ddd, J=12.5, 8.0, 3.9 Hz, 1H), 3.55-3.45 (m, 1H), 3.27-3.16 (m, 2H), 1.96-1.85 (ddt, J=12.8, 8.9, 4.8 Hz, 1H), 1.83-1.73 (dt, J=13.4, 4.2 Hz, 1H), 1.53-1.38 (m, 2H), 1.28-1.22 (d, J=6.6 Hz, 3H).

Following a procedure analogous to that described for Example 36, using in step ii the appropriate boronic ester or boronic acid, Examples 37-38 have been prepared.

### Examples 37-38

### Example 37: 6-{4-[(2-methyl-4-{[4-(trifluoromethoxy)phenyl]Amino}piperidin-1-yl)sulfonyl]phenyl}-2,3-dihydro-1H-isoindol-1-one

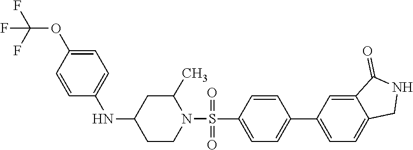 [SMILES: CC1CC(Nc2ccc(OC(F)(F)F)cc2)CCN1S(=O)(=O)c1ccc(-c2ccc3c(c2)C(=O)NC3)cc1]

MS(ES⁺) m/z 546.3 (M+H)⁺.

¹H NMR (400 MHz, DMSO) δ 8.70-8.65 (s, 1H), 8.07-7.98 (m, 4H), 7.98-7.89 (m, 2H), 7.77-7.69 (m, 1H), 7.01-6.93 (d, J=8.3 Hz, 2H), 6.63-6.47 (m, 2H), 5.81-5.74 (d, J=7.5 Hz, 1H), 4.48-4.43 (s, 2H), 3.80-3.68 (ddd, J=12.5, 7.9, 3.9 Hz, 1H), 3.53-3.41 (m, 1H), 3.30-3.14 (m, 2H), 1.97-1.86 (td, J=8.7, 4.4 Hz, 1H), 1.85-1.74 (dt, J=13.5, 4.3 Hz, 1H), 1.52-1.40 (m, 2H), 1.29-1.23 (d, J=6.6 Hz, 3H).

Building block: step ii: 6-(4,4,5,5-tetramethyl-1,3,2-dioxaborolan-2-yl)-2,3-dihydro-1H-isoindol-1-one.

### Example 38: N-methyl-4′-[(2-methyl-4-{[4-(trifluoromethoxy)phenyl]Amino}piperidin-1-yl)sulfonyl]-[1,1′-biphenyl]-3-carboxamide

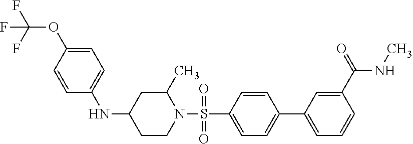 [SMILES: CNC(=O)c1cccc(-c2ccc(S(=O)(=O)N3CCC(Nc4ccc(OC(F)(F)F)cc4)CC3C)cc2)c1]

MS(ES⁺) m/z 548.3 (M+H)⁺

¹H NMR (400 MHz, DMSO) δ 8.65-8.57 (m, 1H), 8.24-8.19 (t, J=1.8 Hz, 1H), 8.03-7.98 (m, 2H), 7.96-7.89 (m, 4H), 7.66-7.58 (t, J=7.8 Hz, 1H), 7.01-6.95 (d, J=8.3 Hz, 2H), 6.55-6.48 (m, 2H), 5.80-5.76 (d, J=7.6 Hz, 1H), 3.78-3.69 (ddd, J=12.6, 8.1, 4.0 Hz, 1H), 3.53-3.45 (m, 1H), 3.28-3.17 (m, 2H), 2.89-2.80 (d, J=4.5 Hz, 3H), 1.96-1.86 (ddt, J=12.7, 8.8, 4.8 Hz, 1H), 1.83-1.73 (dt, J=13.9, 4.6 Hz, 1H), 1.53-1.41 (m, 2H), 1.29-1.23 (d, J=6.6 Hz, 3H).

Building block: step ii: [3-(methylcarbamoyl)phenyl]boronic acid.

### Example 39: 4′-{[trans-4-{[4-(trifluoromethoxy)phenyl]Amino}cyclohexyl]sulfonyl}-[1,1′-biphenyl]-4-carbonitrile

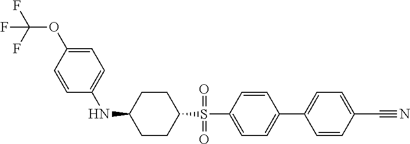 [SMILES: N#Cc1ccc(-c2ccc(S(=O)(=O)[C@H]3CC[C@H](Nc4ccc(OC(F)(F)F)cc4)CC3)cc2)cc1]

i) To a solution of tert-butyl N-[(1s,4s)-4-hydroxycyclohexyl]carbamate (50.0 g) and triethyl amine (42 mL) in CH₂Cl₂ (200 mL) was added drop wise at 0° C. a solution of methanesulfonyl chloride (23 mL) in CH₂Cl₂ (200 mL). The reaction mixture was stirred overnight at room temperature. The reaction mixture was quenched by pouring into a saturated aqueous NaHCO₃ solution. The product was extracted into ethyl acetate and the combined organic layers were washed with brine, dried over MgSO₄, filtered and concentrated under reduced pressure to give tert-butyl N-[(1s,4s)-4-(methanesulfonyloxy)cyclohexyl]carbamate as an off-white solid (69.3 g) which was used in the next step without further purification.
ii) To a suspension of the product obtained in the previous step (68.1 g) and Cs₂CO₃ (189.1 g) in acetone (600 mL) was added at room temperature, 4-bromothiophenol (79.0 g). The reaction mixture was stirred overnight at 60° C. After completion the reaction mixture was filtered, and the filtrate was concentrated under reduced pressure. The residue was dissolved in a water/ethyl acetate mixture and the product was extracted into ethyl acetate. The combined organic layers were washed with brine, dried over MgSO₄, filtered, and concentrated under reduced pressure. The obtained brown oil was crystalized from acetonitrile to give a white solid. The solid was washed with cold heptane to give tert-butyl N-[trans-4-[(4-bromophenyl)sulfanyl]cyclohexyl]carbamate (38.0 g) as a white solid.
iii) To a solution of the product obtained in the previous step (2.8 g) in ethyl acetate (6 mL) was added mCPBA (3.1 g) and the reaction mixture was stirred overnight at room temperature. After completion the reaction mixture was poured into water and the product was extracted into ethyl acetate. The combined organic layers were washed with aqueous 1N NaOH solution, water, brine, dried over MgSO₄ filtered, and concentrated under reduced pressure. The obtained white solid was purified on SiO₂, using 0% to 100% ethyl acetate in heptane as the eluent, to give tert-butyl N-[trans-4-(4-bromobenzenesulfonyl)cyclohexyl]carbamate (1.7 g) as a white solid.
iv) Under a nitrogen atmosphere, Pd(PPh₃)₄ (14 mg) was added to a solution of the product obtained in the previous step (0.5 g), 4-cyanophenylboronic acid (176 mg) and aqueous 2N K₂CO₃ (6 mL) in a mixture of toluene (13 mL) and ethanol (1.5 mL). The reaction mixture was stirred for 1 hour at 90° C. in a microwave. After cooling to room temperature, the reaction mixture was poured into water and the product was extracted into ethyl acetate. The combined organic layer were washed with water, brine, dried over MgSO₄, filtered, and concentrated under reduced pressure to give tert-buty N-[trans-4-({4′-cyano-[1,1′-biphenyl]-4-yl}sulfonyl)cyclohexyl]carbamate (0.5 g) as a brown solid, which was used in the next step without further purification.
v) To a solution of the product obtained in the previous step (500 mg) in ethyl acetate (15 mL) was added at room temperature, an aqueous 2N HCl solution (5.7 mL). The reaction mixture was stirred overnight at room temperature. After completion the reaction mixture concentrated under reduced pressure to give 4′-{[trans-4-aminocyclohexyl]sulfonyl}-[1,1′-biphenyl]-4-carbonitrile hydrochoride (500 mg) as a yellow solid, which was used in the next step without further purification.
vi) Under a nitrogen atmosphere, Pd₂(dba)₃ (9 mg) was added to a suspension of the product obtained in the previous step (60 mg), 1-bromo-4-(trifluoromethoxy)benzene (46 mg), sodium tert-butoxide (38 mg) and xantphos (11 mg) in toluene (15 mL). The reaction mixture was stirred overnight at 120° C. in a microwave. After cooling to room temperature, the reaction mixture was diluted with water and ethyl acetate and the product was extracted into ethyl acetate. The combined organic layer were washed with water, brine, dried over MgSO₄, filtered, and concentrated under reduced pressure. The obtained brown oil was purified on C18, using 10% to 90% acetonitrile in water as the eluent, to give the title compound 4′-{[trans-4-{[4-(trifluoromethoxy)phenyl]Amino}cyclohexyl]sulfonyl}-[1,1′-biphenyl]-4-carbonitrile (21 mg) as a white solid. MS(ES⁺) m/z 501.0 (M+H)⁺.

¹H NMR (500 MHz, CDCl₃) δ 8.05-7.99 (m, 2H), 7.84-7.79 (m, 4H), 7.77-7.73 (m, 2H), 7.05-7.00 (m, 2H), 6.54-6.48 (m, 2H), 3.55-3.44 (s, 1H), 3.26-3.17 (m, 1H), 3.06-2.95 (tt, J=12.3, 3.5 Hz, 1H), 2.34-2.19 (m, 4H), 1.75-1.63 (qd, J=13.2, 3.4 Hz, 2H), 1.23-1.09 (tdd, J=13.2, 11.1, 3.3 Hz, 2H).

Following a procedure analogous to that described for Example 39, using in step iv the appropriate boronic ester or boronic acid and in step vi the appropriate (hetero)aryl halide, Examples 40-43 have been prepared.

### Examples 40-43

### Example 40: 4′-{[trans-4-{[3-(trifluoromethoxy)phenyl]Amino}cyclohexyl]sulfonyl}-[1,1′-biphenyl]-4-carbonitrile

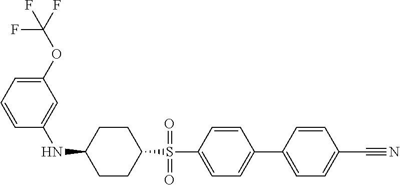 [SMILES: N#Cc1ccc(-c2ccc(S(=O)(=O)[C@H]3CC[C@H](Nc4cccc(OC(F)(F)F)c4)CC3)cc2)cc1]

MS(ES⁺) m/z 502.0 (M+H)⁺.

Building block: step iv: (4-cyanophenyl)boronic acid; step vi: 1-bromo-3-(trifluoromethoxy)benzene.

### Example 41: N-[trans-4-(4-{3-methylimidazo[1,2-a]pyridin-6-yl}benzenesulfonyl)cyclohexyl]-4-(trifluoromethoxy)aniline

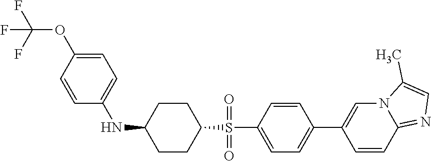 [SMILES: Cc1cnc2ccc(-c3ccc(S(=O)(=O)[C@H]4CC[C@H](Nc5ccc(OC(F)(F)F)cc5)CC4)cc3)cn12]

MS(ES⁺) m/z 530.3 (M+H)⁺.

¹H NMR (400 MHz, DMSO) δ 8.75 (s, 1H), 8.15-8.08 (m, 2H), 8.00-7.94 (m, 2H), 7.75 (s, 2H), 7.54 (s, 1H), 7.00 (d, J=8.5 Hz, 2H), 6.63-6.55 (m, 2H), 5.73 (d, J=8.0 Hz, 1H), 3.38-3.34 (m, 1H), 3.17-3.13 (m, 1H), 2.57 (s, 3H), 2.09-1.94 (m, 4H), 1.61-1.47 (m, 2H), 1.26-1.10 (m, 2H).

Building blocks: step iv: {3-methylimidazo[1,2-a]pyridin-6-yl}boronic acid; step vi: 1-iodo-4-(trifluoromethoxy)benzene.

### Example 42: N-[trans-4-(4-{3-methylimidazo[1,2-a]pyridin-6-yl}benzenesulfonyl)cyclohexyl]-5-(trifluoromethoxy)pyridin-2-amine

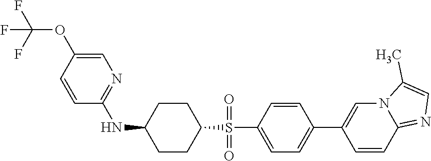 [SMILES: Cc1cnc2ccc(-c3ccc(S(=O)(=O)[C@H]4CC[C@H](Nc5ccc(OC(F)(F)F)cn5)CC4)cc3)cn12]

MS(ES⁺) m/z 502.0 (M+H)⁺.

¹H NMR (400 MHz, DMSO) δ 8.71-8.66 (m, 1H), 8.14-8.06 (m, 2H), 7.99-7.92 (m, 3H), 7.68 (dd, J=9.4, 1.0 Hz, 1H), 7.63 (dd, J=9.4, 1.7 Hz, 1H), 7.43 (d, J=1.0 Hz, 1H), 7.42-7.37 (m, 1H), 6.80 (d, J=7.4 Hz, 1H), 6.49 (dd, J=9.1, 0.7 Hz, 1H), 3.59-3.54 (m, 1H), 3.37-3.33 (m, 1H), 2.55 (s, 3H), 2.10-1.97 (m, 4H), 1.52-1.39 (m, 2H), 1.29-1.16 (m, 2H).

Building block: step iv: {3-methylimidazo[1,2-a]pyridin-6-yl}boronic acid; step vi: 2-chloro-5-(trifluoromethoxy)pyridine.

### Example 43: N-[trans-4-(4-{imidazo[1,2-a]pyridin-6-yl}benzenesulfonyl)cyclohexyl]-4-(trifluoromethoxy)aniline

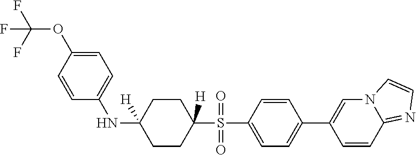 [SMILES: O=S(=O)(c1ccc(-c2ccc3nccn3c2)cc1)[C@H]1CC[C@H](Nc2ccc(OC(F)(F)F)cc2)CC1]

MS(ES⁺) m/z 516.3 (M+H)⁺.

¹H NMR (400 MHz, DMSO) δ 9.11 (dd, J=1.8, 1.1 Hz, 1H), 8.06-8.02 (m, 2H), 8.01 (s, 1H), 7.99-7.94 (m, 2H), 7.72 (d, J=9.4 Hz, 1H), 7.68 (dd, J=9.5, 1.8 Hz, 1H), 7.66 (d, J=1.2 Hz, 1H), 7.04-6.97 (m, 2H), 6.64-6.55 (m, 2H), 5.73 (d, J=8.0 Hz, 1H), 3.42-3.37 (m, 1H), 3.19-3.12 (m, 1H), 2.10-1.95 (m, 4H), 1.61-1.48 (m, 2H), 1.26-1.10 (m, 2H).

Building block: step iv: 6-(4,4,5,5-tetramethyl-1,3,2-dioxaborolan-2-yl)imidazo[1,2-a]pyridine; step vi: 1-iodo-4-(trifluoromethoxy)benzene.

### Example 44: 5-(4-{[trans-4-({4-[(trifluoromethyl)sulfanyl]phenyl}Amino)cyclohexyl]sulfonyl}phenyl)-1H-indole-3-carbonitrile

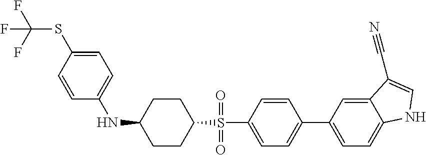 [SMILES: N#Cc1c[nH]c2ccc(-c3ccc(S(=O)(=O)[C@H]4CC[C@H](Nc5ccc(SC(F)(F)F)cc5)CC4)cc3)cc12]

i) A solution of methanesulfonyl chloride (1.3 mL) in CH₂Cl₂ (10 mL) was added dropwise to a solution of 4-hydroxycyclohexanone monoethylene ketal (2.0 g) and triethyl amine (2.3 mL) in CH₂Cl₂ (15 mL) at 0° C. The reaction mixture was stirred overnight at room temperature. After completion the reaction mixture was quenched by pouring into a saturated aqueous NaHCO₃ solution. The layers were separated, and the aqueous layers were washed with CH₂C₂. The combined organic layers were washed brine, dried over MgSO₄, filtered, and concentrated under reduced pressure. The obtained yellow oil was dissolved in heptane, the precipitated solid was filtered off and the filtrate was reduced under reduced pressure to give 1,4-dioxaspiro[4.5]decan-8-yl methanesulfonate (3.3 g) as an oil, which slowly solidified. The product was used in the next step without further purification.
ii) 4-bromobenzene-1-thiol (2.64 g) was added to a suspension of the product obtained in the previous step (3.3 g) and cesium carbonate (5.46 g) in acetone (50 mL). The reaction mixture was stirred overnight at 60° C. After cooling to room temperature, the reaction mixture was filtered, and the solids were washed with ethyl acetate. The filtrate was concentrated under reduced pressure and the obtained oil was purified on SiO₂, using 0% to 60% ethyl acetate in heptane as the eluent, giving 8-[(4-bromophenyl)sulfanyl]-1,4-dioxaspiro[4.5]decane (3.68 g) as a white solid.
iii) 3-chloroperbenzoic acid (9.64 g) was added to a solution of the product obtained in the previous step (3.68 g) in ethyl acetate (50 mL) and the reaction mixture was stirred overnight at room temperature. The reaction mixture was washed with a saturated NaHCO₃ solution, and the combined organic layers were washed with brine, dried over MgSO₄, filtered, and concentrated under reduced pressure. The residue was purified on SiO₂, using 0% to 100% ethyl acetate in heptane as the eluent, giving 8-[4-bromobenzenesulfonyl)-1,4-dioxaspiro[4.5]decane (3.68 g) as a white solid.
iv) To a solution of the product obtained in the previous step (3.5 g) in THF (50 mL) was added at room temperature an aqueous 2N HCl solution (39 mL) and the reaction mixture was stirred overnight at room temperature. After completion, the reaction mixture was washed with ethyl acetate and the combined organic layers were washed with brine, dried over MgSO₄, filtered and concentrated under reduced pressure to give 4-(4-bromobenzenesulfonyl)cyclohexan-1-one (3.0 g) as a white solid which was used in the next step without further purification.
v) To a suspension of the product obtained in the previous step (505 mg) and 4-[(trifluoromethyl)sulfanyl]aniline (390 mg) in a mixture of HOAc (1 mL) in CH30H (11 mL) was added 2-methylpyridine borane complex (180 mg) at 0° C. After removal of the ice bath the reaction mixture was stirred overnight at room temperature. The reaction mixture was concentrated under reduced pressure and an aqueous 2N HCl solution (5 mL) was added at 0° C. After stirring for 1 h at 0° C., an aqueous 5N NaOH solution (5 mL) was added, and the product was extracted into ethyl acetate. The organic layer was washed with brine, dried over MgSO₄, and concentrated under reduced pressure. The residue was purified on SiO₂, using 0% to 50% ethyl acetate in heptane as the eluent, giving N-[trans-4-(4-(bromobenzenesulfonyl)cyclohexyl]-4-[(trifluoromethyl)sulfanyl]aniline (532 mg) as a white solid.
vi) Under a nitrogen atmosphere, Pd(PPh₃)₄ (66 mg) was added to a solution of the product obtained in the previous step (530 mg), 5-(4,4,5,5-tetramethyl-1,3,2-dioxaborolan-2-yl)-1H-indole-3-carbonitrile (360 mg) and NaHCO₃ (462 mg) in a mixture of 1,4-dioxane (8 mL) and ethanol (2 mL). The reaction mixture was stirred for 1 hours at 120° C. in a microwave. After cooling to room temperature, the reaction mixture was poured into water and the product was extracted into ethyl acetate. The combined organic layers were washed with water, brine, dried over MgSO₄, filtered, and concentrated under reduced pressure. The residue was purified on SiO₂, using 0% to 80% ethyl acetate in heptane as the eluent, giving the title compound 5-(4-{[trans-4-({4-[(trifluoromethyl)sulfanyl]phenyl}Amino)cyclohexyl]sulfonyl}phenyl)-1H-indole-3-carbonitrile (248 mg) as a white solid. MS(ES⁺) m/z 556.3 (M+H)⁺.

¹H NMR (400 MHz, DMSO) δ 12.37 (s, 1H), 8.35 (s, 1H), 8.09-8.03 (m, 2H), 8.01 (t, J=1.2, 1.2 Hz, 1H), 7.95-7.89 (m, 2H), 7.75-7.66 (m, 2H), 7.35-7.27 (m, 2H), 6.69-6.61 (m, 2H), 6.27 (d, J=7.7 Hz, 1H), 3.40-3.34 (m, 1H), 3.28-3.15 (m, 1H), 2.09-1.95 (m, 4H), 1.63-1.48 (m, 2H), 1.27-1.13 (m, 2H).

Following a procedure analogous to that described for Example 44, using in step v the appropriate aniline and in step vi the appropriate boronic ester or boronic acid, Examples 45-46 have been prepared.

### Example 45: N-[trans-4-(4-{imidazo[1,2-a]pyridin-6-yl}benzenesulfonyl)cyclohexyl]-4-[(trifluoromethyl)sulfanyl]aniline

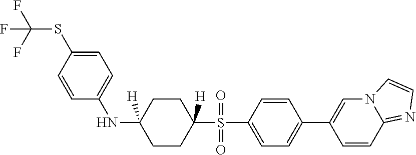 [SMILES: O=S(=O)(c1ccc(-c2ccc3nccn3c2)cc1)[C@H]1CC[C@H](Nc2ccc(SC(F)(F)F)cc2)CC1]

MS(ES⁺) m/z 532.3 (M+H).

¹H NMR (400 MHz, DMSO) δ 9.11 (s, 1H), 8.06-8.02 (m, 2H), 8.01 (s, 1H), 7.99-7.94 (m, 2H), 7.72 (d, J=9.4 Hz, 1H), 7.68 (dd, J=9.4, 1.8 Hz, 1H), 7.66 (d, J=1.2 Hz, 1H), 7.35-7.28 (m, 2H), 6.69-6.61 (m, 2H), 6.27 (d, J=7.8 Hz, 1H), 3.41-3.34 (m, 1H), 3.27-3.18 (m, 1H), 2.10-1.95 (m, 4H), 1.65-1.46 (m, 2H), 1.30-1.11 (m, 2H).

Building block: step v: 4-[(trifluoromethyl)sulfanyl]aniline step vi: 6-(4,4,5,5-tetramethyl-1,3,2-dioxaborolan-2-yl)imidazo[1,2-a]pyridine.

### Example 46: N-[trans-4-(4-{imidazo[1,2-a]pyridin-6-yl}benzenesulfonyl)cyclohexyl]-3-[(trifluoromethyl)sulfanyl]aniline

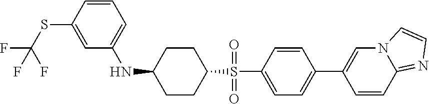 [SMILES: O=S(=O)(c1ccc(-c2ccc3nccn3c2)cc1)[C@H]1CC[C@H](Nc2cccc(SC(F)(F)F)c2)CC1]

MS(ES⁺) m/z 532.3 (M+H)⁺.

¹H NMR (400 MHz, DMSO) δ 9.14-9.08 (m, 1H), 8.06-8.02 (m, 2H), 8.01 (s, 1H), 8.01-7.93 (m, 2H), 7.75-7.63 (m, 3H), 7.17 (dd, J=8.3, 7.6 Hz, 1H), 6.86 (s, 1H), 6.82-6.72 (m, 2H), 5.94 (d, J=7.9 Hz, 1H), 3.40-3.34 (m, 1H), 3.26-3.17 (m, 1H), 2.08-1.94 (m, 4H), 1.63-1.50 (m, 2H), 1.26-1.12 (m, 2H).

Building block: step v: 3-[(trifluoromethyl)sulfanyl]aniline step vi: 6-(4,4,5,5-tetramethyl-1,3,2-dioxaborolan-2-yl)imidazo[1,2-a]pyridine.

### Example 47: 5 N-[trans-4-{4-[3-(propan-2-yl)imidazo[1,2-a]pyridin-6-yl]benzenesulfonyl}cyclohexyl]-3-[(trifluoromethyl)sulfanyl]aniline

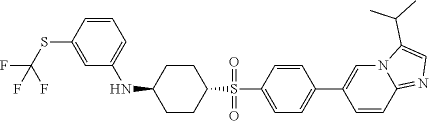 [SMILES: CC(C)c1cnc2ccc(-c3ccc(S(=O)(=O)[C@H]4CC[C@H](Nc5cccc(SC(F)(F)F)c5)CC4)cc3)cn12]

i) Following a procedure analogous to that described for Example 44, step ii, using 3-[(trifluoromethyl)sulfanyl]aniline, N-[trans-4-(4-bromobenzenesulfonyl)cyclohexyl]-3-[(trifluoromethyl)sulfanyl]aniline has been prepared as a white solid.
ii) To a suspension of the product obtained in the previous step (150 mg), bis(pinacolato)diboron (152 mg) and potassium acetate (103 mg) in cyclopentyl methyl ether (2 mL), purged with N₂ gas, was added PdCl₂(dppf)·CH₂Cl₂ (11 mg). The reaction mixture was heated for 2 hours at 100° C. in a microwave. After cooling to room temperature, the reaction mixture was poured into water and the product was extracted into ethyl acetate. The organic layer was washed with brine, dried over MgSO₄ and concentrated under reduced pressure giving N-[trans-4-[4-(4,4,5,5-tetramethyl-1,3,2-dioxaborolan-2-yl)benzenesulfonyl]cyclohexyl]-3-[(trifluoromethyl(sulfanyl]aniline as a brown oil (295 mg) which was used in the next step without further purification.
iii) Under a nitrogen atmosphere, Pd(PPh₃)₄ (31 mg) was added to a suspension of the product obtained in the previous step (295 mg), 6-bromo-3-(propan-2-yl)imidazo[1,2-a]pyridine (143 mg), and NaHCO₃ (228 mg) in a mixture of 1,4-dioxane (6 mL) and water (1.5 mL). The reaction mixture was stirred for 2 hours at 100° C. in a microwave. After cooling to room temperature, the reaction mixture was diluted with water and ethyl acetate and the product was extracted into ethyl acetate. The combined organic layers were washed with water, brine, dried over MgSO₄, filtered, concentrated under reduced pressure. The residue was purified on C18, using 20% to 50% acetonitrile in water as the eluent, to give the title compound N-[trans-4-{4-[3-(propan-2-yl)imidazo[1,2-a]pyridin-6-yl]benzenesulfonyl}cyclohexyl]-3-[(trifluoromethyl)sulfanyl]aniline (112 mg) as a white solid. MS(ES⁺) m/z 574.3 (M+H)⁺.

¹H NMR (400 MHz, DMSO) δ 8.73 (s, 1H), 8.14-8.06 (m, 2H), 8.00-7.91 (m, 2H), 7.69 (dd, J=9.4, 1.0 Hz, 1H), 7.63 (dd, J=9.4, 1.8 Hz, 1H), 7.46 (s, 1H), 7.19-7.13 (m, 1H), 6.86 (s, 1H), 6.83-6.72 (m, 2H), 5.94 (d, J=7.9 Hz, 1H), 3.51 (h, J=6.8, 6.8, 6.8, 6.8, 6.8 Hz, 1H), 3.40-3.34 (m, 1H), 3.29-3.17 (m, 1H), 2.07-1.95 (m, 4H), 1.64-1.50 (m, 2H), 1.36 (d, J=6.8 Hz, 6H), 1.26-1.10 (m, 2H).

Following a procedure analogous to that described for Example 47, using in step i the appropriate aniline and in step iii the appropriate (hetero)aryl halide, Examples 48-50 have been prepared.

### Example 48: N-[trans-4-{4-[8-methyl-3-(propan-2-yl)imidazo[1,2-a]pyridin-6-yl]benzenesulfonyl}cyclohexyl]-4-[(trifluoromethyl)sulfanyl]aniline

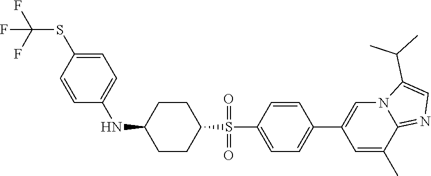 [SMILES: Cc1cc(-c2ccc(S(=O)(=O)[C@H]3CC[C@H](Nc4ccc(SC(F)(F)F)cc4)CC3)cc2)cn2c(C(C)C)cnc12]

MS(ES⁺) m/z 588.3 (M+H)⁺

¹H NMR (400 MHz, DMSO) δ 8.59 (s, 1H), 8.13-8.05 (m, 2H), 7.99-7.91 (m, 2H), 7.49 (t, J=1.5, 1.5 Hz, 1H), 7.41 (s, 1H), 7.35-7.27 (m, 2H), 6.69-6.61 (m, 2H), 6.28 (d, J=7.8 Hz, 1H), 3.49 (hept, J=6.7, 6.7, 6.7, 6.7, 6.6, 6.6 Hz, 1H), 3.41-3.34 (m, 1H), 3.23 (dd, J=7.6, 3.7 Hz, OH), 2.57 (s, 3H), 2.02 (t, J=16.0, 16.0 Hz, 4H), 1.63-1.48 (m, 2H), 1.36 (d, J=6.8 Hz, 6H), 1.21 (dd, J=21.9, 9.2 Hz, 3H).

Building block: step i: 4-[(trifluoromethyl)sulfanyl]aniline step iii: 6-bromo-8-methyl-3-(propan-2-yl)imidazo[1,2-a]pyridine.

### Example 49: 4-(4-{[trans-4-({4-[(trifluoromethyl)sulfanyl]phenyl}Amino)cyclohexyl]sulfonyl}phenyl)pyridine-2-carboxamide

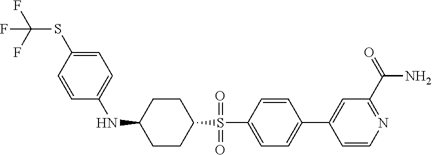 [SMILES: NC(=O)c1cc(-c2ccc(S(=O)(=O)[C@H]3CC[C@H](Nc4ccc(SC(F)(F)F)cc4)CC3)cc2)ccn1]

MS(ES⁺) m/z 536.3 (M+H)⁺

¹H NMR (400 MHz, DMSO) δ 8.78 (dd, J=5.1, 0.8 Hz, 1H), 8.38 (dd, J=1.9, 0.8 Hz, 1H), 8.23 (d, J=2.6 Hz, 1H), 8.20-8.13 (m, 2H), 8.06-7.98 (m, 3H), 7.77 (d, J=2.6 Hz, 1H), 7.35-7.27 (m, 2H), 6.69-6.61 (m, 2H), 6.27 (d, J=7.8 Hz, 1H), 3.45-3.34 (m, 1H), 3.26-3.18 (m, 1H), 2.08-1.95 (m, 4H), 1.62-1.49 (m, 2H), 1.27-1.14 (m, 2H).

Building block: step i: 4-[(trifluoromethyl)sulfanyl]aniline step iii: 4-bromopyridine-2-carboxamide.

### Example 50: 6-(4-{[trans-4-({4-[(trifluoromethyl)sulfanyl]phenyl}Amino)cyclohexyl]sulfonyl}phenyl)-2H,3H-[1,2,4]triazolo[4,3-a]pyridin-3-one

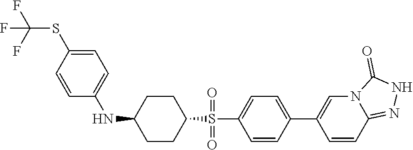 [SMILES: O=c1[nH]nc2ccc(-c3ccc(S(=O)(=O)[C@H]4CC[C@H](Nc5ccc(SC(F)(F)F)cc5)CC4)cc3)cn12]

MS(ES⁺) m/z 549.3 (M+H)⁺.

¹H NMR (400 MHz, DMSO) δ 12.61 (s, 1H), 8.25 (t, J=1.5, 1.5 Hz, 1H), 8.09-8.02 (m, 2H), 7.96-7.88 (m, 2H), 7.67 (dd, J=9.8, 1.8 Hz, 1H), 7.39 (dd, J=9.8, 1.1 Hz, 1H), 7.36-7.28 (m, 2H), 6.69-6.61 (m, 2H), 6.27 (d, J=7.8 Hz, 1H), 3.38 (s, 1H), 3.22 (dt, J=8.1, 3.9, 3.9 Hz, 1H), 2.09-1.94 (m, 4H), 1.63-1.46 (m, 2H), 1.29-1.11 (m, 2H).

Building block: step i: 4-[(trifluoromethyl)sulfanyl]aniline step iii: 6-bromo-2-[(4-methoxyphenyl)methyl]-2H,3H-[1,2,4]triazolo[4,3-a]pyridin-3-one.

### Example 51: {4-[3-(propan-2-yl)imidazo[1,2-a]pyridin-6-yl]phenyl}[trans-4-({4-[(trifluoromethyl)sulfanyl]phenyl}Amino)cyclohexyl](imino)-λ⁶-sulfanone

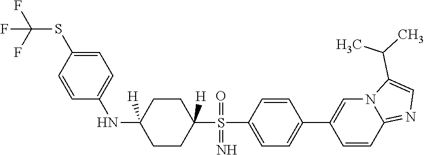 [SMILES: CC(C)c1cnc2ccc(-c3ccc(S(=N)(=O)[C@H]4CC[C@H](Nc5ccc(SC(F)(F)F)cc5)CC4)cc3)cn12]

i) To a suspension of 8-[(4-bromophenyl)sulfanyl]-1,4-dioxaspiro[4.5]decane (Example 44, step ii (5 g) and ammonium carbamate (1.8 g) in a mixture of methanol (10 mL) and acetonitrile (10 mL) was added at room temperature (diacetoxyiodo)benzene (10.2 g). The reaction mixture was stirred at room temperature for 1 hour in a flask open to the atmosphere. After completion, the reaction mixture was concentrated under reduced pressure. At this stage, the cis/trans mixture could be separated on C18, using 10% to 100% acetonitrile in water, to give (4-bromophenyl)({1,4-diazaspiro[4,5]decan-8-yl})(imino)-λ⁶-sulfanylone (5.3 g).
ii) To a solution of the product obtained in the previous step (5.3 g) in THF (100 mL) was added an aqueous 2N HCl solution (110 mL) and the reaction mixture was stirred overnight at room temperature. After completion, the organic solvent was removed under reduced pressure and the remaining aqueous layer was washed with EtOAc. The combined organic layers were washed with water, brine, dried over MgSO₄, filtered and concentrated under reduced pressure to give 4-[(4-bromophenyl)oxo-λ⁶-sulfanyl]cyclohexan-1-one as an yellowish oil (4.6 g), which was used in the next step without further purification.
iii) To a solution of the product obtained in the previous step (1.0 g), 4-[(trifluoromethyl)sulfanyl]aniline (794 mg) and HOAc (1 mL) in methanol (20 mL) was added at 0° C., 2-methylpyridine borane complex (440 mg). The reaction mixture was stirred overnight at room temperature. After completion the reaction mixture was cooled to 0° C. and an aqueous solution of 2N HCl (3 mL) was added. After stirring for 1 hour at 0° C., an aqueous solution of 5N NaOH (6.6 mL) was added. The product was extracted into EtOAc and the combined organic layers were washed with water, brine, dried over MgSO₄, filtered and concentrated under reduced pressure. The residue was purified on SiO₂, using 0% to 95% ethyl acetate in heptane as the eluent, giving (4-bromophenyl)[trans-4-({4-[(trifluoromethyl)sulfanyl]phenyl}Amino)cyclohexyl](imino)-λ⁶-sulfanone (400 mg) as a white solid.
iv) To a suspension of the product obtained in the previous step (300 mg), bis(pinacolato)diboron (206 mg) and potassium acetate (133 mg) in cyclopentyl methyl ether (15 mL), purged with N₂ gas, was added PdCl₂(dppf)·CH₂Cl₂ (22 mg). The reaction mixture was heated for 2 hours at 110° C. in a microwave. After cooling to room temperature, the reaction mixture was poured into water and the product was extracted into ethyl acetate. The organic layer was washed with brine, dried over MgSO₄ and concentrated under reduced pressure giving {trans-4-({4-[(trifluoromethyl)sulfanyl]phenyl}Amino)cyclohexyl][4-(4,4,5,5-tetramethyl-1,3,2-dioxaborolan-2-yl)phenyl](imino)-λ⁶-sulfanone as a brown oil (384 mg) which was used in the next step without further purification.
v) Under a nitrogen atmosphere, Pd(PPh₃)₄ (13 mg) was added to a suspension of the product obtained in the previous step (192 mg), 6-bromo-3-(propan-2-yl)imidazo[1,2-a]pyridine (93 mg), and NaHCO₃ (90 mg) in a mixture of 1,4-dioxane (10 mL) and water (4 mL). The reaction mixture was stirred for 2 hours at 110° C. in a microwave. After cooling to room temperature, the reaction mixture was diluted with water and ethyl acetate and the product was extracted into ethyl acetate. The combined organic layers were washed with water, brine, dried over MgSO₄, filtered, concentrated under reduced pressure. The residue was purified on C18, using 10% to 50% acetonitrile in water-as the eluent, to give the title compound {4-[3-(propan-2-yl)imidazo[1,2-a]pyridin-6-yl]phenyl}[trans-4-({4-[(trifluoromethyl)sulfanyl]phenyl}Amino)cyclohexyl(imino)-λ⁶-sulfanone (40 mg) as a white solid. MS(ES⁺) m/z 573.4 (M+H)⁺.

¹H NMR (400 MHz, DMSO) δ 8.70 (s, 1H), 8.08-8.00 (m, 2H), 7.99-7.92 (m, 2H), 7.69 (dd, J=9.4, 0.9 Hz, 1H), 7.62 (dd, J=9.4, 1.8 Hz, 1H), 7.46 (d, J=0.7 Hz, 1H), 7.34-7.26 (m, 2H), 6.67-6.59 (m, 2H), 6.25 (d, J=7.7 Hz, 1H), 4.26 (s, 1H), 3.52 (p, J=6.8, 6.8, 6.8, 6.8 Hz, 1H), 3.21-3.05 (m, 2H), 2.10-1.96 (m, 4H), 1.60-1.42 (m, 2H), 1.36 (d, J=6.8 Hz, 6H), 1.26-1.11 (m, 2H).

### Example 52: (4-{imidazo[1,2-a]pyridin-6-yl}phenyl)[trans-4-({4-[(trifluoromethyl)sulfanyl]phenyl}Amino)cyclohexyl](imino)-λ⁶-sulfanone

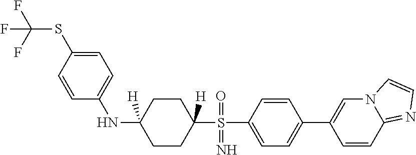 [SMILES: N=S(=O)(c1ccc(-c2ccc3nccn3c2)cc1)[C@H]1CC[C@H](Nc2ccc(SC(F)(F)F)cc2)CC1]

i) Under a nitrogen atmosphere, Pd(PPh₃)₄ (21 mg) was added to a solution of (4-bromophenyl)[trans-4-({4-[(trifluoromethyl)sulfanyl]phenyl}Amino)cyclohexyl](imino)-λ⁶-sulfanone (Example 51, step iii (100 mg), 5 6-(4,4,5,5-tetramethyl-1,3,2-dioxaborolan-2-yl)imidazo[1,2-a]pyridine (48 mg) and NaHCO₃ (45 mg) in a mixture of 1,4-dioxane (10 mL) and ethanol (3 mL). The reaction mixture was stirred for 2 hours at 110° C. in a microwave. After cooling to room temperature, the reaction mixture was poured into water and the product was extracted into ethyl acetate. The combined organic layers were washed with water, brine, dried over MgSO₄, filtered, and concentrated under reduced pressure. The residue was purified on C18, using 10% to 50% acetonitrile in water as the eluent, giving the title compound (4-{imidazo[1,2-a]pyridin-6-yl}phenyl)[trans-4-({4-[trifluoromethyl)sulfanyl]phenyl}Amino)cyclohexyl](imino)-λ⁶-sulfanone (26 mg) as a white solid. MS(ES⁺) m/z 531.3 (M+H)⁺.

¹H NMR (400 MHz, DMSO) δ 9.09 (s, 1H), 8.01 (s, 1H), 8.00-7.93 (m, 4H), 7.71 (d, J=9.4 Hz, 1H), 7.69-7.63 (m, 2H), 7.35-7.27 (m, 2H), 6.67-6.59 (m, 2H), 6.25 (d, J=7.8 Hz, 1H), 4.27 (s, 1H), 3.20-3.05 (m, 2H), 2.11-1.96 (m, 4H), 1.59-1.41 (m, 2H), 1.26-1.11 (m, 2H).

### Example 53: 6-(4-{[trans-4-({4-[(trifluoromethyl)sulfanyl]phenyl}Amino)cyclohexyl]sulfonimidoyl}phenyl)-2H,3H-[1,2,4]triazolo[4,3-a]pyridin-3-one

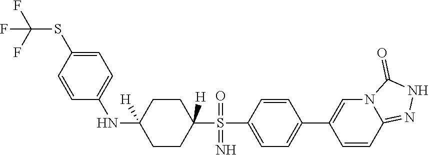 [SMILES: N=S(=O)(c1ccc(-c2ccc3n[nH]c(=O)n3c2)cc1)[C@H]1CC[C@H](Nc2ccc(SC(F)(F)F)cc2)CC1]

i) Following a procedure analogous to that described for Example 51, step v, using 6-bromo-2-[(4-methoxyphenyl)methyl]-2H,3H-[1,2,4]triazolo[4,3-a]pyridin-3-one (130 mg), 6-(4-{[trans-4-({4-[(trifluoromethyl)sulfanyl]phenyl}Amino)cyclohexyl]oxo-λ⁶-sulfanyl}phenyl)-2H,3H-[1,2,4]triazolo[4,3-a]pyridin-3-one (91 mg) has been prepared as a white solid.
ii) A solution of the product obtained in the previous step (91 mg) and L-cycteine (21 mg) in trifluoroacetic acid (2.6 mL) was stirred overnight at 70° C. After cooling to room temperature the reaction mixture was concentrated under reduced pressure and the residue was purified on C18, using 0% to 10% acetonitril in water as the eluent, giving the title compound 6-(4-{[trans-4-({4-[(trifluoromethyl)sulfanyl]phenyl}Amino)cyclohexyl]oxo-λ⁶-sulfanyl}phenyl)-2H,3H-[1,2,4]triazolo[4,3-a]pyridin-3-one (28 mg) as a white solid. MS(ES⁺) m/z 548.3 (M+H)⁺.

¹H NMR (400 MHz, DMSO) δ 12.60 (s, 1H), 8.21 (t, J=1.5, 1.5 Hz, 1H), 8.03-7.95 (m, 2H), 7.95-7.88 (m, 2H), 7.67 (dd, J=9.9, 1.8 Hz, 1H), 7.39 (dd, J=9.9, 1.1 Hz, 1H), 7.35-7.26 (m, 2H), 6.67-6.59 (m, 2H), 6.25 (d, J=7.7 Hz, 1H), 4.26 (s, 1H), 3.19-3.04 (m, 2H), 2.06-1.94 (m, 4H), 1.57-1.40 (m, 2H), 1.26-1.14 (m, 2H).

### Example 54: (4-{imidazo[1,2-a]pyridin-6-yl}phenyl)[trans-4-{[5-(trifluoromethoxy)pyridin-2-yl]Amino}cyclohexyl](imino)-λ⁶-sulfanone

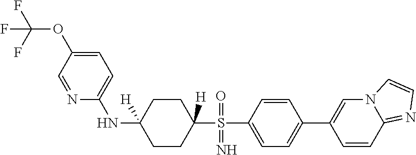 [SMILES: N=S(=O)(c1ccc(-c2ccc3nccn3c2)cc1)[C@H]1CC[C@H](Nc2ccc(OC(F)(F)F)cn2)CC1]

i) Under a nitrogen atmosphere, Pd(PPh₃)₄ (299 mg) was added to a suspension of tert-butyl N-[trans-4-[(4-bromophenyl)sulfanyl]cyclohexyl]carbamate (Example 39, step ii (2.0 g), 6-(4,4,5,5-tetramethyl-1,3,2-dioxaborolan-2-yl)imidazo[1,2-a]pyridine (1.64 g) and NaHCO₃ (2.61 g) in a mixture of 1,4-dioxane (12 mL) and water (3 mL). The reaction mixture was stirred for 2 hours at 120° C. in a microwave. After cooling to room temperature, the product was extracted into EtOAc and the organic layer was dried over MgSO₄, filtered, and concentrated under reduced pressure. The obtained brown solid was purified on C18, using 10% to 100% acetonitrile in water as the eluent, giving tert-butyl N-[trans-4-[(4-{imidazo[1,2-a]pyridin-6-yl}phenyl)sulfanyl]cyclohexyl]carbamate as a white solid (2.0 g).
ii) To a solution of the product obtained in the previous step (2.0 g) in ethyl acetate (50 mL) was added an 5N HCl solution in 2-propanol (9.44 mL) and the reaction mixture was stirred overnight at room temperature. After completion, the reaction mixture concentrated under reduced pressure to give trans-4-[(4-{imidazo[1,2-a]pyridin-6-yl}phenyl)sulfanyl]cyclohexan-1-amine hydrochloride as an yellow oil (1.9 g), which was used in the next step without further purification.
iii) Under a nitrogen atmosphere, Pd₂(dba)₃ (76 mg) was added to a suspension of the product obtained in the previous step (300 mg), 2-chloro-5-(trifluoromethoxy)pyridine (165 mg), t-BuONa (481 mg) and Xantphos (121 mg) in toluene (5 mL). The reaction mixture was stirred overnight at 100° C. in a microwave. After cooling to room temperature, the reaction mixture was dried over MgSO₄, filtered, and concentrated under reduced pressure. The residue was purified on C18, using 10% to 100% CH3CN in water as the eluent, to give the title compound N-[trans-4-[(4-{imidazo[1,2-a]pyridin-6-yl}phenyl)sulfanyl]cyclohexyl]-5-(trifluoromethoxy)pyridin-2-amine (90 mg) as a white solid.
iv) To a suspension of the product obtained in the previous step (90 mg) and ammonium carbamate (22 mg) in methanol (5 mL) was added at room temperature (diacetoxyiodo)benzene (126 mg). The reaction mixture was stirred at room temperature for 1 hour in a flask open to the atmosphere. After completion, the reaction mixture was concentrated under reduced pressure. The obtained brown solid was purified on C18, using 10% to 90% acetonitrile in water as the eluent, giving the title compound (4-{imidazo[1,2-a]pyridin-6-yl}phenyl)[trans-4-{[5-(trifluoromethoxy)pyridin-2-yl]Amino}cyclohexyl](imino)-λ⁶-sulfanone (22 mg) as a white solid. MS(ES⁺) m/z 516.3 (M+H)⁺.

¹H NMR (400 MHz, DMSO) δ 9.09 (dd, J=1.8, 1.1 Hz, 1H), 8.00 (s, 1H), 7.98-7.94 (m, 5H), 7.73-7.63 (m, 3H), 7.43-7.35 (m, 1H), 6.77 (d, J=7.5 Hz, 1H), 6.48 (d, J=9.4 Hz, 1H), 4.27 (s, 1H), 3.57-3.49 (m, 1H), 3.12-3.08 (m, 1H), 2.05-1.99 (m, 4H), 1.52-1.33 (m, 2H), 1.27-1.12 (m, 2H)

Following a procedure analogous to that described for Example 13, using in step i and step iii the appropriate (hetero)aryl halide, Examples 55-71 have been prepared.

### Examples 55-71

### Example 55: N-(1-{4-[8-methyl-3-(propan-2-yl)imidazo[1,2-a]pyridin-6-yl]benzenesulfonyl}piperidin-4-yl)-5-(trifluoromethoxy)pyridin-2-amine

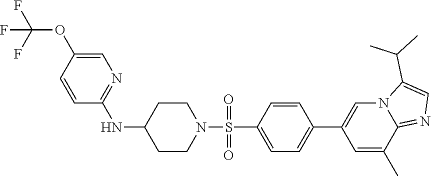 [SMILES: Cc1cc(-c2ccc(S(=O)(=O)N3CCC(Nc4ccc(OC(F)(F)F)cn4)CC3)cc2)cn2c(C(C)C)cnc12]

MS(ES⁺) m/z 574.4 (M+H)⁺.

¹H NMR (400 MHz, DMSO) δ 8.61-8.55 (m, 1H), 8.10-8.03 (m, 2H), 7.98-7.92 (d, J=2.9 Hz, 1H), 7.89-7.81 (m, 2H), 7.51-7.45 (s, 1H), 7.45-7.38 (m, 2H), 6.91-6.84 (d, J=7.2 Hz, 1H), 6.54-6.46 (d, J=9.2 Hz, 1H), 3.73-3.62 (m, 1H), 3.62-3.54 (m, 2H), 3.54-3.42 (dt, J=13.5, 6.8 Hz, 1H), 2.63-2.58 (m, 2H), 2.58-2.56 (s, 3H), 2.02-1.93 (m, 2H), 1.59-1.43 (m, 2H), 1.38-1.36 (s, 3H), 1.36-1.33 (s, 3H).

Building block: step i: 2-chloro-5-(trifluoromethoxy)pyridine; step iii: 6-bromo-8-methyl-3-(propan-2-yl)imidazo[1,2-a]pyridine.

### Example 56: N-(1-{4-[3-(propan-2-yl)-[1,2,4]triazolo[4,3-a]pyridin-6-yl]benzenesulfonyl}piperidin-4-yl)-5-(trifluoromethoxy)pyridin-2-amine

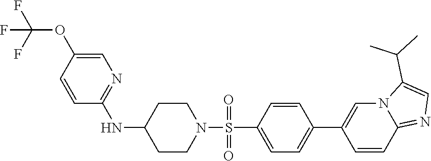 [SMILES: CC(C)c1cnc2ccc(-c3ccc(S(=O)(=O)N4CCC(Nc5ccc(OC(F)(F)F)cn5)CC4)cc3)cn12]

MS(ES⁺) m/z 561.4 (M+H)⁺.

¹H NMR (400 MHz, DMSO) δ 8.87-8.82 (t, J=1.3 Hz, 1H), 8.14-8.05 (m, 2H), 7.96-7.91 (d, J=2.9 Hz, 1H), 7.91-7.85 (m, 3H), 7.80-7.75 (m, 1H), 7.45-7.37 (ddd, J=9.1, 2.9, 1.1 Hz, 1H), 6.89-6.83 (d, J=7.2 Hz, 1H), 6.54-6.47 (d, J=9.2 Hz, 1H), 3.78-3.70 (dt, J=13.8, 6.9 Hz, 1H), 3.70-3.63 (m, 1H), 3.63-3.54 (m, 2H), 2.64-2.54 (m, 2H), 2.02-1.92 (m, 2H), 1.57-1.47 (m, 2H), 1.45-1.43 (s, 3H), 1.43-1.41 (s, 3H).

Building block: step i: 2-chloro-5-(trifluoromethoxy)pyridine; step iii: 6-bromo-3-(propan-2-yl)-[1,2,4]triazolo[4,3-a]pyridine.

### Example 57: N-(1-{4-[8-methyl-3-(propan-2-yl)-[1,2,4]triazolo[4,3-a]pyridin-6-yl]benzenesulfonyl}piperidin-4-yl)-5-(trifluoromethoxy)pyridin-2-amine

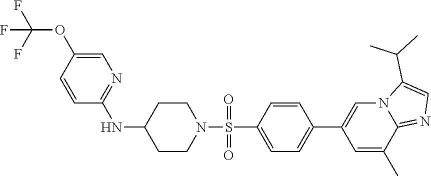 [SMILES: Cc1cc(-c2ccc(S(=O)(=O)N3CCC(Nc4ccc(OC(F)(F)F)cn4)CC3)cc2)cn2c(C(C)C)cnc12]

MS(ES⁺) m/z 575.4 (M+H)⁺.

¹H NMR (400 MHz, DMSO) δ 8.75-8.68 (t, J=1.3 Hz, 1H), 8.15-8.05 (m, 2H), 7.97-7.92 (d, J=2.9 Hz, 1H), 7.92-7.84 (m, 2H), 7.64-7.59 (t, J=1.4 Hz, 1H), 7.45-7.38 (m, 1H), 6.90-6.84 (d, J=7.2 Hz, 1H), 6.55-6.47 (d, J=9.1 Hz, 1H), 3.76-3.68 (m, 1H), 3.67-3.62 (m, 1H), 3.62-3.54 (m, 2H), 2.62-2.59 (s, 3H), 2.59-2.54 (m, 2H), 2.02-1.93 (m, 2H), 1.56-1.46 (m, 2H), 1.45-1.43 (s, 3H), 1.43-1.39 (s, 3H).

Building block: step i: 2-chloro-5-(trifluoromethoxy)pyridine; step iii: 6-bromo-8-methyl-3-(propan-2-yl)-[1,2,4]triazolo[4,3-a]pyridine.

### Example 58: 4-{4-[(4-{[5-(trifluoromethoxy)pyridin-2-yl]amino}piperidin-1-yl)sulfonyl]phenyl}pyridine-2-carboxamide

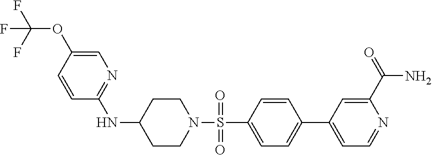 [SMILES: NC(=O)c1cc(-c2ccc(S(=O)(=O)N3CCC(Nc4ccc(OC(F)(F)F)cn4)CC3)cc2)ccn1]

MS(ES⁺) m/z 522.3 (M+H)⁺.

¹H NMR (400 MHz, DMSO) δ 8.80-8.75 (dd, J=5.1, 0.8 Hz, 1H), 8.40-8.36 (dd, J=2.1, 0.8 Hz, 1H), 8.26-8.20 (d, J=2.8 Hz, 1H), 8.18-8.10 (m, 2H), 8.05-7.99 (dd, J=5.2, 2.0 Hz, 1H), 7.97-7.93 (d, J=2.9 Hz, 1H), 7.93-7.88 (m, 2H), 7.80-7.75 (d, J=2.8 Hz, 1H), 7.44-7.38 (dtd, J=9.1, 1.9, 1.0 Hz, 1H), 6.89-6.84 (d, J=7.3 Hz, 1H), 6.52-6.47 (dd, J=9.2, 0.7 Hz, 1H), 3.75-3.63 (m, 1H), 3.62-3.52 (m, 2H), 2.67-2.59 (m, 2H), 2.01-1.91 (m, 2H), 1.55-1.42 (m, 2H).

Building block: step i: 2-chloro-5-(trifluoromethoxy)pyridine; step iii: 4-bromopyridine-2-carboxamide.

### Example 59: N-(1-{4-[3-(propan-2-yl)-[1,2,4]triazolo[4,3-a]pyrazin-6-yl]benzenesulfonyl}piperidin-4-yl)-5-(trifluoromethoxy)pyridin-2-amine

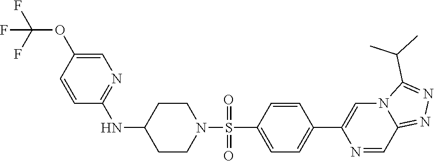 [SMILES: CC(C)c1nnc2cnc(-c3ccc(S(=O)(=O)N4CCC(Nc5ccc(OC(F)(F)F)cn5)CC4)cc3)cn12]

MS(ES⁺) m/z 562.4 (M+H)⁺.

¹H NMR (400 MHz, DMSO) δ 9.55-9.49 (d, J=1.6 Hz, 1H), 9.26-9.20 (d, J=1.7 Hz, 1H), 8.50-8.38 (m, 2H), 7.95-7.93 (d, J=2.9 Hz, 1H), 7.93-7.88 (m, 2H), 7.44-7.38 (m, 1H), 6.87-6.82 (d, J=7.2 Hz, 1H), 6.53-6.46 (d, J=9.2 Hz, 1H), 3.85-3.73 (hept, J=6.9 Hz, 1H), 3.73-3.62 (m, 1H), 3.62-3.53 (m, 2H), 2.66-2.57 (m, 2H), 2.02-1.91 (m, 2H), 1.55-1.49 (m, 2H), 1.49-1.46 (s, 3H), 1.46-1.43 (s, 3H).

Building block: step i: 2-chloro-5-(trifluoromethoxy)pyridine; step iii: 6-chloro-3-(propan-2-yl)-[1,2,4]triazolo[4,3-a]pyrazine.

### Example 60: N-[1-(4-{3-methyl-[1,2,4]triazolo[4,3-a]pyridin-6-yl}benzenesulfonyl)piperidin-4-yl]-5-(trifluoromethoxy)pyridin-2-amine

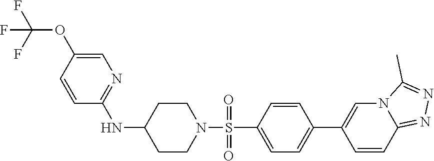 [SMILES: Cc1nnc2ccc(-c3ccc(S(=O)(=O)N4CCC(Nc5ccc(OC(F)(F)F)cn5)CC4)cc3)cn12]

MS(ES⁺) m/z 562.4 (M+H)⁺.

¹H NMR (400 MHz, DMSO) δ 8.81-8.76 (t, J=1.4 Hz, 1H), 8.15-8.07 (m, 2H), 7.98-7.93 (d, J=2.8 Hz, 1H), 7.91-7.87 (m, 2H), 7.86-7.84 (d, J=1.0 Hz, 1H), 7.81-7.76 (m, 1H), 7.44-7.39 (ddd, J=9.1, 3.0, 1.2 Hz, 1H), 6.88-6.85 (d, J=7.2 Hz, 1H), 6.53-6.48 (d, J=9.2 Hz, 1H), 3.74-3.62 (m, 1H), 3.62-3.54 (m, 2H), 2.80-2.77 (s, 3H), 2.64-2.56 (m, 2H), 2.05-1.92 (m, 2H), 1.59-1.42 (m, 2H).

Building block: step i: 2-chloro-5-(trifluoromethoxy)pyridine; step iii: 6-bromo-3-methyl-[1,2,4]triazolo[4,3-a]pyridine.

### Example 61: N-(1-{4-[8-methyl-3-(propan-2-yl)imidazo[1,2-a]pyridin-6-yl]benzenesulfonyl}piperidin-4-yl)-5-[(trifluoromethyl)sulfanyl]pyridin-2-amine

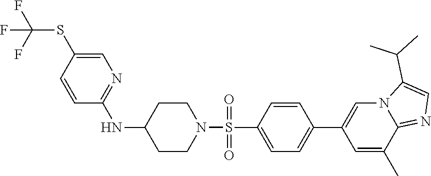 [SMILES: Cc1cc(-c2ccc(S(=O)(=O)N3CCC(Nc4ccc(SC(F)(F)F)cn4)CC3)cc2)cn2c(C(C)C)cnc12]

MS(ES⁺) m/z 590.3 (M+H)⁺.

¹H NMR (400 MHz, DMSO) δ 8.61-8.55 (s, 1H), 8.17-8.11 (d, J=2.4 Hz, 1H), 8.10-8.04 (m, 2H), 7.88-7.81 (m, 2H), 7.61-7.55 (dd, J=8.8, 2.4 Hz, 1H), 7.49-7.46 (t, J=1.4 Hz, 1H), 7.43-7.39 (d, J=0.8 Hz, 1H), 7.36-7.30 (d, J=7.2 Hz, 1H), 6.57-6.51 (dd, J=8.8, 0.8 Hz, 1H), 3.82-3.70 (m, 1H), 3.65-3.55 (m, 2H), 3.54-3.45 (dt, J=13.6, 6.8 Hz, 1H), 2.64-2.58 (m, 2H), 2.58-2.56 (s, 3H), 2.02-1.94 (m, 2H), 1.60-1.45 (m, 2H), 1.38-1.36 (s, 3H), 1.36-1.33 (s, 3H).

Building blocks: step i: 2-chloro-5-[(trifluoromethyl)sulfanyl]pyridine; step iii: 6-bromo-8-methyl-3-(propan-2-yl)imidazo[1,2-a]pyridine.

### Example 62: N-[1-(4-{imidazo[1,2-a]pyridin-6-yl}benzenesulfonyl)piperidin-4-yl]-5-[(trifluoromethyl)sulfanyl]pyridin-2-amine

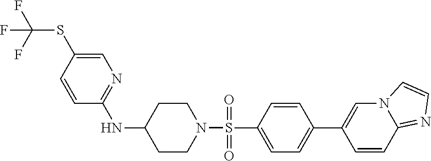 [SMILES: O=S(=O)(c1ccc(-c2ccc3nccn3c2)cc1)N1CCC(Nc2ccc(SC(F)(F)F)cn2)CC1]

MS(ES⁺) m/z 534.3 (M+H)⁺.

¹H NMR (400 MHz, DMSO) δ 9.14-9.07 (dd, J=1.8, 1.1 Hz, 1H), 8.17-8.11 (d, J=2.4 Hz, 1H), 8.04-7.98 (m, 3H), 7.90-7.84 (m, 2H), 7.74-7.70 (m, 1H), 7.70-7.65 (m, 2H), 7.60-7.56 (dd, J=8.8, 2.5 Hz, 1H), 7.34-7.27 (d, J=7.3 Hz, 1H), 6.57-6.51 (d, J=9.0 Hz, 1H), 3.84-3.70 (m, 1H), 3.65-3.55 (m, 2H), 2.67-2.56 (m, 2H), 2.03-1.93 (m, 2H), 1.57-1.46 (m, 2H).

Building blocks: step i: 2-chloro-5-[(trifluoromethyl)sulfanyl]pyridine; step iii: 6-bromoimidazo[1,2-a]pyridine.

### Example 63: 4-(4-{[4-({5-[(trifluoromethyl)sulfanyl]pyridin-2-yl}amino)piperidin-1-yl]sulfonyl}phenyl)pyridine-2-carboxamide

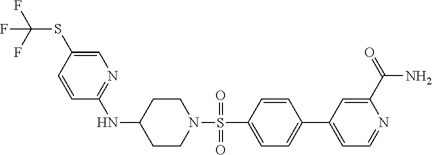 [SMILES: NC(=O)c1cc(-c2ccc(S(=O)(=O)N3CCC(Nc4ccc(SC(F)(F)F)cn4)CC3)cc2)ccn1]

MS(ES⁺) m/z 538.3 (M+H)⁺.

¹H NMR (400 MHz, DMSO) δ 8.82-8.75 (dd, J=5.2, 0.8 Hz, 1H), 8.41-8.32 (dd, J=1.8, 0.8 Hz, 1H), 8.28-8.20 (d, J=2.7 Hz, 1H), 8.20-8.10 (m, 3H), 8.06-7.98 (dd, J=5.2, 1.9 Hz, 1H), 7.96-7.88 (m, 2H), 7.82-7.75 (d, J=2.8 Hz, 1H), 7.62-7.55 (dd, J=8.9, 2.5 Hz, 1H), 7.43-7.32 (d, J=7.4 Hz, 1H), 6.57-6.53 (d, J=8.8 Hz, 1H), 3.86-3.71 (m, 1H), 3.67-3.54 (m, 2H), 2.66-2.59 (m, 2H), 1.99-1.93 (m, 2H), 1.57-1.44 (m, 2H).

Building blocks: step i: 2-chloro-5-[(trifluoromethyl)sulfanyl]pyridine; step iii: 4-bromopyridine-2-carboxamide.

### Example 64: N-(1-{4-[3-(propan-2-yl)imidazo[1,2-a]pyridin-6-yl]benzenesulfonyl}piperidin-4-yl)-5-[(trifluoromethyl)sulfanyl]pyridin-2-amine

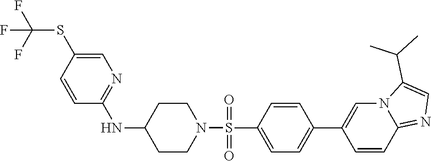 [SMILES: CC(C)c1cnc2ccc(-c3ccc(S(=O)(=O)N4CCC(Nc5ccc(SC(F)(F)F)cn5)CC4)cc3)cn12]

MS(ES⁺) m/z 576.3 (M+H)⁺.

¹H NMR (400 MHz, DMSO) δ 8.77-8.69 (t, J=1.4 Hz, 1H), 8.18-8.11 (d, J=2.4 Hz, 1H), 8.11-8.05 (m, 2H), 7.91-7.83 (m, 2H), 7.74-7.67 (m, 1H), 7.66-7.61 (m, 1H), 7.61-7.54 (dd, J=8.9, 2.4 Hz, 1H), 7.47-7.46 (s, 1H), 7.34-7.30 (d, J=7.2 Hz, 1H), 6.58-6.52 (d, J=8.9 Hz, 1H), 3.82-3.70 (m, 1H), 3.66-3.56 (m, 2H), 3.56-3.47 (dt, J=13.6, 6.8 Hz, 1H), 2.65-2.55 (m, 2H), 2.02-1.94 (m, 2H), 1.62-1.45 (m, 2H), 1.39-1.36 (s, 3H), 1.36-1.34 (s, 3H).

Building blocks: step i: 2-chloro-5-[(trifluoromethyl)sulfanyl]pyridine; step iii: 6-bromo-3-(propan-2-yl)imidazo[1,2-a]pyridine.

### Example 65: N-(1-{4-[3-(propan-2-yl)-[1,2,4]triazolo[4,3-a]pyridin-6-yl]benzenesulfonyl}piperidin-4-yl)-5-[(trifluoromethyl)sulfanyl]pyridin-2-amine

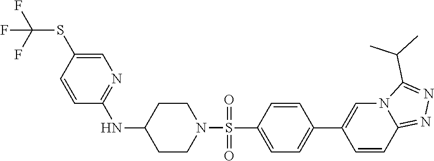 [SMILES: CC(C)c1nnc2ccc(-c3ccc(S(=O)(=O)N4CCC(Nc5ccc(SC(F)(F)F)cn5)CC4)cc3)cn12]

MS(ES⁺) m/z 577.3 (M+H)⁺.

¹H NMR (400 MHz, DMSO) δ 8.88-8.83 (t, J=1.4 Hz, 1H), 8.17-8.13 (d, J=2.4 Hz, 1H), 8.13-8.06 (m, 2H), 7.91-7.84 (m, 3H), 7.80-7.75 (m, 1H), 7.62-7.55 (dd, J=8.9, 2.5 Hz, 1H), 7.35-7.28 (d, J=7.2 Hz, 1H), 6.57-6.50 (dd, J=8.8, 0.8 Hz, 1H), 3.81-3.69 (dt, J=13.7, 6.8 Hz, 2H), 3.64-3.56 (m, 2H), 2.65-2.56 (m, 2H), 2.02-1.93 (m, 2H), 1.59-1.45 (m, 2H), 1.45-1.43 (s, 3H), 1.43-1.41 (s, 3H).

Building blocks: step i: 2-chloro-5-[(trifluoromethyl)sulfanyl]pyridine; step iii: 6-bromo-3-(propan-2-yl)-[1,2,4]triazolo[4,3-a]pyridine.

### Example 66: N-(1-{4-[3-(propan-2-yl)-[1,2,4]triazolo[4,3-a]pyrazin-6-yl]benzenesulfonyl}piperidin-4-yl)-5-[(trifluoromethyl)sulfanyl]pyridin-2-amine

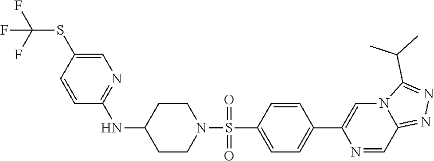 [SMILES: CC(C)c1nnc2cnc(-c3ccc(S(=O)(=O)N4CCC(Nc5ccc(SC(F)(F)F)cn5)CC4)cc3)cn12]

MS(ES⁺) m/z 578.3 (M+H)⁺.

¹H NMR (400 MHz, DMSO) δ 9.56-9.48 (d, J=1.5 Hz, 1H), 9.26-9.20 (d, J=1.7 Hz, 1H), 8.48-8.39 (m, 2H), 8.17-8.10 (d, J=2.4 Hz, 1H), 7.96-7.88 (m, 2H), 7.63-7.53 (dd, J=8.9, 2.5 Hz, 1H), 7.37-7.25 (d, J=7.3 Hz, 1H), 6.55-6.52 (d, J=8.9 Hz, 1H), 3.85-3.69 (m, 2H), 3.64-3.54 (m, 2H), 2.66-2.56 (m, 2H), 2.01-1.92 (m, 2H), 1.58-1.49 (m, 2H), 1.48-1.46 (s, 3H), 1.46-1.45 (s, 3H).

Building blocks: step i: 2-chloro-5-[(trifluoromethyl)sulfanyl]pyridine; step iii: 6-chloro-3-(propan-2-yl)-[1,2,4]triazolo[4,3-a]pyrazine.

### Example 67: N-[1-(4-{3-methyl-[1,2,4]triazolo[4,3-a]pyridin-6-yl}benzenesulfonyl)piperidin-4-yl]-5-[(trifluoromethyl)sulfanyl]pyridin-2-amine

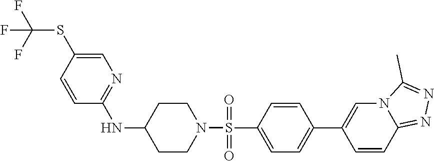 [SMILES: Cc1nnc2ccc(-c3ccc(S(=O)(=O)N4CCC(Nc5ccc(SC(F)(F)F)cn5)CC4)cc3)cn12]

MS(ES⁺) m/z 549.3 (M+H)⁺. ¹H NMR (400 MHz, DMSO) δ 8.79-8.76 (t, J=1.4 Hz, 1H), 8.15-8.13 (d, J=2.4 Hz, 1H), 8.12-8.08 (m, 2H), 7.91-7.87 (m, 2H), 7.86-7.84 (d, J=1.0 Hz, 1H), 7.80-7.76 (m, 1H), 7.61-7.56 (dd, J=8.9, 2.5 Hz, 1H), 7.33-7.29 (d, J=7.2 Hz, 1H), 3.81-3.68 (m, 1H), 3.64-3.55 (m, 2H), 2.84-2.75 (s, 3H), 2.66-2.55 (m, 2H), 2.03-1.93 (m, 2H), 1.61-1.43 (m, 2H).

Building blocks: step i: 2-chloro-5-[(trifluoromethyl)sulfanyl]pyridine; step iii: 6-bromo-3-methyl-[1,2,4]triazolo[4,3-a]pyridine.

### Example 68: N-(1-{4-[8-methyl-3-(propan-2-yl)-[1,2,4]triazolo[4,3-a]pyridin-6-yl]benzenesulfonyl}piperidin-4-yl)-5-[(trifluoromethyl)sulfanyl]pyridin-2-amine

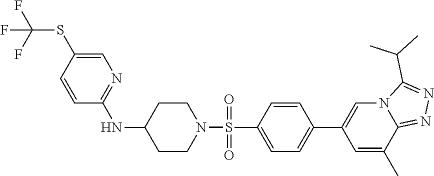 [SMILES: Cc1cc(-c2ccc(S(=O)(=O)N3CCC(Nc4ccc(SC(F)(F)F)cn4)CC3)cc2)cn2c(C(C)C)nnc12]

MS(ES⁺) m/z 591.3 (M+H)⁺.

¹H NMR (400 MHz, DMSO) δ 8.74-8.68 (s, 1H), 8.17-8.12 (d, J=2.4 Hz, 1H), 8.12-8.04 (m, 2H), 7.91-7.85 (m, 2H), 7.62-7.55 (m, 2H), 7.34-7.28 (d, J=7.2 Hz, 1H), 6.57-6.50 (d, J=8.9 Hz, 1H), 3.82-3.65 (m, 2H), 3.65-3.55 (m, 2H), 2.61-2.60 (s, 3H), 2.60-2.54 (m, 2H), 2.03-1.93 (m, 2H), 1.59-1.46 (m, 2H), 1.45-1.43 (s, 3H), 1.43-1.40 (s, 3H).

Building blocks: step i: 2-chloro-5-[(trifluoromethyl)sulfanyl]pyridine; step iii: 6-bromo-8-methyl-3-(propan-2-yl)-[1,2,4]triazolo[4,3-a]pyridine.

### Example 69: N-(1-{4-[8-methyl-3-(propan-2-yl)imidazo[1,2-a]pyridin-6-yl]benzenesulfonyl}piperidin-4-yl)-4-(trifluoromethoxy)pyridin-2-amine

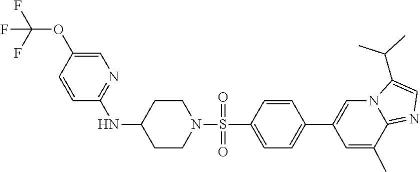 [SMILES: Cc1cc(-c2ccc(S(=O)(=O)N3CCC(Nc4ccc(OC(F)(F)F)cn4)CC3)cc2)cn2c(C(C)C)cnc12]

MS(ES⁺) m/z 574.3 (M+H)⁺.

¹H NMR (400 MHz, DMSO) δ 8.60-8.54 (s, 1H), 8.09-8.02 (m, 2H), 8.02-7.97 (d, J=5.8 Hz, 1H), 7.88-7.82 (m, 2H), 7.51-7.43 (t, J=1.5 Hz, 1H), 7.43-7.39 (s, 1H), 6.98-6.91 (d, J=7.3 Hz, 1H), 6.43-6.39 (m, 1H), 6.38-6.31 (s, 1H), 3.78-3.65 (m, 1H), 3.62-3.53 (m, J=11.7, 4.1 Hz, 2H), 3.53-3.43 (dt, J=13.5, 6.8 Hz, 1H), 2.64-2.58 (m, 2H), 2.58-2.55 (s, 3H), 2.00-1.94 (m, 2H), 1.57-1.44 (m, 2H), 1.38-1.36 (s, 3H), 1.36-1.33 (s, 3H).

Building blocks: step i: 2-chloro-4-(trifluoromethoxy)pyridine; step iii: 6-bromo-8-methyl-3-(propan-2-yl)imidazo[1,2-a]pyridine.

### Example 70: N-(1-{4-[3-(propan-2-yl)-[1,2,4]triazolo[4,3-a]pyridin-6-yl]benzenesulfonyl}piperidin-4-yl)-4-(trifluoromethoxy)pyridin-2-amine

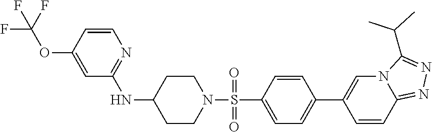 [SMILES: CC(C)c1nnc2ccc(-c3ccc(S(=O)(=O)N4CCC(Nc5cc(OC(F)(F)F)ccn5)CC4)cc3)cn12]

MS(ES⁺) m/z 561.3 (M+H)⁺.

¹H NMR (400 MHz, DMSO) δ 8.87-8.82 (t, J=1.4 Hz, 1H), 8.12-8.07 (m, 2H), 8.01-7.97 (d, J=5.8 Hz, 1H), 7.91-7.85 (m, 3H), 7.80-7.74 (m, 1H), 6.98-6.88 (d, J=7.3 Hz, 1H), 6.44-6.40 (m, 1H), 6.37-6.32 (s, 1H), 3.81-3.66 (m, 2H), 3.63-3.52 (dt, J=12.5, 4.1 Hz, 2H), 2.66-2.56 (m, 2H), 2.03-1.93 (m, 2H), 1.56-1.47 (m, 2H), 1.45-1.43 (s, 3H), 1.43-1.41 (s, 3H).

Building blocks: step i: 2-chloro-4-(trifluoromethoxy)pyridine; step iii: 6-bromo-3-(propan-2-yl)-[1,2,4]triazolo[4,3-a]pyridine.

### Example 71: N-(1-{4-[8-methyl-3-(propan-2-yl)-[1,2,4]triazolo[4,3-a]pyridin-6-yl]benzenesulfonyl}piperidin-4-yl)-4-(trifluoromethoxy)pyridin-2-amine

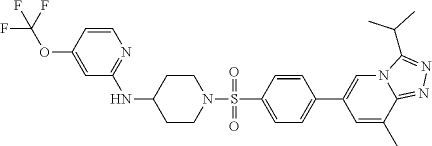 [SMILES: Cc1cc(-c2ccc(S(=O)(=O)N3CCC(Nc4cc(OC(F)(F)F)ccn4)CC3)cc2)cn2c(C(C)C)nnc12]

MS(ES⁺) m/z 575.3 (M+H)⁺.

¹H NMR (400 MHz, DMSO) δ 8.74-8.68 (dd, J=1.4, 0.8 Hz, 1H), 8.13-8.05 (m, 2H), 8.03-7.97 (d, J=5.8 Hz, 1H), 7.92-7.83 (m, 2H), 7.63-7.58 (t, J=1.4 Hz, 1H), 6.98-6.90 (d, J=7.2 Hz, 1H), 6.46-6.39 (m, 1H), 6.38-6.32 (s, 1H), 3.78-3.65 (dt, J=13.7, 6.9 Hz, 2H), 3.64-3.54 (m, 2H), 2.61-2.60 (s, 3H), 2.60-2.55 (m, 2H), 2.03-1.92 (m, 2H), 1.58-1.45 (m, 2H), 1.45-1.43 (s, 3H), 1.43-1.41 (s, 3H).

Building blocks: step i: 2-chloro-4-(trifluoromethoxy)pyridine; step iii: 6-bromo-8-methyl-3-(propan-2-yl)-[1,2,4]triazolo[4,3-a]pyridine.

Following a procedure analogous to that described for Example 47, using in step i the appropriate aniline and in step iii the appropriate boronic ester or boronic acid, Example 72 has been prepared.

### Example 72: N-[trans-4-(4-{3-methyl-1H-pyrazolo[3,4-b]pyridin-5-yl}benzenesulfonyl)cyclohexyl]-4-[(trifluoromethyl)sulfanyl]aniline

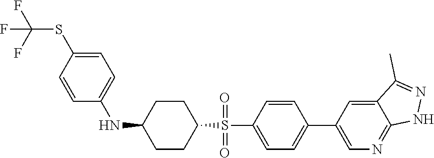 [SMILES: Cc1n[nH]c2ncc(-c3ccc(S(=O)(=O)[C@H]4CC[C@H](Nc5ccc(SC(F)(F)F)cc5)CC4)cc3)cc12]

MS(ES⁺) m/z 547.3 (M+H)⁺.

¹H NMR (400 MHz, DMSO) δ 13.51-13.31 (s, 1H), 8.97-8.90 (d, J=2.2 Hz, 1H), 8.68-8.63 (d, J=2.2 Hz, 1H), 8.16-8.07 (m, 2H), 8.02-7.93 (m, 2H), 7.36-7.29 (m, 2H), 6.70-6.60 (m, 2H), 6.33-6.24 (d, J=7.7 Hz, 1H), 3.42-3.35 (m, 1H), 3.29-3.17 (m, 1H), 2.61-2.56 (s, 3H), 2.07-1.95 (m, 4H), 1.64-1.49 (m, 2H), 1.32-1.14 (m, 2H).

Building blocks: step i: 4-[(trifluoromethyl)sulfanyl]aniline; step iii: 5-bromo-3-methyl-1H-pyrazolo[3,4-b]pyridine.

Following a procedure analogous to that described for Example 39, using in step iv the appropriate boronic ester or boronic acid and in step vi the appropriate (hetero)aryl halide, Example 73 has been prepared.

### Example 73: N-[trans-4-(4-{imidazo[1,2-a]pyridin-6-yl}benzenesulfonyl)cyclohexyl]-5-(trifluoromethoxy)pyridin-2-amine

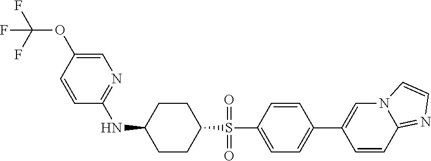 [SMILES: O=S(=O)(c1ccc(-c2ccc3nccn3c2)cc1)[C@H]1CC[C@H](Nc2ccc(OC(F)(F)F)cn2)CC1]

MS(ES⁺) m/z 517.3 (M+H)⁺.

¹H NMR (400 MHz, DMSO) δ 9.16-9.11 (t, J=1.5 Hz, 1H), 8.06-8.00 (m, 3H), 7.99-7.94 (m, 3H), 7.74-7.70 (m, 1H), 7.70-7.66 (m, 1H), 7.66-7.65 (d, J=1.2 Hz, 1H), 3.63-3.49 (m, 1H), 3.40-3.34 (m, 1H), 2.11-1.96 (m, 4H), 1.58-1.37 (m, 2H), 1.32-1.15 (m, 2H).

Building block: step iv: 6-(4,4,5,5-tetramethyl-1,3,2-dioxaborolan-2-yl)imidazo[1,2-a]pyridine; step vi: 2-chloro-5-(trifluoromethoxy)pyridine.

Following a procedure analogous to that described for Example 23, using in Example 3, step i, the appropriate aryl halide, Example 74 has been prepared.

### Example 74: 6-(4-{[4-({5-[(trifluoromethyl)sulfanyl]pyridin-2-yl}amino)piperidin-1-yl]sulfonyl}phenyl)-2H,3H-[1,2,4]triazolo[4,3-a]pyridin-3-one

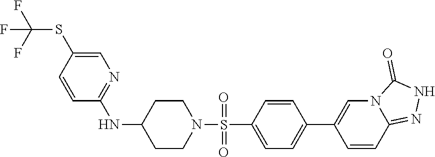 [SMILES: O=c1[nH]nc2ccc(-c3ccc(S(=O)(=O)N4CCC(Nc5ccc(SC(F)(F)F)cn5)CC4)cc3)cn12]

MS(ES⁺) m/z 535.3 (M+H)⁺.

1H NMR (400 MHz, DMSO) δ 12.72-12.46 (s, 1H), 8.25-8.19 (t, J=1.4 Hz, 1H), 8.17-8.11 (d, J=2.4 Hz, 1H), 8.05-7.97 (m, 2H), 7.86-7.78 (m, 2H), 7.70-7.62 (dd, J=9.9, 1.8 Hz, 1H), 7.62-7.53 (dd, J=8.8, 2.5 Hz, 1H), 7.43-7.35 (dd, J=9.8, 1.1 Hz, 1H), 7.34-7.28 (d, J=7.2 Hz, 1H), 6.57-6.49 (d, J=8.9 Hz, 1H), 3.82-3.69 (m, 1H), 3.64-3.52 (m, 2H), 2.65-2.56 (m, 2H), 2.02-1.92 (m, 2H), 1.61-1.42 (m, 2H).

Building block: step i: 2-chloro-5-[(trifluoromethyl)sulfanyl]pyridine.

### Human CCR6-CRE Reporter Assay

293FT cells were transfected with 2 constructs using TransIT-293 (Mirus). The first construct (pGL4.29, Promega) expressed the luciferase reporter gene luc2P in response to the binding of cAMP-induced CREB to the CREelement in its promoter. The second construct contained the open reading frame of human CCR6 in the pUNO1 backbone under the control of an enhanced CMV promoter(InVivoGen).48 h after transfection, cells were harvested and diluted to 750,000 cells/ml in culture medium (DMEM, 10% FBS, 1× pen/strep(Gibco 15140)). Compounds were 2.5-fold serially diluted in DMSO. Further dilutions were made in assay medium (DMEM, 10% FBS, 1× pen/strep, 6 μM Forskolin, 6 nM CCL20). 4 μl of the compound solution was added to a white MW384 plate, followed by 201 of the cell suspension. Final DMSO concentration in the assay was 0.1%. The MW384 plate was placed in an incubator at 37° C., 5% CO2 for 5 h. Luciferase activity was determined by addition of 24 μl of a 2.5× diluted BriteLite luciferase solution(Perkin Elmer), followed by luminescence measurement using a VICTOR Plate reader (Perkin Elmer). To deselect compounds that did not specifically bind CCR6, the assay was also performed with transfected 293FT cells in which CCR6 was replaced with an empty vector using the same backbone. The relative Log IC50 was determined with GraphPad Prism 9 using a four-parameter dose-response model.

### CHO-K1 CCR6 Chemotaxis Assay Towards CCL20

Cells overexpressing human CCR6 were used in the chemotaxis assay (DiscoverX, cAMP Hunter™ CHO-K1 CCR6 Gi Cell Line). Growth medium of these cells consisted of DMEM/F12, non-essential Amino acids (1×NEAA, Gibco 11140050), 10% FBS and 1× pen/strep (Gibco 15140). For the chemotaxis assay, Corning Transwell plates were used with a pore size of 8 m (Corning 351164). Cells were harvested using trypsin and diluted to 4*10\hat{ }6 cells/ml in chemotaxis medium (DMEM, 0.25% BSA, pen/strep). Compounds were four-fold serially diluted in DMSO. Further dilutions were made in chemotaxis medium. Cells were incubated with test compounds for 30 min at 37° C. prior to initiation of the assay (2*10\hat{ }6 cells/ml). Final DMSO concentration in the assay was 0.2%. Wells of the Transwell plates were filled with 200 μl chemotaxis medium containing 100 ng/ml CCL20 (R&D systems 360-MP). 50 μl of the cell/compound suspension was added to the inserts of the Transwell plates (100,000 cells/insert). The Transwell plates were placed in an incubator at 37° C., 5% CO₂ for 4 h. After 4 h, the number of migrated cells was quantified using CellTiter Glo (Promega). Percentage inhibition values were calculated based on the low and high signal obtained with 0.2% DMSO in chemotaxis medium without and with CCL20, respectively. The relative Log IC50 was determined with GraphPad Prism 9 using a four-parameter dose-response model.

### CD4+ T Cells Chemotaxis Assay Towards CCL20

Human CD4+ T cells were isolated from buffy coats of healthy donors using aCD4+ T Cell Isolation Kit(Miltenyi Biotec 130-096-533). Isolated T cells were stimulated overnight with anti-CD3 and anti-CD28 antibodies (Biolegend 300314/302934) in growth medium (RPMI1640, 10% heat-inactivated FBS, 1× pen/strep (Gibco 15140)). For the chemotaxis assay, Corning Transwell plates were used with a pore size of 5 m (Corning 3388). T cells were counted and diluted to 6*10\hat{ }6 cells/ml in chemotaxis medium (RPMI1640, 1% BSA, pen/strep). Compounds were four-fold serially diluted in DMSO. Further dilutions were made in chemotaxis medium. Cells were incubated with test compounds for 30 min at 37° C. prior to initiation of the assay (3*10\hat{ }6 cells/ml). Final DMSO concentration in the assay was 0.2%. Wells of the Transwell plates were filled with 200 μl chemotaxis medium containing 150 ng/ml CCL20 (R&D systems 360-MP). 50 μl of the cell/compound suspension was added to the inserts of the Transwell plates (150,000 cells/insert). The Transwell plates were placed in an incubator at 37° C., 5% CO2 for 3 h. After 3 h, the number of migrated cells was quantified using CellTiter Glo (Promega). Percentage inhibition values were calculated based on the low and high signal obtained with 0.2% DMSO in chemotaxis medium without and with CCL20, respectively. The relative Log IC50 was determined with GraphPad Prism 9 using a four-parameter dose-response model.

The tables below show the data for selected compounds:

TABLE 1
CCR6-CRE reporter assay

| Example | CCR6-CRE | | |
|---|---|---|---|
| Number | reporter | | |
| 1 | 7.4 | 13 | 7.1 |
| 2 | 6.4 | 14 | 7.2 |
| 3 | 6.9 | 15 | 7.3 |
| 4 | 7.0 | 16 | 7.1 |
| 5 | 6.9 | 17 | 7.0 |
| 6 | 6.9 | 18 | 6.7 |
| 7 | 6.7 | 19 | 6.9 |
| 8 | 6.9 | 20 | 7.3 |
| 9 | 7.1 | 21 | 7.5 |
| 10 | 5.5 | 22 | 6.6 |
| 11 | 6.7 | 23 | 6.9 |
| 12 | 6.8 | 24 | 7.1 |
| 28 | 6.9 | 25 | 6.2 |
| 29 | 7.2 | 26 | 6.3 |
| 30 | 7.1 | 27 | 6.7 |
| 31 | 7.3 | 45 | 7.1 |
| 32 | 7.1 | 46 | 7.2 |
| 33 | 7.0 | 47 | 7.0 |
| 34 | 7.2 | 48 | 7.1 |
| 35 | 5.6 | 49 | 7.2 |
| 36 | 6.4 | 50 | 7.1 |
| 37 | 6.6 | 51 | 7.1 |
| 38 | 6.9 | 52 | 6.8 |
| 39 | 7.0 | 53 | 6.5 |
| 40 | 6.4 | 54 | 6.2 |
| 41 | 6.8 | 55 | 7.1 |
| 42 | 6.8 | 56 | 7.5 |
| 43 | 7.1 | 57 | 7.4 |
| 44 | 7.7 | 58 | 7.2 |
| 62 | 7.3 | 59 | 7.2 |
| 63 | 7.1 | 60 | 7.4 |
| 64 | 7.4 | 61 | 7.2 |
| 65 | 7.6 | 3 | 7.0 |
| 66 | 7.5 | 8 | 5.3 |
| 67 | 7.4 | 11 | 7.6 |
| 68 | 7.2 | 15 | 6.9 |
| 69 | 5.9 | 17 | 7.5 |
| 70 | 5.5 | 18 | 6.1 |
| 71 | 6.0 | 19 | 6.3 |
| 72 | 7.4 | 20 | 7.0 |
| 73 | 6.9 | 21 | 7.1 |
| 74 | 7.1 | 23 | 7.2 |
| | | 28 | 6.5 |
| | | 29 | 6.6 |
| | | 30 | 7.8 |
| | | 32 | 6.8 |

TABLE 2
CHO-K1 CCR6 chemotaxis assay

| Example | CHO-K1 CCR6 | | |
|---|---|---|---|
| Number | chemotaxis | | |
| 42 | 7.1 | 33 | 7.7 |
| 43 | 7.1 | 39 | 7.0 |
| 44 | 8.2 | 41 | 7.2 |
| 46 | 6.1 | 14 | 7.7 |
| 51 | 6.1 | 15 | 8.0 |
| 57 | 8.1 | 23 | 7.4 |
| 63 | 7.8 | 24 | 7.5 |
| | | 29 | 6.9 |
| | | 39 | 8.1 |
| | | 55 | 8.7 |
| | | 59 | 8.1 |

TABLE 3
CD4+ T cells chemotaxis assay

| Example | CD4+ T-cell | | |
|---|---|---|---|
| Number | chemotaxis | | |
| 1 | 7.3 | 65 | 8.3 |
| 4 | 7.7 | 66 | 8.8 |
| 6 | 7.3 | 67 | 7.9 |
| 13 | 8.0 | 68 | 8.7 |
| | | 72 | 8.4 |
| | | 74 | 7.9 |

TABLE 4
Activity in human CCR6-CRE reporter assay

| | X = O | | X = S | |
|---|---|---|---|---|
| | R1 = CH₃ | R1 = CF₃ | R1 = CH₃ | R1 = CF₃ |
| 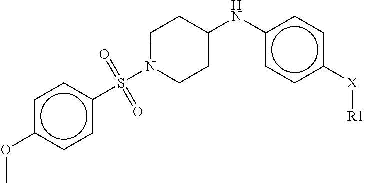 | >20 uM (A) | 1.7 uM | >20 uM | 1.5 uM |
| 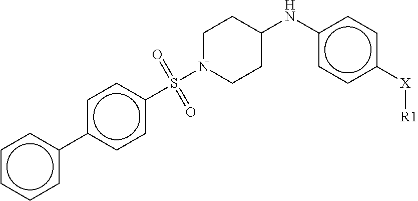 | >20 uM | 1.1 uM (Example 25) | >20 uM | 1.3 uM |
| 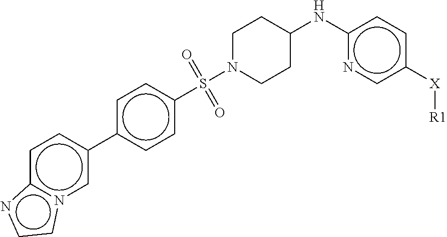 | 1.4 uM | 0.15 uM (Example 12) | 0.61 uM | 0.050 uM (Example 62) |
| 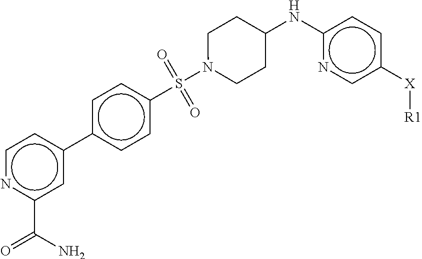 | >20 uM | 0.062 uM (Example 58) | 0.19 uM | 0.074 uM (Example 63) |

(A) Refers to compound 10 of TAWARAISHI TAISUKE ET AL: “Identification of a novel series of potent and selective CCR6 inhibitors as biological probes”, BIOORGANIC & MEDICINAL CHEMISTRY LETTERS, ELSEVIER, AMSTERDAM NL, vol. 28, no. 18, 30 Jul. 2018 (2018-07-30), pages 3067-3072, XP085456366, ISSN: 0960-894X, DOI: 10.1016/J.BMCL.2018.07.042

Aspect 1. A compound of formula (I)

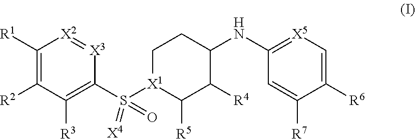

wherein
X¹ is CH or N;
X² is CH or N;
X³ is CH or N;
X⁴ is O or NH;
X⁵ is CH or N;
R¹ is aryl, heterocyclyl, or heteroaryl, wherein aryl, heterocyclyl, and heteroaryl are optionally substituted with one or more R^{1a};
R^{1a} is C₁₋₆alkyl, oxo, cyano, carbamoyl, C₁₋₆alkylcarbamoyl-, C₁₋₆alkoxyC₁₋₆alkyl-, C₃₋₆cycloalkyl, or heterocyclyl;
R² is hydrogen or halogen;
R³ is hydrogen or halogen;
R⁴ is hydrogen, halogen or C₁₋₆alkyl;
R⁵ is hydrogen, halogen or C₁₋₆alkyl;
R⁶ is —OR^{6a}, —SR^{6b}, or hydrogen;
R^{6a} is C₁₋₆haloalkyl;
R^{6b} is C₁₋₆haloalkyl;
R⁷ is —OR^{7a}, —SR^{7b}, or hydrogen;
R^{7a} is C₁₋₆haloalkyl;
R^{7b} is C₁₋₆haloalkyl;
provided that R⁶ and R⁷ must be different, and R⁶ or R⁷ is hydrogen, and pharmaceutically acceptable salts thereof.

Aspect 2. The compound of aspect 1 wherein
X² is CH;
X³ is CH;
R² is hydrogen;
R³ is hydrogen;
R⁴ is hydrogen;
R⁵ is hydrogen or C₁₋₆alkyl.

Aspect 3. The compound of aspect 1 or 2 wherein X¹ is CH.

Aspect 4. The compound of aspect 1 or 2 wherein X¹ is N.

Aspect 5. The compound of any one of aspects 1 to 4 wherein X⁴ is O.

Aspect 6. The compound of any one of aspects 1 to 4 wherein X⁴ is NH.

Aspect 7. The compound of any one of aspects 1 to 6 wherein X⁵ is CH.

Aspect 8. The compound of any one of aspects 1 to 6 wherein X⁵ is N.

Aspect 9. The compound of any one of aspects 1 to 8 wherein R¹ is imidazopyridinyl, triazolopyridinyl, phenyl, indolyl, isoindolinyl, or indazolyl, wherein imidazopyridinyl, triazolopyridinyl, phenyl, indolyl, isoindolinyl, and indazolyl, are optionally substituted with one or more R^{1a}.

Aspect 10. The compound of any one of aspects 1 to 9 wherein R¹ is indazolyl, indolyl, indazolyl, imidazopyridinyl, or triazolopyridinyl, wherein indazolyl, indolyl, indazolyl, imidazopyridinyl, and triazolopyridinyl, are optionally substituted with one or more R^{1a}.

Aspect 11. The compound of any one of aspects 1 to 10 wherein R¹ is indazolyl, indolyl, indazolyl, imidazopyridinyl, or triazolopyridinyl, wherein indazolyl, indolyl, indazolyl, imidazopyridinyl, and triazolopyridinyl, are optionally substituted with one or more cyano, methyl, oxo, isopropyl, or cyclopentyl.

Aspect 12. The compound of any one of aspects 1 to 9 wherein R¹ is isopropylimidazopyridinyl, oxo-triazolopyridinyl, imidazopyridinyl, cyanophenyl, methylimidazopyridinyl, cyano-indolyl, oxoisoindolinyl, carbamoylphenyl, (methylcarbamoyl)phenyl, methyl-indazolyl, phenyl, cyano-indazolyl, cyanoimidazopyridinyl, methyl-triazolopyridinyl, (methoxymethyl)-triazolopyridinyl, or cyclopentyl-triazolopyridinyl.

Aspect 13. The compound of any of the aspects 1 to 9 and 12 wherein R¹ is (3-isopropylimidazo[1,2-a]pyridin-6-yl), (3-oxo-2H-[1,2,4]triazolo[4,3-a]pyridin-6-yl), imidazo[1,2-a]pyridin-6-yl, (4-cyanophenyl), (3-methylimidazo[1,2-a]pyridin-6-yl), (3-cyano-1H-indol-5-yl), (3-isopropyl-8-methyl-imidazo[1,2-a]pyridin-6-yl), (2-carbamoyl-4-pyridyl), (3-oxoisoindolin-5-yl), (3-carbamoylphenyl), [3-(methylcarbamoyl)phenyl], (3-methyl-1H-indazol-5-yl), phenyl, (3-cyano-1H-indazol-5-yl), (3-cyanoimidazo[1,2-a]pyridin-6-yl), (2-methyl-[1,2,4]triazolo[1,5-a]pyridin-6-yl), (2-methyl-[1,2,4]triazolo[1,5-a]pyridin-7-yl), [3-(methoxymethyl)-[1,2,4]triazolo[4,3-a]pyridin-6-yl], or (3-cyclopentyl-[1,2,4]triazolo[4,3-a]pyridin-6-yl).

Aspect 14. The compound of any one of aspects 1 to 9, and 12 wherein R¹ is cyano-indazolyl, cyano-indolyl, methyl-indazolyl, oxo-triazolopyridinyl, isopropylimidazopyridinyl, or cyclopentyl-triazolopyridinyl.

Aspect 15. The compound of any of the claims 1 to 9, and 12, wherein R¹ is (3-cyano-1H-indol-5-yl), (3-methyl-1H-indazol-5-yl), (3-cyano-1H-indazol-5-yl), (3-oxo-2H-[1,2,4]triazolo[4,3-a]pyridin-6-yl), (3-isopropylimidazo[1,2-a]pyridin-6-yl), or cyclopentyl-triazolopyridinyl.

Aspect 16. The compound of any one of aspects 1 to 15 wherein R^{1a} is isopropyl, methyl, oxo, cyano, carbamoyl, methylcarbamoyl, or methoxymethyl.

Aspect 17. The compound of any one of aspects 1 to 16, wherein R^{1a} is cyano, methyl, oxo, isopropyl, or cyclopentyl.

Aspect 18. The compound of any one of aspects 1 to 17 wherein R⁶ is —SCF₃, —OCF₃, —OCHF₂, or hydrogen.

Aspect 19. The compound of any one of aspects 1 to 18 wherein R⁶ is —SCF₃, or —OCF₃.

Aspect 20. The compound of any one of aspects 1 to 19 wherein R^{6a} is —CF₃, or —CHF₂.

Aspect 21. The compound of any one of aspects 1 to 20 wherein R^{6b} is —CF₃.

Aspect 22. The compound of any one of aspects 1 to 21 wherein R⁷ is —OCF₃, —SCF₃, —OCHF₂, or hydrogen.

Aspect 23. The compound of any of aspects 1 to 22 wherein R⁷ is hydrogen.

Aspect 24. The compound of any one of aspects 1 to 23 wherein R^{7a} is —CF₃, or —CHF₂.

Aspect 25. The compound of any one of aspects 1 to 23 wherein R^{7b} is —CF₃.

Aspect 26. The compound of any one of aspects 1 to 25 wherein R⁵ is hydrogen or methyl.

Aspect 27. The compound of any one of aspects 1 to 26, wherein R¹ is indazolyl, indolyl, indazolyl, imidazopyridinyl, or triazolopyridinyl, wherein indazolyl, indolyl, indazolyl, imidazopyridinyl, and triazolopyridinyl, are optionally substituted with one or more cyano, methyl, oxo, isopropyl, or cyclopentyl, R⁶ is —SCF₃, or —OCF₃; and R², R³, R⁴, R⁵, R⁷, X¹, X², X³, X⁴, and X⁵ are as defined in aspects 1 to 26.

Aspect 28. The compound of any one of aspects 1 to 27 wherein the compound is of formula (I′)

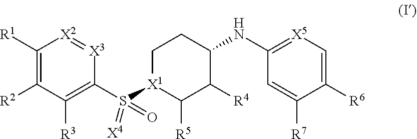

or a pharmaceutically acceptable salt thereof and R¹, R², R³, R⁴, R⁵, R⁶, R⁷, X², X³, X⁴, and X⁵ are as defined in any one of aspects 1 to 27 and X¹ is CH.

Aspect 29. The compound of any one of aspects 1 to 27 wherein the compound is of formula (I″)

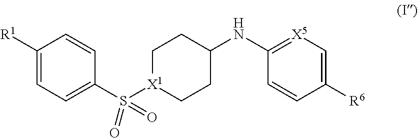

or a pharmaceutically acceptable salt thereof and R¹, X¹, and X⁵ are as defined in any one of aspects 1 to 27, and R⁶ is as defined in aspects 19.

Aspect 30. The compound of any one of aspects 1 to 29 selected from:
{4-[3-(propan-2-yl)imidazo[1,2-a]pyridin-6-yl]phenyl}[trans-4-({4-[(trifluoromethyl)sulfanyl]phenyl}Amino)cyclohexyl](imino)-λ⁶-sulfanone;
6-(4-{[trans-4-({4-[(trifluoromethyl)sulfanyl]phenyl}Amino)cyclohexyl]sulfonimidoyl}phenyl)-2H,3H-[1,2,4]triazolo[4,3-a]pyridin-3-one;
(4-{imidazo[1,2-a]pyridin-6-yl}phenyl)[trans-4-({4-[(trifluoromethyl)sulfanyl]phenyl}Amino)cyclohexyl](imino)-λ⁶-sulfanone;
(4-{imidazo[1,2-a]pyridin-6-yl}phenyl)[trans-4-{[5-(trifluoromethoxy)pyridin-2-yl]Amino}cyclohexyl](imino)-λ⁶-sulfanone;
4′-{[trans-4-{[4-(trifluoromethoxy)phenyl]Amino}cyclohexyl]sulfonyl}-[1,1′-biphenyl]-4-carbonitrile;
N-[trans-4-(4-{3-methylimidazo[1,2-a]pyridin-6-yl}benzenesulfonyl)cyclohexyl]-4-(trifluoromethoxy)aniline;
N-[trans-4-(4-{3-methylimidazo[1,2-a]pyridin-6-yl}benzenesulfonyl)cyclohexyl]-5-(trifluoromethoxy)pyridin-2-amine;
5-(4-{[trans-4-({4-[(trifluoromethyl)sulfanyl]phenyl}Amino)cyclohexyl]sulfonyl}phenyl)-1H-indole-3-carbonitrile;
N-[trans-4-(4-{imidazo[1,2-a]pyridin-6-yl}benzenesulfonyl)cyclohexyl]-4-(trifluoromethoxy)aniline;
6-(4-{[trans-4-({4-[(trifluoromethyl)sulfanyl]phenyl}Amino)cyclohexyl]sulfonyl}phenyl)-2H,3H-[1,2,4]triazolo[4,3-a]pyridin-3-one;
N-[trans-4-(4-{imidazo[1,2-a]pyridin-6-yl}benzenesulfonyl)cyclohexyl]-4-[(trifluoromethyl)sulfanyl]aniline;
N-[trans-4-{4-[8-methyl-3-(propan-2-yl)imidazo[1,2-a]pyridin-6-yl]benzenesulfonyl}cyclohexyl]-4-[(trifluoromethyl)sulfanyl]aniline;
4-(4-{[trans-4-({4-[(trifluoromethyl)sulfanyl]phenyl}Amino)cyclohexyl]sulfonyl}phenyl)pyridine-2-carboxamide;
4′-[(4-{[5-(trifluoromethoxy)pyridin-2-yl]Amino}piperidin-1-yl)sulfonyl]-[1,1′-biphenyl]-4-carbonitrile;
6-{4-[(4-{[5-(trifluoromethoxy)pyridin-2-yl]Amino}piperidin-1-yl)sulfonyl]phenyl}-2,3-dihydro-1H-isoindol-1-one;
4′-[(4-{[5-(trifluoromethoxy)pyridin-2-yl]Amino}piperidin-1-yl)sulfonyl]-[1,1′-biphenyl]-3-carboxamide;
5-{4-[(4-{[5-(trifluoromethoxy)pyridin-2-yl]Amino}piperidin-1-yl)sulfonyl]phenyl}-1H-indole-3-carbonitrile;
4′-[(4-{[4-(trifluoromethoxy)phenyl]Amino}piperidin-1-yl)sulfonyl]-[1,1′-biphenyl]-4-carbonitrile;
N-methyl-4′-[(4-{[4-(trifluoromethoxy)phenyl]Amino}piperidin-1-yl)sulfonyl]-[1,1′-biphenyl]-3-carboxamide;
6-{4-[(4-{[4-(trifluoromethoxy)phenyl]Amino}piperidin-1-yl)sulfonyl]phenyl}-2,3-dihydro-1H-isoindol-1-one;
4′-[(4-{[4-(trifluoromethoxy)phenyl]Amino}piperidin-1-yl)sulfonyl]-[1,1′-biphenyl]-3-carboxamide;
1-[4-(3-methyl-1H-indazol-5-yl)benzenesulfonyl]-N-[4-(trifluoromethoxy)phenyl]piperidin-4-amine;
5-{4-[(4-{[4-(trifluoromethoxy)phenyl]Amino}piperidin-1-yl)sulfonyl]phenyl}-1H-indole-3-carbonitrile;
5-{4-[(4-{[4-(trifluoromethoxy)phenyl]Amino}piperidin-1-yl)sulfonyl]phenyl}-1H-indazole-3-carbonitrile;
1-{[1,1′-biphenyl]-4-sulfonyl}-N-[4-(trifluoromethoxy)phenyl]piperidin-4-amine;
1-{[1,1′-biphenyl]-4-sulfonyl}-2-methyl-N-[4-(trifluoromethoxy)phenyl]piperidin-4-amine;
4′-[(2-methyl-4-{[4-(trifluoromethoxy)phenyl]Amino}piperidin-1-yl)sulfonyl]-[1,1′-biphenyl]-4-carbonitrile;
6-{4-[(2-methyl-4-{[4-(trifluoromethoxy)phenyl]Amino}piperidin-1-yl)sulfonyl]phenyl}-2,3-dihydro-1H-isoindol-1-one;
N-methyl-4′-[(2-methyl-4-{[4-(trifluoromethoxy)phenyl]Amino}piperidin-1-yl)sulfonyl]-[1,1′-biphenyl]-3-carboxamide;
N-[4-(difluoromethoxy)phenyl]-1-[4-(3-methyl-1H-indazol-5-yl)benzenesulfonyl]piperidin-4-amine;
6-{4-[(4-{[4-(difluoromethoxy)phenyl]Amino}piperidin-1-yl)sulfonyl]phenyl}-2,3-dihydro-1H-isoindol-1-one;
5-{4-[(4-{[4-(difluoromethoxy)phenyl]Amino}piperidin-1-yl)sulfonyl]phenyl}-1H-indole-3-carbonitrile;
1-{[1,1′-biphenyl]-4-sulfonyl}-N-[4-(difluoromethoxy)phenyl]piperidin-4-amine;
1-(4-{imidazo[1,2-a]pyridin-6-yl}benzenesulfonyl)-N-[4-(trifluoromethoxy)phenyl]piperidin-4-amine;
1-(4-{3-methylimidazo[1,2-a]pyridin-6-yl}benzenesulfonyl)-N-[4-(trifluoromethoxy)phenyl]piperidin-4-amine;
6-{4-[(4-{[4-(trifluoromethoxy)phenyl]Amino}piperidin-1-yl)sulfonyl]phenyl}imidazo[1,2-a]pyridine-3-carbonitrile;
6-{4-[(4-{[4-(trifluoromethoxy)phenyl]Amino}piperidin-1-yl)sulfonyl]phenyl}-2H,3H-[1,2,4]triazolo[4,3-a]pyridin-3-one;
1-(4-{2-methyl-[1,2,4]triazolo[1,5-a]pyridin-6-yl}benzenesulfonyl)-N-[4-(trifluoromethoxy)phenyl]piperidin-4-amine;
1-(4-{2-methyl-[1,2,4]triazolo[1,5-a]pyridin-7-yl}benzenesulfonyl)-N-[4-(trifluoromethoxy)phenyl]piperidin-4-amine;
1-{4-[3-(propan-2-yl)imidazo[1,2-a]pyridin-6-yl]benzenesulfonyl}-N-[4-(trifluoromethoxy)phenyl]piperidin-4-amine;
N-(1-{4-[3-(propan-2-yl)imidazo[1,2-a]pyridin-6-yl]benzenesulfonyl}piperidin-4-yl)-5-(trifluoromethoxy)pyridin-2-amine;
N-[1-(4-{imidazo[1,2-a]pyridin-6-yl}benzenesulfonyl)piperidin-4-yl]-5-(trifluoromethoxy)pyridin-2-amine;
1-{4-[3-(propan-2-yl)imidazo[1,2-a]pyridin-6-yl]benzenesulfonyl}-N-{4-[(trifluoromethyl)sulfanyl]phenyl}piperidin-4-amine;
1-{4-[3-(methoxymethyl)-[1,2,4]triazolo[4,3-a]pyridin-6-yl]benzenesulfonyl}-N-[4-(trifluoromethoxy)phenyl]piperidin-4-amine;
6-{4-[(4-{[5-(trifluoromethoxy)pyridin-2-yl]Amino}piperidin-1-yl)sulfonyl]phenyl}-2H,3H-[1,2,4]triazolo[4,3-a]pyridin-3-one;
1-(4-{imidazo[1,2-a]pyridin-6-yl}benzenesulfonyl)-N-{4-[(trifluoromethyl)sulfanyl]phenyl}piperidin-4-amine;
1-(4-{3-cyclopentyl-[1,2,4]triazolo[4,3-a]pyridin-6-yl}benzenesulfonyl)-N-[4-(trifluoromethoxy)phenyl]piperidin-4-amine;
1-{4-[8-methyl-3-(propan-2-yl)imidazo[1,2-a]pyridin-6-yl]benzenesulfonyl}-N-{4-[(trifluoromethyl)sulfanyl]phenyl}piperidin-4-amine;
4-(4-{[4-({4-[(trifluoromethyl)sulfanyl]phenyl}Amino)piperidin-1-yl]sulfonyl}phenyl)pyridine-2-carboxamide;
4′-{[trans-4-{[3-(trifluoromethoxy)phenyl]Amino}cyclohexyl]sulfonyl}-[1,1′-biphenyl]-4-carbonitrile;
N-[trans-4-(4-{imidazo[1,2-a]pyridin-6-yl}benzenesulfonyl)cyclohexyl]-3-[(trifluoromethyl)sulfanyl]aniline;
N-[trans-4-{4-[3-(propan-2-yl)imidazo[1,2-a]pyridin-6-yl]benzenesulfonyl}cyclohexyl]-3-[(trifluoromethyl)sulfanyl]aniline;
6-{4-[(4-{[3-(trifluoromethoxy)phenyl]Amino}piperidin-1-yl)sulfonyl]phenyl}-2,3-dihydro-1H-isoindol-1-one
   or
4′-[(4-{[3-(difluoromethoxy)phenyl]Amino}piperidin-1-yl)sulfonyl]-[1,1′-biphenyl]-4-carbonitrile.

Aspect 31. The compound of any one of aspects 1 to 30 selected from:
{4-[3-(propan-2-yl)imidazo[1,2-a]pyridin-6-yl]phenyl}[trans-4-({4-[(trifluoromethyl)sulfanyl]phenyl}Amino)cyclohexyl](imino)-λ⁶-sulfanone;
6-(4-{[trans-4-({4-[(trifluoromethyl)sulfanyl]phenyl}Amino)cyclohexyl]sulfonimidoyl}phenyl)-2H,3H-[1,2,4]triazolo[4,3-a]pyridin-3-one;
(4-{imidazo[1,2-a]pyridin-6-yl}phenyl)[trans-4-({4-[(trifluoromethyl)sulfanyl]phenyl}Amino)cyclohexyl](imino)-λ⁶-sulfanone;
(4-{imidazo[1,2-a]pyridin-6-yl}phenyl)[trans-4-{[5-(trifluoromethoxy)pyridin-2-yl]Amino}cyclohexyl](imino)-λ⁶-sulfanone;
4′-{[trans-4-{[4-(trifluoromethoxy)phenyl]Amino}cyclohexyl]sulfonyl}-[1,1′-biphenyl]-4-carbonitrile;
N-[trans-4-(4-{3-methylimidazo[1,2-a]pyridin-6-yl}benzenesulfonyl)cyclohexyl]-4-(trifluoromethoxy)aniline;
N-[trans-4-(4-{3-methylimidazo[1,2-a]pyridin-6-yl}benzenesulfonyl)cyclohexyl]-5-(trifluoromethoxy)pyridin-2-amine;
5-(4-{[trans-4-({4-[(trifluoromethyl)sulfanyl]phenyl}Amino)cyclohexyl]sulfonyl}phenyl)-1H-indole-3-carbonitrile;
N-[trans-4-(4-{imidazo[1,2-a]pyridin-6-yl}benzenesulfonyl)cyclohexyl]-4-(trifluoromethoxy)aniline;
6-(4-{[trans-4-({4-[(trifluoromethyl)sulfanyl]phenyl}Amino)cyclohexyl]sulfonyl}phenyl)-2H,3H-[1,2,4]triazolo[4,3-a]pyridin-3-one;
N-[trans-4-(4-{imidazo[1,2-a]pyridin-6-yl}benzenesulfonyl)cyclohexyl]-4-[(trifluoromethyl)sulfanyl]aniline;
N-[trans-4-{4-[8-methyl-3-(propan-2-yl)imidazo[1,2-a]pyridin-6-yl]benzenesulfonyl}cyclohexyl]-4-[(trifluoromethyl)sulfanyl]aniline;
4-(4-{[trans-4-({4-[(trifluoromethyl)sulfanyl]phenyl}Amino)cyclohexyl]sulfonyl}phenyl)pyridine-2-carboxamide;
4′-[(4-{[5-(trifluoromethoxy)pyridin-2-yl]Amino}piperidin-1-yl)sulfonyl]-[1,1′-biphenyl]-4-carbonitrile;
6-{4-[(4-{[5-(trifluoromethoxy)pyridin-2-yl]Amino}piperidin-1-yl)sulfonyl]phenyl}-2,3-dihydro-1H-isoindol-1-one;
4′-[(4-{[5-(trifluoromethoxy)pyridin-2-yl]Amino}piperidin-1-yl)sulfonyl]-[1,1′-biphenyl]-3-carboxamide;
5-{4-[(4-{[5-(trifluoromethoxy)pyridin-2-yl]Amino}piperidin-1-yl)sulfonyl]phenyl}-1H-indole-3-carbonitrile;
4′-[(4-{[4-(trifluoromethoxy)phenyl]Amino}piperidin-1-yl)sulfonyl]-[1,1′-biphenyl]-4-carbonitrile;
N-methyl-4′-[(4-{[4-(trifluoromethoxy)phenyl]Amino}piperidin-1-yl)sulfonyl]-[1,1′-biphenyl]-3-carboxamide;
6-{4-[(4-{[4-(trifluoromethoxy)phenyl]Amino}piperidin-1-yl)sulfonyl]phenyl}-2,3-dihydro-1H-isoindol-1-one;
4′-[(4-{[4-(trifluoromethoxy)phenyl]Amino}piperidin-1-yl)sulfonyl]-[1,1′-biphenyl]-3-carboxamide;
1-[4-(3-methyl-1H-indazol-5-yl)benzenesulfonyl]-N-[4-(trifluoromethoxy)phenyl]piperidin-4-amine;
5-{4-[(4-{[4-(trifluoromethoxy)phenyl]Amino}piperidin-1-yl)sulfonyl]phenyl}-1H-indole-3-carbonitrile;
5-{4-[(4-{[4-(trifluoromethoxy)phenyl]Amino}piperidin-1-yl)sulfonyl]phenyl}-1H-indazole-3-carbonitrile;
1-{[1,1′-biphenyl]-4-sulfonyl}-N-[4-(trifluoromethoxy)phenyl]piperidin-4-amine;
1-{[1,1′-biphenyl]-4-sulfonyl}-2-methyl-N-[4-(trifluoromethoxy)phenyl]piperidin-4-amine;
4′-[(2-methyl-4-{[4-(trifluoromethoxy)phenyl]Amino}piperidin-1-yl)sulfonyl]-[1,1′-biphenyl]-4-carbonitrile;
6-{4-[(2-methyl-4-{[4-(trifluoromethoxy)phenyl]Amino}piperidin-1-yl)sulfonyl]phenyl}-2,3-dihydro-1H-isoindol-1-one;
N-methyl-4′-[(2-methyl-4-{[4-(trifluoromethoxy)phenyl]Amino}piperidin-1-yl)sulfonyl]-[1,1′-biphenyl]-3-carboxamide;
N-[4-(difluoromethoxy)phenyl]-1-[4-(3-methyl-1H-indazol-5-yl)benzenesulfonyl]piperidin-4-amine;
6-{4-[(4-{[4-(difluoromethoxy)phenyl]Amino}piperidin-1-yl)sulfonyl]phenyl}-2,3-dihydro-1H-isoindol-1-one;
5-{4-[(4-{[4-(difluoromethoxy)phenyl]Amino}piperidin-1-yl)sulfonyl]phenyl}-1H-indole-3-carbonitrile;
1-{[1,1′-biphenyl]-4-sulfonyl}-N-[4-(difluoromethoxy)phenyl]piperidin-4-amine;
1-(4-{imidazo[1,2-a]pyridin-6-yl}benzenesulfonyl)-N-[4-(trifluoromethoxy)phenyl]piperidin-4-amine;
1-(4-{3-methylimidazo[1,2-a]pyridin-6-yl}benzenesulfonyl)-N-[4-(trifluoromethoxy)phenyl]piperidin-4-amine;
6-{4-[(4-{[4-(trifluoromethoxy)phenyl]Amino}piperidin-1-yl)sulfonyl]phenyl}imidazo[1,2-a]pyridine-3-carbonitrile;
6-{4-[(4-{[4-(trifluoromethoxy)phenyl]Amino}piperidin-1-yl)sulfonyl]phenyl}-2H,3H-[1,2,4]triazolo[4,3-a]pyridin-3-one;
1-(4-{2-methyl-[1,2,4]triazolo[1,5-a]pyridin-6-yl}benzenesulfonyl)-N-[4-(trifluoromethoxy)phenyl]piperidin-4-amine;
1-(4-{2-methyl-[1,2,4]triazolo[1,5-a]pyridin-7-yl}benzenesulfonyl)-N-[4-(trifluoromethoxy)phenyl]piperidin-4-amine;
1-{4-[3-(propan-2-yl)imidazo[1,2-a]pyridin-6-yl]benzenesulfonyl}-N-[4-(trifluoromethoxy)phenyl]piperidin-4-amine;
N-(1-{4-[3-(propan-2-yl)imidazo[1,2-a]pyridin-6-yl]benzenesulfonyl}piperidin-4-yl)-5-(trifluoromethoxy)pyridin-2-amine;
N-[1-(4-{imidazo[1,2-a]pyridin-6-yl}benzenesulfonyl)piperidin-4-yl]-5-(trifluoromethoxy)pyridin-2-amine;
1-{4-[3-(propan-2-yl)imidazo[1,2-a]pyridin-6-yl]benzenesulfonyl}-N-{4-[(trifluoromethyl)sulfanyl]phenyl}piperidin-4-amine;
1-{4-[3-(methoxymethyl)-[1,2,4]triazolo[4,3-a]pyridin-6-yl]benzenesulfonyl}-N-[4-(trifluoromethoxy)phenyl]piperidin-4-amine;
6-{4-[(4-{[5-(trifluoromethoxy)pyridin-2-yl]Amino}piperidin-1-yl)sulfonyl]phenyl}-2H,3H-[1,2,4]triazolo[4,3-a]pyridin-3-one;
1-(4-{imidazo[1,2-a]pyridin-6-yl}benzenesulfonyl)-N-{4-[(trifluoromethyl)sulfanyl]phenyl}piperidin-4-amine;
1-(4-{3-cyclopentyl-[1,2,4]triazolo[4,3-a]pyridin-6-yl}benzenesulfonyl)-N-[4-(trifluoromethoxy)phenyl]piperidin-4-amine;
1-{4-[8-methyl-3-(propan-2-yl)imidazo[1,2-a]pyridin-6-yl]benzenesulfonyl}-N-{4-[(trifluoromethyl)sulfanyl]phenyl}piperidin-4-amine;
4-(4-{[4-({4-[(trifluoromethyl)sulfanyl]phenyl}Amino)piperidin-1-yl]sulfonyl}phenyl)pyridine-2-carboxamide;
4′-{[trans-4-{[3-(trifluoromethoxy)phenyl]Amino}cyclohexyl]sulfonyl}-[1,1′-biphenyl]-4-carbonitrile;
N-[trans-4-(4-{imidazo[1,2-a]pyridin-6-yl}benzenesulfonyl)cyclohexyl]-3-[(trifluoromethyl)sulfanyl]aniline;
N-[trans-4-{4-[3-(propan-2-yl)imidazo[1,2-a]pyridin-6-yl]benzenesulfonyl}cyclohexyl]-3-[(trifluoromethyl)sulfanyl]aniline;
6-{4-[(4-{[3-(trifluoromethoxy)phenyl]Amino}piperidin-1-yl)sulfonyl]phenyl}-2,3-dihydro-11H-isoindol-1-one
4′-[(4-{[3-(difluoromethoxy)phenyl]Amino}piperidin-1-yl)sulfonyl]-[1,1′-biphenyl]-4-carbonitrile
N-(1-{4-[8-methyl-3-(propan-2-yl)imidazo[1,2-a]pyridin-6-yl]benzenesulfonyl}piperidin-4-yl)-5-(trifluoromethoxy)pyridin-2-amine
N-(1-{4-[3-(propan-2-yl)-[1,2,4]triazolo[4,3-a]pyridin-6-yl]benzenesulfonyl}piperidin-4-yl)-5-(trifluoromethoxy)pyridin-2-amine
N-(1-{4-[8-methyl-3-(propan-2-yl)-[1,2,4]triazolo[4,3-a]pyridin-6-yl]benzenesulfonyl}piperidin-4-yl)-5-(trifluoromethoxy)pyridin-2-amine
4-{4-[(4-{[5-(trifluoromethoxy)pyridin-2-yl]amino}piperidin-1-yl)sulfonyl]phenyl}pyridine-2-carboxamide
N-(1-{4-[3-(propan-2-yl)-[1,2,4]triazolo[4,3-a]pyrazin-6-yl]benzenesulfonyl}piperidin-4-yl)-5-(trifluoromethoxy)pyridin-2-amine
N-[1-(4-{3-methyl-[1,2,4]triazolo[4,3-a]pyridin-6-yl}benzenesulfonyl)piperidin-4-yl]-5-(trifluoromethoxy)pyridin-2-amine
N-(1-{4-[8-methyl-3-(propan-2-yl)imidazo[1,2-a]pyridin-6-yl]benzenesulfonyl}piperidin-4-yl)-5-[(trifluoromethyl)sulfanyl]pyridin-2-amine
N-[1-(4-{imidazo[1,2-a]pyridin-6-yl}benzenesulfonyl)piperidin-4-yl]-5-[(trifluoromethyl)sulfanyl]pyridin-2-amine
4-(4-{[4-({5-[(trifluoromethyl)sulfanyl]pyridin-2-yl}amino)piperidin-1-yl]sulfonyl}phenyl)pyridine-2-carboxamide
N-(1-{4-[3-(propan-2-yl)imidazo[1,2-a]pyridin-6-yl]benzenesulfonyl}piperidin-4-yl)-5-[(trifluoromethyl)sulfanyl]pyridin-2-amine
N-(1-{4-[3-(propan-2-yl)-[1,2,4]triazolo[4,3-a]pyridin-6-yl]benzenesulfonyl}piperidin-4-yl)-5-[(trifluoromethyl)sulfanyl]pyridin-2-amine
N-(1-{4-[3-(propan-2-yl)-[1,2,4]triazolo[4,3-a]pyrazin-6-yl]benzenesulfonyl}piperidin-4-yl)-5-[(trifluoromethyl)sulfanyl]pyridin-2-amine
N-[1-(4-{3-methyl-[1,2,4]triazolo[4,3-a]pyridin-6-yl}benzenesulfonyl)piperidin-4-yl]-5-[(trifluoromethyl)sulfanyl]pyridin-2-amine
N-(1-{4-[8-methyl-3-(propan-2-yl)-[1,2,4]triazolo[4,3-a]pyridin-6-yl]benzenesulfonyl}piperidin-4-yl)-5-[(trifluoromethyl)sulfanyl]pyridin-2-amine
N-(1-{4-[8-methyl-3-(propan-2-yl)imidazo[1,2-a]pyridin-6-yl]benzenesulfonyl}piperidin-4-yl)-4-(trifluoromethoxy)pyridin-2-amine
N-(1-{4-[3-(propan-2-yl)-[1,2,4]triazolo[4,3-a]pyridin-6-yl]benzenesulfonyl}piperidin-4-yl)-4-(trifluoromethoxy)pyridin-2-amine
N-(1-{4-[8-methyl-3-(propan-2-yl)-[1,2,4]triazolo[4,3-a]pyridin-6-yl]benzenesulfonyl}piperidin-4-yl)-4-(trifluoromethoxy)pyridin-2-amine
N-[trans-4-(4-{3-methyl-1H-pyrazolo[3,4-b]pyridin-5-yl}benzenesulfonyl)cyclohexyl]-4-[(trifluoromethyl)sulfanyl]aniline
N-[trans-4-(4-{imidazo[1,2-a]pyridin-6-yl}benzenesulfonyl)cyclohexyl]-5-(trifluoromethoxy)pyridin-2-amine or
6-(4-{[4-({5-[(trifluoromethyl)sulfanyl]pyridin-2-yl}amino)piperidin-1-yl]sulfonyl}phenyl)-2H,3H-[1,2,4]triazolo[4,3-a]pyridin-3-one.
Aspect 32. The compound of any one of aspects 1 to 31 selected from:
5-[4-[4-[4-(trifluoromethylsulfanyl)anilino]cyclohexyl]sulfonylphenyl]-1H-indole-3-carbonitrile;
5-[4-[[4-[[5-(trifluoromethoxy)-2-pyridyl]Amino]-1-piperidyl]sulfonyl]phenyl]-1H-indole-3-carbonitrile;
1-[4-(3-methyl-1H-indazol-5-yl)phenyl]sulfonyl-N-[4-(trifluoromethoxy)phenyl]piperidin-4-amine;
5-[4-[[4-[4-(trifluoromethoxy)anilino]-1-piperidyl]sulfonyl]phenyl]-1H-indole-3-carbonitrile;
5-[4-[[4-[4-(trifluoromethoxy)anilino]-1-piperidyl]sulfonyl]phenyl]-1H-indazole-3-carbonitrile;
6-[4-[[4-[4-(trifluoromethoxy)anilino]-1-piperidyl]sulfonyl]phenyl]-2H-[1,2,4]triazolo[4,3-a]pyridin-3-one;
1-[4-(3-isopropylimidazo[1,2-a]pyridin-6-yl)phenyl]sulfonyl-N-[4-(trifluoromethoxy)phenyl]piperidin-4-amine;
or
1-[4-(3-cyclopentyl-[1,2,4]triazolo[4,3-a]pyridin-6-yl)phenyl]sulfonyl-N-[4-(trifluoromethoxy)phenyl]piperidin-4-amine.

Aspect 33. The compound of any one of aspects 1 to 32 selected from:
5-[4-[4-[4-(trifluoromethylsulfanyl)anilino]cyclohexyl]sulfonylphenyl]-1H-indole-3-carbonitrile;
5-[4-[[4-[[5-(trifluoromethoxy)-2-pyridyl]Amino]-1-piperidyl]sulfonyl]phenyl]-1H-indole-3-carbonitrile;
1-[4-(3-methyl-1H-indazol-5-yl)phenyl]sulfonyl-N-[4-(trifluoromethoxy)phenyl]piperidin-4-amine;
5-[4-[[4-[4-(trifluoromethoxy)anilino]-1-piperidyl]sulfonyl]phenyl]-1H-indole-3-carbonitrile;
5-[4-[[4-[4-(trifluoromethoxy)anilino]-1-piperidyl]sulfonyl]phenyl]-1H-indazole-3-carbonitrile;
6-[4-[[4-[4-(trifluoromethoxy)anilino]-1-piperidyl]sulfonyl]phenyl]-2H-[1,2,4]triazolo[4,3-a]pyridin-3-one;
1-[4-(3-isopropylimidazo[1,2-a]pyridin-6-yl)phenyl]sulfonyl-N-[4-(trifluoromethoxy)phenyl]piperidin-4-amine;
1-[4-(3-cyclopentyl-[1,2,4]triazolo[4,3-a]pyridin-6-yl)phenyl]sulfonyl-N-[4-(trifluoromethoxy)phenyl]piperidin-4-amine
N-[1-(4-{3-methyl-[1,2,4]triazolo[4,3-a]pyridin-6-yl}benzenesulfonyl)piperidin-4-yl]-5-[(trifluoromethyl)sulfanyl]pyridin-2-amine or
6-(4-{[4-({5-[(trifluoromethyl)sulfanyl]pyridin-2-yl}amino)piperidin-1-yl]sulfonyl}phenyl)-2H,3H-[1,2,4]triazolo[4,3-a]pyridin-3-one.

Aspect 34. A process of preparation of a compound of formula (I) or (I′) according to any one of aspects 1 to 33, or a pharmaceutically acceptable salt thereof, wherein X¹ is N, and X⁴ is O, comprising:
reacting compound of formula (II), wherein R⁵ and R⁴ are as defined in any one of aspects 1 to 33,

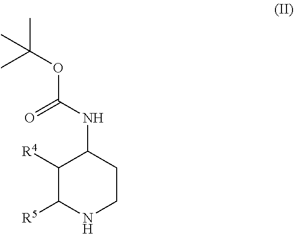

with compound of formula (III), wherein X², X³ R², and R³, are as defined in any one of aspects 1 to 33,

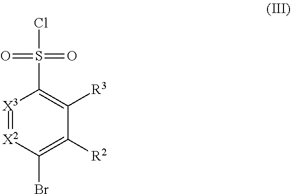

to form compound (IV), wherein R², R³, R⁴, R⁵, X², and X³ are as defined in any one of aspects 1 to 33,

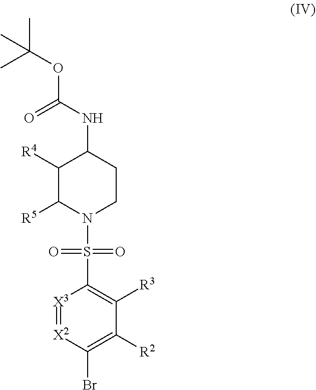

reacting said compound (IV) with compound of formula (V), wherein R¹ is as defined in any one of aspects 1 to 33, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

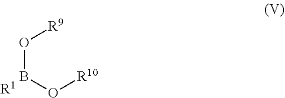

to form compound of formula (VI), wherein R¹, R², R³, R⁴, R⁵, X², and X³ are as defined in any one of aspects 1 to 33,

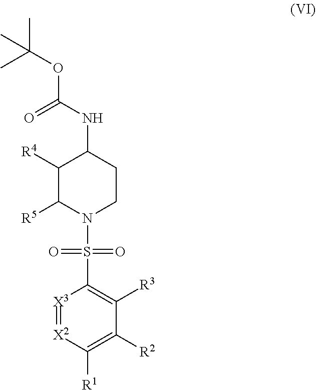

reacting said compound of formula (VI) with an acid, to form compound of formula (VII), wherein R¹, R², R³, R⁴, R⁵, X², and X³ are as defined in any one of aspects 1 to 33,

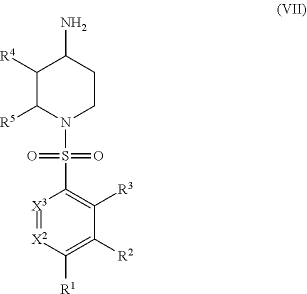

reacting said compound of formula (VII) with compound of formula (VIII), wherein R⁸ is a halogen and R⁶, R⁷, and X⁵ are as defined in any one of aspects 1 to 33,

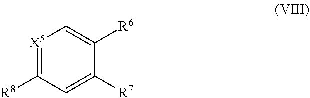

to form compound of formula (I).

Aspect 35. A process of preparation of a compound of formula (I) or (I′) according to any one of aspects 1 to 33, or a pharmaceutically acceptable salt thereof, wherein X¹ is N and X⁴ is O, comprising:
reacting compound of formula (IX), wherein R⁴ and R⁵ are as defined in any one of aspects 1 to 33,

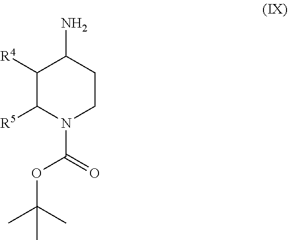

with compound of formula (VIII), wherein R is a halogen and R¹, R⁷, and X⁵ are as defined in any one of aspects 1 to 33,

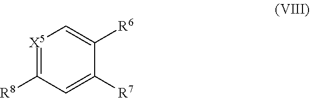

to form compound of formula (X), wherein wherein R⁴ R⁶, R⁷, X⁵, and R⁵ are as defined in any one of aspects 1 to 33,

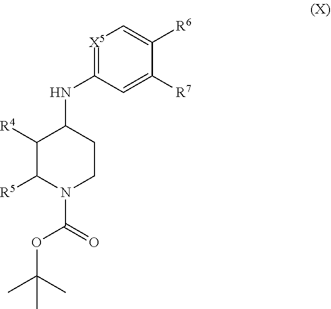

reacting said compound of formula (X) with acid to form compound of formula (XI), wherein R⁴ R⁶, R⁷, X⁵, and R⁵ are as defined in any one of aspects 1 to 33,

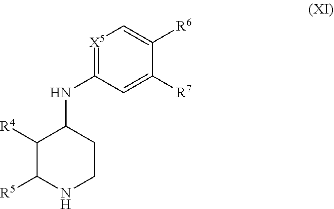

reacting said compound of formula (XI) with compound of formula (III), wherein X², X³, R², and R³, are as defined in any one of aspects 1 to 33,

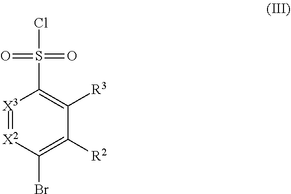

to form compound of formula (XII), wherein X², X³, R², R³, R⁴ R⁶, R⁷, X⁵, and R⁵ are as defined in any one of aspects 1 to 33,

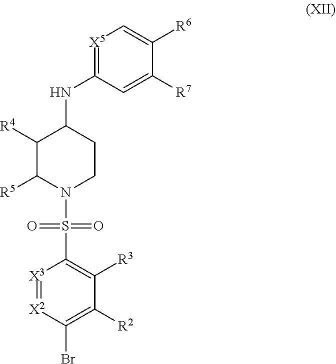

reacting said compound of formula (XII) with compound of formula (V), wherein R¹ is as defined in any one of aspects 1 to 33, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

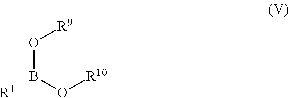

to form compound of formula (I).

Aspect 36. A process of preparation of a compound of formula (I) or (I′) according to any one of aspects 1 to 33, or a pharmaceutically acceptable salt thereof, wherein X¹ is N and X⁴ is O, comprising:
reacting compound of formula (XVIII), wherein R⁵ and R⁴ are as defined in any one of aspects 1 to 33,

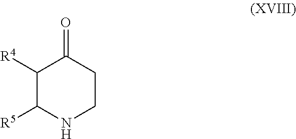

with compound of formula (XVII), wherein R¹, R², R³, X², and X³ are as defined in any one of aspects 1 to 33,

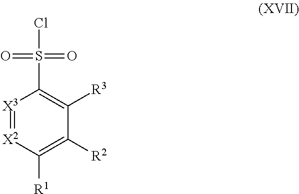

to form compound of formula (XX), wherein R¹, R², R³, R⁴, R⁵, X², and X³ are as defined in any one of aspects 1 to 33,

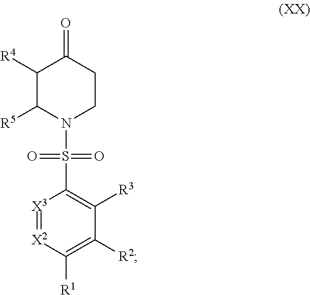

reacting said compound of formula (XX) with compound of formula (XVI), wherein X⁵, R⁶, and R⁷ are as defined in any one of aspects 1 to 33,

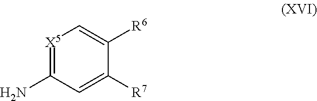

to form compound of formula (I);
or
reacting compound of formula (XVIII), wherein R⁵ and R⁴ are as defined in any one of aspects 1 to 33,

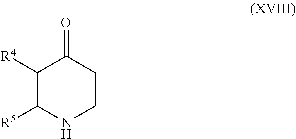

with compound of formula (III), wherein X², X³, R², and R³, are as defined in any one of aspects 1 to 33,

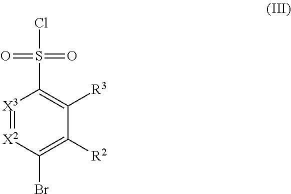

to form compound of formula (XIX), wherein R², R³, R⁴, R⁵, X², and X³ are as defined in any one of aspects 1 to 33,

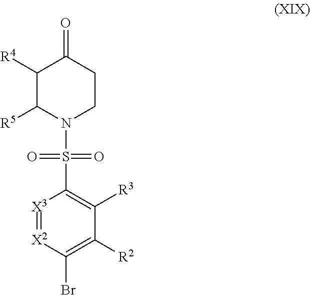

reacting said compound of formula (XIX) with said compound of formula (XVI), to form compound of formula (XII), wherein X², X³, R², R³, R⁴ R⁶, R⁷, X⁵, and R⁵ are as defined in any one of aspects 1 to 33,

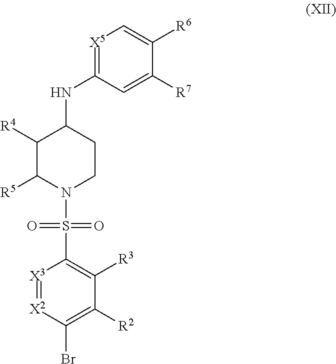

reacting said compound of formula (XII) with compound of formula (V), wherein R¹ is as defined in any one of aspects 1 to 33, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

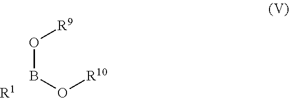

to form compound of formula (I).

Aspect 37. A process of preparation of a compound of formula (I) or (I′) according to any one of aspects 1 to 33, or a pharmaceutically acceptable salt thereof, wherein X¹ is C and X⁴ is O, comprising:
reacting compound of formula (XXI), wherein R⁴ and R⁵ are as defined in any one of aspects 1 to 33,

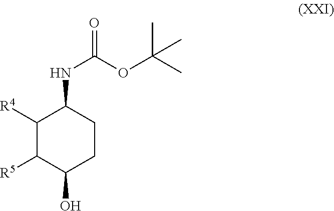

with masyl chloride, to form compound of formula (XXII), wherein R⁴ and R⁵ are as defined in any one of aspects 1 to 33,

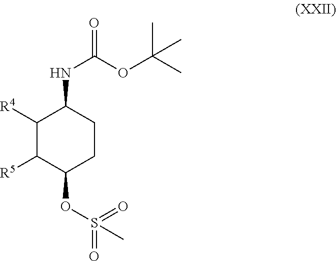

reacting said compound of formula (XXII) with compound of formula (XXIII), wherein R², R³, X², and X³ are as defined in any one of aspects 1 to 33,

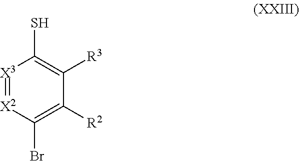

to form compound of formula (XXIV), wherein R², R³, R⁴, R⁵, X², and X³ are as defined in any one of aspects 1 to 33,

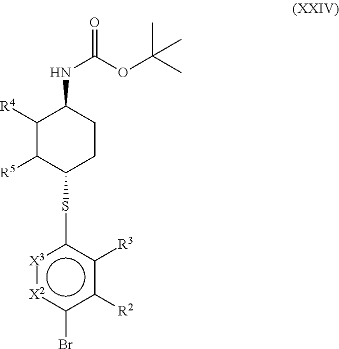

reacting said compound of formula (XXIV) with an oxidant, to form compound of formula (XXV), wherein R², R³, R⁴, R⁵, X², and X³ are as defined in any one of aspects 1 to 33,

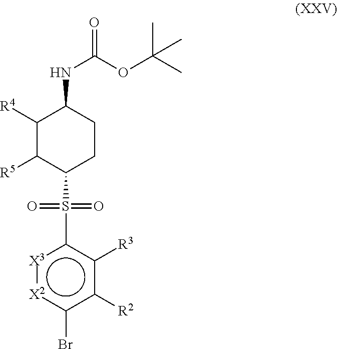

reacting said compound of formula (XXV) with compound of formula (V), wherein R¹ is as defined in any one of aspects 1 to 33, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

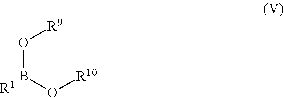

to form compound of formula (XXVI), wherein R¹, R², R³, R⁴, R⁵, X², and X³ are as defined in any one of aspects 1 to 33,

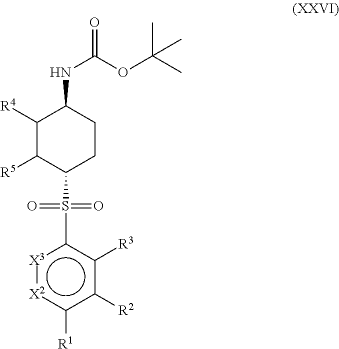

reacting said compound of formula (XXVI) with acid, to form compound of formula (XXVII), wherein R¹, R², R³, R⁴, R⁵, X², and X³ are as defined in any one of aspects 1 to 33,

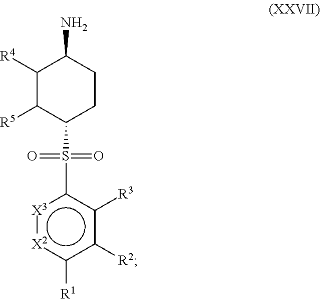

reacting said compound of formula (XXVII) with compound of formula (VIII), wherein R⁸ is a halogen and R⁶, R⁷, and X⁵ are as defined in any one of aspects 1 to 33,

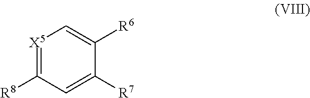

to form compound of formula (I).

Aspect 38. A process of preparation of a compound of formula (I) or (I′) according to any one of aspects 1 to 33, or a pharmaceutically acceptable salt thereof, wherein X¹ is C and X⁴ is O, comprising:
reacting compound of formula (XXV), wherein R², R³, R⁴, R⁵, X², and X³ are as defined in any one of aspects 1 to 33,

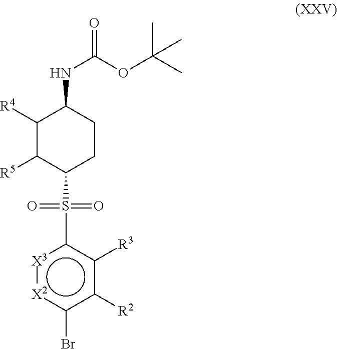

with acid to form compound of formula (XXVIII), wherein R², R³, R⁴, R⁵, X², and X³ are as defined in any one of aspects 1 to 33,

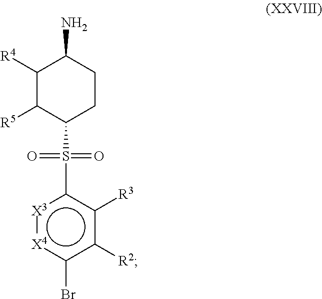

reacting said compound of formula (XXVIII) with compound of formula (VIII), wherein R⁸ is a halogen and R⁶, R⁷, and X⁵ are as defined in any one of aspects 1 to 33,

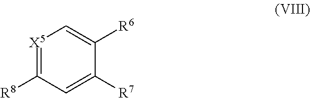

to form compound of formula (XXIX), wherein R², R³, R⁴, R⁵, R⁶, R⁷, X⁵, X², and X³ are as defined in any one of aspects 1 to 33,

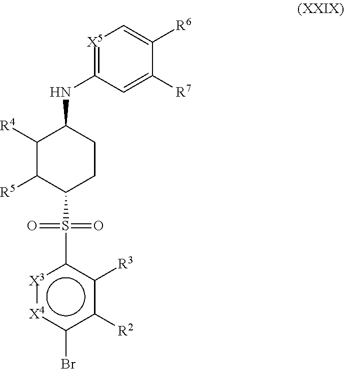

reacting said compound of formula (XXIX) with with compound of formula (V), wherein R¹ is as defined in any one of aspects 1 to 33, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

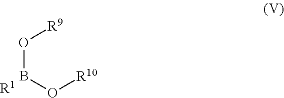

to form compound of formula (I).

Aspect 39. A process of preparation of a compound of formula (I) or (I′) according to any one of aspects 1 to 33, or a pharmaceutically acceptable salt thereof, wherein X¹ is C and X⁴ is O, comprising:
reacting compound of formula (XXXI), wherein R⁴ and R⁵ are as defined in any one of aspects 1 to 33,

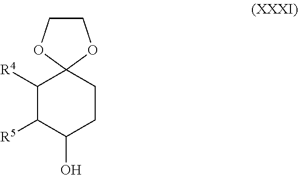

with mesyl chloride, to form compound of formula (XXXII), wherein R⁴ and R⁵ are as defined in any one of aspects 1 to 33,

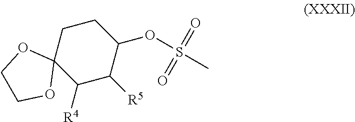

reacting said compound of formula (XXXII) with compound of formula (XXIII), wherein R², R³, X², and X³ are as defined in any one of aspects 1 to 33,

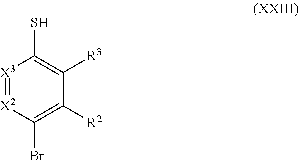

to form compound of formula (XXXIII), wherein R², R³, R⁴, R⁵, X², and X³ are as defined in any one of aspects 1 to 33,

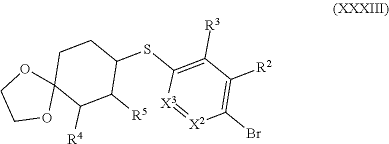

reacting said compound (XXXIII) with an oxidant, to form compound of formula (XXXIV), wherein R², R³, R⁴, R⁵, X², and X³ are as defined in any one of aspects 1 to 33,

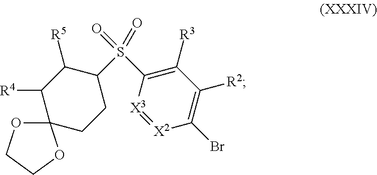

reacting said compound (XXXIV) with an acid, to form compound of formula (XXXV), wherein R², R³, R⁴, R⁵, X², and X³ are as defined in any one of aspects 1 to 33,

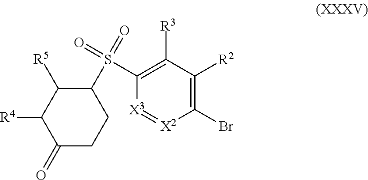

reacting said compound of formula (XXXV) with compound of formula (XVI), wherein X⁵, R⁶, and R⁷ are as defined in any one of aspects 1 to 33,

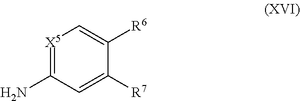

to form compound of formula (XXIX), wherein R², R³, R⁴, R⁵, R⁶, R⁷, X⁵, X², and X³ are as defined in any one of aspects 1 to 33,

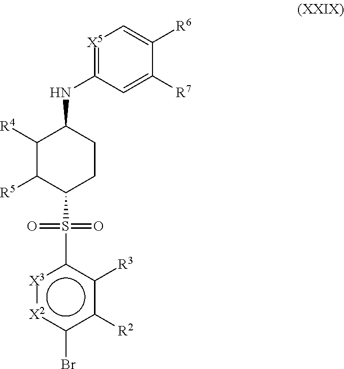

reacting said compound of formula (XXIX) with with compound of formula (V), wherein R¹ is as defined in any one of aspects 1 to 33, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

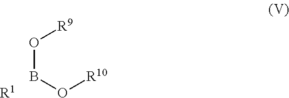

to form compound of formula (I).

Aspect 40. A process of preparation of a compound of formula (I) or (I′) according to any one of aspects 1 to 33, or a pharmaceutically acceptable salt thereof, wherein X¹ is C and X⁴ is N, comprising:
reacting compound of formula (XXXIII), wherein R², R³, R⁴, R⁵, X², and X³ are as defined in any one of aspects 1 to 33,

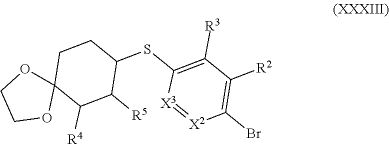

with an acid to form compound of formula (XXXVI), wherein R², R³, R⁴, R⁵, X², and X³ are as defined in any one of aspects 1 to 33,

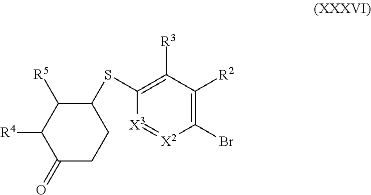

reacting said compound of formula (XXXVI) with compound of formula (XVI), wherein X⁵, R⁶, and R⁷ are as defined in any one of aspects 1 to 33,

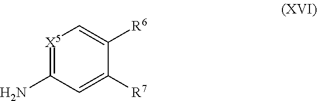

to form compound of formula (XXXVII), wherein R², R³, R⁴, R⁵, R⁶, R⁷, X⁵, X², and X³ are as defined in any one of aspects 1 to 33,

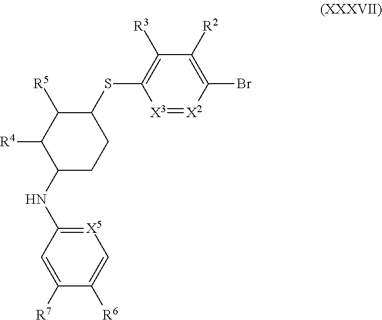

reacting said compound of formula (XXXVII) with ammonium carbamate and (diacetoxyiodo)benzene, to form compound of formula (XXXVIII), wherein R², R³, R⁴, R⁵, R⁶, R⁷, X⁵, X², and X³ are as defined in any one of aspects 1 to 33,

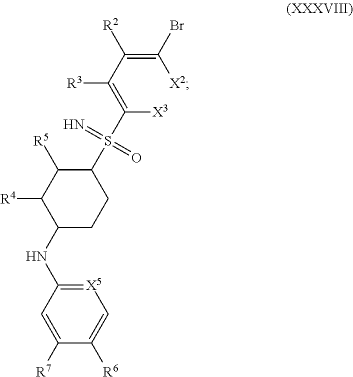

reacting said compound of formula (XXXVIII) with compound of formula (V), wherein R¹ is as defined in any one of aspects 1 to 33, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

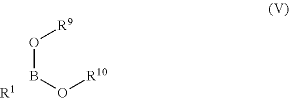

to form compound of formula (I).

Aspect 41. A process of preparation of a compound of formula (I) or (I′) according to any one of aspects 1 to 33, or a pharmaceutically acceptable salt thereof, wherein X¹ and X⁴ are N, comprising:
reacting compound of formula (XI), wherein R⁴ R⁶, R⁷, X⁵, and R⁵ are as defined in any one of aspects 1 to 33,

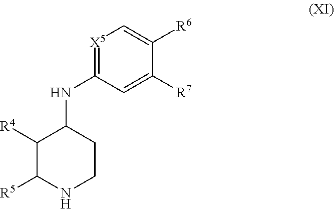

with compound of formula (XXIII), wherein R² R³, X², and X³ are as defined in any one of aspects 1 to 33,

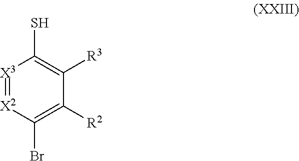

to form compound of formula (XL), wherein R², R³, X², X³, R⁴ R⁶, R⁷, X⁵, and R⁵ are as defined in any one of aspects 1 to 33,

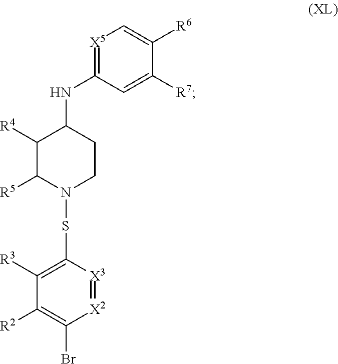

reacting said compound (XL) with ammonium carbamate and (diacetoxyiodo)benzene, to form compound of formula (XLI), wherein R² R³, X², X³, R⁴ R⁶, R⁷, X⁵, and R⁵ are as defined in any one of aspects 1 to 33,

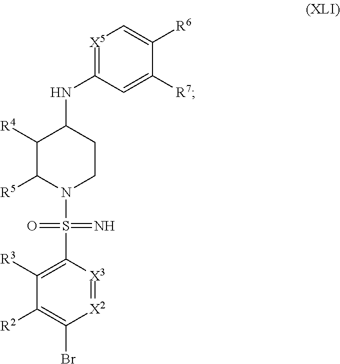

reacting said compound (XLI) with di-tert-butyl dicarbonate and a base, to form compound of formula (XLII), wherein R², R³, X², X³, R⁴ R⁶, R⁷, X⁵, and R⁵ are as defined in any one of aspects 1 to 33,

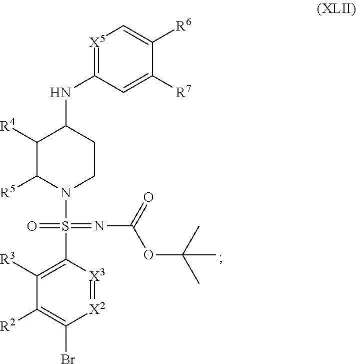

reacting said compound (XLII) with compound of formula (V), wherein R¹ is as defined in any one of aspects 1 to 33, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

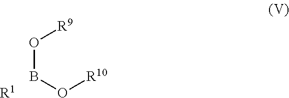

to form compound of formula (XLIII), wherein R¹, R², R³, X², X³, R⁴ R⁶, R⁷, X⁵, and R⁵ are as defined in any one of aspects 1 to 33,

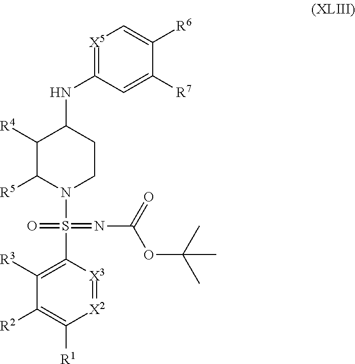

reacting said compound of formula (XLIII) with an acid to form compound of formula (I).

Aspect 42. A process of preparation of a compound of formula (I) or (I′) according to any one of aspects 1 to 33, or a pharmaceutically acceptable salt thereof, wherein X¹ is C and X⁴ is N, comprising:
reacting compound of formula (XXIV), wherein R², R³, R⁴, R⁵, X², and X³ are as defined in any one of aspects 1 to 33,

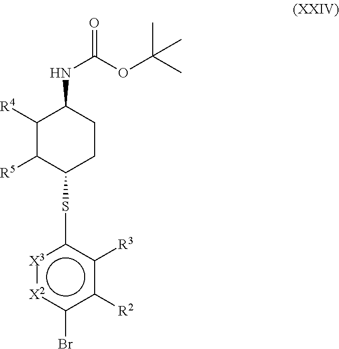

with compound of formula (V), wherein R¹ is as defined in any one of aspects 1 to 33, and R⁹ and R¹⁰ are hydrogen, or R⁹ and R¹⁰ are independently selected from C₁₋₆ alkyl, or R⁹ and R¹⁰ taken together with the oxygen atom to which they are attached, form a 3- to 14-membered heterocyclyl optionally substituted with one or more C₁₋₆alkyl, in particular optionally substituted with four C₁₋₆alkyl,

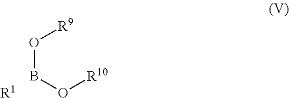

to form compound of formula (XLIV), wherein R¹, R², R³, R⁴, R⁵, X², and X³ are as defined in any one of aspects 1 to 33,

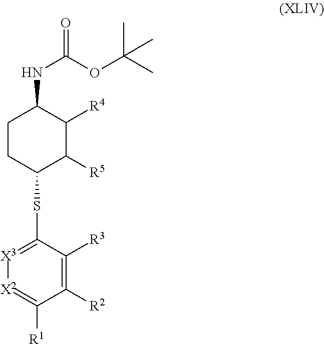

reacting said compound of formula (XLIV) with acid to form compound of formula (XLV), wherein R¹, R², R³, R⁴, R⁵, X², and X³ are as defined in any one of aspects 1 to 33,

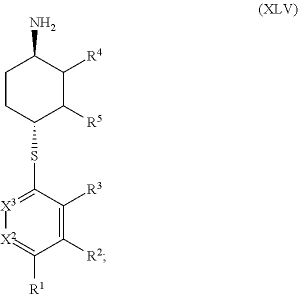

reacting said compound of formula (XLV) with compound of formula (VIII), wherein R⁸ is a halogen and R⁶, R⁷, and X⁵ are as defined in any one of aspects 1 to 33,

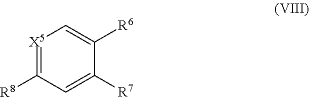

to form compound of formula (XLVI), wherein R¹, R², R³, R⁴, R⁵, R⁶, R⁷, X⁵, X², and X³ are as defined in any one of aspects 1 to 33,

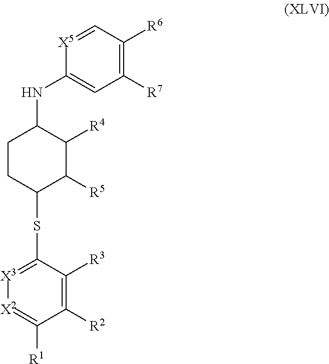

reacting said compound of formula (XLVI) with ammonium carbamate and (diacetoxyiodo)benzene to form compound of formula (I).

Aspect 43. A compound according to any one of aspects 1 to 33, or a pharmaceutically acceptable salt thereof, when manufactured according to any one of aspects 34 to 42.

Aspect 44. A compound according to any one of aspects 1 to 33, or a pharmaceutically acceptable salt thereof, for use as a therapeutically active substance.

Aspect 45. A pharmaceutical composition comprising a compound according to any one of aspects 1 to 33, or a pharmaceutically acceptable salt thereof, and a pharmaceutically acceptable excipient.

Aspect 46. The pharmaceutical composition according to aspect 45, further comprising an additional therapeutic agent.

Aspect 47. A compound according to any one of aspects 1 to 33, or a pharmaceutically acceptable salt thereof, for use in the treatment, prevention and/or delay of progression of an inflammatory autoimmune disease.

Aspect 48. A compound according to any one of aspects 1 to 33, or a pharmaceutically acceptable salt thereof, for use in the treatment, prevention and/or delay of progression of psoriatic diseases, asthma, Inflammatory Bowel Diseases (IBD), Crohn's disease, Obstructive Lung Diseases ulcerative colitis, rheumatoid arthritis, systemic lupus erythematosus and multiple sclerosis.

Aspect 49. A compound according to any one of aspects 1 to 33, or a pharmaceutically acceptable salt thereof, for use in the treatment, prevention and/or delay of progression of psoriatic diseases, asthma, Crohn's disease, ulcerative colitis, rheumatoid arthritis, systemic lupus erythematosus and multiple sclerosis.

Aspect 50. The use of a compound according to any one of aspects 1 to 33, or a pharmaceutically acceptable salt thereof, for the preparation of a medicament for the treatment, prevention and/or delay of an inflammatory autoimmune disease.

Aspect 51. The use of a compound according to any one of aspects 1 to 33, or a pharmaceutically acceptable salt thereof, for the preparation of a medicament for the treatment, prevention and/or delay of psoriatic diseases, asthma, Crohn's disease, ulcerative colitis, rheumatoid arthritis, systemic lupus erythematosus and multiple sclerosis.

Aspect 52. A method for the treatment, prevention and/or delay of progression of an inflammatory autoimmune disease, which method comprises administering a therapeutically effective amount of a compound according to any one of aspects 1 to 33, or a pharmaceutically acceptable salt thereof.

Aspect 53. A method for the treatment, prevention and/or delay of progression of psoriatic diseases, asthma, Inflammatory Bowel Diseases (IBD), Crohn's disease, Obstructive Lung Diseases ulcerative colitis, rheumatoid arthritis, systemic lupus erythematosus or multiple sclerosis, which method comprises administering a therapeutically effective amount of a compound according to any one of aspects 1 to 33, or a pharmaceutically acceptable salt thereof.

1. A compound of formula (I) 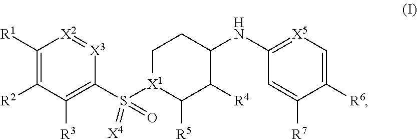
wherein
X¹ is CH or N;
X² is CH or N;
X³ is CH or N;
X⁴ is O or NH;
X⁵ is CH or N;
R¹ is aryl, heterocyclyl, or heteroaryl, wherein aryl, heterocyclyl, and heteroaryl are optionally substituted with one or more R^{1a} wherein R^{1a} is selected from C₁₋₆alkyl, oxo, cyano, carbamoyl, C₁₋₆alkylcarbamoyl-, C₁₋₆alkoxyC₁₋₆alkyl-, C₃₋₆cycloalkyl, and heterocyclyl;
R² is hydrogen or halogen;
R³ is hydrogen or halogen;
R⁴ is hydrogen, halogen or C₁₋₆alkyl;
R⁵ is hydrogen, halogen or C₁₋₆alkyl;
R⁶ is —OR^{6a}, —SR^{6b}, or hydrogen wherein R^{6a} is C₁₋₆haloalkyl and R^{6b} is C₁₋₆haloalkyl; and
R⁷ is —OR^{7a}, —SR^{7b}, or hydrogen wherein R^{7a} is C₁₋₆haloalkyl and R^{7b} is C₁₋₆haloalkyl and provided that R⁶ and R⁷ are different, and one of R⁶ or R⁷ is hydrogen,
or a pharmaceutically acceptable salt thereof.

2. The compound of claim 1 wherein
X² is CH;
X³ is CH;
R² is hydrogen;
R³ is hydrogen;
R⁴ is hydrogen;
R⁵ is hydrogen or C₁₋₆alkyl.

3. The compound of claim 1, wherein X¹ is CH.

4. The compound of claim 1, wherein X¹ is N.

5. The compound claim 1, wherein X⁴ is O.

6. The compound claim 1, wherein X⁴ is NH.

7. The compound claim 1, wherein X⁵ is CH.

8. The compound claim 1, wherein X⁵ is N.

9. The compound of claim 1, wherein R¹ is selected from imidazopyridinyl, triazolopyridinyl, phenyl, indolyl, isoindolinyl, and indazolyl, wherein imidazopyridinyl, triazolopyridinyl, phenyl, indolyl, isoindolinyl, and indazolyl, are optionally substituted with one or more R^{1a}.

10. The compound claim 1, wherein R¹ is selected from isopropylimidazopyridinyl, oxo-triazolopyridinyl, imidazopyridinyl, cyanophenyl, methylimidazopyridinyl, cyano-indolyl, oxoisoindolinyl, carbamoylphenyl, (methylcarbamoyl)phenyl, methyl-indazolyl, phenyl, cyano-indazolyl, cyanoimidazopyridinyl, methyl-triazolopyridinyl, (methoxymethyl)-triazolopyridinyl, and cyclopentyl-triazolopyridinyl.

11. The compound of claim 1, wherein R¹ is selected from (3-isopropylimidazo[1,2-a]pyridin-6-yl), (3-oxo-2H-[1,2,4]triazolo[4,3-a]pyridin-6-yl), imidazo[1,2-a]pyridin-6-yl, (4-cyanophenyl), (3-methylimidazo[1,2-a]pyridin-6-yl), (3-cyano-1H-indol-5-yl), (3-isopropyl-8-methyl-imidazo[1,2-a]pyridin-6-yl), (2-carbamoyl-4-pyridyl), (3-oxoisoindolin-5-yl), (3-carbamoylphenyl), [3-(methylcarbamoyl)phenyl], (3-methyl-1H-indazol-5-yl), phenyl, (3-cyano-1H-indazol-5-yl), (3-cyanoimidazo[1,2-a]pyridin-6-yl), (2-methyl-[1,2,4]triazolo[1,5-a]pyridin-6-yl), (2-methyl-[1,2,4]triazolo[1,5-a]pyridin-7-yl), [3-(methoxymethyl)-[1,2,4]triazolo[4,3-a]pyridin-6-yl], and (3-cyclopentyl-[1,2,4]triazolo[4,3-a]pyridin-6-yl).

12. The compound claim 1, wherein R¹ is selected from cyano-indazolyl, cyano-indolyl, methyl-indazolyl, oxo-triazolopyridinyl, isopropylimidazopyridinyl, and cyclopentyl-triazolopyridinyl.

13. The compound claim 1, wherein R¹ is selected from (3-cyano-1H-indol-5-yl), (3-methyl-1H-indazol-5-yl), (3-cyano-1H-indazol-5-yl), (3-oxo-2H-[1,2,4]triazolo[4,3-a]pyridin-6-yl), (3-isopropylimidazo[1,2-a]pyridin-6-yl), and cyclopentyl-triazolopyridinyl.

14. The compound claim 1, wherein R^{1a} is isopropyl, methyl, oxo, cyano, carbamoyl, methylcarbamoyl, or methoxymethyl.

15. The compound claim 1, wherein R⁶ is —SCF₃, —OCF₃, —OCHF₂, or hydrogen.

16. The compound claim 1, wherein R⁶ is —SCF₃, or —OCF₃.

17. The compound claim 1, wherein R^{6a} is —CF₃, or —CHF₂.

18. The compound claim 1, wherein R^{6b} is —CF₃.

19. The compound claim 1, wherein R⁷ is —OCF₃, —SCF₃, —OCHF₂, or hydrogen.

20. The compound claim 1, wherein R⁷ is hydrogen.

21. The compound claim 1, wherein R^{7a} is —CF₃, or —CHF₂.

22. The compound claim 1, wherein R^{7b} is —CF₃.

23. The compound claim 1, wherein R⁵ is hydrogen or methyl.

24. The compound claim 1, wherein the compound is of formula (I′) 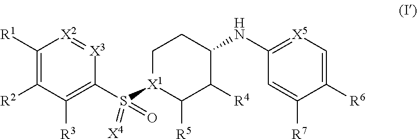
or a pharmaceutically acceptable salt thereof, and R¹, R², R³, R⁴, R⁵, R⁶, R⁷, X², X³, X⁴, and X⁵ are as defined in claim 1, and X¹ is CH.

25. The compound of claim 1, wherein the compound is of formula (I″) 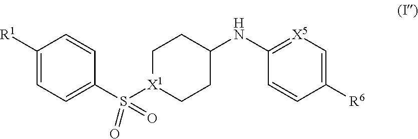
or a pharmaceutically acceptable salt thereof, and R¹, X¹, and X⁵ are as defined in claim 1, and R⁶ —SCF₃ or —OCF₃.

26. The compound of claim 1, wherein the compound is selected from:
{4-[3-(propan-2-yl)imidazo[1,2-a]pyridin-6-yl]phenyl}[trans-4-({4-[(trifluoromethyl)sulfanyl]phenyl}Amino)cyclohexyl](imino)-λ⁶-sulfanone;
6-(4-{[trans-4-({4-[(trifluoromethyl)sulfanyl]phenyl}Amino)cyclohexyl]sulfonimidoyl}phenyl)-2H,3H-[1,2,4]triazolo[4,3-a]pyridin-3-one;
(4-{imidazo[1,2-a]pyridin-6-yl}phenyl)[trans-4-({4-[(trifluoromethyl)sulfanyl]phenyl}Amino)cyclohexyl](imino)-λ⁶-sulfanone;
(4-{imidazo[1,2-a]pyridin-6-yl}phenyl)[trans-4-{[5-(trifluoromethoxy)pyridin-2-yl]Amino}cyclohexyl](imino)-λ⁶-sulfanone;
4′-{[trans-4-{[4-(trifluoromethoxy)phenyl]Amino}cyclohexyl]sulfonyl}-[1,1′-biphenyl]-4-carbonitrile;
N-[trans-4-(4-{3-methylimidazo[1,2-a]pyridin-6-yl}benzenesulfonyl)cyclohexyl]-4-(trifluoromethoxy)aniline;
N-[trans-4-(4-{3-methylimidazo[1,2-a]pyridin-6-yl}benzenesulfonyl)cyclohexyl]-5-(trifluoromethoxy)pyridin-2-amine;
5-(4-{[trans-4-({4-[(trifluoromethyl)sulfanyl]phenyl}Amino)cyclohexyl]sulfonyl}phenyl)-1H-indole-3-carbonitrile;
N-[trans-4-(4-{imidazo[1,2-a]pyridin-6-yl}benzenesulfonyl)cyclohexyl]-4-(trifluoromethoxy)aniline;
6-(4-{[trans-4-({4-[(trifluoromethyl)sulfanyl]phenyl}Amino)cyclohexyl]sulfonyl}phenyl)-2H,3H-[1,2,4]triazolo[4,3-a]pyridin-3-one;
N-[trans-4-(4-{imidazo[1,2-a]pyridin-6-yl}benzenesulfonyl)cyclohexyl]-4-[(trifluoromethyl)sulfanyl]aniline;
N-[trans-4-{4-[8-methyl-3-(propan-2-yl)imidazo[1,2-a]pyridin-6-yl]benzenesulfonyl}cyclohexyl]-4-[(trifluoromethyl)sulfanyl]aniline;
4-(4-{[trans-4-({4-[(trifluoromethyl)sulfanyl]phenyl}Amino)cyclohexyl]sulfonyl}phenyl)pyridine-2-carboxamide;
4′-[(4-{[5-(trifluoromethoxy)pyridin-2-yl]Amino}piperidin-1-yl)sulfonyl]-[1,1′-biphenyl]-4-carbonitrile;
6-{4-[(4-{[5-(trifluoromethoxy)pyridin-2-yl]Amino}piperidin-1-yl)sulfonyl]phenyl}-2,3-dihydro-1H-isoindol-1-one;
4′-[(4-{[5-(trifluoromethoxy)pyridin-2-yl]Amino}piperidin-1-yl)sulfonyl]-[1,1′-biphenyl]-3-carboxamide;
5-{4-[(4-{[5-(trifluoromethoxy)pyridin-2-yl]Amino}piperidin-1-yl)sulfonyl]phenyl}-1H-indole-3-carbonitrile;
4′-[(4-{[4-(trifluoromethoxy)phenyl]Amino}piperidin-1-yl)sulfonyl]-[1,1′-biphenyl]-4-carbonitrile;
N-methyl-4′-[(4-{[4-(trifluoromethoxy)phenyl]Amino}piperidin-1-yl)sulfonyl]-[1,1′-biphenyl]-3-carboxamide;
6-{4-[(4-{[4-(trifluoromethoxy)phenyl]Amino}piperidin-1-yl)sulfonyl]phenyl}-2,3-dihydro-1H-isoindol-1-one;
4′-[(4-{[4-(trifluoromethoxy)phenyl]Amino}piperidin-1-yl)sulfonyl]-[1,1′-biphenyl]-3-carboxamide;
1-[4-(3-methyl-1H-indazol-5-yl)benzenesulfonyl]-N-[4-(trifluoromethoxy)phenyl]piperidin-4-amine;
5-{4-[(4-{[4-(trifluoromethoxy)phenyl]Amino}piperidin-1-yl)sulfonyl]phenyl}-1H-indole-3-carbonitrile;
5-{4-[(4-{[4-(trifluoromethoxy)phenyl]Amino}piperidin-1-yl)sulfonyl]phenyl}-1H-indazole-3-carbonitrile;
1-{[1,1′-biphenyl]-4-sulfonyl}-N-[4-(trifluoromethoxy)phenyl]piperidin-4-amine;
1-{[1,1′-biphenyl]-4-sulfonyl}-2-methyl-N-[4-(trifluoromethoxy)phenyl]piperidin-4-amine;
4′-[(2-methyl-4-{[4-(trifluoromethoxy)phenyl]Amino}piperidin-1-yl)sulfonyl]-[1,1′-biphenyl]-4-carbonitrile;
6-{4-[(2-methyl-4-{[4-(trifluoromethoxy)phenyl]Amino}piperidin-1-yl)sulfonyl]phenyl}-2,3-dihydro-1H-isoindol-1-one;
N-methyl-4′-[(2-methyl-4-{[4-(trifluoromethoxy)phenyl]Amino}piperidin-1-yl)sulfonyl]-[1,1′-biphenyl]-3-carboxamide;
N-[4-(difluoromethoxy)phenyl]-1-[4-(3-methyl-1H-indazol-5-yl)benzenesulfonyl]piperidin-4-amine;
6-{4-[(4-{[4-(difluoromethoxy)phenyl]Amino}piperidin-1-yl)sulfonyl]phenyl}-2,3-dihydro-1H-isoindol-1-one;
5-{4-[(4-{[4-(difluoromethoxy)phenyl]Amino}piperidin-1-yl)sulfonyl]phenyl}-1H-indole-3-carbonitrile;
1-{[1,1′-biphenyl]-4-sulfonyl}-N-[4-(difluoromethoxy)phenyl]piperidin-4-amine;
1-(4-{imidazo[1,2-a]pyridin-6-yl}benzenesulfonyl)-N-[4-(trifluoromethoxy)phenyl]piperidin-4-amine;
1-(4-{3-methylimidazo[1,2-a]pyridin-6-yl}benzenesulfonyl)-N-[4-(trifluoromethoxy)phenyl]piperidin-4-amine;
6-{4-[(4-{[4-(trifluoromethoxy)phenyl]Amino}piperidin-1-yl)sulfonyl]phenyl}imidazo[1,2-a]pyridine-3-carbonitrile;
6-{4-[(4-{[4-(trifluoromethoxy)phenyl]Amino}piperidin-1-yl)sulfonyl]phenyl}-2H,3H-[1,2,4]triazolo[4,3-a]pyridin-3-one;
1-(4-{2-methyl-[1,2,4]triazolo[1,5-a]pyridin-6-yl}benzenesulfonyl)-N-[4-(trifluoromethoxy)phenyl]piperidin-4-amine;
1-(4-{2-methyl-[1,2,4]triazolo[1,5-a]pyridin-7-yl}benzenesulfonyl)-N-[4-(trifluoromethoxy)phenyl]piperidin-4-amine;
1-{4-[3-(propan-2-yl)imidazo[1,2-a]pyridin-6-yl]benzenesulfonyl}-N-[4-(trifluoromethoxy)phenyl]piperidin-4-amine;
N-(1-{4-[3-(propan-2-yl)imidazo[1,2-a]pyridin-6-yl]benzenesulfonyl}piperidin-4-yl)-5-(trifluoromethoxy)pyridin-2-amine;
N-[1-(4-{imidazo[1,2-a]pyridin-6-yl}benzenesulfonyl)piperidin-4-yl]-5-(trifluoromethoxy)pyridin-2-amine;
1-{4-[3-(propan-2-yl)imidazo[1,2-a]pyridin-6-yl]benzenesulfonyl}-N-{4-[(trifluoromethyl)sulfanyl]phenyl}piperidin-4-amine;
1-{4-[3-(methoxymethyl)-[1,2,4]triazolo[4,3-a]pyridin-6-yl]benzenesulfonyl}-N-[4-(trifluoromethoxy)phenyl]piperidin-4-amine;
6-{4-[(4-{[5-(trifluoromethoxy)pyridin-2-yl]Amino}piperidin-1-yl)sulfonyl]phenyl}-2H,3H-[1,2,4]triazolo[4,3-a]pyridin-3-one;
1-(4-{imidazo[1,2-a]pyridin-6-yl}benzenesulfonyl)-N-{4-[(trifluoromethyl)sulfanyl]phenyl}piperidin-4-amine;
1-(4-{3-cyclopentyl-[1,2,4]triazolo[4,3-a]pyridin-6-yl}benzenesulfonyl)-N-[4-(trifluoromethoxy)phenyl]piperidin-4-amine;
1-{4-[8-methyl-3-(propan-2-yl)imidazo[1,2-a]pyridin-6-yl]benzenesulfonyl}-N-{4-[(trifluoromethyl)sulfanyl]phenyl}piperidin-4-amine;
4-(4-{[4-({4-[(trifluoromethyl)sulfanyl]phenyl}Amino)piperidin-1-yl]sulfonyl}phenyl)pyridine-2-carboxamide;
4′-{[trans-4-{[3-(trifluoromethoxy)phenyl]Amino}cyclohexyl]sulfonyl}-[1,1′-biphenyl]-4-carbonitrile;
N-[trans-4-(4-{imidazo[1,2-a]pyridin-6-yl}benzenesulfonyl)cyclohexyl]-3-[(trifluoromethyl)sulfanyl]aniline;
N-[trans-4-{4-[3-(propan-2-yl)imidazo[1,2-a]pyridin-6-yl]benzenesulfonyl}cyclohexyl]-3-[(trifluoromethyl)sulfanyl]aniline;
6-{4-[(4-{[3-(trifluoromethoxy)phenyl]Amino}piperidin-1-yl)sulfonyl]phenyl}-2,3-dihydro-11H-isoindol-1-one,
and
4′-[(4-{[3-(difluoromethoxy)phenyl]Amino}piperidin-1-yl)sulfonyl]-[1,1′-biphenyl]-4-carbonitrile;
or a pharmaceutically acceptable salt thereof.

27. The compound claim 1, wherein the compound is selected from:
{4-[3-(propan-2-yl)imidazo[1,2-a]pyridin-6-yl]phenyl}[trans-4-({4-[(trifluoromethyl)sulfanyl]phenyl}Amino)cyclohexyl](imino)-λ⁶-sulfanone;
6-(4-{[trans-4-({4-[(trifluoromethyl)sulfanyl]phenyl}Amino)cyclohexyl]sulfonimidoyl}phenyl)-2H,3H-[1,2,4]triazolo[4,3-a]pyridin-3-one;
(4-{imidazo[1,2-a]pyridin-6-yl}phenyl)[trans-4-({4-[(trifluoromethyl)sulfanyl]phenyl}Amino)cyclohexyl](imino)-λ⁶-sulfanone;
(4-{imidazo[1,2-a]pyridin-6-yl}phenyl)[trans-4-{[5-(trifluoromethoxy)pyridin-2-yl]Amino}cyclohexyl](imino)-λ⁶-sulfanone;
4′-{[trans-4-{[4-(trifluoromethoxy)phenyl]Amino}cyclohexyl]sulfonyl}-[1,1′-biphenyl]-4-carbonitrile;
N-[trans-4-(4-{3-methylimidazo[1,2-a]pyridin-6-yl}benzenesulfonyl)cyclohexyl]-4-(trifluoromethoxy)aniline;
N-[trans-4-(4-{3-methylimidazo[1,2-a]pyridin-6-yl}benzenesulfonyl)cyclohexyl]-5-(trifluoromethoxy)pyridin-2-amine;
5-(4-{[trans-4-({4-[(trifluoromethyl)sulfanyl]phenyl}Amino)cyclohexyl]sulfonyl}phenyl)-1H-indole-3-carbonitrile;
N-[trans-4-(4-{imidazo[1,2-a]pyridin-6-yl}benzenesulfonyl)cyclohexyl]-4-(trifluoromethoxy)aniline;
6-(4-{[trans-4-({4-[(trifluoromethyl)sulfanyl]phenyl}Amino)cyclohexyl]sulfonyl}phenyl)-2H,3H-[1,2,4]triazolo[4,3-a]pyridin-3-one;
N-[trans-4-(4-{imidazo[1,2-a]pyridin-6-yl}benzenesulfonyl)cyclohexyl]-4-[(trifluoromethyl)sulfanyl]aniline;
N-[trans-4-{4-[8-methyl-3-(propan-2-yl)imidazo[1,2-a]pyridin-6-yl]benzenesulfonyl}cyclohexyl]-4-[(trifluoromethyl)sulfanyl]aniline;
4-(4-{[trans-4-({4-[(trifluoromethyl)sulfanyl]phenyl}Amino)cyclohexyl]sulfonyl}phenyl)pyridine-2-carboxamide;
4′-[(4-{[5-(trifluoromethoxy)pyridin-2-yl]Amino}piperidin-1-yl)sulfonyl]-[1,1′-biphenyl]-4-carbonitrile;
6-{4-[(4-{[5-(trifluoromethoxy)pyridin-2-yl]Amino}piperidin-1-yl)sulfonyl]phenyl}-2,3-dihydro-1H-isoindol-1-one;
4′-[(4-{[5-(trifluoromethoxy)pyridin-2-yl]Amino}piperidin-1-yl)sulfonyl]-[1,1′-biphenyl]-3-carboxamide;
5-{4-[(4-{[5-(trifluoromethoxy)pyridin-2-yl]Amino}piperidin-1-yl)sulfonyl]phenyl}-1H-indole-3-carbonitrile;
4′-[(4-{[4-(trifluoromethoxy)phenyl]Amino}piperidin-1-yl)sulfonyl]-[1,1′-biphenyl]-4-carbonitrile;
N-methyl-4′-[(4-{[4-(trifluoromethoxy)phenyl]Amino}piperidin-1-yl)sulfonyl]-[1,1′-biphenyl]-3-carboxamide;
6-{4-[(4-{[4-(trifluoromethoxy)phenyl]Amino}piperidin-1-yl)sulfonyl]phenyl}-2,3-dihydro-1H-isoindol-1-one;
4′-[(4-{[4-(trifluoromethoxy)phenyl]Amino}piperidin-1-yl)sulfonyl]-[1,1′-biphenyl]-3-carboxamide;
1-[4-(3-methyl-1H-indazol-5-yl)benzenesulfonyl]-N-[4-(trifluoromethoxy)phenyl]piperidin-4-amine;
5-{4-[(4-{[4-(trifluoromethoxy)phenyl]Amino}piperidin-1-yl)sulfonyl]phenyl}-1H-indole-3-carbonitrile;
5-{4-[(4-{[4-(trifluoromethoxy)phenyl]Amino}piperidin-1-yl)sulfonyl]phenyl}-1H-indazole-3-carbonitrile;
1-{[1,1′-biphenyl]-4-sulfonyl}-N-[4-(trifluoromethoxy)phenyl]piperidin-4-amine;
1-{[1,1′-biphenyl]-4-sulfonyl}-2-methyl-N-[4-(trifluoromethoxy)phenyl]piperidin-4-amine;
4′-[(2-methyl-4-{[4-(trifluoromethoxy)phenyl]Amino}piperidin-1-yl)sulfonyl]-[1,1′-biphenyl]-4-carbonitrile;
6-{4-[(2-methyl-4-{[4-(trifluoromethoxy)phenyl]Amino}piperidin-1-yl)sulfonyl]phenyl}-2,3-dihydro-1H-isoindol-1-one;
N-methyl-4′-[(2-methyl-4-{[4-(trifluoromethoxy)phenyl]Amino}piperidin-1-yl)sulfonyl]-[1,1′-biphenyl]-3-carboxamide;
N-[4-(difluoromethoxy)phenyl]-1-[4-(3-methyl-1H-indazol-5-yl)benzenesulfonyl]piperidin-4-amine;
6-{4-[(4-{[4-(difluoromethoxy)phenyl]Amino}piperidin-1-yl)sulfonyl]phenyl}-2,3-dihydro-1H-isoindol-1-one;
5-{4-[(4-{[4-(difluoromethoxy)phenyl]Amino}piperidin-1-yl)sulfonyl]phenyl}-1H-indole-3-carbonitrile;
1-{[1,1′-biphenyl]-4-sulfonyl}-N-[4-(difluoromethoxy)phenyl]piperidin-4-amine;
1-(4-{imidazo[1,2-a]pyridin-6-yl}benzenesulfonyl)-N-[4-(trifluoromethoxy)phenyl]piperidin-4-amine;
1-(4-{3-methylimidazo[1,2-a]pyridin-6-yl}benzenesulfonyl)-N-[4-(trifluoromethoxy)phenyl]piperidin-4-amine;
6-{4-[(4-{[4-(trifluoromethoxy)phenyl]Amino}piperidin-1-yl)sulfonyl]phenyl}imidazo[1,2-a]pyridine-3-carbonitrile;
6-{4-[(4-{[4-(trifluoromethoxy)phenyl]Amino}piperidin-1-yl)sulfonyl]phenyl}-2H,3H-[1,2,4]triazolo[4,3-a]pyridin-3-one;
1-(4-{2-methyl-[1,2,4]triazolo[1,5-a]pyridin-6-yl}benzenesulfonyl)-N-[4-(trifluoromethoxy)phenyl]piperidin-4-amine;
1-(4-{2-methyl-[1,2,4]triazolo[1,5-a]pyridin-7-yl}benzenesulfonyl)-N-[4-(trifluoromethoxy)phenyl]piperidin-4-amine;
1-{4-[3-(propan-2-yl)imidazo[1,2-a]pyridin-6-yl]benzenesulfonyl}-N-[4-(trifluoromethoxy)phenyl]piperidin-4-amine;
N-(1-{4-[3-(propan-2-yl)imidazo[1,2-a]pyridin-6-yl]benzenesulfonyl}piperidin-4-yl)-5-(trifluoromethoxy)pyridin-2-amine;
N-[1-(4-{imidazo[1,2-a]pyridin-6-yl}benzenesulfonyl)piperidin-4-yl]-5-(trifluoromethoxy)pyridin-2-amine;
1-{4-[3-(propan-2-yl)imidazo[1,2-a]pyridin-6-yl]benzenesulfonyl}-N-{4-[(trifluoromethyl)sulfanyl]phenyl}piperidin-4-amine;
1-{4-[3-(methoxymethyl)-[1,2,4]triazolo[4,3-a]pyridin-6-yl]benzenesulfonyl}-N-[4-(trifluoromethoxy)phenyl]piperidin-4-amine;
6-{4-[(4-{[5-(trifluoromethoxy)pyridin-2-yl]Amino}piperidin-1-yl)sulfonyl]phenyl}-2H,3H-[1,2,4]triazolo[4,3-a]pyridin-3-one;
1-(4-{imidazo[1,2-a]pyridin-6-yl}benzenesulfonyl)-N-{4-[(trifluoromethyl)sulfanyl]phenyl}piperidin-4-amine;
1-(4-{3-cyclopentyl-[1,2,4]triazolo[4,3-a]pyridin-6-yl}benzenesulfonyl)-N-[4-(trifluoromethoxy)phenyl]piperidin-4-amine;
1-{4-[8-methyl-3-(propan-2-yl)imidazo[1,2-a]pyridin-6-yl]benzenesulfonyl}-N-{4-[(trifluoromethyl)sulfanyl]phenyl}piperidin-4-amine;
4-(4-{[4-({4-[(trifluoromethyl)sulfanyl]phenyl}Amino)piperidin-1-yl]sulfonyl}phenyl)pyridine-2-carboxamide;
4′-{[trans-4-{[3-(trifluoromethoxy)phenyl]Amino}cyclohexyl]sulfonyl}-[1,1′-biphenyl]-4-carbonitrile;
N-[trans-4-(4-{imidazo[1,2-a]pyridin-6-yl}benzenesulfonyl)cyclohexyl]-3-[(trifluoromethyl)sulfanyl]aniline;
N-[trans-4-{4-[3-(propan-2-yl)imidazo[1,2-a]pyridin-6-yl]benzenesulfonyl}cyclohexyl]-3-[(trifluoromethyl)sulfanyl]aniline;
6-{4-[(4-{[3-(trifluoromethoxy)phenyl]Amino}piperidin-1-yl)sulfonyl]phenyl}-2,3-dihydro-1H-isoindol-1-one,
4′-[(4-{[3-(difluoromethoxy)phenyl]Amino}piperidin-1-yl)sulfonyl]-[1,1′-biphenyl]-4-carbonitrile;
N-(1-{4-[8-methyl-3-(propan-2-yl)imidazo[1,2-a]pyridin-6-yl]benzenesulfonyl}piperidin-4-yl)-5-(trifluoromethoxy)pyridin-2-amine;
N-(1-{4-[3-(propan-2-yl)-[1,2,4]triazolo[4,3-a]pyridin-6-yl]benzenesulfonyl}piperidin-4-yl)-5-(trifluoromethoxy)pyridin-2-amine;
N-(1-{4-[8-methyl-3-(propan-2-yl)-[1,2,4]triazolo[4,3-a]pyridin-6-yl]benzenesulfonyl}piperidin-4-yl)-5-(trifluoromethoxy)pyridin-2-amine;
4-{4-[(4-{[5-(trifluoromethoxy)pyridin-2-yl]amino}piperidin-1-yl)sulfonyl]phenyl}pyridine-2-carboxamide,
N-(1-{4-[3-(propan-2-yl)-[1,2,4]triazolo[4,3-a]pyrazin-6-yl]benzenesulfonyl}piperidin-4-yl)-5-(trifluoromethoxy)pyridin-2-amine;
N-[1-(4-{3-methyl-[1,2,4]triazolo[4,3-a]pyridin-6-yl}benzenesulfonyl)piperidin-4-yl]-5-(trifluoromethoxy)pyridin-2-amine;
N-(1-{4-[8-methyl-3-(propan-2-yl)imidazo[1,2-a]pyridin-6-yl]benzenesulfonyl}piperidin-4-yl)-5-[(trifluoromethyl)sulfanyl]pyridin-2-amine;
N-[1-(4-{imidazo[1,2-a]pyridin-6-yl}benzenesulfonyl)piperidin-4-yl]-5-[(trifluoromethyl)sulfanyl]pyridin-2-amine,
4-(4-{[4-({5-[(trifluoromethyl)sulfanyl]pyridin-2-yl}amino)piperidin-1-yl]sulfonyl}phenyl)pyridine-2-carboxamide;
N-(1-{4-[3-(propan-2-yl)imidazo[1,2-a]pyridin-6-yl]benzenesulfonyl}piperidin-4-yl)-5-[(trifluoromethyl)sulfanyl]pyridin-2-amine;
N-(1-{4-[3-(propan-2-yl)-[1,2,4]triazolo[4,3-a]pyridin-6-yl]benzenesulfonyl}piperidin-4-yl)-5-[(trifluoromethyl)sulfanyl]pyridin-2-amine;
N-(1-{4-[3-(propan-2-yl)-[1,2,4]triazolo[4,3-a]pyrazin-6-yl]benzenesulfonyl}piperidin-4-yl)-5-[(trifluoromethyl)sulfanyl]pyridin-2-amine;
N-[1-(4-{3-methyl-[1,2,4]triazolo[4,3-a]pyridin-6-yl}benzenesulfonyl)piperidin-4-yl]-5-[(trifluoromethyl)sulfanyl]pyridin-2-amine;
N-(1-{4-[8-methyl-3-(propan-2-yl)-[1,2,4]triazolo[4,3-a]pyridin-6-yl]benzenesulfonyl}piperidin-4-yl)-5-[(trifluoromethyl)sulfanyl]pyridin-2-amine;
N-(1-{4-[8-methyl-3-(propan-2-yl)imidazo[1,2-a]pyridin-6-yl]benzenesulfonyl}piperidin-4-yl)-4-(trifluoromethoxy)pyridin-2-amine;
N-(1-{4-[3-(propan-2-yl)-[1,2,4]triazolo[4,3-a]pyridin-6-yl]benzenesulfonyl}piperidin-4-yl)-4-(trifluoromethoxy)pyridin-2-amine;
N-(1-{4-[8-methyl-3-(propan-2-yl)-[1,2,4]triazolo[4,3-a]pyridin-6-yl]benzenesulfonyl}piperidin-4-yl)-4-(trifluoromethoxy)pyridin-2-amine;
N-[trans-4-(4-{3-methyl-1H-pyrazolo[3,4-b]pyridin-5-yl}benzenesulfonyl)cyclohexyl]-4-[(trifluoromethyl)sulfanyl]aniline;
N-[trans-4-(4-{imidazo[1,2-a]pyridin-6-yl}benzenesulfonyl)cyclohexyl]-5-(trifluoromethoxy)pyridin-2-amine;
and
6-(4-{[4-({5-[(trifluoromethyl)sulfanyl]pyridin-2-yl}amino)piperidin-1-yl]sulfonyl}phenyl)-2H,3H-[1,2,4]triazolo[4,3-a]pyridin-3-one;
or a pharmaceutically acceptable salt thereof.

28. The compound of claim 1, wherein the compound is selected from:
5-[4-[4-[4-(trifluoromethylsulfanyl)anilino]cyclohexyl]sulfonylphenyl]-1H-indole-3-carbonitrile;
5-[4-[[4-[[5-(trifluoromethoxy)-2-pyridyl]Amino]-1-piperidyl]sulfonyl]phenyl]-1H-indole-3-carbonitrile;
1-[4-(3-methyl-1H-indazol-5-yl)phenyl]sulfonyl-N-[4-(trifluoromethoxy)phenyl]piperidin-4-amine;
5-[4-[[4-[4-(trifluoromethoxy)anilino]-1-piperidyl]sulfonyl]phenyl]-1H-indole-3-carbonitrile;
5-[4-[[4-[4-(trifluoromethoxy)anilino]-1-piperidyl]sulfonyl]phenyl]-1H-indazole-3-carbonitrile;
6-[4-[[4-[4-(trifluoromethoxy)anilino]-1-piperidyl]sulfonyl]phenyl]-2H-[1,2,4]triazolo[4,3-a]pyridin-3-one;
1-[4-(3-isopropylimidazo[1,2-a]pyridin-6-yl)phenyl]sulfonyl-N-[4-(trifluoromethoxy)phenyl]piperidin-4-amine; and
1-[4-(3-cyclopentyl-[1,2,4]triazolo[4,3-a]pyridin-6-yl)phenyl]sulfonyl-N-[4-(trifluoromethoxy)phenyl]piperidin-4-amine,
or a pharmaceutically acceptable salt thereof.

29. The compound of claim 1, wherein the compound is selected from:
5-[4-[4-[4-(trifluoromethylsulfanyl)anilino]cyclohexyl]sulfonylphenyl]-1H-indole-3-carbonitrile;
5-[4-[[4-[[5-(trifluoromethoxy)-2-pyridyl]Amino]-1-piperidyl]sulfonyl]phenyl]-1H-indole-3-carbonitrile;
1-[4-(3-methyl-1H-indazol-5-yl)phenyl]sulfonyl-N-[4-(trifluoromethoxy)phenyl]piperidin-4-amine;
5-[4-[[4-[4-(trifluoromethoxy)anilino]-1-piperidyl]sulfonyl]phenyl]-1H-indole-3-carbonitrile;
5-[4-[[4-[4-(trifluoromethoxy)anilino]-1-piperidyl]sulfonyl]phenyl]-1H-indazole-3-carbonitrile;
6-[4-[[4-[4-(trifluoromethoxy)anilino]-1-piperidyl]sulfonyl]phenyl]-2H-[1,2,4]triazolo[4,3-a]pyridin-3-one;
1-[4-(3-isopropylimidazo[1,2-a]pyridin-6-yl)phenyl]sulfonyl-N-[4-(trifluoromethoxy)phenyl]piperidin-4-amine;
1-[4-(3-cyclopentyl-[1,2,4]triazolo[4,3-a]pyridin-6-yl)phenyl]sulfonyl-N-[4-(trifluoromethoxy)phenyl]piperidin-4-amine;
N-[1-(4-{3-methyl-[1,2,4]triazolo[4,3-a]pyridin-6-yl}benzenesulfonyl)piperidin-4-yl]-5-[(trifluoromethyl)sulfanyl]pyridin-2-amine;
and
6-(4-{[4-({5-[(trifluoromethyl)sulfanyl]pyridin-2-yl}amino)piperidin-1-yl]sulfonyl}phenyl)-2H,3H-[1,2,4]triazolo[4,3-a]pyridin-3-one;
or a pharmaceutically acceptable salt thereof.

30. A process of preparation of a compound of formula (I) or (I′) according to claim 1, or a pharmaceutically acceptable salt thereof, wherein X¹ is N, and X⁴ is O, comprising:
reacting a compound of formula (II), wherein R⁵ and R⁴ are as defined in claim 1, 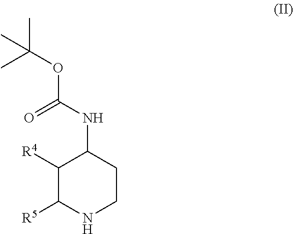
with a compound of formula (III), wherein X², X³, R², and R³, are as defined in claim 1, 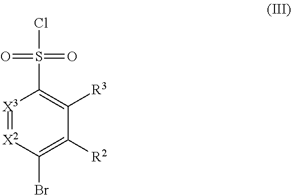
to form a compound of formula (IV), wherein R², R³, R⁴, R⁵, X², and X³ are as defined in claim 1, 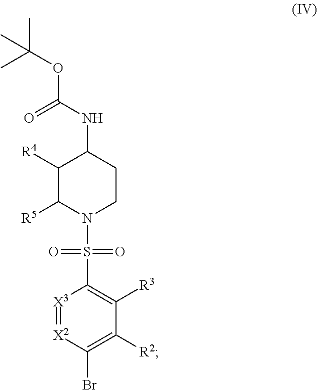
reacting said compound of formula (IV) with a compound of formula (V), wherein R¹ is as defined in claim 1, and R⁹ and R¹⁰ are hydrogen, or and R¹⁰ are each independently C₁₋₆ alkyl, or R⁹ and R¹⁰ taken together with the oxygen atom to which they are attached, form a 3- to 14-membered heterocyclyl optionally substituted with one or more C₁₋₆alkyl, 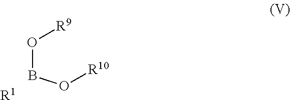
to form a compound of formula (VI), wherein R¹, R², R³, R⁴, Rn, X², and X³ are as defined in claim 1, 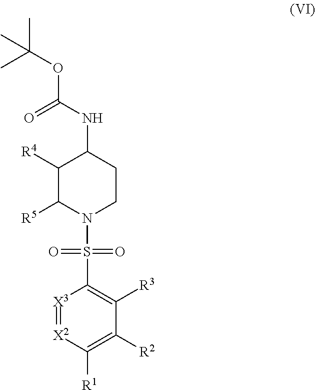
reacting said compound of formula (VI) with an acid, to form a compound of formula (VII), wherein R¹, R², R³, R⁴, R⁵, X², and X³ are as defined in claim 1, 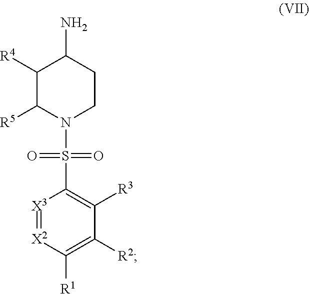
and
reacting said compound of formula (VII) with compound of formula (VIII), wherein R⁸ is a halogen and R⁶, R⁷, and X⁵ are as defined in claim 1, 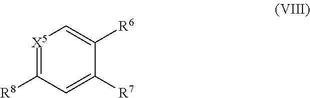
to form the compound of formula (I) or (I′).

31. A process of preparation of a compound of formula (I) or (I′) according to claim 1, or a pharmaceutically acceptable salt thereof, wherein X¹ is N and X⁴ is O, comprising:
reacting a compound of formula (IX), wherein R⁴ and R⁵ are as defined in claim 1, 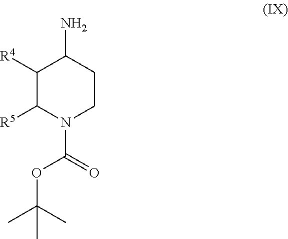
with a compound of formula (VIII), wherein R⁸ is a halogen and R⁶ R⁷, and X⁵ are as defined in claim 1, 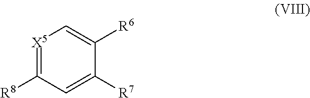
to form a compound of formula (X), wherein R⁴ R⁶, R⁷, X⁵, and R⁵ are as defined in claim 1, 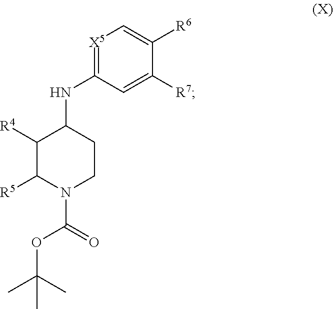
reacting said compound of formula (X) with an acid to form a compound of formula (XI), wherein R⁴ R⁶, R⁷, X⁵, and R⁵ are as defined in claim 1, 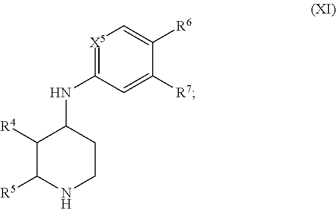
reacting said compound of formula (XI) with compound of formula (III), wherein X², X³, R², and R³, are as defined in claim 1, 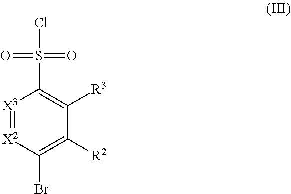
to form compound of formula (XII), wherein X², X³, R², R³, R⁴, R⁶, R⁷, X⁵, and R⁵ are as defined in claim 1, 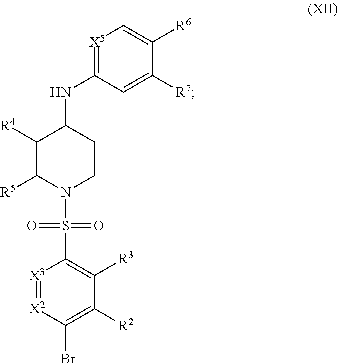
and
reacting said compound of formula (XII) with compound of formula (V), wherein R¹ is as defined in claim 1, and R⁹ and R¹⁰ are hydrogen, or R⁹ and R¹⁰ are each independently C₁₋₆ alkyl, or R⁹ and R¹⁰ taken together with the oxygen atom to which they are attached, form a 3- to 14-membered heterocyclyl optionally substituted with one or more C₁₋₆alkyl, 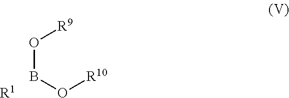
to form the compound of formula (I) or (I′).

32. A process of preparation of a compound of formula (I) or (I′) according to claim 1, or a pharmaceutically acceptable salt thereof, wherein X¹ is N and X⁴ is O, comprising:
reacting a compound of formula (XVIII), wherein R⁵ and R⁴ are as defined in claim 1, 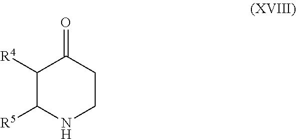
with a compound of formula (XVII), wherein R¹, R², R³, X², and X³ are as defined in claim 1, 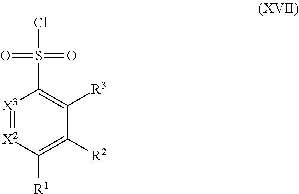
to form a compound of formula (XX), wherein R¹, R², R³, R⁴, R⁵, X², and X³ are as defined in claim 1, 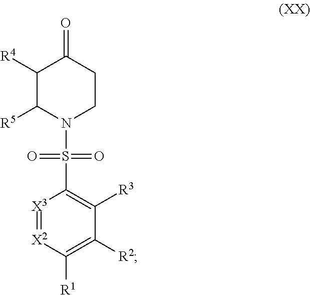
reacting said compound of formula (XX) with a compound of formula (XVI), wherein X⁵, R⁶, and R⁷ are as defined in claim 1, 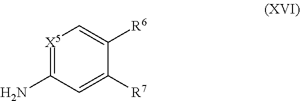
to form compound of formula (I) or (I′);
or
reacting a compound of formula (XVIII), wherein R⁵ and R⁴ are as defined in claim 1, 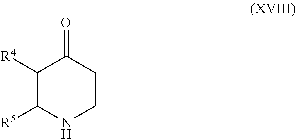
with a compound of formula (III), wherein X², X³, R², and R³, are as defined in claim 1, 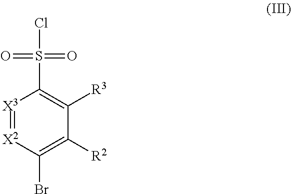
to form a compound of formula (XIX), wherein R², R³, R⁴, R⁵, X², and X³ are as defined in claim 1, 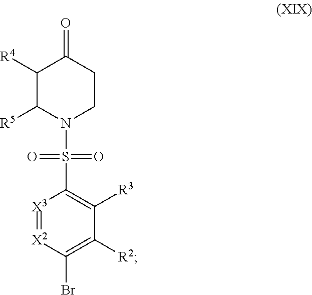
reacting said compound of formula (XIX) with said compound of formula (XVI), to form a compound of formula (XII), wherein X², X³, R², R³, R⁴ R⁶, R⁷, X⁵, and R⁵ are as defined in claim 1, 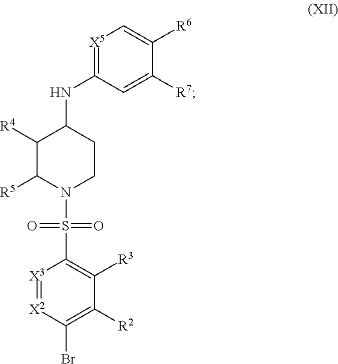
and
reacting said compound of formula (XII) with a compound of formula (V), wherein R¹ is as defined in claim 1, and R⁹ and R¹⁰ are hydrogen, or R⁹ and R¹⁰ are each independently C₁₋₆ alkyl, or R⁹ and R¹⁰ taken together with the oxygen atom to which they are attached, form a 3- to 14-membered heterocyclyl optionally substituted with one or more C₁₋₆alkyl, 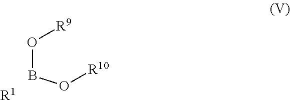
to form the compound of formula (I) or (I′).

33. A process of preparation of a compound of formula (I) or (I′) according to claim 1, or a pharmaceutically acceptable salt thereof, wherein X¹ is C and X⁴ is O, comprising:
reacting a compound of formula (XXI), wherein R⁴ and R⁵ are as defined in claim 1, 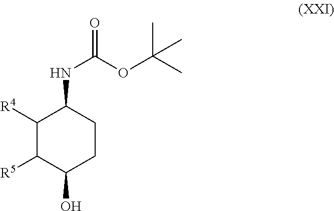
with masyl chloride, to form a compound of formula (XXII), wherein R⁴ and R⁵ are as defined in claim 1, 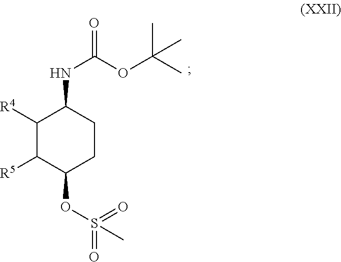
reacting said compound of formula (XXII) with a compound of formula (XXIII), wherein R², R³, X², and X³ are as defined in claim 1, 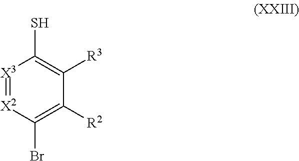
to form a compound of formula (XXIV), wherein R², R³, R⁴, R⁵, X², and X³ are as defined in claim 1, 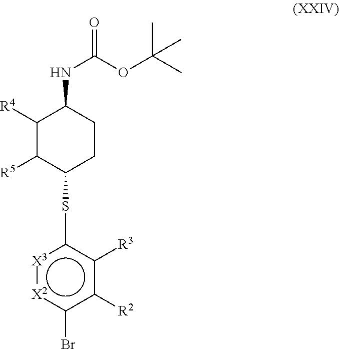
reacting said compound of formula (XXIV) with an oxidant, to form a compound of formula (XXV), wherein R², R³, R⁴, R⁵, X², and X³ are as defined in claim 1, 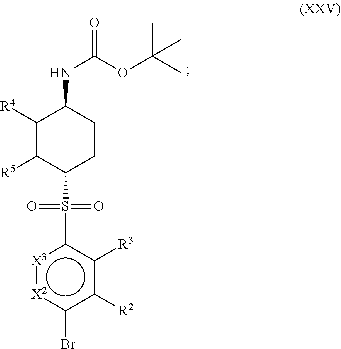
reacting said compound of formula (XXV) with a compound of formula (V), wherein R¹ is as defined in claim 1, and R⁹ and R¹⁰ are hydrogen, or R⁹ and R¹⁰ are each independently C₁₋₆ alkyl, or R⁹ and R¹⁰ taken together with the oxygen atom to which they are attached, form a 3- to 14-membered heterocyclyl optionally substituted with one or more C₁₋₆alkyl, 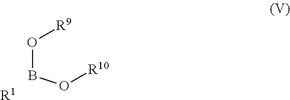
to form compound of formula (XXVI), wherein R¹, R², R³, R⁴, R⁵, X², and X³ are as defined claim 1, 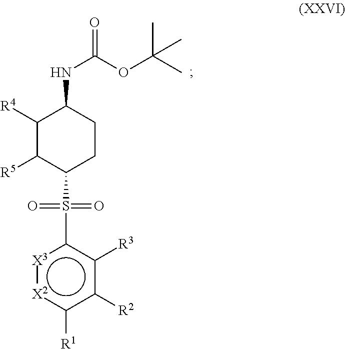
reacting said compound of formula (XXVI) with an acid, to form a compound of formula (XXVII), wherein R¹, R², R³, R⁴, R⁵, X², and X³ are as defined in claim 1, 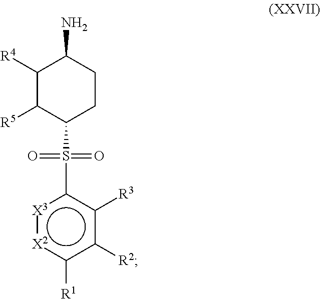
and
reacting said compound of formula (XXVII) with a compound of formula (VIII), wherein R⁸ is a halogen and R⁶, R⁷, and X⁵ are as defined in claim 1, 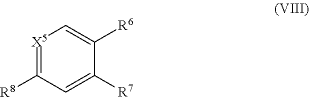
to form the compound of formula (I) or (I′).

34. A process of preparation of a compound of formula (I) or (I′) according to claim 1, or a pharmaceutically acceptable salt thereof, wherein X¹ is C and X⁴ is O, comprising:
reacting a compound of formula (XXV), wherein R², R³, R⁴, R⁵, X², and X³ are as defined in claim 1, 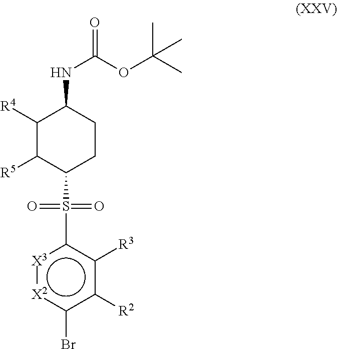
with an acid to form compound of formula (XXVIII), wherein R², R³, R⁴, R⁵, X², and X³ are as defined in claim 1, 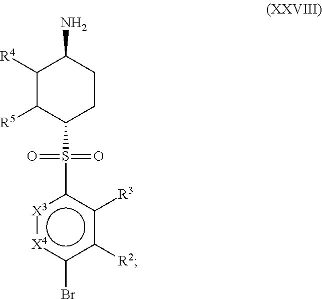
reacting said compound of formula (XXVIII) with a compound of formula (VIII), wherein R⁸ is a halogen and R⁶, R⁷, and X⁵ are as defined in claim 1, 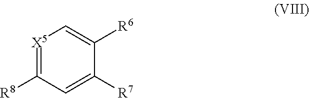
to form a compound of formula (XXIX), wherein R², R³, R⁴, R⁵, R⁶ R⁷, X⁵, X², and X³ are as defined in claim 1, 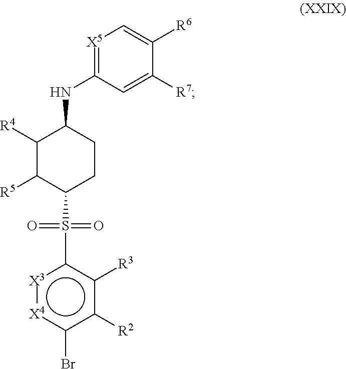
and
reacting said compound of formula (XXIX) with a compound of formula (V), wherein R¹ is as defined in claim 1, and R⁹ and R¹⁰ are hydrogen, or R⁹ and R¹⁰ are each independently C₁₋₆ alkyl, or R⁹ and R¹⁰ taken together with the oxygen atom to which they are attached, form a 3- to 14-membered heterocyclyl optionally substituted with one or more C₁₋₆alkyl, 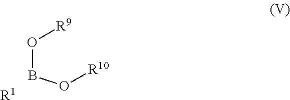
to form the compound of formula (I) or (I′).

35. A process of preparation of a compound of formula (I) or (I′) according to claim 1, or a pharmaceutically acceptable salt thereof, wherein X¹ is C and X⁴ is O, comprising:
reacting a compound of formula (XXXI), wherein R⁴ and R⁵ are as defined in claim 1, 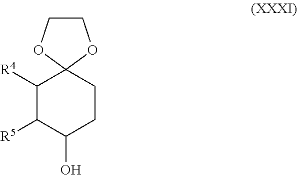
with mesyl chloride, to form a compound of formula (XXXII), wherein R⁴ and R⁵ are as defined in claim 1, 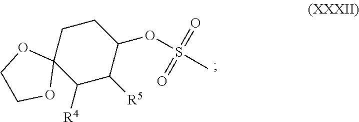
reacting said compound of formula (XXXII) with a compound of formula (XXIII), wherein R², R³, X², and X³ are as defined in claim 1, 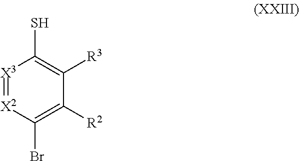
to form compound of formula (XXXIII), wherein R² R³ R⁴ R⁵, X², and X³ are as defined in claim 1, 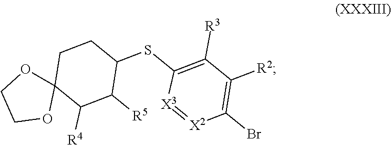
reacting said compound (XXXIII) with an oxidant, to form a compound of formula (XXXIV), wherein R², R³, R⁴, R⁵, X², and X³ are as defined in claim 1, 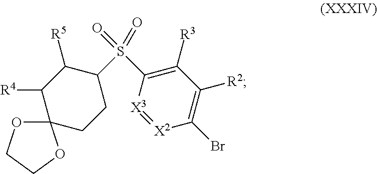
reacting said compound (XXXIV) with an acid, to form a compound of formula (XXXV), wherein R², R³, R⁴, R⁵, X², and X³ are as defined in claim 1, 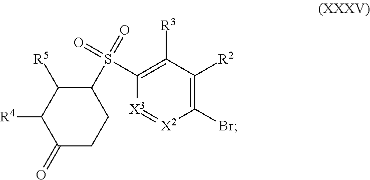
reacting said compound of formula (XXXV) with a compound of formula (XVI), wherein X⁵, R⁶, and R⁷ are as defined in claim 1, 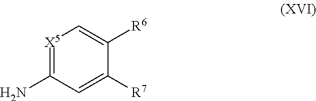
to form a compound of formula (XXIX), wherein R², R³, R⁴, R⁵, R⁶, R⁷, X⁵, X², and X³ are as defined in claim 1, 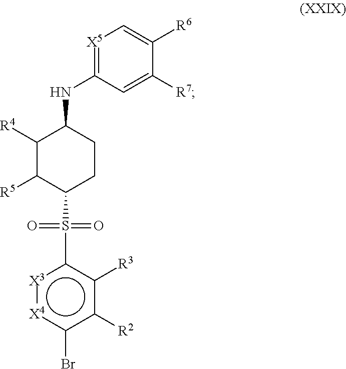
and
reacting said compound of formula (XXIX) with a compound of formula (V), wherein R¹ is as defined in claim 1, and R⁹ and R¹⁰ are hydrogen, or R⁹ and R¹⁰ are each independently C₁₋₆ alkyl, or R⁹ and R¹⁰ taken together with the oxygen atom to which they are attached, form a 3- to 14-membered heterocyclyl optionally substituted with one or more C₁₋₆alkyl, 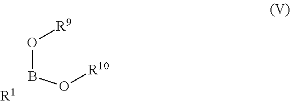
to form the compound of formula (I) or (I′).

36. A process of preparation of a compound of formula (I) or (I′) according to claim 1, or a pharmaceutically acceptable salt thereof, wherein X¹ is C and X⁴ is N, comprising:
reacting a compound of formula (XXXIII), wherein R², R³, R⁴, R⁵, X², and X³ are as defined in claim 1, 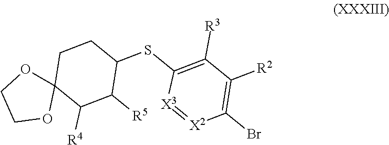
with an acid to form a compound of formula (XXXVI), wherein R², R³, R⁴, R⁵, X², and X³ are as defined in claim 1, 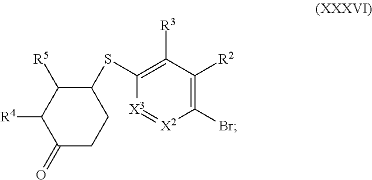
reacting said compound of formula (XXXVI) with a compound of formula (XVI), wherein X⁵, R⁶, and R⁷ are as defined in claim 1, 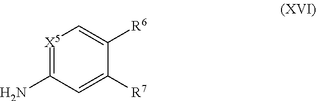
to form a compound of formula (XXXVII), wherein R², R³, R⁴, R⁵, R⁶, R⁷, X⁵, X², and X³ are as defined in claim 1, 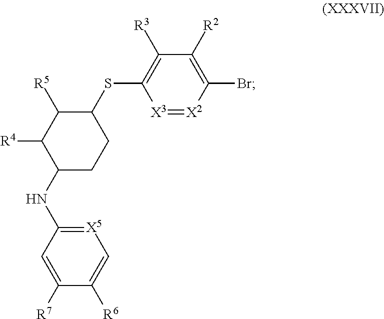
reacting said compound of formula (XXXVII) with ammonium carbamate and (diacetoxyiodo)benzene, to form a compound of formula (XXXVIII), wherein R², R³, R⁴, R⁵, R⁶, R⁷, X⁵, X², and X³ are as defined in claim 1, 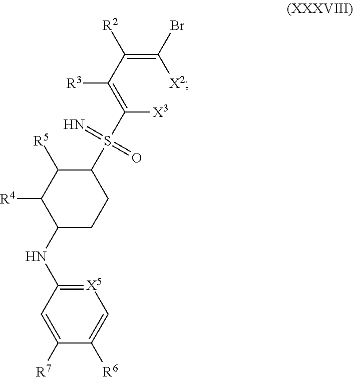
and
reacting said compound of formula (XXXVIII) with a compound of formula (V), wherein R¹ is as defined in claim 1, and R⁹ and R¹⁰ are hydrogen, or R⁹ and R¹⁰ are each independently C₁₋₆ alkyl, or R⁹ and R¹⁰ taken together with the oxygen atom to which they are attached, form a 3- to 14-membered heterocyclyl optionally substituted with one or more C₁₋₆alkyl, 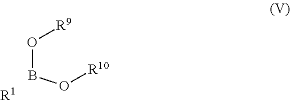
to form the compound of formula (I) or (I′).

37. A process of preparation of a compound of formula (I) or (I′) according to claim 1, or a pharmaceutically acceptable salt thereof, wherein X¹ and X⁴ are N, comprising:
reacting a compound of formula (XI), wherein R⁴ R⁶, R⁷, X⁵, and R⁵ are as defined in claim 1, 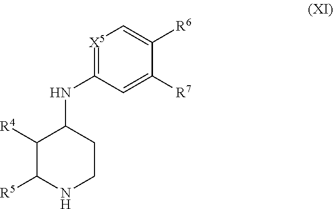
with a compound of formula (XXIII), wherein R², R³, X², and X³ are as defined in claim 1, 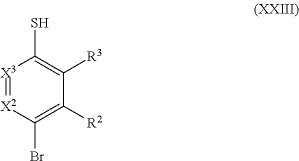
to form a compound of formula (XL), wherein R², R³, X², X³, R⁴ R⁶, R⁷, X⁵, and R⁵ are as defined in claim 1, 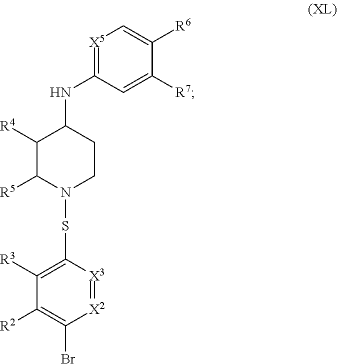
reacting said compound (XL) with ammonium carbamate and (diacetoxyiodo)benzene, to form compound of formula (XLI), wherein R², R³, X², X³, R⁴ R⁶, R⁷, X⁵, and R⁵ are as defined in claim 1, 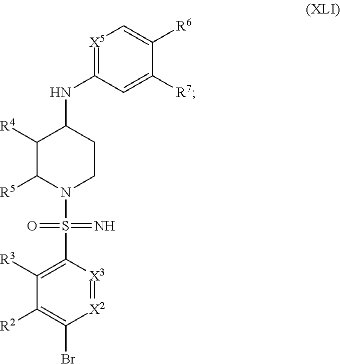
reacting said compound (XLI) with di-tert-butyl dicarbonate and a base, to form a compound of formula (XLII), wherein R², R³, X², X³, R⁴, R⁶, R⁷, X⁵, and R⁵ are as defined in 1, 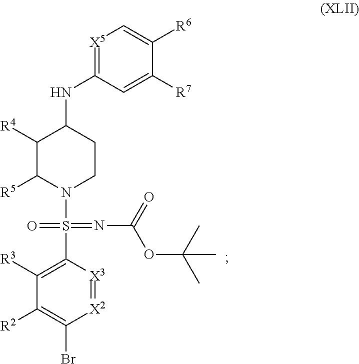
and
reacting said compound (XLII) with a compound of formula (V), wherein R¹ is as defined in claim 1, and R⁹ and R¹⁰ are hydrogen, or R⁹ and R¹⁰ are each independently C₁₋₆ alkyl, or R⁹ and R¹⁰ taken together with the oxygen atom to which they are attached, form a 3- to 14-membered heterocyclyl optionally substituted with one or more C₁₋₆alkyl, 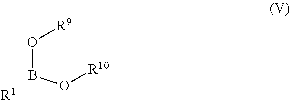
to form a compound of formula (XLIII), wherein R¹, R², R³, X², X³, R⁴ R⁶, R⁷, X⁵, and R⁵ are as defined in claim 1, 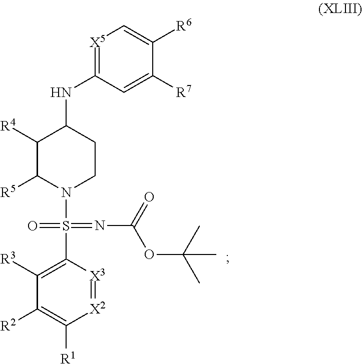
and
reacting said compound of formula (XLIII) with an acid to form the compound of formula (I) or (I′).

38. A process of preparation of a compound of formula (I) or (I′) according to claim 1, or a pharmaceutically acceptable salt thereof, wherein X¹ is C and X⁴ is N, comprising:
reacting a compound of formula (XXIV), wherein R², R³, R⁴, R⁵, X², and X³ are as defined in claim 1, 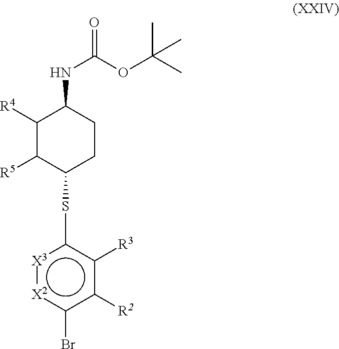
with a compound of formula (V), wherein R¹ is as defined in claim 1, and R⁹ and R¹⁰ are hydrogen, or R⁹ and R¹⁰ are each independently C₁₋₆ alkyl, or R⁹ and R¹⁰ taken together with the oxygen atom to which they are attached, form a 3- to 14-membered heterocyclyl optionally substituted with one or more C₁₋₆alkyl, 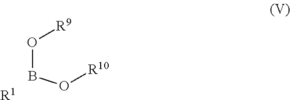
to form a compound of formula (XLIV), wherein R¹, R², R³, R⁴, R⁵, X², and X³ are as defined in claim 1, 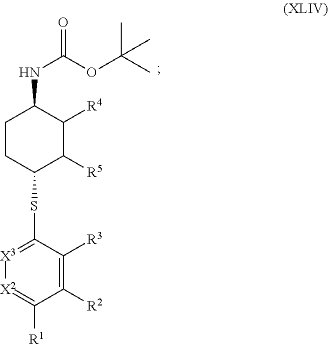
reacting said compound of formula (XLIV) with an acid to form a compound of formula (XLV), wherein R¹, R², R³, R⁴, R⁵, X², and X³ are as defined in claim 1, 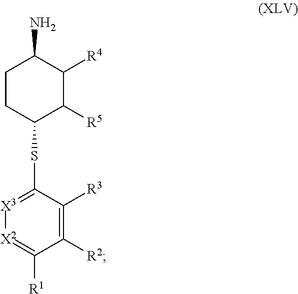
reacting said compound of formula (XLV) with a compound of formula (VIII), wherein R⁸ is a halogen, and R⁶, R⁷, and X⁵ are as defined in claim 1, 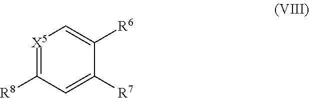
to form a compound of formula (XLVI), wherein R¹, R² R³, R⁴, R⁵, R⁶, R⁷, X⁵, X², and X³ are as defined in claim 1, 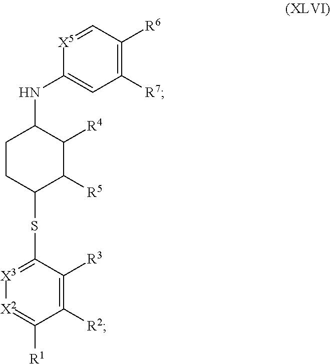
and
reacting said compound of formula (XLVI) with ammonium carbamate and (diacetoxyiodo)benzene to form the compound of formula (I) or (I′).

39. A compound according to claim 1, or a pharmaceutically acceptable salt thereof, when manufactured according to claim 30.

40. (canceled)

41. A pharmaceutical composition comprising a compound according to claim 1, or a pharmaceutically acceptable salt thereof, and a pharmaceutically acceptable excipient.

42. The pharmaceutical composition according to claim 41, further comprising an additional therapeutic agent.

43-47. (canceled)

48. A method for the treatment of an inflammatory autoimmune disease in a human subject in need thereof, which method comprises administering to said human subject a therapeutically effective amount of a compound according to claim 1, or a pharmaceutically acceptable salt thereof.

49. A method for the treatment of psoriatic diseases, asthma, Inflammatory Bowel Diseases (IBD), Crohn's disease, Obstructive Lung Diseases ulcerative colitis, rheumatoid arthritis, systemic lupus erythematosus or multiple sclerosis in a human subject in need thereof, which method comprises administering to said human subject a therapeutically effective amount of a compound according to claim 1, or a pharmaceutically acceptable salt thereof.

50. (canceled)

51. The process of claim 30, wherein R⁹ and R¹⁰ are taken together with the oxygen atom to which they are attached to form a 3- to 14-membered heterocyclyl optionally substituted with four C₁₋₆alkyl.

52. The process of claim 31, wherein R⁹ and R¹⁰ are taken together with the oxygen atom to which they are attached to form a 3- to 14-membered heterocyclyl optionally substituted with four C₁₋₆alkyl.

53. The process of claim 32, wherein R⁹ and R¹⁰ are taken together with the oxygen atom to which they are attached to form a 3- to 14-membered heterocyclyl optionally substituted with four C₁₋₆alkyl.

54. The process of claim 33, wherein R⁹ and R¹⁰ are taken together with the oxygen atom to which they are attached to form a 3- to 14-membered heterocyclyl optionally substituted with four C₁₋₆alkyl.

55. The process of claim 34, wherein R⁹ and R¹⁰ are taken together with the oxygen atom to which they are attached to form a 3- to 14-membered heterocyclyl optionally substituted with four C₁₋₆alkyl.

56. The process of claim 35, wherein R⁹ and R¹⁰ are taken together with the oxygen atom to which they are attached to form a 3- to 14-membered heterocyclyl optionally substituted with four C₁₋₆alkyl.

57. The process of claim 36, wherein R⁹ and R¹⁰ are taken together with the oxygen atom to which they are attached to form a 3- to 14-membered heterocyclyl optionally substituted with four C₁₋₆alkyl.

58. The process of claim 37, wherein R⁹ and R¹⁰ are taken together with the oxygen atom to which they are attached to form a 3- to 14-membered heterocyclyl optionally substituted with four C₁₋₆alkyl.

59. The process of claim 38, wherein R⁹ and R¹⁰ are taken together with the oxygen atom to which they are attached to form a 3- to 14-membered heterocyclyl optionally substituted with four C₁₋₆alkyl.
